(19) United States
(12) Patent Application Publication
(10) Pub. No.: US 2004/0038325 A1
(43) Pub. Date: Feb. 26, 2004

(54) Adhesion molecules

(76) Inventors: Christopher Benjamin Phelps, London (GB); Richard Joseph Fagan, London (GB); Alex Gutteridge, Haslingfield (GB)

Correspondence Address: FROMMER LAWRENCE & HAUG, 745 FIFTH AVENUE- 10TH FL., NEW YORK, NY, 10151, US

(21) Appl. No.: 10/346,863

(22) Filed: Jan. 17, 2003

Related U.S. Application Data
(63) Continuation-in-part of application No. PCT/GB01/03318, filed on Jul. 24, 2001.

(30) Foreign Application Priority Data
Jul. 24, 2000 (GB) 0018126.3
Oct. 17, 2000 (GB) 0025447.4

(51) Int. Cl. G01N 33/554; G01N 33/569

(52) U.S. Cl. 435/7.32

(57) ABSTRACT

This invention discloses and claims novel proteins, termed KIAA0301, G7c, KIAA0564, NG37, CAB01991.1 and Rv0368c, herein identified as adhesion molecules. Also disclosed and claimed are methods of use of these proteins, and nucleic acid sequences from the encoding genes, in the diagnosis, prevention and treatment of disease.

### REFERENCE TO RELATED APPLICATIONS

This application is a continuation-in-part application of International Patent Application PCT/GB01/03318, filed Jul. 24, 2001 which published as WO 02/08423 on Jan. 31, 2002, and which claims priority to United Kingdom Patent Applications 0018126.3 filed Jul. 24, 2000 and 0025447.4 filed Oct. 17, 2000.

Each of the foregoing applications and patents, each foregoing publication, and each document cited or referenced in each of the foregoing applications and patents, including during the prosecution of each of the foregoing applications and patents (“application and article cited documents”), and any manufacturer's instructions or catalogues for any products cited or mentioned in each of the foregoing applications and patents and articles and in any of the application and article cited documents, are hereby incorporated herein by reference. Furthermore, all documents cited in this text, and all documents cited or referenced in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art.

This invention relates to novel proteins, termed KIAA0301, G7c, KIAA0564, NG37, CAB01991.1, and Rv0368c, herein identified as adhesion molecules and to the use of these proteins and nucleic acid sequences from the encoding genes in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

Recently, a remarkable tool for the evaluation of sequences of unknown function has been developed by the Applicant for the present invention. This tool is a database system, termed the Biopendium search database, that is the subject of co-pending International Patent Application No. PCT/GB01/01105. This database system consists of an integrated data resource created using proprietary technology and containing information generated from an all-by-all comparison of all available protein or nucleic acid sequences.

The aim behind the integration of these sequence data from separate data resources is to combine as much data as possible, relating both to the sequences themselves and to information relevant to each sequence, into one integrated resource. All the available data relating to each sequence, including data on the three-dimensional structure of the encoded protein, if this is available, are integrated together to make best use of the information that is known about each sequence and thus to allow the most educated predictions to be made from comparisons of these sequences. The annotation that is generated in the database and which accompanies each sequence entry imparts a biologically relevant context to the sequence information.

This data resource has made possible the accurate prediction of protein function from sequence alone. Using conventional technology, this is only possible for proteins that exhibit a high degree of sequence identity (above about 20%-30% identity) to other proteins in the same functional family. Accurate predictions are not possible for proteins that exhibit a very low degree of sequence homology to other related proteins of known function.

Introduction to Adhesion Molecules

Adhesion molecules are involved in a range of biological processes, including: embryogenesis (Martin-Bermudo, M. D. et al, Development. 2000 127(12):2607-15; Chen, L. M., et al., J Neurosci. 2000 20(10):3776-84; Zweegman, S., et al, Exp Hematol. 2000 28(4):401-10; Darribere, T., et al., Biol Cell. 2000 92(1):5-25), maintenance of tissue integrity (Eckes, B., et al., J Cell Sci. 2000 113(Pt 13):2455-2462; Buckwalter, J. A., et al., Instr Course Lect. 2000 49:481-9; Frenette, P. S., et al.,. J Exp Med. 2000 191(8):1413-22; Delmas, V., et al, Dev Biol. 1999 216(2):491-506; Humphries, M. J., et al., Trends Pharmacol Sci. 2000 21(1):29-32; Miosge, N., et al, Lab Invest. 1999 79(12):1591-9; Nagaoka T, et al. Am J Pathol 2000 Jul 157:1 237-47; Nwariaku F E, et al. J Trauma 1995 39(2): 285-8; Zhu X, et al. Zhonghua Zheng Xing Shao Shang Wai Ke Za Zhi 1999 15(1): 53-5), leukocyte extravasation/inflammation (Lim, L. H., et al. Am J Respir Cell Mol Biol. 2000 22(6):693-701; Johnston, B., et al., Microcirculation. 2000 7(2):109-18; Mertens, A. V., et al., Clin Exp Allergy. 1993 23(10):868-73; Chcialowski, A., et al., Pol Merkuriusz Lek. 2000 7(43):13-7; Rojas, A. I., et al, Crit Rev Oral Biol Med. 1999 10(3):337-58; Marinova-Mutafchieva, L., et al., Arthritis Rheum. 2000 43(3):638-44; Vijayan, K. V., et al, J Clin Invest. 2000 105(6):793-802; Currie, A. J., et al,. J Immunol. 2000 164(7):3878-86; Rowin, M. E., et al., Inflammation. 2000 24(2):157-73; Johnston, B., et al., J Immunol. 2000 164(6):3337-44; Gerst, J. L., et al., J Neurosci Res. 2000 59(5):680-4; Kagawa, T. F., et al., Proc Natl Acad Sci USA. 2000 97(5):2235-40; Hillan, K. J., et al., Liver. 1999 19(6):509-18; Panes, J., 1999 22(10):514-24; Arao, T., et al., J Clin Endocrinol Metab. 2000 85(1):382-9; Souza, H. S., et al., Gut. 1999 45(6):856-63; Grunstein, M. M., et al., Am J Physiol Lung Cell Mol Physiol. 2000 278(6):L1154-63; Mertens, A. V., et al., Clin Exp Allergy. 1993 23(10):868-73; Berends, C., et al., Clin Exp Allergy. 1993 23(11):926-33; Fernvik, E., et al., Inflammation. 2000 24(1):73-87; Bocchino, V., et al., J Allergy Clin Immunol. 2000 105(1 Pt 1):65-70; Jones S C, et al, Gut 1995 36(5):724-30; Liu C M, et al, Ann Allergy Asthma Immunol 1998 81(2):176-80; McMurray R W Semin Arthritis Rheum 1996 25(4):215-33; Takahashi H, et al Eur J Immunol 1992 22(11): 2879-85; Carlos T, et al J Heart Lung Transplant 1992 11(6): 1103-8; Fabrega E, et al, Transplantation 2000 69(4): 569-73; Zohrens G, et al, Hepatology 1993 18(4): 798-802; Montefort S, et al. Am J Respir Crit Care Med 1994 149(5): 1149-52), oncogenesis (Orr, F. W., et al., Cancer. 2000 88(S12):2912-2918; Zeller, W., et al., J Hematother Stern Cell Res. 1999 8(5):539-46; Okada, T., et al., Clin Exp Metastasis. 1999 17(7):623-9; Mateo, V., et al., Nat Med. 1999 5(11):1277-84; Yamaguchi, K., et al., J Exp Clin Cancer Res. 2000 19(1):113-20; Maeshima, Y., et al., J Biol Chem. 2000 275(28):21340-8; Van Waes, C., et al, Int J Oncol. 2000 16(6):1189-95; Damiano, J. S., et al., Leuk Lymphoma. 2000 38(1-2):71-81; Seftor, R. E., et al, Cancer Metastasis Rev. 1999 18(3):359-75; Shaw, L. M., J Mammary Gland Biol Neoplasia. 1999 4(4):367-76; Weyant, M. J., et al., Clin Cancer Res. 2000 6(3):949-56), angiogenesis (Koch A E, et al Nature 1995 376 (6540): 517-9; Wagener C & Ergun S. Exp Cell Res 2000 261(1): 19-24; Ergun S, et al. Mol Cell 2000 5(2): 311-20), bone resorption (Hartman G D, & Duggan M E. Expert Opin Investig Drugs 2000 9(6): 1281-91; Tanaka Y, et al. J Bone Miner Res 1995 10(10): 1462-9; Lark M W, et al. J Pharmacol Exp Ther 1999 291(2): 612-7; Raynal C, et al Endocrinology 1996 137(6):2347-54; Ilvesaro J M, et al. Exp Cell Res 1998 242(1): 75-83), neurological dysfunction (Ossege L M, et al. Int Immunopharmacol 2001 1:1085-100; Bitsch A, et al, Stroke 1998 29:2129-35; Iadecola C & Alexander M. Curr Opin Neurol 2001 14:89-94; Becker K, et al Stroke 2001 32(1): 206-11; Relton J K, et al Stroke 2001 32(1): 199-205; Hamada Y, et al J Neurochem 1996 66:1525-31), thrombogenesis (Wang, Y. G., et al., J Physiol (Lond). 2000 526(Pt 1):57-68; Matsuno, H., et al., Nippon Yakurigaku Zasshi. 2000 115(3):143-50; Eliceiri, B. P., et al., Cancer J Sci Am. 2000 6(Suppl 3):S245-9; von Beckerath, N., et al., Blood. 2000 95(11):3297-301; Topol, E. J., et al., Am Heart J. 2000 139(6):927-33; Kroll, H., et al., Thromb Haemost. 2000 83(3):392-6), and invasion/adherence of bacterial pathogens to the host cell (Dersch P, et al. EMBO J 1999 18(5): 1199-1213).

The detailed characterisation of the structure and function of several adhesion-receptor families has led to active programs by a number of pharmaceutical companies to develop adhesion molecule antagonists for use in the treatment of diseases involving inflammation, oncology, neurology, immunology and cardiovascular function. Adhesion receptors are involved in virtually every aspect of biology from embryogenesis to apoptosis. They are essential to the structural integrity and homeostatic functioning of most tissues. It is therefore not surprising that defects in adhesion receptors cause disease and that many diseases involve modulation of adhesion molecule function.

The Adhesion molecule family in fact represent at least four distinct families which are unified by their function rather than their structure. Of the four families, three are of pharmaceutical interest due to small molecule tractibility. They are;

1.The integrin family is a superfamily of α and β heterodimeric transmembrane glycoproteins and is the family which has attracted most pharmaceutical interest. Its members are large, heavily glycosylated, heterodimeric proteins composed of one of at least 15 distinct α-subunits in non-covalent linkage with one of at least 8 β-subunits. Adhesion receptors bind ligands expressed on cell surfaces, extracellular matrix molecules, and soluble molecules. Integrins are subcategorised based on their β-subunit usage. The members of this family are summarised below in Table 1.

2.Selectins are a small family of three members P, E and L selectin. They are glycoproteins, selectively expressed on cells related to the vasculature, and contain a lectin-binding domain. The members of this family are described below in Table 2.

3.The immunoglobulin family represents the counter receptor for the integrins and includes the intracellular adhesion molecules (ICAMs) and vascular cell adhesion molecules (VCAMs). Members are composed of variable numbers of globular, immunoglobulin-like, extracellular domains. Some members of the family, for example, PECAM-1 (CD31) and NCAM, mediate homotypic adhesion. Some members of the family, for example ICAM-1 and VCAM-1, mediate adhesion via interactions with integrins. The members of this family are described below in Table 3.

The fourth family of adhesion molecules is the cadherins, which although is not small molecule tractable (currently), it may become tractable in the near future and is certainly a candidate for antibody targeting:

4.The cadherins all contain multiple tandem repeats of the Ca²⁺ binding “Cadherin domain”. The first cadherins to be identified (such as Epithelial/E-Cadherin) are referred to as the classical cadherins and mediate Ca²⁺-dependent cell adhesion. However, more recently identified non-classical members of the Cadherin family are known to be involved in a diverse range of biological processes, such as cell recognition, cell signalling, cell communication, morphogenesis, angiogenesis, and possibly even neurotransmission (Angst B D, et al. J Cell Sci 2001 114(4): 629-639) (See Table 5).

Adhesion molecules have been shown to play a role in diverse physiological functions, many of which can play a role in disease processes. Alteration of their activity is a means to alter the disease phenotype and, as such, the identification of novel adhesion molecules is highly relevant as they may play a role in many diseases, particularly inflammatory disease, oncology, cardiovascular disease and bacterial infection.

A further need in the art is, of course, to treat and prevent the incidence of diseases that are caused by bacterial pathogens. Adhesion molecule-like proteins are expressed in pathogenic organisms where they function in mammalian target cell adherence and penetration. In Yersinia pseudotuberculosis, the cell adhesion molecule-like protein Invasin is required for efficient entry into mammalian cells (Dersch P, et al. EMBO J 1999 18(5): 1199-1213). A family of proteins called Intimins (which are highly similar to Invasins) are involved in the attachment of a variety of related Gram-negative enteric pathogens to host cells (Jerse A E, et al. Proc Natl Acad Sci USA 1990 87:7839-7843; Schauer D B, et al. Infect Immun 1993 61:4654-4661) (See Table 6). Therapeutic or diagnostic agents that are specific for the adhesion molecule-like proteins that are expressed in pathogenic organisms would be of great value in the diagnosis and treatment of diseases caused by these organisms.

TABLE 1
Integrins:

| Integrin | | |
|---|---|---|
| Receptor | Ligand | Distribution |
| β1 (CD29) | | |
| α1β1 | Laminin, Collagen | Activated T cells, fibroblasts |
| α2β1 | Collagen, Laminin | Activated T cells, endothelial cells, platelets, |
| | | basophils. |
| α3β1 | Laminin, Collagen, | Basement membrane |
| | Fibronectin | |
| α4β1 | VCAM-1 (domains 1 and | Lymphocytes, monocytes, eosinophils, |
| | 4), Fibronectin | (CS-1basophils, mast cells, NK cells |
| | | domain), MadCAM-1 |
| α5β1 | Fibronectin | Lymphocytes, monocytes, endothelial cells, |
| | | basophils, |
| | | mast cells, fibroblasts |
| α6β1 | Laminin | Platelets, T cells, eosinophils, monocytes, |
| | | endothelial cells |
| α9β1 | Tenascin, VCAM-1, | Airway epithelial cells, smooth muscle cells, |
| | Osteopontin | neutrophils |
| αVβ1 | Vitronectin, fibronectin | Platelets, B cells. |
| β2(CD18) | | |
| LFA-1 | ICAM-1, 2, 3 | All leukocytes |
| (CD11a/CD18) | | |
| Mac-1 | ICAM-1, Fibrinogen, LPS | Granulocytes, monocytes |
| (CD11b/CD18) | | |
| αD | ICAM-3, VCAM-1 | Tissue macrophages, monocytes, CD8+ T |
| | | cells eosinophils |
| β3(CD61) | | |
| GpIIb/IIIa | Fibrinogen, | Vitronectin, Platelets, endothelial cells |
| | Fibronectin, vWF | |
| αV/IIIa | Vitronectin, | Fibrinogen, Platelets, |
| | vWF, | Laminin, |
| | Thrombospondin, | |
| | Osteopontin | |
| β7 | | |
| α4β7 | MAdCAM-1, VCAM-1, | Subset of memory T cells, eosinophils, |
| (LPAM-1) | Firbonectin | (CS-1basophils, endothelial cells |
| | domain) | |
| αEβ7 | E-cadherin | Intestinal intraepithelial lymphocytes. |

TABLE 2
Selectins:

| Receptor | Ligand | Distribution |
|---|---|---|
| E-selectin | Sialyl-LewisX, | L- Activated endothelial cells |
| | selectin, LFA-1, ESL-1, | |
| | PSGL-1 | |
| L-selectin | GlyCAM-1, MAdCAM-1, | Resting leukocytes |
| | CD34, Sialyl LewisX, | |
| | E-selectin, P-selectin | |
| P-selectin | Sialyl-LewisX, | L- Activated endothelial cells, activated platelets |
| | selectin, PSGL-1 | |

TABLE 3
Immunoglobulin superfamily:

| Receptor | Ligand | Distribution |
|---|---|---|
| ICAM-1 | LFA-1 (CD11a/CD18) | Widespread, endothelial cells, fibroblasts, |
| 5 Ig domains | Mac-1 (CD 11b/CD18), | epithelium, monocytes, lymphocytes, dendritic |
| | CD43 | cells, chondrocytes. |
| ICAM-2 | LFA-1 (CD11b) | endothelial cells (high): lymphocytes, |
| 2 Ig domains | | monocytes, basophils, platelets (low). |
| ICAM-3 | LFA-1 (αd/CD18) | Lymphocytes, monocytes, neutrophils, |
| 5 Ig domains | | eosinophils, basophils. |
| VCAM-1 | α4β1, α4β7 | Endothelial cells, monocytes, fibroblasts, |
| 6 or 7 Ig | | dendritic cells, bone marrow stromal cells, |
| domains | | myoblasts. |
| LFA-3 | CD2 | Endothelial cells, leukocytes, epithelial cells |
| 6 Ig domains | | |
| PECAM-1 | CD31, heparin | Endothelial cells (at EC-EC junctions), T cell |
| (CD3l) | | subsets, platelets, neutrophils, eosinophils, |
| | | monocytes, smooth muscle cells, bone marrow |
| | | stem cells. |
| NCAM | NCAM, heparin SO₄ | Neural cells, muscle |
| MadCAM-1 | α4β7, L-selectin | Peyer's patch, mesenteric lymph nodes, |
| 4 Ig domains | | mucosal endothelial cells, spleen. |
| CD2 | CD58, CD59, CD48 | T lymphocytes |

TABLE 4
Cadherin Superfamily

| Receptor | Ligand | Distribution |
|---|---|---|
| Classical Cadherins | | |
| E-Cadherin/ | Homotypic: E-Cadherin | Embryo, epithelium |
| Epithelial | | |
| Cadherin/ | | |
| Uvomorulin | | |
| N-Cadherin/ | Homotypic: N-Cadherin | Neural and muscle tissue |
| Neural | | |
| Cadherin | | |
| VE-Cadherin | Homotypic: VE Cadherin | Endothelial, adherens junctions |
| P-Cadherin | Homotypic: P-Cadherin | Mouse placenta |
| Desmasomal Cadherins | | |
| Desmocollin | Homo- and Heterotpypic | Epithelium, epidervis, myocardium, |
| | | desmosomes |
| Desmoglein | Homo- and Heterotpypic | Epithelium, epidervis, myocardium, |
| | | desmosomes |
| ProtoCadherins | | |
| μ-Cadberin | | Embryo |
| CNR Cadherin | | Nervous system |
| Other | | |
| Cadherins | | |
| 7-TM | Homotypic: | 7-TM |
| Cadherin | -Cadherin | |
| Flamingo | | |
| T-Cadherin | | |
| Fat | | Human epithelium |

TABLE 6
Invasins/Intimins

| Receptor | Ligand | Distribution |
|---|---|---|
| Invasin | Integrins | Yersinia pseudotuberulosis |
| Intimin | | Gram-negative bacterium (including |
| | | Eseherichia coli) |

### THE INVENTION

The invention is based on the discovery that the KIAA0301 protein, G7c protein, KIAA0564 protein, NG37 protein, CAB01991.1 protein and Rv0368c protein function as adhesion molecules.

Cell adhesion molecules mediate cell-cell adhesion. Cell adhesion molecules bind cells together by one of three possible mechanisms:

(1) Adhesion molecules on one cell bind adhesion molecules of an identical type on adjacent cells (homotypic adhesion);

(2) Adhesion molecules on one cell bind adhesion molecules of a different type on adjacent cells (heterotypic adhesion);

(3) Adhesion molecules on adjacent cells are linked to one another by secreted multivalent linker molecules.

When a molecule is described herein as possessing activity as an adhesion molecule, this is intended to mean that the described molecules mediate cell-cell adhesion by one or more of these mechanisms.

For the KIAA0301 protein, it has been found that a region including residues 1832-2036 of this protein sequence adopts an equivalent fold to residues 4 to 193 of the α2 Integrin I-domain (PDB code 1AOX:A). α2 Integrin is known to function as an adhesion molecule, and the I-domain is critical for this function. Furthermore, the divalent metal ion binding residues Ser153, Ser155 and Asp254 of the α2 Integrin I-domain are conserved as Ser1843, Ser 1845 and Asp1948 in KIAA0301, respectively. This relationship is not just to the α2 Integrin I-domain, but rather to the I-domain family as a whole. It has been found that a region whose boundaries extend between, at the most, residue 1832 and residue 2036, and at the least, residue 1836 and residue 1950 of KIAA0301 adopts an equivalent fold to to a range of other I-domains including the MAC-1 (PDB code: 1IDO) and LFA-1 (PDB code: 1ZOO:A) I-domains. Furthermore, the divalent metal ion binding residues Ser139, Ser141 and Asp239 of the LFA-1 I-domain (1ZOO:A) are conserved as Ser1843, Ser 1845 and Asp1948 in KIAA0301, respectively. Thus, by reference to the Genome Threader™ alignment of KIAA0301 with the α2 Integrin (1AOX:A) and LFA-1 (1ZOO:A) I-domains, Ser1843, Ser1845 and Asp1948 of KIAA0301 are predicted to form a metal ion binding triad. KIAA0301 also shows conservation of residues to the metal ion binding triad of the MAC-1 I-domain (1IDO). The MAC-1 (1IDO) triad differs from the triad found in the α2 Integrin (1AOX:A) and LFA-1 (1ZOO:A) I-domains because the third metal ion ligand is a Threonine rather than an Aspartate. Nonetheless, by reference to the Genome Threader™ alignment of KIAA0301 with MAC-1 (1IDO), the MAC-1 (1IDO) triad Ser142, Ser144 and Thr201 is conserved as Ser1843, Ser1845 and Thr1912 in KIAA0301. Thus KIAA0301 has two non-mutually exclusive metal ion binding triads predicted; (Ser1843, Ser1845 and Asp1948) and (Ser1843, Ser1845 and Thr1912).

The combination of equivalent fold and conservation of divalent metal ion binding residues allows the functional annotation of this region of KIAA0301, and therefore proteins that include this region, as possessing adhesion molecule activity.

For the G7c protein, it has been found that a region including residues 10-126 of this protein sequence adopts an equivalent fold to residues 3 to 119 of the LFA-1 I-domain (PDB code 1LFA:B). LFA-1 is known to function as an adhesion molecule, and the I-domain is critical for this function. Furthermore, the divalent metal ion binding residues Ser139, Ser141 and Asp239 of the LFA-1 I-domain are conserved as Thr25 (conservative substitution), Ser27 and Asp119 in G7c, respectively. This relationship is not just to the LFA-1 I-domain, but rather to the I-domain family as a whole. It has been found that a region whose boundaries extend between, at the most, residue 10 and residue 126, and at the least, residue 20 and residue 105 of G7c adopts an equivalent fold to to a range of other I-domains including the MAC-1 I-domain (PDB code 1JLM). Furthermore the divalent metal ion binding residues of the MAC-1 I-domain are conserved as Thr25 (conservative substitution), Ser27 and Asp119 in G7c. Thus by reference to the Genome Threader™ alignment with LFA-1 (1LFA:B) and MAC-1 (1JLM), residues Thr25, Ser27 and Asp119 of G7c are predicted to form a metal ion binding triad. The combination of equivalent fold and conservation of divalent metal ion binding residues allows the functional annotation of this region of G7c, and therefore proteins that include this region, as possessing adhesion molecule activity.

For the KIAA0564 protein, it has been found that a region including residues 1248-1403 of this protein sequence adopts an equivalent fold to residues 2 to 140 of the LFA-1 I-domain (PDB code 1LFA:A). LFA-1 is known to function as an adhesion molecule, and the I-domain is critical for this function. Furthermore, the divalent metal ion binding residues Ser139, Ser141 and Asp239 of the LFA-1 I-domain are conserved as Ser1258, Ser1260 and Asp1367 in KIAA0564, respectively. This relationship is not just to the LFA-1 I-domain, but rather to the I-domain family as a whole. It has been found that a region whose boundaries extend between, at the most, residue 1248 and residue 1432, and at the least, residue 1253 and residue 1403 of KIAA0564 adopts an equivalent fold to to a range of other I-domains including the MAC-1 I-domain (PDB code 1BHO:2). Furthermore the divalent metal ion binding residues of the MAC-1 I-domain (1BHO:2) are conserved as Ser1258, Ser1260 and Asp1367 in KIAA0564. Thus by reference to the Genome Threader™ alignment with LFA-1 (1LFA:A) and MAC-1 (1BHO:2), residues Ser1258, Ser1260 and Asp1367 of KIAA0564 are predicted to form a metal ion binding triad. The combination of equivalent fold and conservation of divalent metal ion binding residues allows the functional annotation of this region of KIAA0564, and therefore proteins that include this region, as possessing adhesion molecule activity.

For the NG37 protein, it has been found that a region including residues 308-424 of this protein sequence adopts an equivalent fold to residues 3 to 119 of the LFA-1 I-domain (PDB code 1CQP:A). LFA-1 is known to function as an adhesion molecule, and the I-domain is critical for this function. Furthermore, the divalent metal ion binding residues Ser139, Ser141 and Asp239 of the LFA-1 I-domain are conserved as Thr323 (conservative substitution), Ser325 and Asp417 in NG37, respectively. The combination of equivalent fold and conservation of divalent metal ion binding residues allows the functional annotation of this region of NG37, and therefore proteins that include this region, as possessing adhesion molecule activity.

For the CAB01991.1 protein, it has been found that a region including residues 484-646 of this protein sequence adopts an equivalent fold to residues 5 to 179 of the LFA-1 I-domain (PDB code 1CQP:A). LFA-1 is known to function as an adhesion molecule, and the I-domain is critical for this function. Furthermore, the divalent metal ion binding residues Ser139, Ser141 and Asp239 of the LFA-1 I-domain are conserved as Ser491, Ser493 and Asp579 in CAB01991.1, respectively. This relationship is not just to the LFA-1 I-domain, but rather to the I-domain family as a whole. It has been found that a region whose boundaries extend between, at the most, residue 482 and residue 646, and at the least, residue 484 and residue 646 of CAB01991.1 adopts an equivalent fold to to a range of other I-domains including the MAC-1 I-domain (PDB code 1BHO:2). Furthermore the divalent metal ion binding residues of the MAC-1 I-domain (1BHO:2) are conserved as Ser491, Ser493 and Asp579 in CAB01991.1. Thus by reference to the Genome Threader™ alignment with LFA-1 (1CQP:A) and MAC-1 (1BHO:2), residues Ser491, Ser493 and Asp579 of CAB01991.1 are predicted to form a metal ion binding triad. The combination of equivalent fold and conservation of divalent metal ion binding residues allows the functional annotation of this region of CAB01991.1, and therefore proteins that include this region, as possessing adhesion molecule activity.

For the Rv0368c protein, it has been found that a region including residues 230-370 of this protein sequence adopts an equivalent fold to residues 8 to 157 of the α2 Integrin I-domain (PDB code 1AOX:A). The α2 Integrin is known to function as an adhesion molecule, and the I-domain is critical for this function. Furthermore, the divalent metal ion binding residues Ser153, Ser155 and Asp254 of the α2 Integrin I-domain are conserved as Ser237, Ser239 and Asp330 in Rv0368c, respectively. This relationship is not just to the α2 Integrin I-domain, but rather to the I-domain family as a whole. It has been found that a region whose boundaries extend between, at the most, residue 230 and residue 370, and at the least, residue 230 and residue 339 of Rv0368c adopts an equivalent fold to to a range of other I-domains including the LFA-1 (1DGQ:A), α1 Integrin (1QC5:A), and MAC-1 (1IDO) I-domains.

Furthermore, the divalent metal ion binding residues Ser139, Ser141 and Asp239 of the LFA-1 I-domain (1DGQ:A) are conserved as Ser237, Ser 239 and Asp330 in Rv0368c, respectively. Furthermore, the divalent metal ion binding residues Ser542, Ser544 and Asp143 of the α1 Integrin (1QC5:A) are conserved as Ser237, Ser 239 and Asp330 in Rv0368c, respectively. Thus, by reference to the Genome Threader™ alignment of Rv0368c with the α2Integrin (1AOX:A), LFA-1 (1DGQ:A) and α1 Integrin (1QC5:A) I-domains, Ser237, Ser239 and Asp330 of Rv0368c are predicted to form a metal ion binding triad. Rv0368c also shows conservation of residues to the metal ion binding triad of the MAC-1 I-domain (1IDO). The MAC-1 (1IDO) triad differs from the triad found in the α2Integrin (1AOX:A), LFA-1 (1DGQ:A) and α1 Integrin (1QC5:A) I-domains because the third metal ion ligand is a Threonine rather than an Aspartate. Nonetheless, by reference to the Genome Threader™ alignment of Rv0368c with MAC-1 (1IDO), the MAC-1 (1IDO) triad Ser142, Ser144 and Thr201 is conserved as Ser237, Ser239 and Thr302 in Rv0368c. Thus Rv0368c has two non-mutually exclusive metal ion binding triads predicted; (Ser237, Ser239 and Asp330) and (Ser237, Ser239 and Thr302).

The combination of equivalent fold and conservation of divalent metal ion binding residues allows the functional annotation of this region of Rv0368c, and therefore proteins that include this region, as possessing adhesion molecule activity.

In a first aspect, the invention provides a polypeptide, which polypeptide:

(i) has the amino acid sequence as recited in SEQ ID NO:2, SEQ ID NO:4, SEQ ID NO:6, SEQ ID NO:8, SEQ ID NO:10, or SEQ ID NO:12;

(ii) is a fragment thereof having adhesion molecule activity or having an antigenic determinant in common with the polypeptides of (i); or

(iii) is a functional equivalent of (i) or (ii).

The polypeptide having the sequence recited in SEQ ID NO:2 is referred to hereafter as “the AD1 polypeptide”.

According to this aspect of the invention, a preferred polypeptide fragment according to part ii) above includes the region of the AD1 polypeptide that is predicted as that responsible for adhesion molecule activity (hereafter, the “AD1 adhesion molecule region”), or is a variant thereof that possesses the divalent metal ion binding trio (Ser1843, Ser1845 and Asp1912, or equivalent residues) or the trio (Ser1843, Ser1845 and Thr1912, or equivalent residues). As defined herein, the AD1 adhesion molecule region is considered to extend between, at the most, residue 1832 and residue 2036, and at the least, residue 1836 and residue 1950 of the AD1 polypeptide sequence.

The polypeptide having the sequence recited in SEQ ID NO:4 is referred to hereafter as “the AD2 polypeptide”.

According to this aspect of the invention, a preferred polypeptide fragment according to part ii) above includes the region of the AD2 polypeptide that is predicted as that responsible for adhesion molecule activity (hereafter, the “AD2 adhesion molecule region”), or is a variant thereof that possesses the divalent metal ion binding residues Thr25, Ser27 and Asp119, or equivalent residues. As defined herein, the AD2 adhesion molecule region is considered to extend between, at the most, residue 10 and residue 126, and at the least, residue 20 and residue 105 of the AD2 polypeptide sequence.

The polypeptide having the sequence recited in SEQ ID NO:6 is referred to hereafter as “the AD3 polypeptide”.

According to this aspect of the invention, a preferred polypeptide fragment according to part ii) above includes the region of the AD3 polypeptide that is predicted as that responsible for adhesion molecule activity (hereafter, the “AD3 adhesion molecule region”), or is a variant thereof that possesses the divalent metal ion binding residues Ser1258, Ser1260 and Asp1367, or equivalent residues. As defined herein, the AD3 adhesion molecule region is considered to extend between, at the most, residue 1248 and residue 1432, and at the least, residue 1253 and residue 1403 of the AD3 polypeptide sequence.

The polypeptide having the sequence recited in SEQ ID NO:8 is referred to hereafter as “the AD4 polypeptide”.

According to this aspect of the invention, a preferred polypeptide fragment according to part ii) above includes the region of the AD4 polypeptide that is predicted as that responsible for adhesion molecule activity (hereafter, the “AD4 adhesion molecule region”), or is a variant thereof that possesses the divalent metal ion binding residues Thr323, Ser325 and Asp417, or equivalent residues. As defined herein, the AD4 adhesion molecule region is considered to extend between residue 308 and residue 424 of the AD4 polypeptide sequence.

The polypeptide having the sequence recited in SEQ ID NO:10 is referred to hereafter as “the AD5 polypeptide”.

According to this aspect of the invention, a preferred polypeptide fragment according to part ii) above includes the region of the AD5 polypeptide that is predicted as that responsible for adhesion molecule activity (hereafter, the “AD5 adhesion molecule region”), or is a variant thereof that possesses the divalent metal ion binding residues Ser491, Ser493 and Asp579, or equivalent residues. As defined herein, the AD5 adhesion molecule region is considered to extend between, at the most, residue 482 and residue 646, and at the least, residue 484 and residue 646 of the AD5 polypeptide sequence.

The polypeptide having the sequence recited in SEQ ID NO:12 is referred to hereafter as “the AD6 polypeptide”.

According to this aspect of the invention, a preferred polypeptide fragment according to part ii) above includes the region of the AD6 polypeptide that is predicted as that responsible for adhesion molecule activity (hereafter, the “AD6 adhesion molecule region”), or is a variant thereof that possesses either the trio of divalent metal ion binding residues: Ser237, Ser239 and Asp330, or equivalent residues; or the trio of Ser237, Ser239 and Thr302, or equivalent residues. As defined herein, the AD6 adhesion molecule region is considered to extend between, at the most, residue 230 and residue 370, and at the least, residue 230 and residue 339 of the AD6 polypeptide sequence.

This aspect of the invention also includes fusion proteins that incorporate polypeptide fragments and variants of these polypeptide fragments as defined above, provided that said fusion proteins possess activity as an adhesion molecule.

In a second aspect, the invention provides a purified nucleic acid molecule that encodes a polypeptide of the first aspect of the invention. Preferably, the purified nucleic acid molecule has the nucleic acid sequence as recited in SEQ ID NO:1 (encoding the AD1 polypeptide), SEQ ID NO:3 (encoding the AD2 polypeptide), SEQ ID NO:5 (encoding the AD3 polypeptide), SEQ ID NO:7 (encoding the AD4 polypeptide), in SEQ ID NO:9 (encoding the AD5 polypeptide), or SEQ ID NO:11 (encoding the AD6 polypeptide) or is a redundant equivalent or fragment of any one of these sequences. A preferred nucleic acid fragment is one that encodes a polypeptide fragment according to part ii) above, preferably a polypeptide fragment that includes the AD1 adhesion molecule region, the AD2 adhesion molecule region, the AD3 adhesion molecule region, the AD4 adhesion molecule region, the AD5 adhesion molecule region or the AD6 adhesion molecule region, or that encodes a variant of these fragments as this term is defined above.

In a third aspect, the invention provides a purified nucleic acid molecule which hydridizes under high stringency conditions with a nucleic acid molecule of the second aspect of the invention.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to, and which preferably inhibits the adhesion molecule activity of, a polypeptide of the first aspect of the invention.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonise) the level of expression of the gene or the activity of the polypeptide. Importantly, the identification of the function of the region defined herein as the AD1, AD2, AD3, AD4, AD5 and AD6 adhesion molecule regions of the AD1, AD2, AD3, AD4, AD5 and AD6 polypeptides, respetively, allows for the design of screening methods capable of identifying compounds that are effective in the treatment and/or diagnosis of diseases in which adhesion molecules are implicated.

In an eighth aspect, the invention provides a polypeptide of the first aspect of the invention, or a nucleic acid molecule of the second or third aspect of the invention, or a vector of the fourth aspect of the invention, or a ligand of the fifth aspect of the invention, or a compound of the sixth aspect of the invention, for use in therapy or diagnosis. These molecules may also be used in the manufacture of a medicament for the treatment of cardiovascular diseases including atherosclerosis, ischaemia, restenosis, reperfusion injury, sepsis, haematological diseases such as leukaemia, blood clotting disorders, such as thrombosis, cancer including lung, prostate, breast, colorectal and brain tumours, metastasis, inflammatory diseases such as rhinitis, gastrointestinal diseases, including inflammatory bowel disease, ulcerative colitis, Crohn's disease, respiratory diseases including asthma, chronic obstructive pulmonary disease (COPD), respiratory distress syndrome, pulmonary fibrosis, immune disorders, including autoimmune diseases, rheumatoid arthritis, transplant rejection, allergy, liver diseases such as cirrhosis, endocrine diseases, such as diabetes, bone diseases such as osteoporosis, neurological diseases including stroke, multiple sclerosis, spinal cord injury, burns and wound healing, bacteria infections, particularly Mycobacterium tuberculosis infection, and virus infections.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

The adhesion molecules whose sequences are presented in SEQ ID NO:10 and SEQ ID NO:12 are implicated herein in the pathogenicity of the organism Mycobacterium tuberculosis. Accordingly, ligands to this polypeptide, and in particular, to the AD5 and AD6 adhesion molecule regions, of the AD5 and AD6 polypeptides respectively, as these regions are defined herein, are likely to be effective in controlling disease caused by this organism. Furthermore, these polypeptides, and in particular, polypeptide fragments including the AD5 or AD6 adhesion molecule regions of the AD5 and AD6 polypeptide sequences provide a potential component for a vaccine against this organism and the diseases that it causes.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as an adhesion molecule. The invention also provides for the use of a nucleic acid molecule according to the second or third aspects of the invention to express a protein that possesses adhesion molecule activity. The invention also provides a method for effecting cell-cell adhesion, said method utilising a polypeptide of the first aspect of the invention.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, for use in the manufacture of a medicament for the diagnosis or treatment of a disease, such as herpes virus infection.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be using in screening regimes for the identification of compounds that are effective in the treatment or diagnosis of such a disease.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of the those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N. Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro- protein that can be activated by cleavage of the pre-, pro- or prepro- portion to produce an active mature polypeptide. In such polypeptides, the pre-, pro- or prepro- sequence may be a leader or secretory sequence or may be a sequence that is employed for purification of the mature polypeptide sequence. The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the AD1, AD2, AD3, AD4, AD5 or AD6 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the AD1, AD2, AD3, AD4, AD5 or AD6 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions.

Such mutants also include polypeptides in which one or more of the amino acid residues includes a substituent group;

Typically, greater than 30% identity between two polypeptides (preferably, over a specified region) is considered to be an indication of functional equivalence. Preferably, functionally equivalent polypeptides of the first aspect of the invention have a degree of sequence identity with the AD1, AD2, AD3, AD4, AD5 or AD6 polypeptide, or with active fragments thereof, of greater than 30%. More preferred polypeptides have degrees of identity of greater than 40%, 50%, 60%, 70%, 80%, 90%, 95%, 98% or 99%, respectively with the AD1, AD2, AD3, AD4, AD5 or AD6 polypeptide, or with active fragments thereof.

Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1.

In the present case, preferred active fragments of the AD1 polypeptide are those that include the AD1 adhesion molecule region and which possess either the metal binding trio of residues Ser1843, Ser1845 and Asp1948, or the alternative trio of Ser1843, Ser1845 and Thr1912, or equivalent residues. By “equivalent residues” is meant residues that are equivalent to the residues that bind the divalent metal ion may replace one or more of the three metal ion binding residues, provided that the adhesion molecule region retains activity as an adhesion molecule. For example Ser1843 or Ser1845 may be replaced by a Threonine. Asp1948 may be replaced by a Glutamate. Thr1912 may be replaced by a Serine. Accordingly, this aspect of the invention includes polypeptides that have degrees of identity of greater than 30%, preferably, greater than 40%, 50%, 60%, 70%, 80%,90%, 95%, 98% or 99%, respectively, with the adhesion molecule region of the AD1 polypeptide and which possess either the trio of Ser1843, Ser1845 and Asp1948 or the alternative trio Ser1843, Ser1845 and Thr1912, or equivalent residues. As discussed above, the AD1 adhesion molecule region is considered to extend between, at the most, residue 1832 and residue 2036, and at the least, residue 1836 and residue 1950 of the AD1 polypeptide sequence.

In the present case, preferred active fragments of the AD2 polypeptide are those that include the AD2 adhesion molecule region and which possess the metal binding residues Thr25, Ser27 and Asp119 or equivalent residues. By “equivalent residues” is meant residues that are equivalent to the residues that bind the divalent metal ion may replace one or more of the three metal ion binding residues, provided that the adhesion molecule region retains activity as an adhesion molecule. For example, Thr25 may be replaced by a Serine. Ser27 may be replaced by a Threonine. Asp119 may be replaced by a Glutamate. Accordingly, this aspect of the invention includes polypeptides that have degrees of identity of greater than 30%, preferably, greater than 40%, 50%, 60%, 70%, 80%,90%, 95%, 98% or 99%, respectively, with the AD2 adhesion molecule region and which possess the metal binding residues Thr25, Ser27 and Asp119, or equivalent residues. As discussed above, the AD2 adhesion molecule region is considered to extend between, at the most, residue 10 and residue 126, and at the least, residue 20 and residue 105 of the AD2 polypeptide sequence.

In the present case, preferred active fragments of the AD3 polypeptide are those that include the AD3 adhesion molecule region and which possess the metal binding residues Ser1258, Ser1260 and Asp1367 or equivalent residues. By “equivalent residues” is meant residues that are equivalent to the residues that bind the divalent metal ion may replace one or more of the three metal ion binding residues, provided that the adhesion molecule region retains activity as an adhesion molecule. For example, Ser1258 or Ser1260, or both may be replaced by a Threonine. Asp1367 may be replaced by a Glutamate. Accordingly, this aspect of the invention includes polypeptides that have degrees of identity of greater than 30%, preferably, greater than 40%, 50%, 60%, 70%, 80%, 90%, 95%, 98% or 99%, respectively, with the AD3 adhesion molecule region and which possess the metal binding residues Ser1258, Ser1260 and Asp1367, or equivalent residues. As discussed above, the AD3 adhesion molecule region is considered to extend between, at the most, residue 1248 and residue 1432, and at the least, residue 1253 and residue 1403 of the AD3 polypeptide sequence.

In the present case, preferred active fragments of the AD4 polypeptide are those that include the AD4 adhesion molecule region and which possess the metal binding residues Thr323, Ser325 and Asp417 or equivalent residues. By “equivalent residues” is meant residues that are equivalent to the residues that bind the divalent metal ion may replace one or more of the three metal ion binding residues, provided that the adhesion molecule region retains activity as an adhesion molecule. For example Thr323 may be replaced by a Serine. Ser325 may be replaced by a Threonine. Asp417 may be replaced by a Glutamate. Accordingly, this aspect of the invention includes polypeptides that have degrees of identity of greater than 30%, preferably, greater than 40%, 50%, 60%, 70%, 80%,90%, 95%, 98% or 99%, respectively, with AD4 the adhesion molecule region and which possess the metal binding residues Thr323, Ser325 and Asp417, or equivalent residues. As discussed above, the AD4 adhesion molecule region is considered to extend between residue 308 and residue 424 of the AD4 polypeptide sequence.

In the present case, preferred active fragments of the AD5 polypeptide are those that include the AD5 adhesion molecule region and which possess the metal binding residues Ser491, Ser493 and Asp579 or equivalent residues. By “equivalent residues” is meant residues that are equivalent to the residues that bind the divalent metal ion may replace one or more of the three metal ion binding residues, provided that the adhesion molecule region retains activity as an adhesion molecule. For example, Ser491 or Ser493 or both may be replaced by a Threonine. Asp579 may be replaced by a Glutamate. Accordingly, this aspect of the invention includes polypeptides that have degrees of identity of greater than 30%, preferably, greater than 40%, 50%, 60%, 70%, 80%,90%, 95%, 98% or 99%, respectively, with the AD5 adhesion molecule region and which possess the metal binding residues Ser491, Ser493 and Asp579, or equivalent residues. As discussed above, the AD5 adhesion molecule region is considered to extend between, at the most, residue 482 and residue 646, and at the least, residue 484 and residue 646 of the AD5 polypeptide sequence.

In the present case, preferred active fragments of the AD6 polypeptide are those that include the AD6 adhesion molecule region and which possess the trio of metal binding residues Ser237, Ser239 and Asp330 or the alternative trio of Ser237, Ser239 and Thr302, or equivalent residues. By “equivalent residues” is meant residues that are equivalent to the residues that bind the divalent metal ion may replace one or more of the three metal ion binding residues, provided that the adhesion molecule region retains activity as an adhesion molecule. For example, Ser237 or Ser239 or both may be replaced by a Threonine. Asp330 may be replaced by a Glutamate. Thr302 may be replaced by a Serine. Accordingly, this aspect of the invention includes polypeptides that have degrees of identity of greater than 30%, preferably, greater than 40%, 50%, 60%, 70%, 80%,90%, 95%, 98% or 99%, respectively, with the AD6 adhesion molecule region and which possess the trio of metal binding residues Ser237, Ser239 and Asp330 or the alternative trio of Ser237, Ser239 and Thr302, or equivalent residues As discussed above, the AD6 adhesion molecule region is considered to extend between, at the most, residue 230 and residue 370, and at the least, residue 230 and residue 339 of the AD6 polypeptide sequence.

The functionally-equivalent polypeptides of the first aspect of the invention may also be polypeptides which have been identified using one or more techniques of structural alignment. For example, the Inpharmatica Genome Threader™ technology that forms one aspect of the search tools used to generate the Biopendium search database may be used (see co-pending International patent application PCT/GB01/01105) to identify polypeptides of presently-unknown function which, while having low sequence identity as compared to the AD1, AD2, AD3, AD4, AD5 or AD6 polypeptides, are predicted to have adhesion molecule activity, by virtue of sharing significant structural homology with the AD1, AD2, AD3, AD4, AD5 or AD6 polypeptide sequences.

By “significant structural homology” is meant that the Inpharmatica Genome Threader™ predicts two proteins, or protein regions, to share structural homology with a certainty of 80% and above. The certainty value of the Inpharmatica Genome Threader™ is calculated as follows. A set of comparisons was initially performed using the Inpharmatica Genome Threader™ exclusively using sequences of known structure. Some of the comparisons were between proteins that were known to be related (on the basis of structure). A neural network was then trained on the basis that it needed to best distinguish between the known relationships and known not-relationships taken from the CATH structure classification (www.biochem.ucl.ac.uk/bsm/cath). This resulted in a neural network score between 0 and 1. However, again as the number of proteins that are related and the number that are unrelated were known, it was possible to partition the neural network results into packets and calculate empirically the percentage of the results that were correct. In this manner, any genuine prediction in the Biopendium search database has an attached neural network score and the percentage confidence is a reflection of how successful the Inpharmatica Genome Threader™ was in the training/testing set.

Structural homologues of AD1 should share structural homology with the AD1 adhesion molecule region and possess the metal binding trio Ser1843, Ser1845 and Asp1948 or the alternative trio Ser1843, Ser1845 and Thr1912, or equivalent residues. Such structural homologues are predicted to have adhesion molecule activity by virtue of sharing significant structural homology with this polypeptide sequence and possessing the metal ion binding residues.

Structural homologues of AD2 should share structural homology with the AD2 adhesion molecule region and possess the metal binding trio Thr25, Ser27 and Asp119, or equivalent residues. Such structural homologues are predicted to have adhesion molecule activity by virtue of sharing significant structural homology with this polypeptide sequence and possessing the metal ion binding residues.

Structural homologues of AD3 should share structural homology with the AD3 adhesion molecule region and possess the metal binding trio Ser1258, Ser1260 and Asp1367, or equivalent residues. Such structural homologues are predicted to have adhesion molecule activity by virtue of sharing significant structural homology with this polypeptide sequence and possessing the metal ion binding residues.

Structural homologues of AD4 should share structural homology with the AD4 adhesion molecule region and possess the metal binding trio Thr323, Ser325 and Asp417, or equivalent residues. Such structural homologues are predicted to have adhesion molecule activity by virtue of sharing significant structural homology with this polypeptide sequence and possessing the metal ion binding residues.

Structural homologues of AD5 should share structural homology with the AD5 adhesion molecule region and possess the metal binding trio Ser491, Ser493 and Asp579, or equivalent residues. Such structural homologues are predicted to have adhesion molecule activity by virtue of sharing significant structural homology with this polypeptide sequence and possessing the metal ion binding residues.

Structural homologues of AD6 should share structural homology with the AD6 adhesion molecule region and possess the metal binding trio Ser237, Ser239 and Asp330 or the alternative trio of Ser237, Ser239 and Thr302, or equivalent residues. Such structural homologues are predicted to have adhesion molecule activity by virtue of sharing significant structural homology with this polypeptide sequence and possessing the metal ion binding residues.

The polypeptides of the first aspect of the invention also include fragments of the AD1, AD2, AD3, AD4, AD5 and AD6 polypeptides, functional equivalents of the fragments of the AD1, AD2, AD3, AD4, AD5 and AD6 polypeptides, and fragments of the functional equivalents of the AD1, AD2, AD3, AD4, AD5 and AD6 polypeptides, provided that those functional equivalents and fragments retain adhesion molecule activity or have an antigenic determinant in common with the AD1, AD2, AD3, AD4, AD5 or AD6 polypeptides.

As used herein, the term “fragment” refers to a polypeptide having an amino acid sequence that is the same as part, but not all, of the amino acid sequence of the AD1, AD2, AD3, AD4, AD5 or AD6 polypeptides or one of its functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant.

Preferred polypeptide fragments according to this aspect of the invention are fragments that include a region defined herein as the AD1, AD2, AD3, AD4, AD5 or AD6 adhesion molecule region of the AD1, AD2, AD3, AD4, AD5 and AD6 polypeptides, respectively. These regions are the regions that have been annotated as adhesion molecules. For the AD1 polypeptide, this region is considered to extend between, at the most, residue 1832 and residue 2036, and at the least, residue 1836 and residue 1950. This region of the AD2 polypeptide is considered to extend between, at the most, residue 10 and residue 126, and at the least, residue 20 and residue 105. This region of the AD3 polypeptide is considered to extend between, at the most, residue 1248 and residue 1432, and at the least, residue 1253 and residue 1403. This region of the AD4 polypeptide is considered to extend between residue 308 and residue 424. This region of the AD5 polypeptide is considered to extend between, at the most, residue 482 and residue 646, and at the least, residue 484 and residue 646. This region of the AD6 polypeptide is considered to extend between, at the most, residue 230 and residue 370, and at the least, residue 230 and residue 339.

Variants of this fragment are included as embodiments of this aspect of the invention, provided that these variants possess activity as an adhesion molecule.

In one respect, the term “variant” is meant to include extended or truncated versions of this polypeptide fragment.

For extended variants, it is considered highly likely that the adhesion molecule region of the AD1, AD2, AD3, AD4, AD5 and AD6 polypeptide will fold correctly and show adhesion molecule activity if additional residues C terminal and/or N terminal of these boundaries in the AD1, AD2, AD3, AD4, AD5 or AD6 polypeptide sequences are included in the polypeptide fragment. For example, an additional 5, 10, 20, 30, 40 or even 50 or more amino acid residues from the AD1, AD2, AD3, AD4, AD5 or AD6 polypeptide sequence, or from a homologous sequence, may be included at either or both the C terminal and/or N terminal of the boundaries of the adhesion molecule regions of the AD1, AD2, AD3, AD4, AD5 or AD6 polypeptide, without prejudicing the ability of the polypeptide fragment to fold correctly and exhibit adhesion molecule activity.

For truncated variants of the AD1 polypeptide, one or more amino acid residues may be deleted at either or both the C terminus or the N terminus of the adhesion molecule region of the AD1 polypeptide, although the metal ion binding trio (Ser1843, Ser1845 and Asp1948) or the alternative trio (Ser1843, Ser1845 and Thr1912), or equivalent residues should be maintained intact; deletions should not extend so far into the polypeptide sequence that any of these residues are deleted.

For truncated variants of the AD2 polypeptide, one or more amino acid residues may be deleted at either or both the C terminus or the N terminus of the adhesion molecule region of the AD2 polypeptide, although the metal ion binding residues (Thr25, Ser27 and Asp119, or equivalent residues) should be maintained intact; deletions should not extend so far into the polypeptide sequence that any of these residues are deleted.

For truncated variants of the AD3 polypeptide, one or more amino acid residues may be deleted at either or both the C terminus or the N terminus of the adhesion molecule region of the AD3 polypeptide, although the metal ion binding residues (Ser1258, Ser1260 and Asp1367, or equivalent residues) should be maintained intact; deletions should not extend so far into the polypeptide sequence that any of these residues are deleted.

For truncated variants of the AD4 polypeptide, one or more amino acid residues may be deleted at either or both the C terminus or the N terminus of the adhesion molecule region of the AD4 polypeptide, although the metal ion binding residues (Thr323, Ser325 and Asp417, or equivalent residues) should be maintained intact; deletions should not extend so far into the polypeptide sequence that any of these residues are deleted.

For truncated variants of the AD5 polypeptide, one or more amino acid residues may be deleted at either or both the C terminus or the N terminus of the adhesion molecule region of the AD5 polypeptide, although the metal ion binding residues (Ser491, Ser493 and Asp579, or equivalent residues) should be maintained intact; deletions should not extend so far into the polypeptide sequence that any of these residues are deleted.

For truncated variants of the AD6 polypeptide, one or more amino acid residues may be deleted at either or both the C terminus or the N terminus of the adhesion molecule region of the AD6 polypeptide, although the metal ion binding trio (Ser237, Ser239 and Asp330) or the alternative trio (Ser237, Ser239 and Thr302), or equivalent residues should be maintained intact; deletions should not extend so far into the polypeptide sequence that any of these residues are deleted.

In a second respect, the term “variant” includes homologues of the polypeptide fragments described above, that possess significant sequence homology with the adhesion molecule region of the AD1 polypeptide and which possess the metal ion binding trio (Ser1843, Ser1845 and Asp1948) or the alternative trio (Ser1843, Ser1845 and Thr1912), or equivalent residues, provided that said variants retain activity as an adhesion molecule.

The term “variant” also includes homologues of the polypeptide fragments described above, that possess significant sequence homology with the adhesion molecule region of the AD2 polypeptide and which possess the metal ion binding residues (Thr25, Ser27 and Asp119 or equivalent residues), provided that said variants retain activity as an adhesion molecule.

The term “variant” also includes homologues of the polypeptide fragments described above, that possess significant sequence homology with the adhesion molecule region of the AD3 polypeptide and which possess the metal ion binding Ser1258, Ser1260 and Asp1367 or equivalent residues), provided that said variants retain activity as an adhesion molecule.

The term “variant” also includes homologues of the polypeptide fragments described above, that possess significant sequence homology with the adhesion molecule region of the AD4 polypeptide and which possess the metal ion binding Thr323, Ser325 and Asp417 or equivalent residues), provided that said variants retain activity as an adhesion molecule.

The term “variant” also includes homologues of the polypeptide fragments described above, that possess significant sequence homology with the adhesion molecule region of the AD5 polypeptide and which possess the metal ion Ser491, Ser493 and Asp579 or equivalent residues), provided that said variants retain activity as an adhesion molecule.

The term “variant” also includes homologues of the polypeptide fragments described above, that possess significant sequence homology with the adhesion molecule region of the AD6 polypeptide and which possess the metal ion binding trio (Ser237, Ser239 and Asp330) or the alternative trio (Ser237, Ser239 and Thr302), or equivalent residues, provided that said variants retain activity as an adhesion molecule.

Homologues include those polypeptide molecules that possess greater than 30% identity with the AD1, AD2, AD3, AD4, AD5 or AD6 adhesion molecule regions, of the AD1, AD2, AD3, AD4, AD5 and AD6 polypeptides, respectively. Percentage identity is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1]. Preferably, variant homologues of polypeptide fragments of this aspect of the invention have a degree of sequence identity with the AD1, AD2, AD3, AD4, AD5 or AD6 adhesion molecule regions, of the AD1, AD2, AD3, AD4, AD5 and AD6 polypeptides, resepctively, of greater than 40%. More preferred variant polypeptides have degrees of identity of greater than 50%, 60%, 70%, 80%, 90%, 95%, 98% or 99%, respectively with the AD], AD2, AD3, AD4, AD5 or AD6 adhesion molecule regions of the AD1, AD2, AD3, AD4, AD5 or AD6 polypeptides, provided that said variants retain activity as an adhesion molecule. Variant polypeptides also include homologues of the truncated forms of the polypeptide fragments discussed above, provided that said variants retain activity as an adhesion molecule.

The polypeptide fragments of the first aspect of the invention may be polypeptide fragments that exhibit significant structural homology with the structure of the polypeptide fragment defined by the AD1, AD2, AD3, AD4, AD5 or AD6 adhesion molecule regions, of the AD1, AD2, AD3, AD4, AD5, or AD6 polypeptide sequences, for example, as identified by the Inpharmatica Genome Threader™. Accordingly, polypeptide fragments that are structural homologues of the polypeptide fragments defined by the AD1, AD2, AD3, AD4, AD5 or AD6 adhesion molecule regions of the AD1, AD2, AD3, AD4, AD5, and AD6 polypeptide sequences should adopt the same fold as that adopted by this polypeptide fragment, as this fold is defined above.

Structural homologues of the polypeptide fragment defined by the AD1 adhesion molecule region should also retain the metal ion binding trio (Ser1843, Ser1845 and Asp1948) or the alternative trio (Ser1843, Ser1845 and Thr1912), or equivalent residues.

Structural homologues of the polypeptide fragment defined by the AD2 adhesion molecule region should also retain the metal ion binding residues Thr25, Ser27 and Asp 119 or equivalent residues.

Structural homologues of the polypeptide fragment defined by the AD3 adhesion molecule should also retain the metal ion binding residues Ser1258, Ser1260 and Asp1367 or equivalent residues.

Structural homologues of the polypeptide fragment defined by the AD4 adhesion molecule region should also retain the metal ion binding residues Thr323, Ser325 and Asp417 or equivalent residues.

Structural homologues of the polypeptide fragment defined by the AD5 adhesion molecule region should also retain the metal ion binding residues Ser491, Ser493 and Asp579 or equivalent residues.

Structural homologues of the polypeptide fragment defined by the AD6 adhesion molecule region should also retain the metal ion binding trio (Ser237, Ser239 and Asp330) or the alternative trio (Ser237, Ser239 and Thr302), or equivalent residues.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre - and/or pro- polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the first aspect of the invention.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239: 1534 (1988); Kabat et al., J. Immunol., 147: 1709 (1991); Queen et al., Proc. Natl Acad. Sci. USA, 86, 10029 (1989); Gorman et al., Proc. Natl Acad. Sci. USA, 88: 34181 (1991); and Hodgson et al., Bio/Technology 9: 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode the polypeptide sequences recited in SEQ ID NO:2, SEQ ID NO:4, SEQ ID NO:6, SEQ ID NO:8, SEQ ID NO:10 or SEQ ID NO:12, and functionally equivalent polypeptides, including active fragments of the AD1, AD2, AD3, AD4, AD5, and AD6 polypeptides, such as a fragment including the AD1, AD2, AD3, AD4, AD5, or AD6 adhesion molecule regions of the AD1, AD2, AD3, AD4, AD5, and AD6 polypeptide sequences, or a homologue thereof.

Nucleic acid molecules encompassing these stretches of sequence form a preferred embodiment of this aspect of the invention.

These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones ,and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes the polypeptide of SEQ ID NO:2, or an active fragment thereof, may be identical to the coding sequence of the nucleic acid molecule shown in SEQ ID NO:1. These molecules also may have a different sequence which, as a result of the degeneracy of the genetic code, encodes the polypeptide SEQ ID NO:2, or an active fragment of the AD1 polypeptide, such as a fragment including the AD1 adhesion molecule region, or a homologue thereof. The AD1 adhesion molecule region is considered to extend between, at most residue 1832 and 2036, and at least, residue 1836 and residue 1950 of the AD1 polypeptide sequence. In SEQ ID NO:1 the AD1 adhesion molecule region is thus encoded by, at the most, a nucleic acid molecule including nucleotide 5495 to nucleotide 6109 and, at the least, by a nucleic acid molecule including nucleotide 5507 to 5851. Nucleic acid molecules encompassing this stretch of sequence, and homologues of this sequence, form a preferred embodiment of this aspect of the invention.

A nucleic acid molecule which encodes the polypeptide of SEQ ID NO:4, or an active fragment thereof, may be identical to the coding sequence of the nucleic acid molecule shown in SEQ ID NO:3. These molecules also may have a different sequence which, as a result of the degeneracy of the genetic code, encodes the polypeptide SEQ ID NO:4, or an active fragment of the AD2 polypeptide, such as a fragment including the AD2 adhesion molecule region, or a homologue thereof. The AD2 adhesion molecule region is considered to extend between, at most residue 10 and 126, and at least, residue 20 and residue 105 of the AD2 polypeptide sequence. In SEQ ID NO:3 the AD2 adhesion molecule region is encoded by, at the most, a nucleic acid molecule including nucleotide 30 to nucleotide 380 and, at the least, by a nucleic acid molecule including nucleotide 60 to 317. Nucleic acid molecules encompassing this stretch of sequence, and homologues of this sequence, form a preferred embodiment of this aspect of the invention.

A nucleic acid molecule which encodes the polypeptide of SEQ ID NO:6, or an active fragment thereof, may be identical to the coding sequence of the nucleic acid molecule shown in SEQ ID NO:5. These molecules also may have a different sequence which, as a result of the degeneracy of the genetic code, encodes the polypeptide SEQ ID NO:6, or an active fragment of the AD3 polypeptide, such as a fragment including the AD3 adhesion molecule region, or a homologue thereof. The AD3 adhesion molecule region is considered to extend between, at most residue 1248 and 1432, and at least, residue 1253 and residue 1403 of the AD3 polypeptide sequence. In SEQ ID NO:5 the AD3 adhesion molecule region is encoded by, at the most, a nucleic acid molecule including nucleotide 3744 to nucleotide 4298 and, at the least, by a nucleic acid molecule including nucleotide 3759 to 4211. Nucleic acid molecules encompassing this stretch of sequence, and homologues of this sequence, form a preferred embodiment of this aspect of the invention.

A nucleic acid molecule which encodes the polypeptide of SEQ ID NO:8, or an active fragment thereof, may be identical to the coding sequence of the nucleic acid molecule shown in SEQ ID NO:7. These molecules also may have a different sequence which, as a result of the degeneracy of the genetic code, encodes the polypeptide SEQ ID NO:8, or an active fragment of the AD4 polypeptide, such as a fragment including the AD4 adhesion molecule region, or a homologue thereof. The AD4 adhesion molecule region is considered to extend between residue 308 and 424 of the AD4 polypeptide sequence. In SEQ ID NO:7 the AD4 adhesion molecule region is encoded by a nucleic acid molecule including nucleotide 922 to nucleotide 1272. Nucleic acid molecules encompassing this stretch of sequence, and homologues of this sequence, form a preferred embodiment of this aspect of the invention.

A nucleic acid molecule which encodes the polypeptide of SEQ ID NO:10, or an active fragment thereof, may be identical to the coding sequence of the nucleic acid molecule shown in SEQ ID NO:9. These molecules also may have a different sequence which, as a result of the degeneracy of the genetic code, encodes the polypeptide SEQ ID NO:10, or an active fragment of the AD5 polypeptide, such as a fragment including the AD5 adhesion molecule region of the AD5 polypeptide sequence, or a homologue thereof. The AD5 adhesion molecule region is considered to extend between, at most residue 482 and 646, and at least, residue 484 and residue 646 of the AD5 polypeptide sequence. In SEQ ID NO:9 the AD5 adhesion molecule region is encoded by, at the most, a nucleic acid molecule including nucleotide 1444 to nucleotide 1938 and, at the least, by a nucleic acid molecule including nucleotide 1450 to 1938. Nucleic acid molecules encompassing this stretch of sequence, and homologues of this sequence, form a preferred embodiment of this aspect of the invention.

A nucleic acid molecule which encodes the polypeptide of SEQ ID NO:12, or an active fragment thereof, may be identical to the coding sequence of the nucleic acid molecule shown in SEQ ID NO:11. These molecules also may have a different sequence which, as a result of the degeneracy of the genetic code, encodes the polypeptide SEQ ID NO:12, or an active fragment of the AD6 polypeptide, such as a fragment including the AD6 adhesion molecule region, or a homologue thereof. The AD6 adhesion molecule region is considered to extend between, at most residue 230 and 370, and at least, residue 230 and 339 of the AD6 polypeptide sequence. In SEQ ID NO:11 the AD6 adhesion molecule region is encoded by, at the most, a nucleic acid molecule including nucleotide 688 to nucleotide 1110 and, at the least, by a nucleic acid molecule including nucleotide 688 to 1017. Nucleic acid molecules encompassing this stretch of sequence, and homologues of this sequence, form a preferred embodiment of this aspect of the invention.

Such nucleic acid molecules that encode the polypeptide of SEQ ID NO:2, SEQ ID NO:4, SEQ ID NO:6, SEQ ID NO:8, SEQ ID NO:10 or SEQ ID NO:12 may include, but are not limited to, the coding sequence for the mature polypeptide by itself; the coding sequence for the mature polypeptide and additional coding sequences, such as those encoding a leader or secretory sequence, such as a pro-, pre- or prepro- polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention.

As discussed above, a preferred fragment of the AD1 polypeptide is a fragment including the AD1 adhesion molecule region, or a homologue thereof. The adhesion molecule region is encoded by, at the most, a nucleic acid molecule including nucleotide 5495 to nucleotide 6109 of SEQ ID NO:1 and, at the least, by a nucleic acid molecule including nucleotide 5507 to 5851 of SEQ ID NO:1.

A preferred fragment of the AD2 polypeptide is a fragment including the AD2 adhesion molecule region, or a homologue thereof. The AD2 adhesion molecule region is encoded by, at the most, a nucleic acid molecule including nucleotide 30 to nucleotide 380 of SEQ ID NO:3 and, at the least, by a nucleic acid molecule including nucleotide 60 to 317 of SEQ ID NO:3.

A preferred fragment of the AD3 polypeptide is a fragment including the AD3 adhesion molecule region, or a homologue thereof. The AD3 adhesion molecule region is encoded by, at the most, a nucleic acid molecule including nucleotide 3744 to nucleotide 4298 of SEQ ID NO:5 and, at the least, by a nucleic acid molecule including nucleotide 3759 to 4211 of SEQ ID NO:5.

A preferred fragment of the AD4 polypeptide is a fragment including the AD4 adhesion molecule region, or a homologue thereof. The AD4 adhesion molecule region is encoded by a nucleic acid molecule including nucleotide 922 to nucleotide 1272 of SEQ ID NO:7.

A preferred fragment of the AD5 polypeptide is a fragment including the AD5 adhesion molecule region, or a homologue thereof. The AD5 adhesion molecule region is encoded by, at the most, a nucleic acid molecule including nucleotide 1444 to nucleotide 1938 of SEQ ID NO:9 and, at the least, by a nucleic acid molecule including nucleotide 1450 to 1938 of SEQ ID NO:9.

A preferred fragment of the AD6 polypeptide is a fragment including the AD6 adhesion molecule region, or a homologue thereof. The AD6 adhesion molecule region is encoded by, at the most, a nucleic acid molecule including nucleotide 688 to nucleotide 1110 of SEQ ID NO:9 and, at the least, by a nucleic acid molecule including nucleotide 688 to 1017 of SEQ ID NO:11.

Functionally equivalent nucleic acid molecules according to the invention may be naturally-occurring variants such as a naturally-occurring allelic variant, or the molecules may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5× Denhardts solution, 10% dextran sulphate, and 20 microgram/ml denatured, sheared salmon sperm DNA, followed by washing the filters in 0.1× SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to a nucleic acid molecule encoding the AD1 polypeptide (SEQ ID NO:2), AD2 polypeptide (SEQ ID NO:4), AD3 polypeptide (SEQ ID NO:6), AD4 polypeptide (SEQ ID NO:8), AD5 polypeptide (SEQ ID NO:10), or AD6 polypeptide (SEQ ID NO:12), and nucleic acid molecules that are substantially complementary to such nucleic acid molecules. A preferred active fragment is a fragment that includes an AD1, AD2, AD3, AD4, AD5 or AD6 adhesion molecule region of the AD1, AD2, AD3, AD4, AD5, and AD6 polypeptide sequences, resepctively. Accordingly, preferred nucleic acid molecules include those that are at least 70% identical over their entire length to a nucleic acid molecule encoding the adhesion molecule region of the AD1, AD2, AD3, AD4, AD5, and AD6 polypeptide sequence.

Percentage identity, as referred to herein, is as determined using BLAST version 2.1.3 using the default parameters specified by the NCBI (the National Center for Biotechnology Information; http://www.ncbi.nlm.nih.gov/).

Preferably, a nucleic acid molecule according to this aspect of the invention comprises a region that is at least 80% identical over its entire length to the nucleic acid molecule having the sequence given in SEQ ID NO:1, to a region including nucleotides 5495-6109 of this sequence, to a region including nucleotides 5507-5851 of this sequence, or a nucleic acid molecule that is complementary to any one of these regions of nucleic acid. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the AD1 polypeptide.

Preferably, a nucleic acid molecule according to this aspect of the invention comprises a region that is at least 80% identical over its entire length to the nucleic acid molecule having the sequence given in SEQ ID NO:3, to a region including nucleotides 30-380 of this sequence, to a region including nucleotides 60-317 of this sequence, or a nucleic acid molecule that is complementary to any one of these regions of nucleic acid. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the AD2 polypeptide.

Preferably, a nucleic acid molecule according to this aspect of the invention comprises a region that is at least 80% identical over its entire length to the nucleic acid molecule having the sequence given in SEQ ID NO:5, to a region including nucleotides 3744-4298 of this sequence, to a region including nucleotides 3759-4211 of this sequence, or a nucleic acid molecule that is complementary to any one of these regions of nucleic acid. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the AD3 polypeptide.

Preferably, a nucleic acid molecule according to this aspect of the invention comprises a region that is at least 80% identical over its entire length to the nucleic acid molecule having the sequence given in SEQ ID NO:7, to a region including nucleotides 922-1272 of this sequence, or a nucleic acid molecule that is complementary to any one of these regions of nucleic acid. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the AD4 polypeptide.

Preferably, a nucleic acid molecule according to this aspect of the invention comprises a region that is at least 80% identical over its entire length to the nucleic acid molecule having the sequence given in SEQ ID NO:9, to a region including nucleotides 1444-1938 of this sequence, to a region including nucleotides 1450-1938 of this sequence, or a nucleic acid molecule that is complementary to any one of these regions of nucleic acid. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the AD5 polypeptide.

Preferably, a nucleic acid molecule according to this aspect of the invention comprises a region that is at least 80% identical over its entire length to the nucleic acid molecule having the sequence given in SEQ ID NO:11, to a region including nucleotides 688-1110 of this sequence, to a region including nucleotides 688-1017 of this sequence, or a nucleic acid molecule that is complementary to any one of these regions of nucleic acid. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the AD6 polypeptide.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the AD1, AD2, AD3, AD4, AD5 or AD6 polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase 1, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the AD1, AD2, AD3, AD4, AD5 or AD6 polypeptides, particularly with an equivalent function to the AD1, AD2, AD3, AD4, AD5 or AD6 adhesion molecule region of the AD1, AD2, AD3, AD4, AD5 or AD6 polypeptides, is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989,1992). Probes comprising at least 15, preferably at least 30, and more preferably at least 50, contiguous bases that correspond to, or are complementary to, nucleic acid sequences from the appropriate encoding gene (SEQ ID NO:1), particularly a region from nucleotides 5495-6109, or from nucleotides 5507-5851 of SEQ ID NO: 1, are particularly useful probes.

Probes comprising at least 15, preferably at least 30, and more preferably at least 50, contiguous bases that correspond to, or are complementary to, nucleic acid sequences from the appropriate encoding gene (SEQ ID NO:3), particularly a region from nucleotides 30-380, or from nucleotides 60-317 of SEQ ID NO:3, are particularly useful probes.

Probes comprising at least 15, preferably at least 30, and more preferably at least 50, contiguous bases that correspond to, or are complementary to, nucleic acid sequences from the appropriate encoding gene (SEQ ID NO:5), particularly a region from nucleotides 3744-4298, or from nucleotides 3759-4211 of SEQ ID NO:5, are particularly useful probes.

Probes comprising at least 15, preferably at least 30, and more preferably at least 50, contiguous bases that correspond to, or are complementary to, nucleic acid sequences from the appropriate encoding gene (SEQ ID NO:7), particularly a region from nucleotides 922-1272 of SEQ ID NO:7, are particularly useful probes.

Probes comprising at least 15, preferably at least 30, and more preferably at least 50, contiguous bases that correspond to, or are complementary to, nucleic acid sequences from the appropriate encoding gene (SEQ ID NO:9), particularly a region from nucleotides 1444-1938, or from nucleotides 1450-1938 of SEQ ID NO:9, are particularly useful probes.

Probes comprising at least 15, preferably at least 30, and more preferably at least 50, contiguous bases that correspond to, or are complementary to, nucleic acid sequences from the appropriate encoding gene (SEQ ID NO:11), particularly a region from nucleotides 688-1110, or from nucleotides 688-1017 of SEQ ID NO:11, are particularly useful probes.

Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., Proc. Natl. Acad. Sci. USA (1988) 85: 8998-9002). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T., et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic. 1: 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

The vectors of the present invention comprise nucleic acid molecules of the invention and may be cloning or expression vectors. The host cells of the invention, which may be transformed, transfested or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al., [supra]. Particularly suitable methods include calcium phosphate transfection, DEAE-dextran mediated transfection, transvection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSport1™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, (1991) Phytochemistry 30, 3861-3863.

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk− or aprt± cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992) Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (DNA Cell Biol. 199312:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

Alternatively, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targetted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′ O -methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (ie. to prevent infection) or therapeutic (ie. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:

a) contacting a sample of tissue from the patient with a nucleic acid probe under stringent conditions that allow the formation of a hybrid complex between a nucleic acid molecule of the invention and the probe;

b) contacting a control sample with said probe under the same conditions used in step a);

c) and detecting the presence of hybrid complexes in said samples;

wherein detection of levels of the hybrid complex in the patient sample that differ from levels of the hybrid complex in the control sample is indicative of disease.

A further aspect of the invention comprises a diagnostic method comprising the steps of:

a) obtaining a tissue sample from a patient being tested for disease;

b) isolating a nucleic acid molecule according to the invention from said tissue sample; and,

c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250: 559-562 (1990), and Trask et al., Trends, Genet. 7:149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M.Chee et al., Science (1996) 274: 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application W095/251116 (Baldeschweiler et al). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;

(b) a polypeptide of the present invention; or

(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease, particularly cardiovascular diseases including atherosclerosis, ischaemia, restenosis, reperfusion injury, sepsis, haematological diseases such as leukaemia, blood clotting disorders, such as thrombosis, cancer including lung, prostate, breast, colorectal and brain tumours, metastasis, inflammatory diseases such as rhinitis, gastrointestinal diseases, including inflammatory bowel disease, ulcerative colitis, Crohn's disease, respiratory diseases including asthma, chronic obstructive pulmonary disease (COPD), respiratory distress syndrome, pulmonary fibrosis, immune disorders, including autoimmune diseases, rheumatoid arthritis, transplant rejection, allergy, liver diseases such as cirrhosis, endocrine diseases, such as diabetes, bone diseases such as osteoporosis, neurological diseases including stroke, multiple sclerosis, spinal cord injury, burns and wound healing, bacteria infections, particularly Mycobacterium tuberculosis infection, and virus infections.

Various aspects and embodiments of the present invention will now be described in more detail by way of example, with particular reference to the AD1, AD2, AD3, AD4, AD5 and AD6 polypeptides.

It will be appreciated that modification of detail may be made without departing from the scope of the invention.

### BRIEF DESCRIPTION OF THE FIGURES

FIG. 1: Front page of the Biopendium™. Search initiated using 1LFA:A.

FIG. 2A: Inpharmatica Genome Threader™ results of search using 1LFA:A. The arrow points to “Not Given”, the BAA20761.1 protein,

FIG. 2B: PSI-Blast results from search using 1LFA:A.

FIG. 3: Redundant Sequence Display page for BAA20761.1.

FIG. 4: NCBI protein report for BAA20761.1 (KIAA0301; AD1).

FIG. 5: PFAM search results for BAA20761.1 (AD1).

FIG. 6A: Inpharmatica Genome Threader™ results of search using BAA20761.1 (AD1) as the query sequence.

FIG. 6B: PSI-Blast results from search using BAA20761.1 (AD1).

FIG. 7A: Genome Threader™ alignment of BAA20761.1 (KIAA0301; AD1) and 1IDO, 1AOX:B, and 1AOX:A.

FIG. 7B: Genome Threader™ alignment of CAB8660.1 (CAB8660.1 is an alternative identifier for BAA20761.1, KIAA0301; AD1) and 1ZOO:A.

FIG. 8A: LigEye for 1AOX:A which illustrates the sites of interaction of the magnesium ion and 1AOX:A.

FIG. 8B: RasMol view of 1AOX:A, the I domain of integrin alpha2-beta1 in complex with a magnesium ion. The coloured balls represent the amino acids in 1AOX that directly interact with the divalent cation and are conserved in BAA20761.1 (KIAA0301; AD1).

FIG. 8C: RasMol view of 1AOX:A, The I-domain of integrin alpha2-beta1 in complex with a magnesium ion. The coloured balls represent the amino acids in 1AOX are conserved in BAA20761.1 (KIAA0301; AD1) as well.

FIG. 8D: Identification of model organism homologues of BAA20761.1

FIG. 8E: Sequence alignment of BAA20761.1 with model organism sequences CAA97671.1, AAF58612.1 and the structure 1AOX:B.

FIG. 9: UniGene report for BAA20761.1 (KIAA0301; AD1).

FIG. 10: SAGE library list.

FIG. 11A: SAGE results for BAA20761.1 (KIAA0301;AD1).

FIG. 11B: InterPro search results for BAA20761.1 (KIAA0301; AD1) as of Jul. 24, 2001.

FIG. 11C: InterPro search results for residues 1832-2036 of BAA20761.1 (KIAA0301; AD1) as of Jul. 24, 2001.

FIG. 11D: The PROSITE profile PS50079 for bipartite nuclear localisation signals has a high false positive rate.

FIG. 11E: The PROSITE profile PS50234 for von Willebrand factor/I-domains only became available in September 2000.

FIG. 11F: NCBI CDD search results for BAA20761.1 (KIAA0301; AD1) as of Jul. 24, 2001.

FIG. 11G: NCBI CDD search results for residues 1832-2036 of BAA20761.1 (KIAA0301; AD1) as of Jul. 24, 2001.

FIG. 11H: The smart profile smart00327 for von Willebrand factor/I-domains only became available on Jun. 30, 2001.

FIG. 12: Front page of the Biopendium™. Search initiated using 1LFA:A

FIG. 13A: Inpharmatica Genome Threader™ results of search using 1LFA:A. The arrow points to G7c (AD2), the protein CAB52192.1

FIG. 13B: PSI-Blast results from search using 1LFA:A.

FIG. 14: Redundant Sequence Display page for CAB52192.1 (AD2).

FIG. 15A: NCBI protein report for G7c (CAB52192.1; AD2)

FIG. 15B: J Immunol report detailing major histocompatibility recombinational hot spot in G7c (AD2) gene

FIG. 16: PFAM search results for CAB52192.1 (AD2).

FIG. 17A: Inpharmatica Genome Threader™ results of search using CAB52192.1 (AD2) as the query sequence.

FIG. 17B: PSI-Blast results from search using CAB52192.1 (AD2). The arrow points to the model organism homologue CAA87336.1.

FIG. 18A: Genome Threader™ alignment of CAB52192.1 (AD2) and 1LFA:B.

FIG. 18B: Alignment of CAB52192.1 (AD2) to CAA87336.1, 1CQP:A and 1CQP:B. 1CQP represents LFA-1 co-crystallised with lovostatin.

FIG. 18C: Genome Threader™ alignment of CAB52192.1 (AD2) and 1JLM.

FIG. 19A: LigEye for 1CQP which illustrates the sites of interaction of the magnesium ion and 1CQP.

FIG. 19B: RasMol view of 1CQP, the I domain of lymphocyte function-associated antigen in complex with a magnesium ion. The coloured balls represent the amino acids in 1CQP that directly interact with the divalent cation.

FIG. 19C: RasMol view of 1CQP, the I domain of lymphocyte function-associated antigen in complex with a magnesium ion. The coloured balls represent the amino acids in 1CQP which are conserved in CAB52192.1 (AD2) as well.

FIG. 20: SAGE library list.

FIG. 21A: SAGE results for CAB52192.1 (AD2).

FIG. 21B: InterPro search results for CAB52192.1 (G7c; AD2) as of Jul. 24, 2001.

FIG. 21C: InterPro search results for residues 10-126 of CAB52192.1 (G7c; AD2) as of Jul. 24, 2001.

FIG. 21D: NCBI CDD search results for CAB52192.1 (G7c; AD2) as of Jul. 24, 2001.

FIG. 21E: NCBI CDD search results for residues 10-126 of CAB52192.1 (G7c; AD2) as of Jul. 24, 2001.

FIG. 22: Front page of the Biopendium™ Target Mining Interface. Search initiated using 1LFA:A.

FIG. 23A: Inpharmatica Genome Threader™ only results of search using 1LFA:A. The arrow points to KIAA0594.

FIG. 23B: PSI-Blast results from search using 1LFA:A.

FIG. 24: Redundant Sequence Display page for KIAA0564.

FIG. 25: NCBI protein report for KIAA0564 (AD3).

FIG. 26: PFAM search results for KIAA0564 (AD3).

FIG. 27A: Inpharmatica Genome Threader™ only results of search using KIAA0564 (AD3). The arrows point to LFA.

FIG. 27B: PSI-Blast results from search using KIAA0564 (AD3).

FIG. 28A: Genome Threader™ alignment of KIAA0564 (BAA25490.1; AD3) and LFA.

FIG. 28B: Genome Threader™ alignment of KIAA0564 (BAA25490.1; AD3) and 1BHO:2.

FIG. 29A: LigEye for 1LFA:A, which illustrates the sites of interaction of the magnesium ion, and 1LFA:A.

FIG. 29B: RasMol view of 1LFA:A, the I domain of integrin CD11 alpha in complex with a magnesium ion. The coloured balls represent the amino acids in 1 LFA that comprise the MIDAS divalent cation site and are conserved in KIAA0564 (AD3).

FIG. 29C: RasMol view of 1LFA:A, the I domain of integrin CD11 alpha in complex with a magnesium ion. The coloured balls represent the amino acids in 1 LFA that are conserved in KIAA0564 (AD3).

FIG. 30A: SAGE results for KIAA0564 (AD3).

FIG. 30B: NCBI CDD search results for KIAA0564 (AD3) as of Jul. 24, 2001.

FIG. 30C: NCBI CDD search results for residues 1248-1432 of KIAA0564 (AD3) as of Jul. 24, 2001.

FIG. 31: Front page of the Biopendium™ Target Mining Interface. Search initiated using 1BHO:1.

FIG. 32A: Inpharmatica Genome Threader™ only results of search using 1BHO:1. The arrow points to NG37.

FIG. 32B: PSI-Blast results from search using 1BHO:1.

FIG. 33: Redundant Sequence Display page for NG37.

FIG. 34: NCBI protein report for NG37 (AD4).

FIG. 35: PFAM search results for NG37 (AD4).

FIG. 36A: Inpharmatica Genome Threader™ only results of search using NG37 (AD4) as the query sequence, arrow points to 1LFA. Reverse maximised Psi-Blast identifies a model organism homologue, arrow points to CAA87336.1.

FIG. 36B: PSI-Blast results from search using NG37 (AD4).

FIG. 37: Genome Threader™ alignment of NG37.1 (AD4), CAA87336.1, 1CQP:A, and 1CQP:B.

FIG. 38A: LigEye for 1CQP:A, which illustrates the sites of interaction of the magnesium ion, and 1CQP:A.

FIG. 38B: RasMol view of 1CQP:A, the I domain of integrin CD11 beta in complex with a magnesium ion. The coloured balls represent the amino acids in 1CQP that comprise the MIDAS divalent cation site and are conserved in NG37 (AD4).

FIG. 38C: RasMol view of 1CQP:A, the I domain of integrin CD11 beta in complex with a magnesium ion. The coloured balls represent the amino acids in 1CQP that are conserved in NG37 (AD4).

FIG. 38D: InterPro search results for NG37 (AD4) as of Jul. 24, 2001.

FIG. 38E: InterPro search results for residues 308-424 of NG37 (AD4) as of Jul. 24, 2001.

FIG. 38F: NCBI CDD search results for NG37 (AD4) as of Jul. 24, 2001.

FIG. 38G: NCBI CDD search results for residues 308-424 of NG37 (AD4) as of Jul. 24, 2001.

FIG. 39: Front page of the Biopendium™ Target Mining Interface. Search initiated using 1AOX:A.

FIG. 40A: Inpharmatica Genome Threader™ only results of search using 1AOX:A. The arrow points to CAB01991.1.

FIG. 40B: PSI-Blast results from search using 1AOX:A.

FIG. 41: Redundant Sequence Display page for CAB01991.1.

FIG. 42: NCBI protein report for CAB01991.1 (AD5).

FIG. 43: PFAM search results for CAB01991.1 (AD5).

FIG. 44A: Inpharmatica Genome Threader™ only results of search using CAB01991.1 (AD5). The arrows point to 1AOX. Reverse maximised Psi-Blast identifies a homologue, AAF11936.1.

FIG. 44B: PSI-Blast results from search using CAB01991.1 (AD5).

FIG. 45A: Sequence alignment of CAB01991.1 (AD5), and AAF11936.1, 1CQP:A and 1CQP:B.

FIG. 45B: Alignment of P71551 (P71551 is another identifier for CAB01991.1; AD5) and 1BHO:2.

FIG. 46A: LigEye for 1CQP:A, which illustrates the sites of interaction of the magnesium ion, and 1CQP:A.

FIG. 46B: RasMol view of 1CQP:A, the I domain of integrin Alpha 2/Beta 1 in complex with a magnesium ion. The coloured balls represent the amino acids in 1CQP that comprise the MIDAS divalent cation site and are conserved in CAB01991.1 (AD5).

FIG. 46C: RasMol view of 1CQP:A, the I domain of integrin Alpha 2/Beta 1 in complex with a magnesium ion. The coloured balls represent the amino acids in 1CQP that are conserved in CAB01991.1 (AD5).

FIG. 46D: NCBI CDD search results for CAB01991.1 (AD5) as of Jul. 24, 2001.

FIG. 46E: NCBI CDD search results for residues 482-646 of CAB01991.1 (AD5) as of Jul. 24, 2001.

FIG. 47: Front page of the Biopendium™ Target Mining Interface. Search initiated using 1AOX:A.

FIG. 48A: Inpharmatica Genome Threader™ only results of search using 1AOX:A. The arrow points to Rv0368c.

FIG. 48B: PSI-Blast results from search using 1AOX:A.

FIG. 49: Redundant Sequence Display page for Rv0368c.

FIG. 50: NCBI protein report for Rv0368c (AD6).

FIG. 51: PFAM search results for Rv0368c (AD6).

FIG. 52A: Inpharmatica Genome Threader™ only results of search using Rv0368c (AD6). The arrows point to 1AOX. Reverse-maximised Psi-Blast identifies a homologue of Rv0368c, arrow points to BAA81233.1

FIG. 52B: PSI-Blast results from search using Rv0368c (AD6).

FIG. 53A: Sequence alignment of CAA17374.1 (Rv0368c; AD6), BAA81233.1 and 1AOX.

FIG. 53B: Alignment of CAA17374.1 (Rv0368c; AD6) and 1DGQ:A, 1IDO and 1QC5:A.

FIG. 54A: LigEye for 1AOX:A, which illustrates the sites of interaction of the magnesium ion, and 1AOX:A.

FIG. 54B: RasMol view of 1AOX:A, the I domain of integrin Alpha 2/Beta 1 in complex with a magnesium ion. The coloured balls represent the amino acids in 1AOX that comprise the MIDAS divalent cation site and are conserved in Rv0368c (AD6).

FIG. 54C: RasMol view of 1AOX:A, the I domain of integrin Alpha 2/Beta 1 in complex with a magnesium ion. The coloured balls represent the amino acids in 1AOX that are conserved in Rv0368c (AD6)

FIG. 54D: InterPro search results for Rv0368c (AD6) as of Jul. 24, 2001.

FIG. 54E: InterPro search results for residues 230-370 of Rv0368c (AD6) as of Jul. 24, 2001.

FIG. 54F: NCBI CDD search results for Rv0368c (AD6) as of Jul. 24, 2001.

FIG. 54G: NCBI CDD search results for residues 230-370 of Rv0368c (AD6) as of Jul. 24, 2001.

### EXAMPLES

### Example 1: KIAA0301 (BAA20761.1; AD1)

In order to initiate a search for novel, distantly related integrins, an archetypal family member, Leukocyte Function Associated Molecule-1 (LFA), alpha subunit is chosen. More specifically, the search is initiated using a structure from the Protein Data Bank (PDB) which is operated by the Research Collaboratory for Structural Bioinformatics.

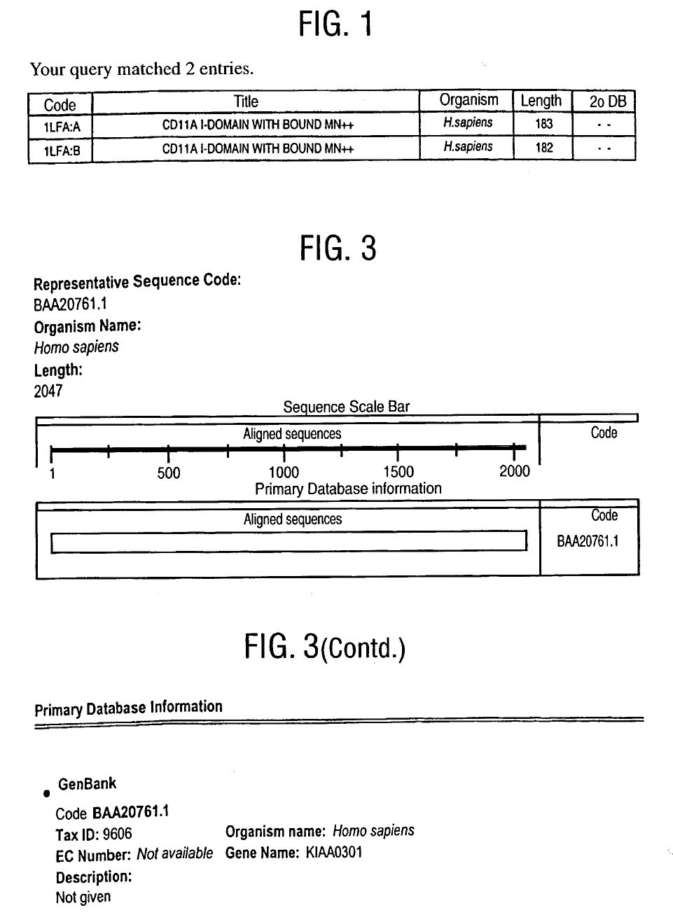

The structure chosen represents the I domain (insertion domain) of LFA-1, PDB code 1LFA:A (FIG. 1). Lymphocyte function-associated antigen 1 (LFA-1) is a leukocyte integrin that supports inflammatory and immune responses by mediating cell adhesion, the trafficking of leukocytes, and the augmentation of signalling through the T cell receptor. This integrin consists of a CD11a and a CD18 chain and binds to the cell surface ligands intercellular adhesion molecule 1 (ICAM-1), ICAM-2, and ICAM-3. Mutational studies indicate that ICAM-1 interacts with LFA-1 through a module of approximately 200 residues designated the I domain that is located in CD11. The I domain is the site of interaction between integrins and intercellular adhesion molecules. Integrin I domains are homologous to the A-domains present in von Willebrand factor, several collagen and complement proteins, and cartilage matrix protein, all proteins with adhesive functions (Huth, J. R., et al., Proc Natl Acad Sci U.S.A. 2000 97(10):5231-6).

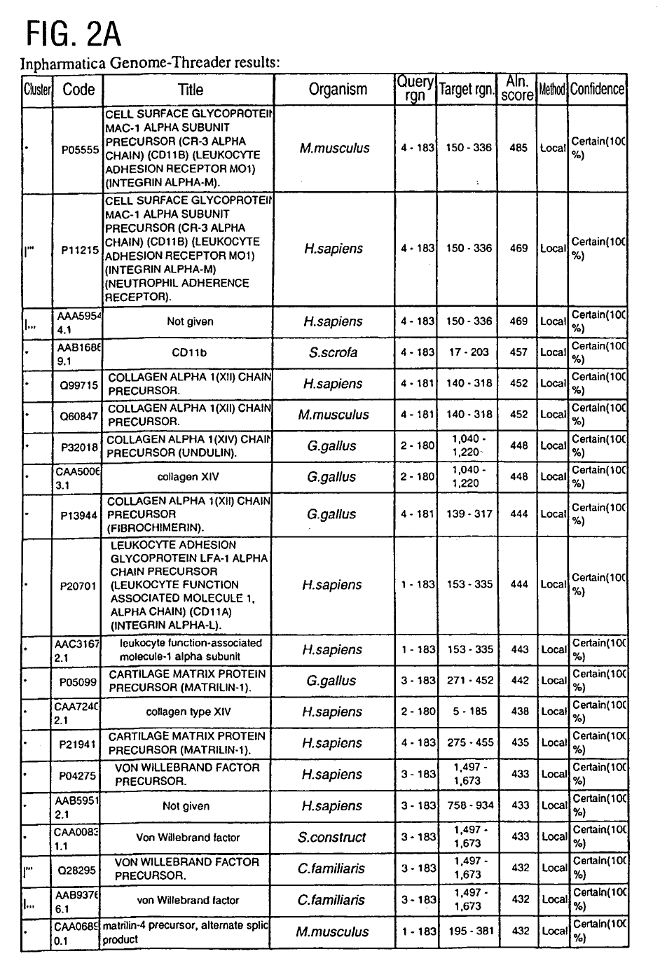

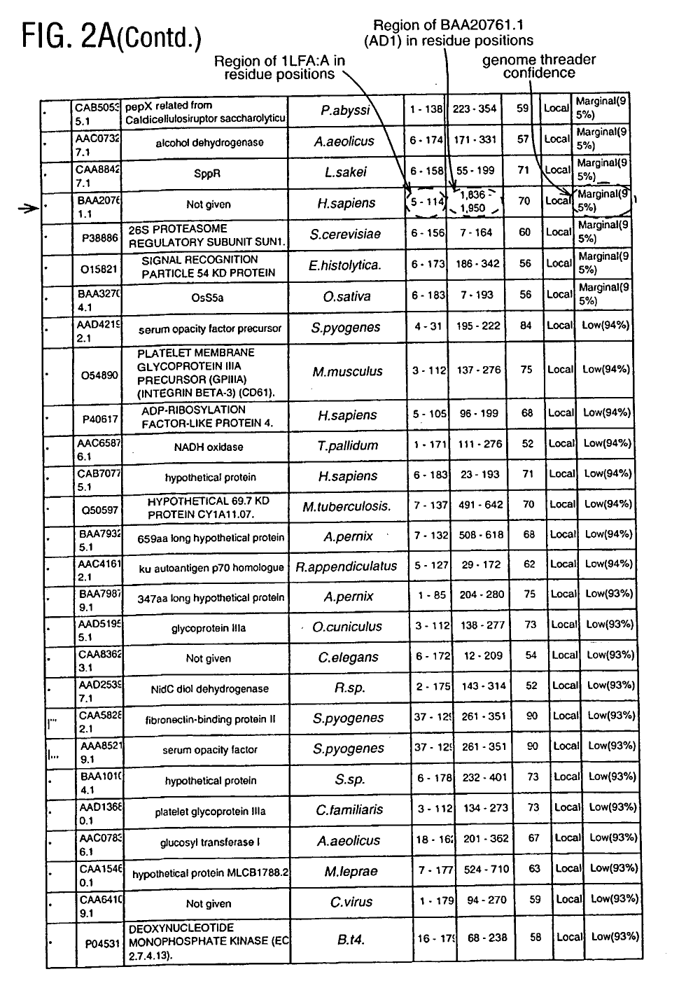

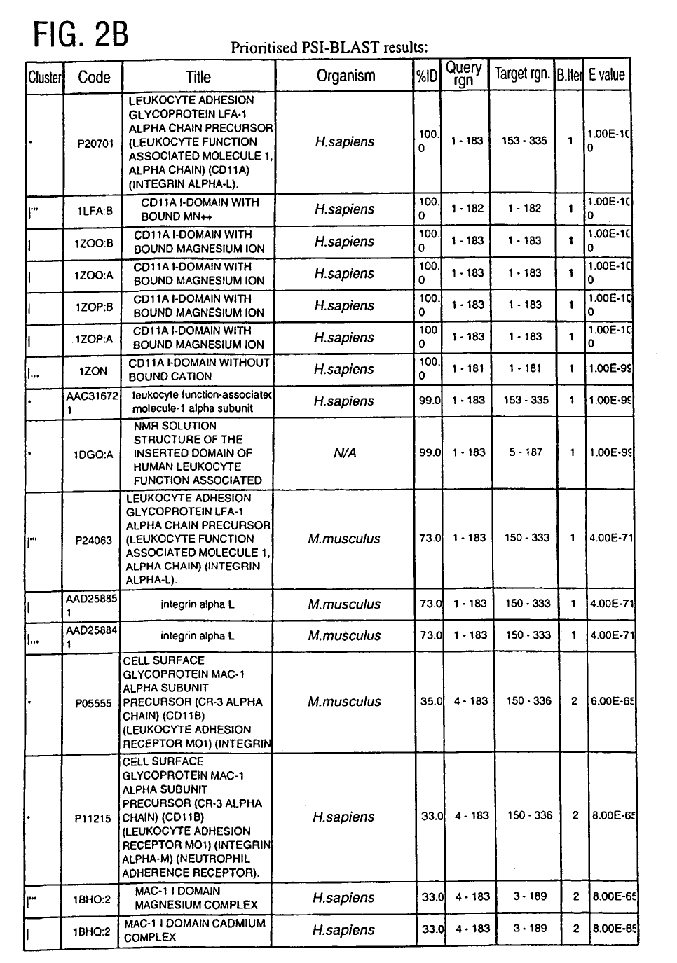

A search of the Biopendium™ for homologues of 1LFA takes place and returns 559 Inpharmatica Genome Threader™ results (selection given in FIG. 2A) and 595 PSI-Blast results (selection in FIG. 2B). The 559 Genome Threader™ results include examples of other I domain containing integrins, such as H. sapiens MAC-1 and LFA-1 as well as Collagen alpha 1 and Von Willebrand Factor. Among the known I domain containing adhesion molecules/proteins appears a protein of apparently unknown function, “Not Given” (BAA20761.1; AD1, FIG. 2A).

The Inpharmatica Genome Threader™ has identified residues 1836-1950 of a sequence, BAA20761.1 (AD1), as having an equivalent structure to residues 5-114 of the I domain of LFA-1 (PDB code 1LFA:A), the known interaction domain between LFA-1 and ICAM. Having a structure similar to this domain suggests that BAA20761.1 (AD1) is a protein that functions in cellular adhesion. The Inpharmatica Genome Threader™ identifies this with 95% confidence.

PSI-Blast (FIG. 2B) is unable to identify this relationship; it is only the Inpharmatica Genome Threader™ that is able to identify residues 1836-1950 of BAA20761.1 (AD1) as having an I domain. PSI-Blast does identify LFA-1 itself and other related integrins with varying degrees of probability (E value) as would be expected.

In order to view what is known in the public domain databases about BAA20761.1 (AD1), the Redundant Sequence Display Page (FIG. 3) is viewed. BAA20761.1 (AD1) is a Homo sapiens sequence, its GenBank protein ID is BAA20761.1, its gene name is KIAA0301 and it is 2047 amino acids in length. There are no associated PROSITE or PRINTS hits for this sequence. PROSITE and PRINTS are databases that help to describe proteins of similar families. Returning zero hits from both databases means that BAA20761.1 (AD1) is unidentifiable as an I domain containing adhesion molecule using PROSITE or PRINTS.

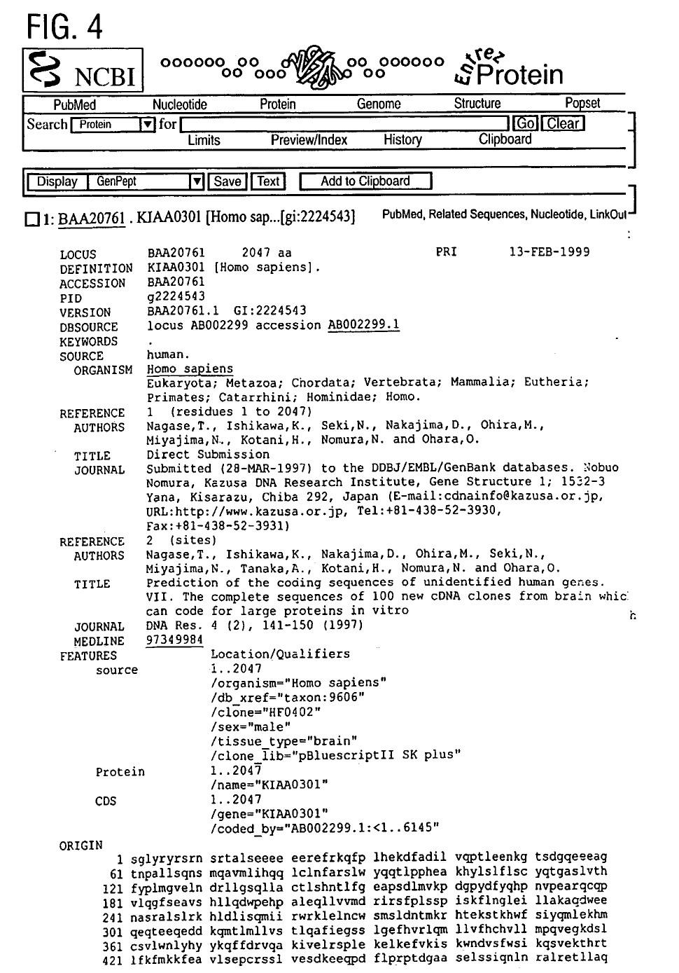

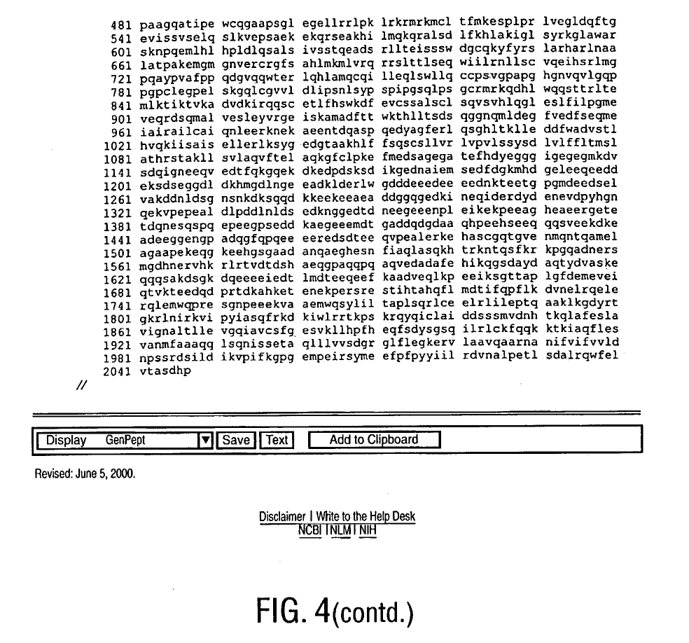

The National Centre for Biotechnology Information (NCBI) GenBank protein database is viewed to examine if there is any further information that is known in the public domain relating to BAA20761.1 (AD1). This is the U.S. public domain database for protein and gene sequence deposition (FIG. 4). BAA20761.1 was cloned by a group of scientists in Chiba, Japan (Nagase, T. et al, (1997) DNA Res. 4(2): 141-150). There is no further annotation for BAA20761.1 except that the BAA20761.1 gene was cloned from brain tissue. The public domain information for this gene does not annotate it as an integrin or an I domain-containing protein, or indeed, contain any suggestion whatsoever for the function of this protein.

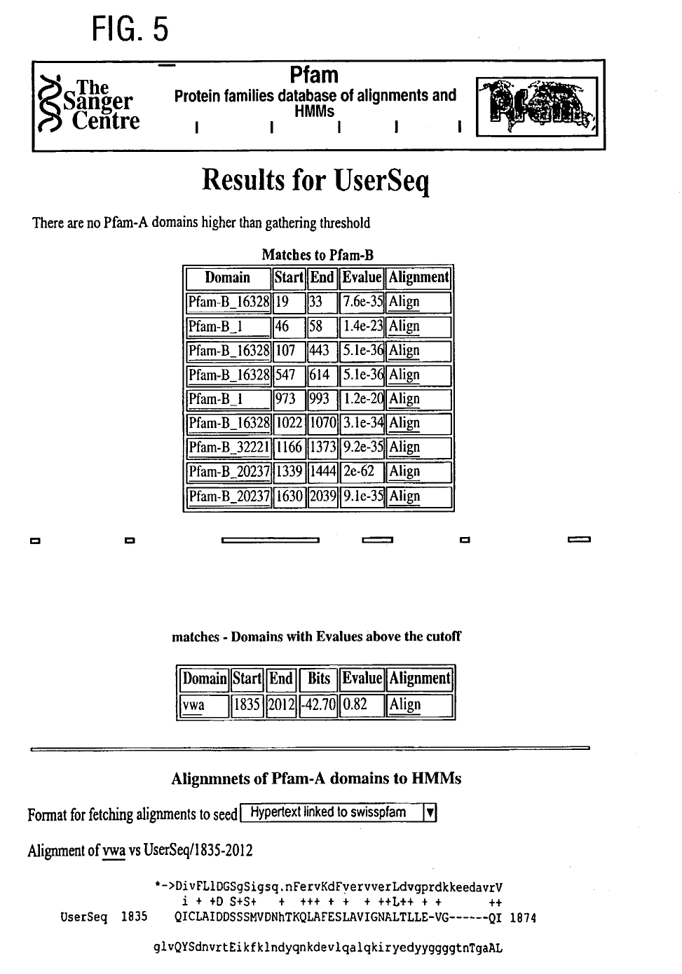

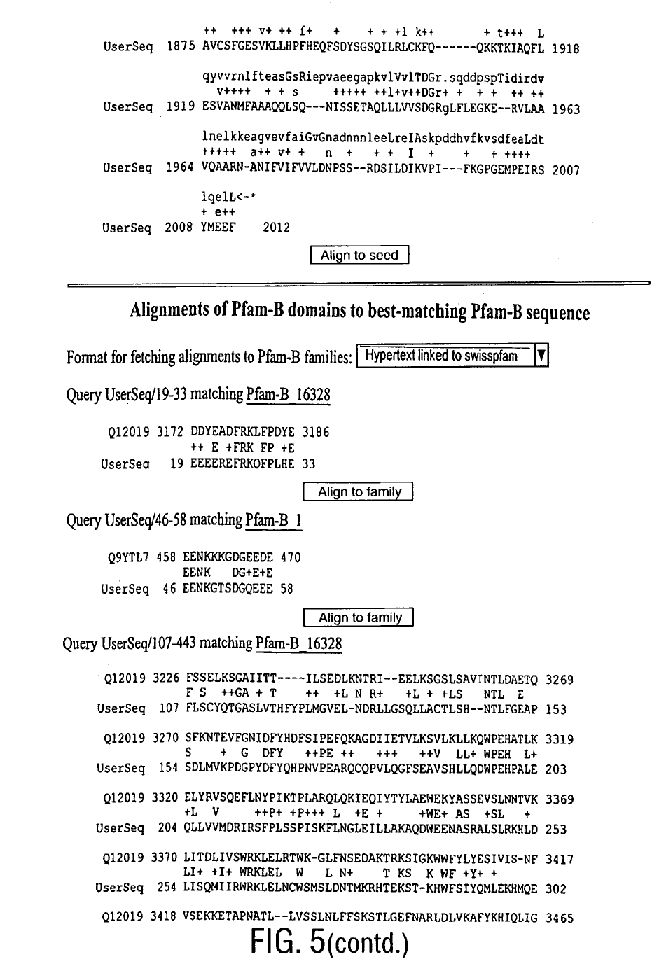

In order to identify whether any other public domain annotation vehicle is able to annotate BAA20761.1 as an I domain containing protein, the BAA20761.1 protein sequence is searched against the Protein Family Database of Alignment and HMM's (PFAM) database (FIG. 5). The results identify that BAA20761.1 has no identifiable PFAMs. It may have a Von Willebrand domain (VWA) but this is below the threshold of credibility: the certainty of this is very low (E=0.82) and as such is not reliable. PFAM does not identify BAA20761.1 (AD 1) as having an I domain.

Therefore using all public domain annotation tools BAA20761.1 (AD1) is not annotated as a protein involved in adhesion through the presence of an I domain. Only the Inpharmatica Genome Threader™ is able to annotate this protein as possessing an I domain.

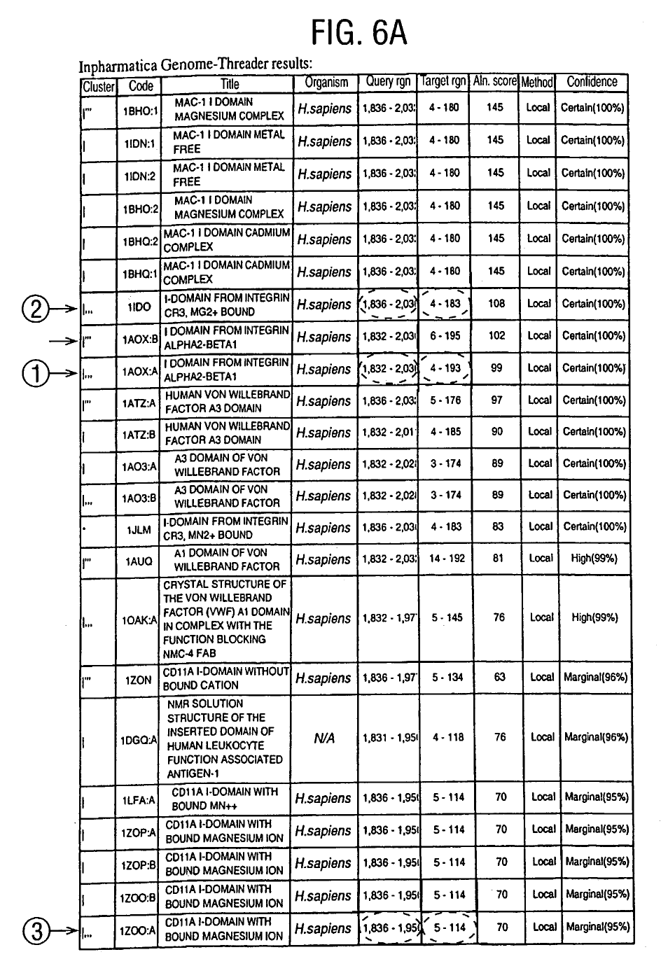

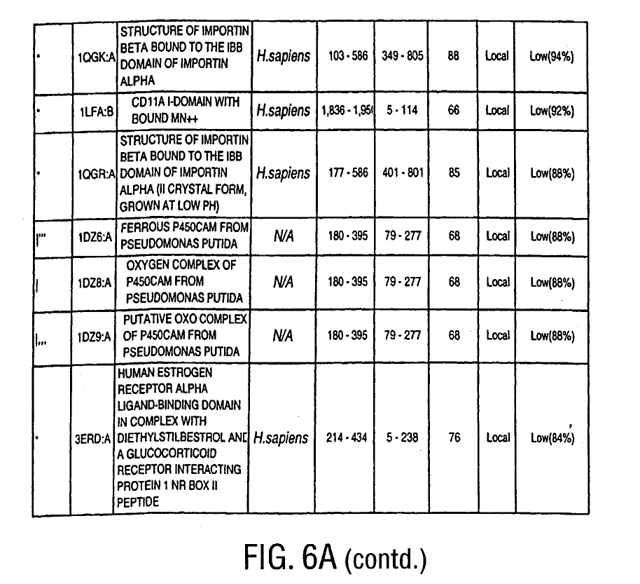

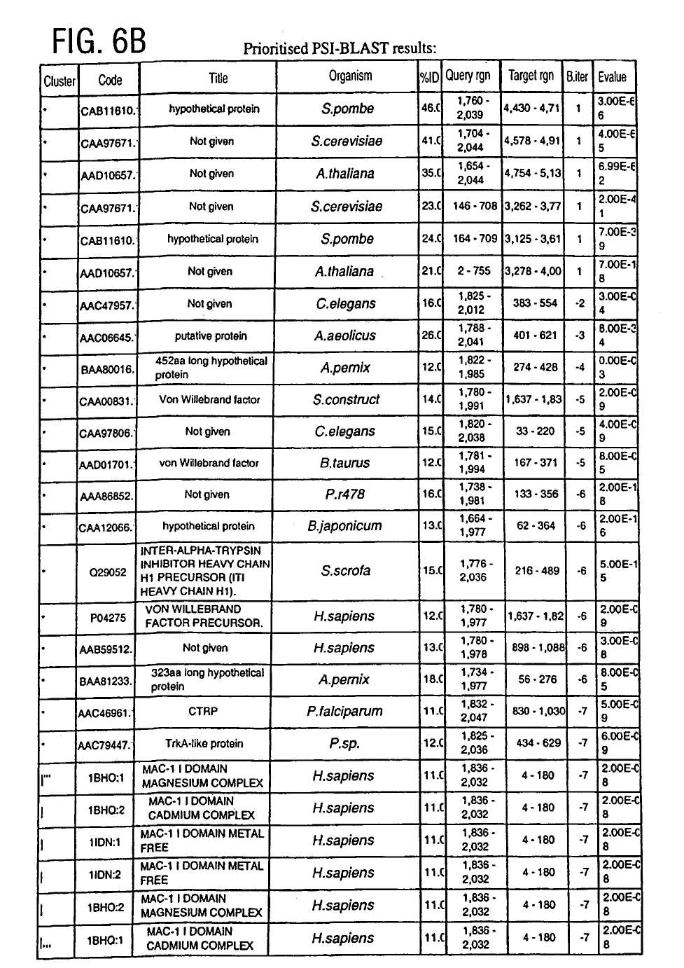

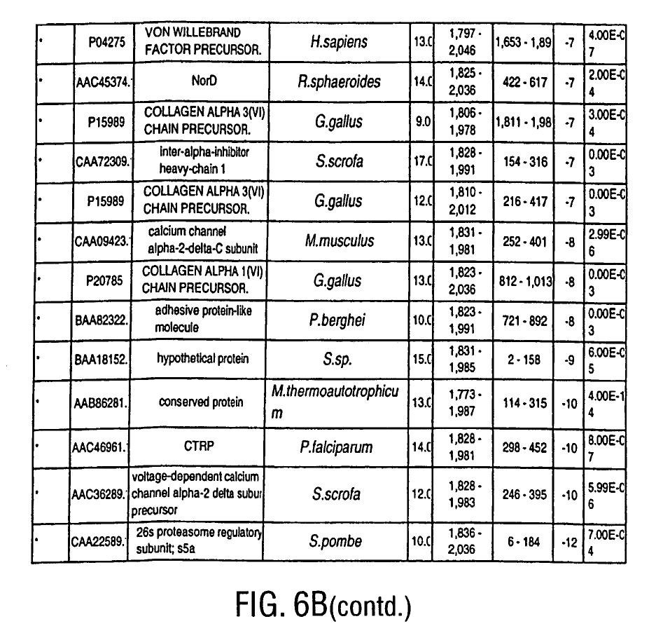

The reverse search is now carried out. BAA20761.1 (KIAA0301; AD1) is now used as the query sequence in the Biopendium™. The Inpharmatica Genome Threader™ identifies 30 bits (FIG. 6A) while PSI-Blast returns 39 hits (FIG. 6B). The Inpharmatica Genome Threader™ (FIG. 6A, arrow \mathring{1}) identifies residues 1832-2036 of BAA20761.1 (AD1) as having a structure the same as the I domain of the integrin Alpha 2-Beta 2 (PDB code: 1AOX:A) with 100% confidence. The Inpharmatica Genome Threader™ (FIG. 6A, arrow \mathring{2}) also identifies residues 1836-2036 of BAA20761.1 (AD1) as having a structure the same as the I-domain of the integrin MAC-1 (PDB code: 1IDO) with 100% confidence. The Inpharmatica Genome Threader™ (FIG. 6A, arrow \mathring{3}) also identifies residues 1836-1950 of BAA20761.1 (AD I) as having a structure the same as the I-domain of the integrin LFA-1 (PDB code: 1ZOO:A) with 95% confidence. Thus a region from residues 1832-1836 to residues 1836-1950 of BAA20761.1 (AD1) has been identified as adopting an equivalent fold to a range of I-domains including those of the adhesion molecules Integrin Alpha2, MAC-1, and LFA-1. Forward PSI-Blast does not return this result. PSI-Blast is only able to identify this relationship in the negative iteration, which the Biopendium™ computes through its all-by-all calculation. It is only the Inpharmatica Genome Threader™ and negative iteration PSI-Blast that is able to identify this relationship.

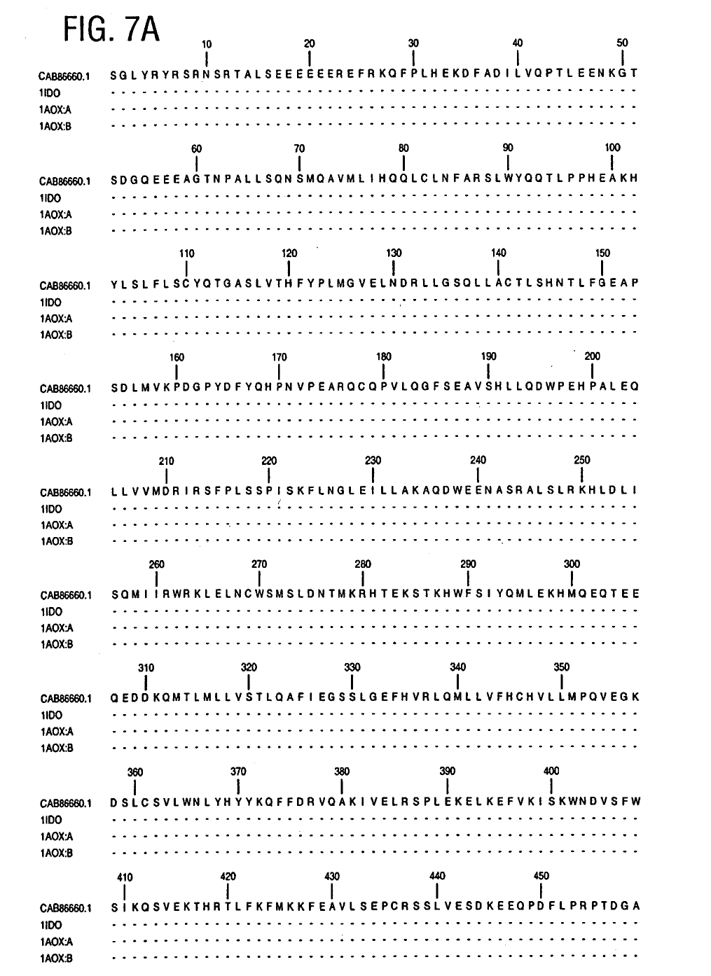

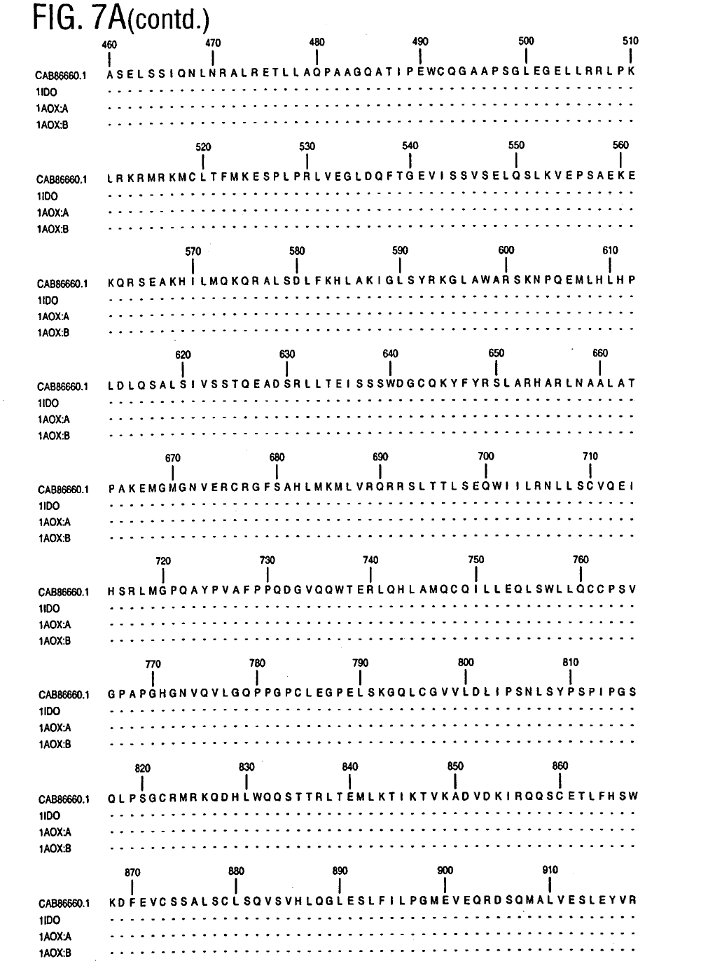

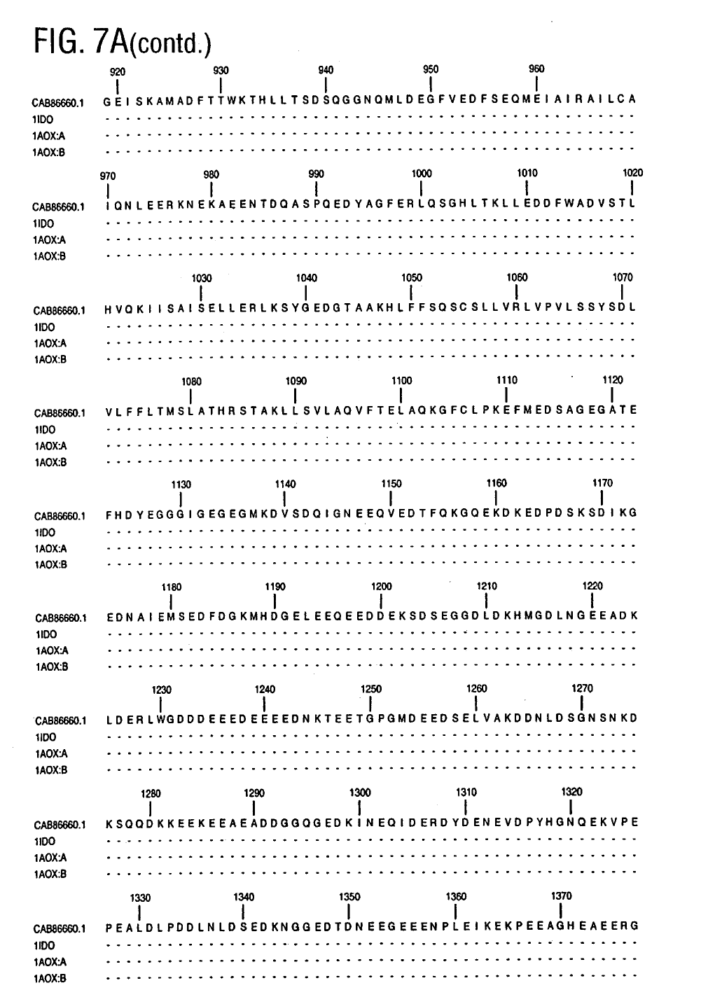

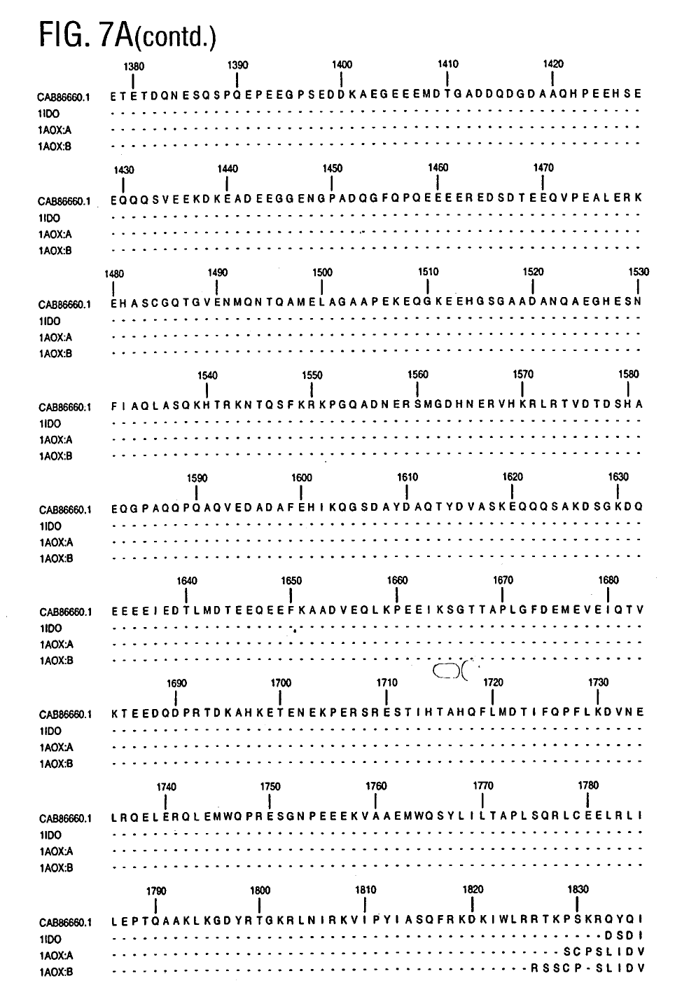

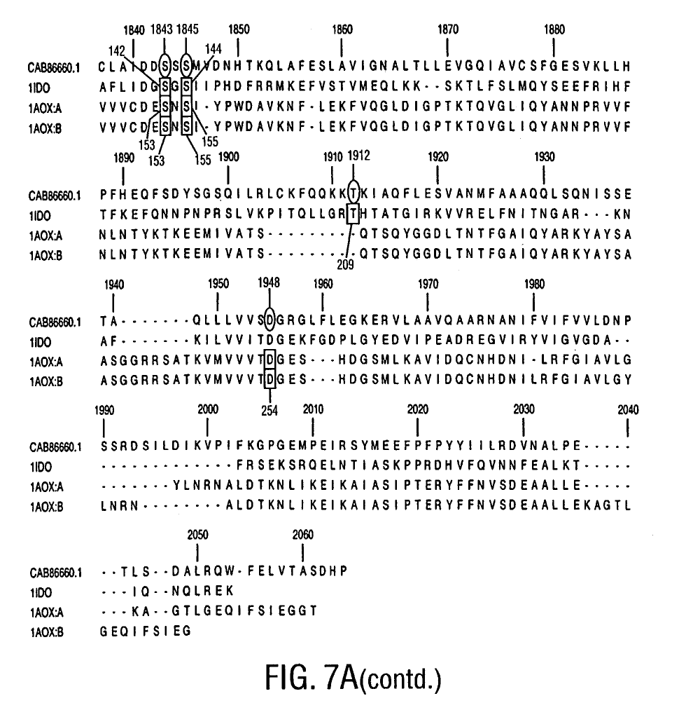

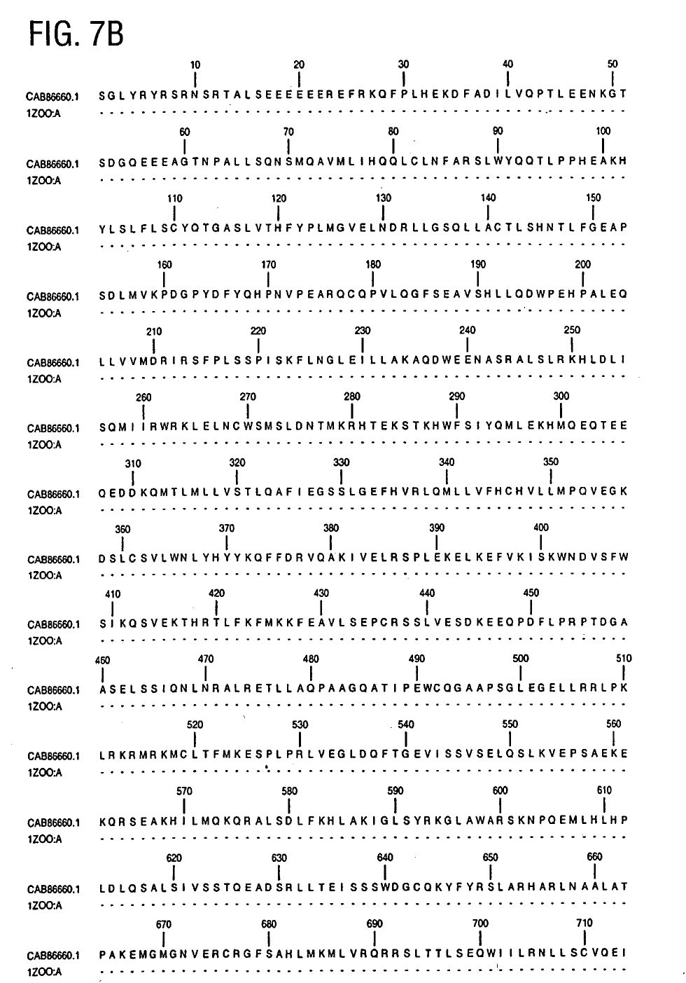

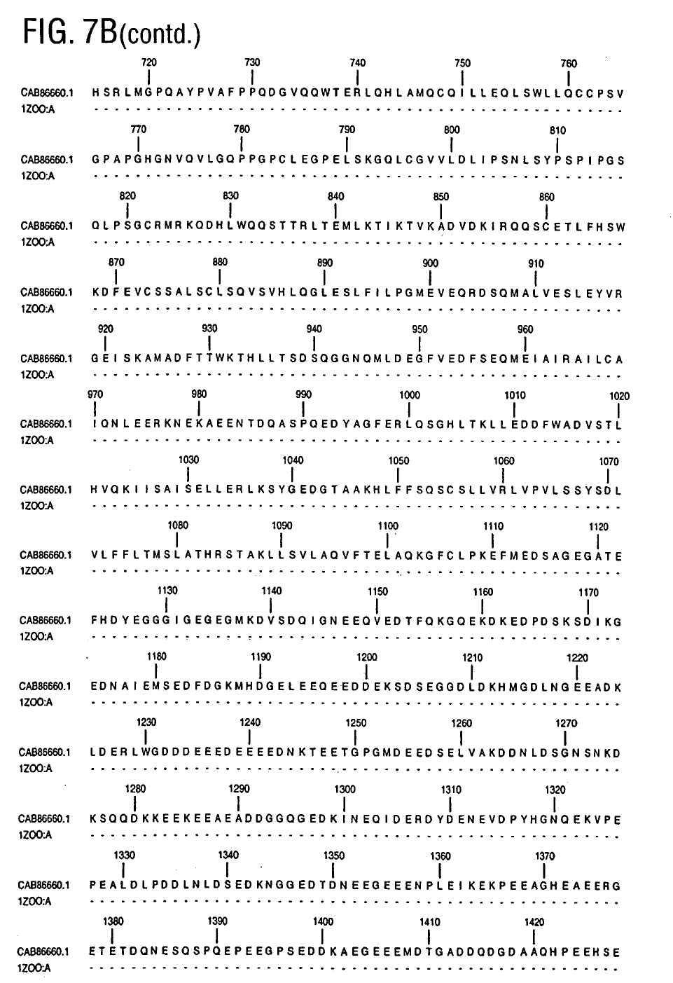

Among the Integrins I domains that the Inpharmatica Genome Threader™ returns is the I domain from Integrin MAC-1 (also known as CR3; PDB code: 1IDO), and the I domain from Integrin Alpha 2-Beta 1 (1AOX:B, 1AOX:A). These are chosen (highlighted) against which to view the sequence alignment of BAA20761.1 (the AD1 polypeptide which also has the identifier CAB86660.1). Viewing the alignment (FIG. 7A) of the query protein against the proteins identified as being of a similar structure helps to visualize the areas of homology. FIG. 7A illustrates the point that the divalent cation binding residues of 1AOX (Ser153, Ser155 and Asp254) are conserved as Ser1843, Ser1845 and Asp1948 in BAA20761.1 (the AD1 polypeptide which also has the identifier CAB86660.1). Furthermore, FIG. 7A also illustrates that the metal ion binding residues of 1IDO (Ser142, Ser144 and Thr209) are conserved as Ser1843, Ser1845 and Thr1912 in BAA20761.1 (the AD1 polypeptide which also has the identifier CAB86660.1). Thus BAA20761.1 (the AD1 polypeptide which also has the identifier CAB86660.1) has two potential metal ion binding triads (Ser1843, Ser1845 and Asp1948) or(Ser1843, Ser1845 and Thr1912).

FIG. 7B shows the Genome Threader™ alignment of BAA20761.1 (the AD1 polypeptide which also has the identifier CAB86660.1) with the Integrin LFA-1 I-domain (1ZOO:A). FIG. 7B illustrates that the metal ion binding residues of 1ZOO:A (Ser139, Ser141 and Asp239) are conserved as Ser1843, Ser1845 and Asp1948 in BAA20761.1 (the AD1 polypeptide which also has the identifier CAB86660.1).

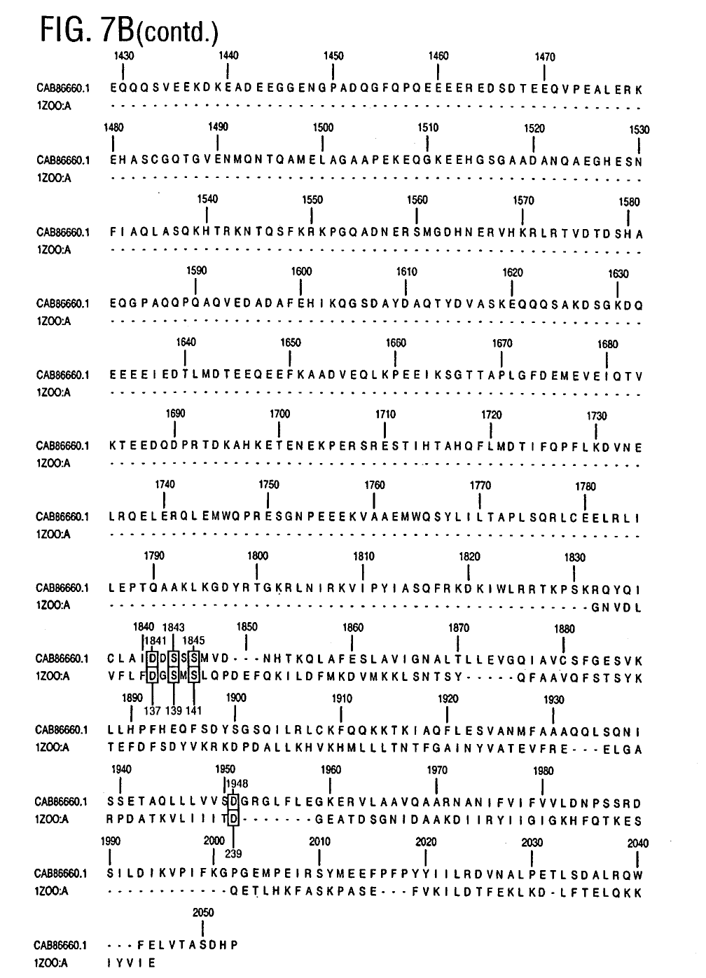

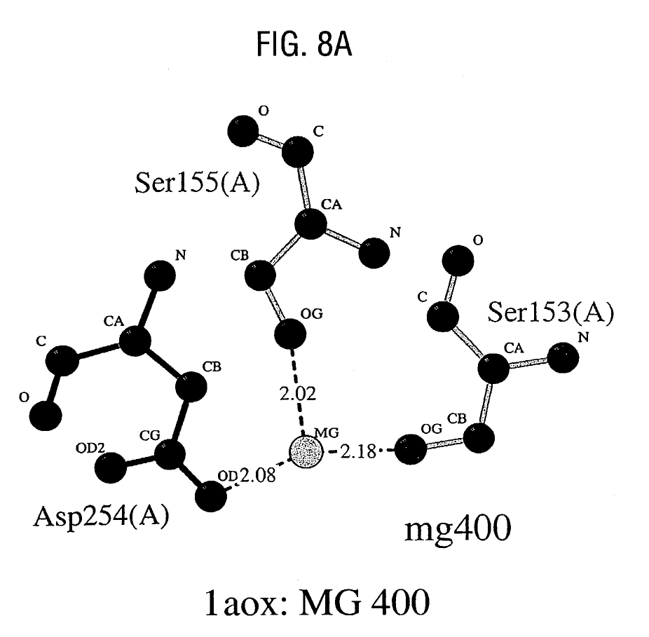

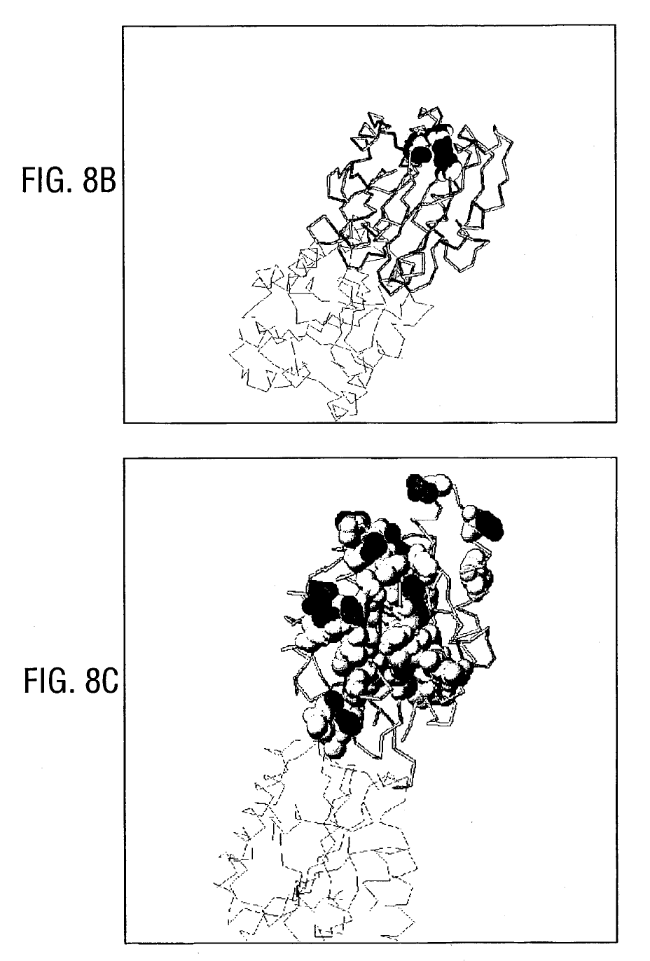

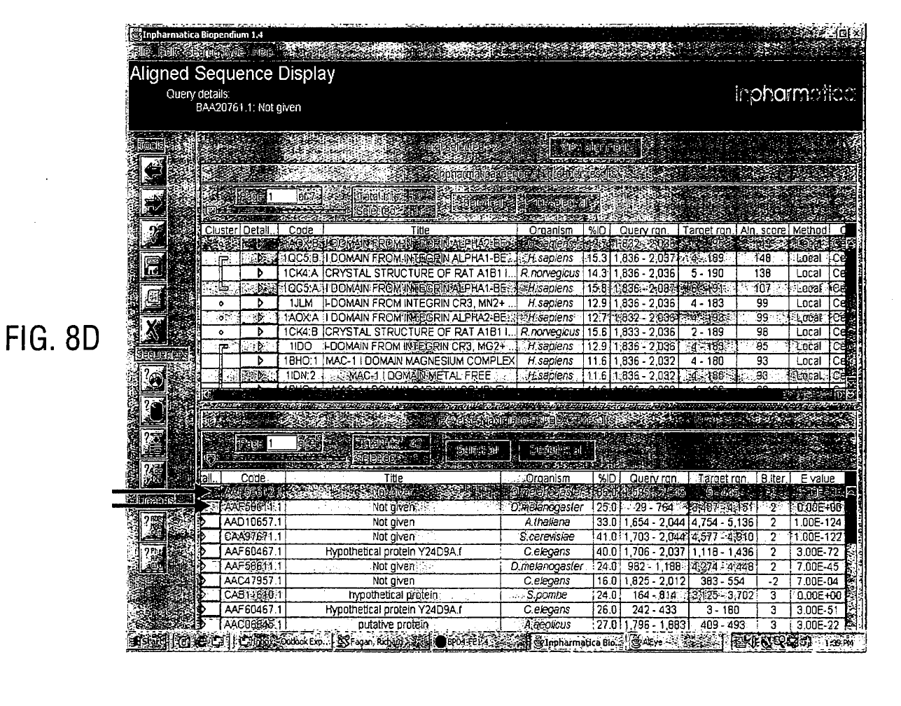

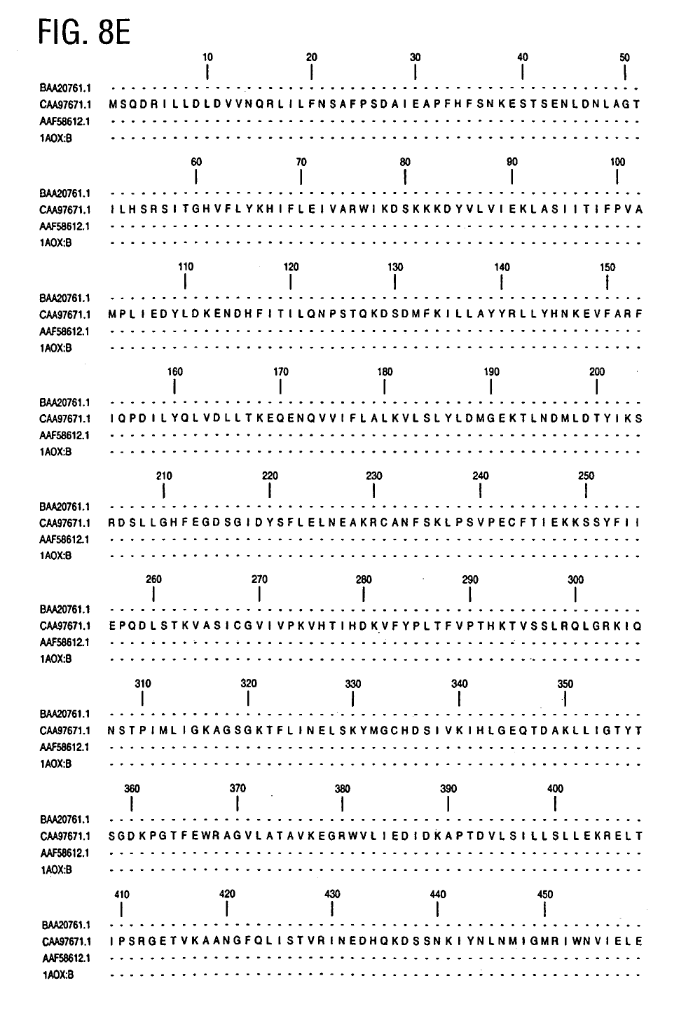

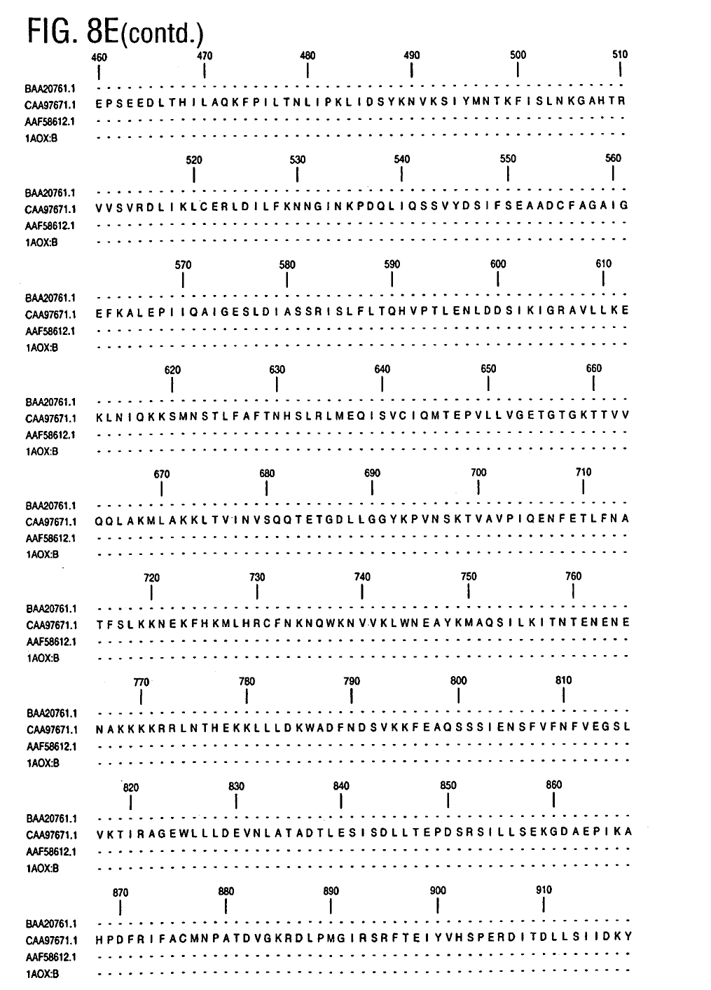

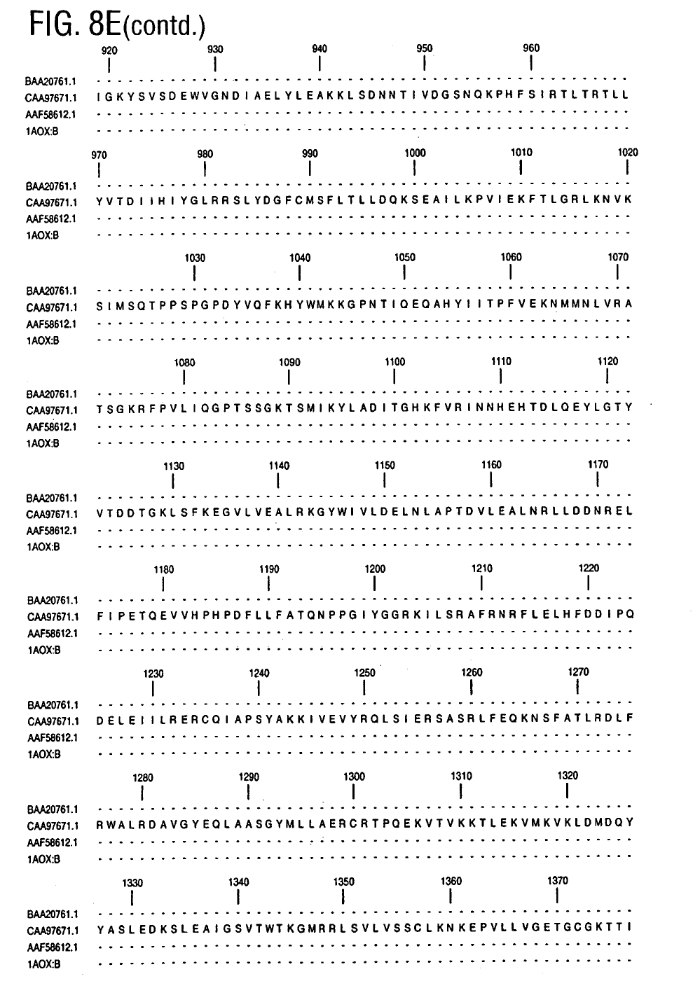

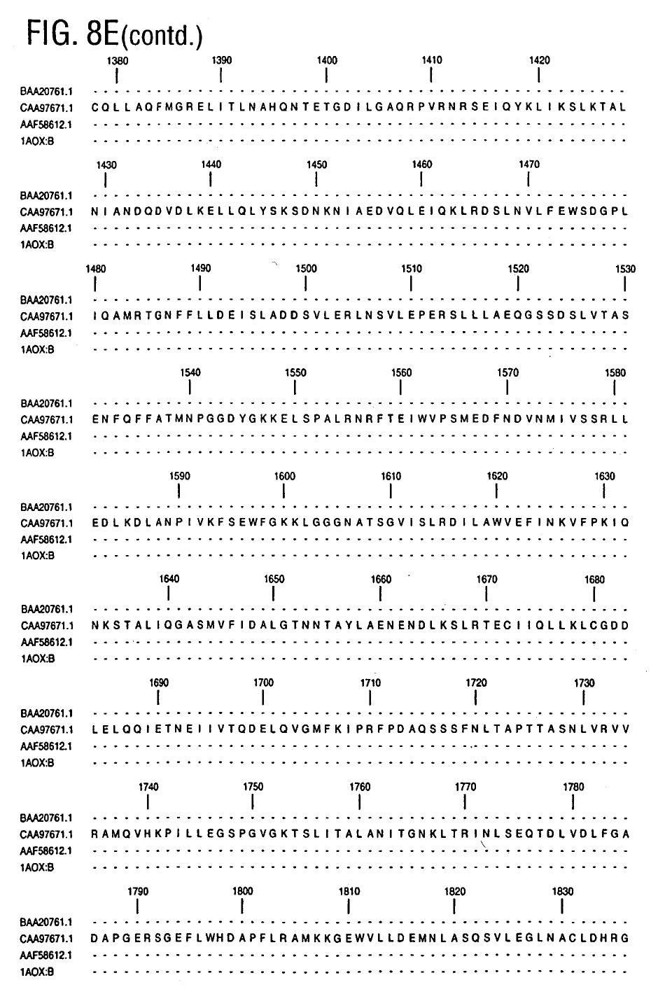

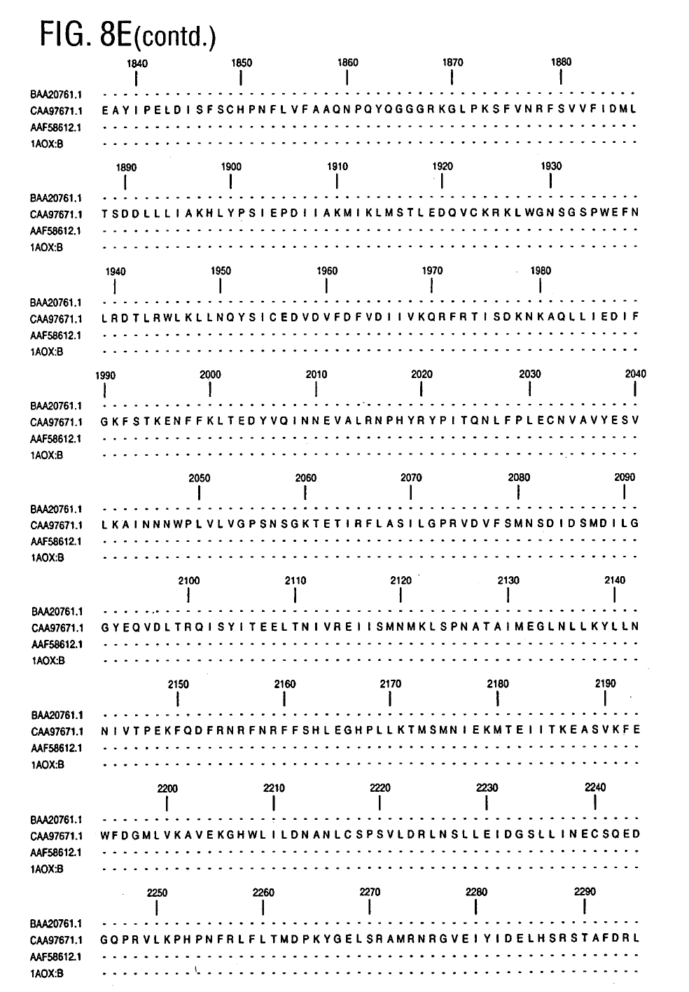

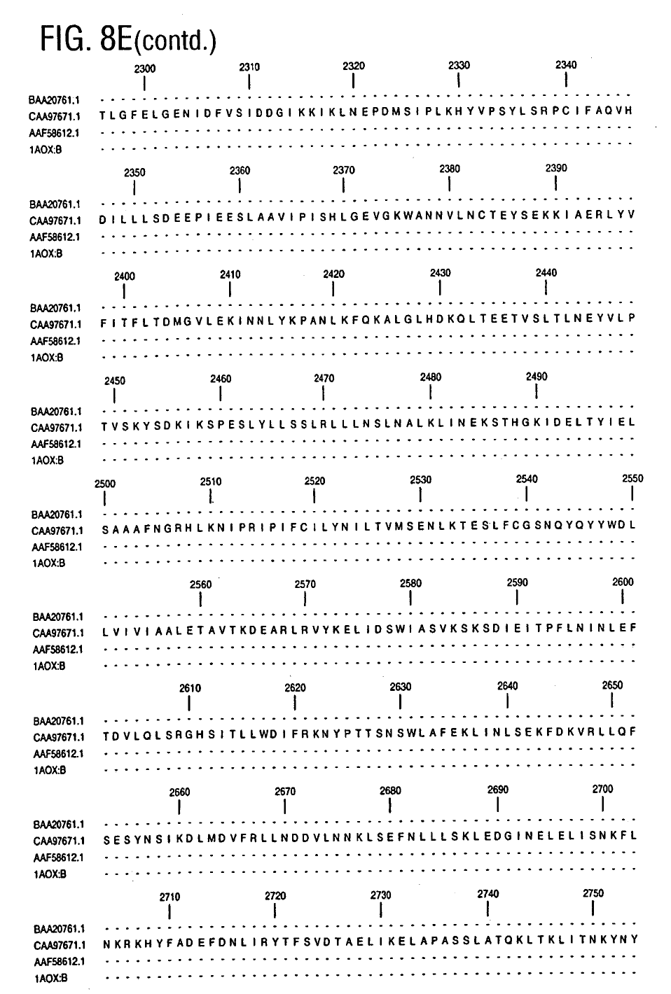

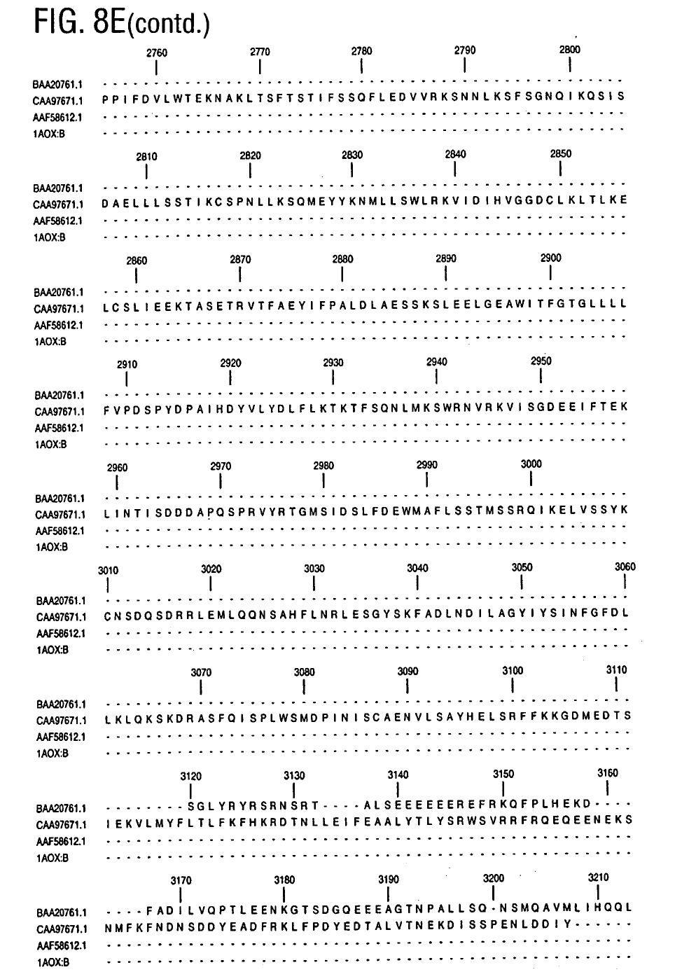

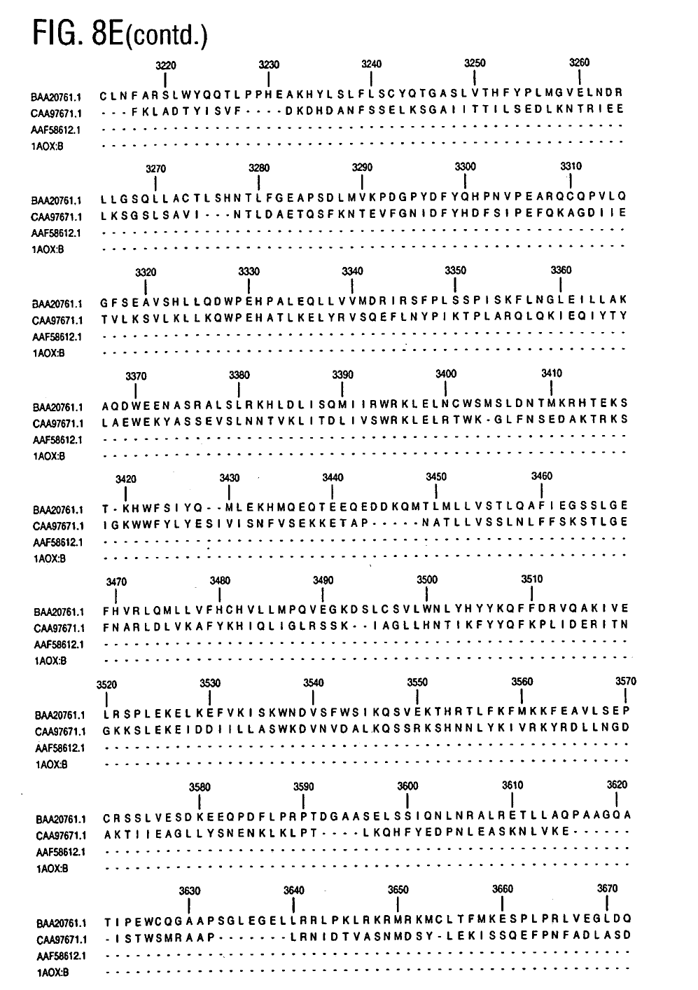

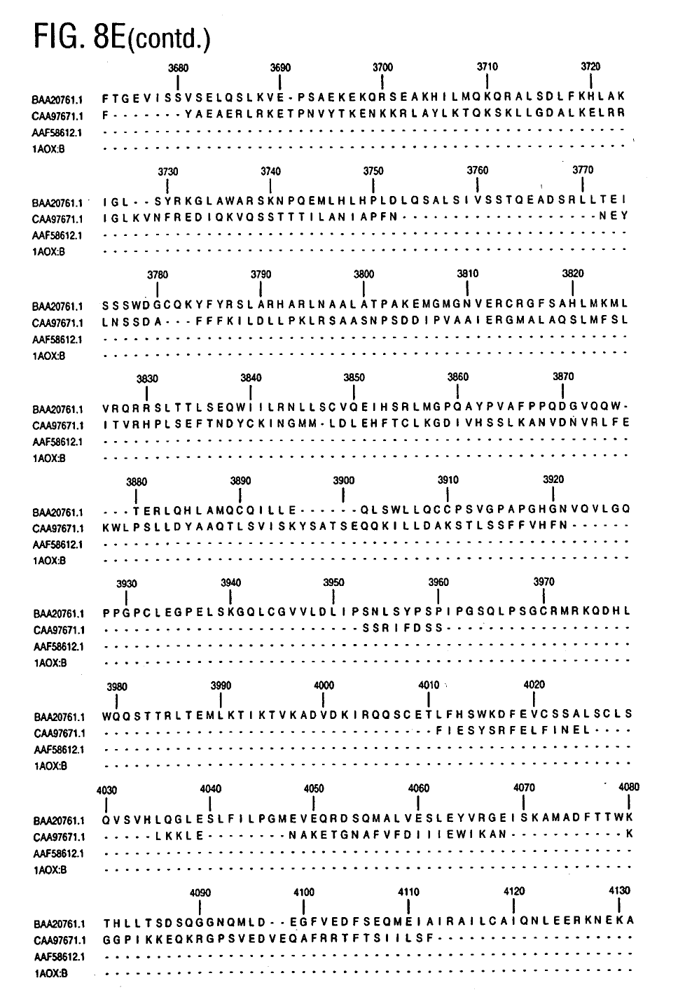

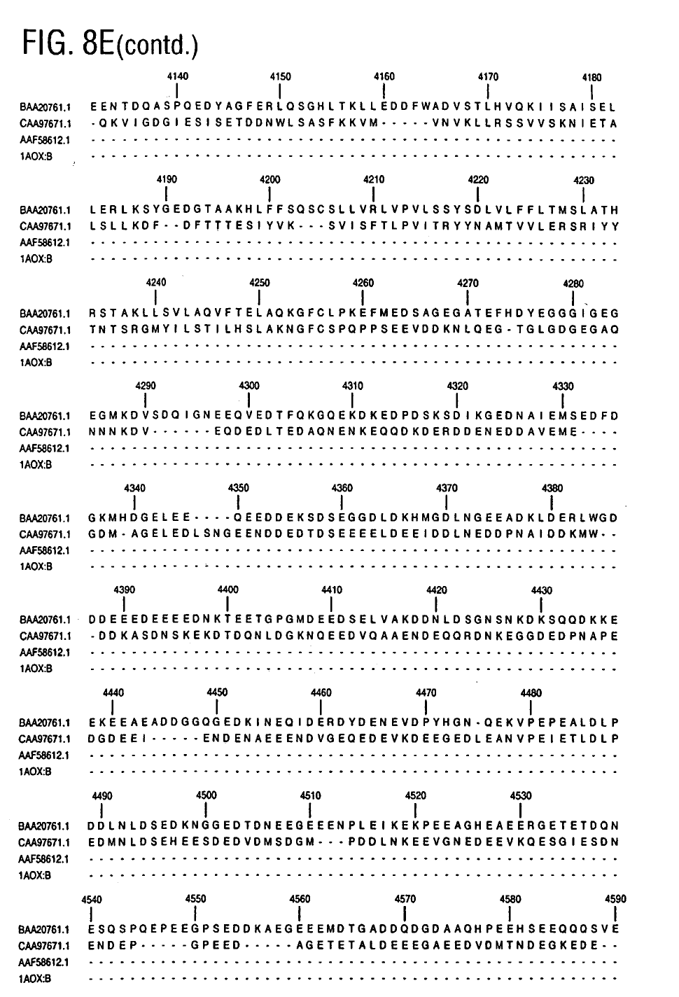

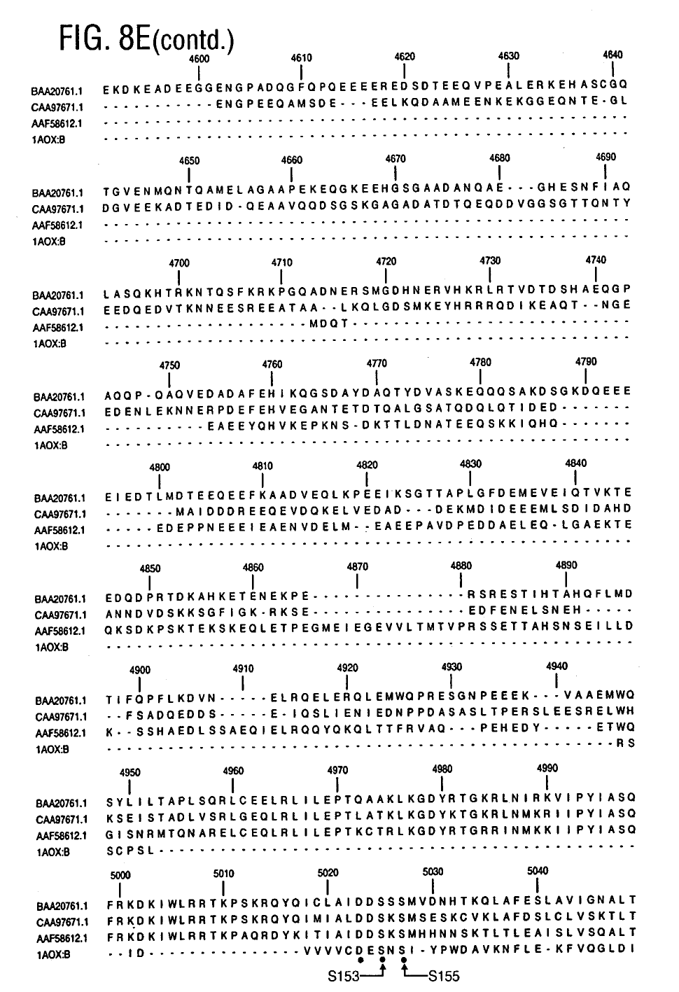

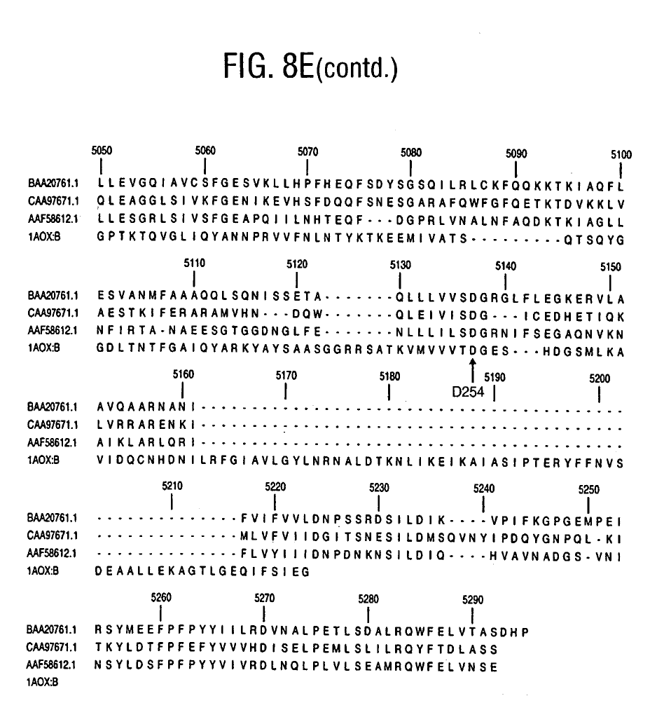

In order to ensure that the protein identified is a homologue of the query sequence, the visualisation program LigEye (FIG. 8A) and RasMol (FIG. 8B) are used. These visualization tools identify the active site of known protein structures by indicating the amino acids with which known small molecule inhibitors interact at the active site. These interactions are through either a direct hydrogen bond or hydrophobic interactions. In this manner one can see if the active site fold/structure is conserved between the identified homologue and the chosen protein of known structure.

This visualisation is shown with 1AOX, which illustrates the sites of interaction of a magnesium ion with the I domain of Integrin Alpha 2-Beta 1 (FIG. 8A). The magnesium ion sees 3 different amino acids in the I domain of 1AOX. The three amino acids of 1AOX (SER153, SER155, and Asp254) are conserved in BAA20761.1 (FIG. 8B, ball structures). FIG. 8C further identifies the amino acids that are conserved in 1AOX and BAA20761.1. The conservation of amino acids indicates that the fold is identical. This indicates that indeed as predicted by the Inpharmatica Genome Threader™, BAA20761.1 (AD1) folds in a similar manner to 1AOX and as such is identified as an I domain containing protein.

Inpharmatica's Reverse Maximised Psi-Blast identifies two homologues of AD1 (FIG. 8D, arrows) in D. melanogaster, AAF58612.1 and AAF58611.1. The sequence identity between these homologues and AD1 is relatively low, 36% and 25% respectively. Neither of these homologues are annotated as having an I domain. FIG. 8E shows an alignment between AD1 (BAA20761.1) CAA97671.1, AAF58612.1 and 1AOX:B. Although these sequences have diverged signifigantly we see that the functional I domain residues of 1AOX, Ser153, Ser155 and Asp254 are absolutely conserved in the homologues. This indicates that these residues are of functional importance to the protein, indicating that BAA20761.1 does indeed contain an I domain. Residues which are essential for the function of a protein will be conserved in homologues of that protein. Thus the precise conservation of identified functional I domain residues strongly supports the annotation of BAA20761.1 as an I domain containing protein.

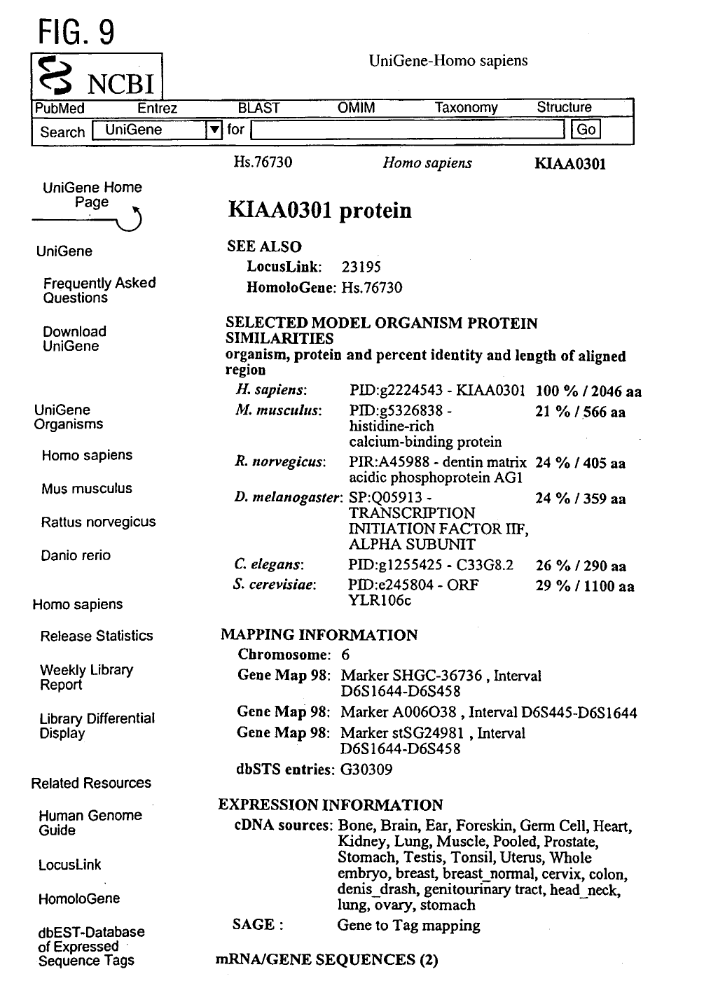

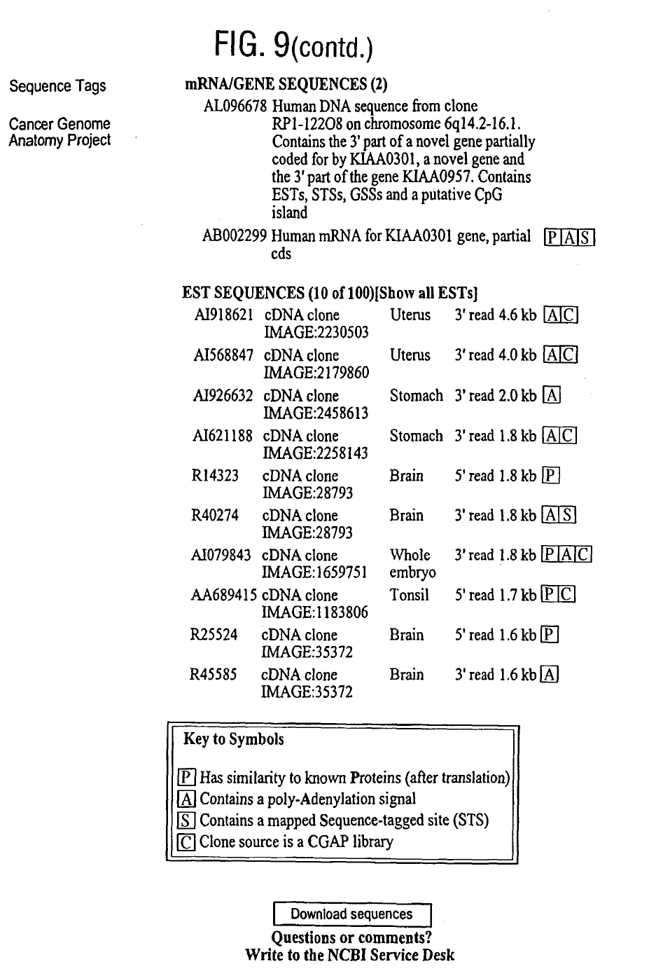

FIG. 9 is a report generated from the NCBI UniGene database. This database is a collection of expressed sequence tags (ESTs). As this is a database of expressed sequences from a range of tissues from the human body, it can be used to give a general tissue distribution for a protein provided that its sequence is present in the database. BAA20761.1 (AD1) is presented in the database and is expressed in a wide range of tissues.

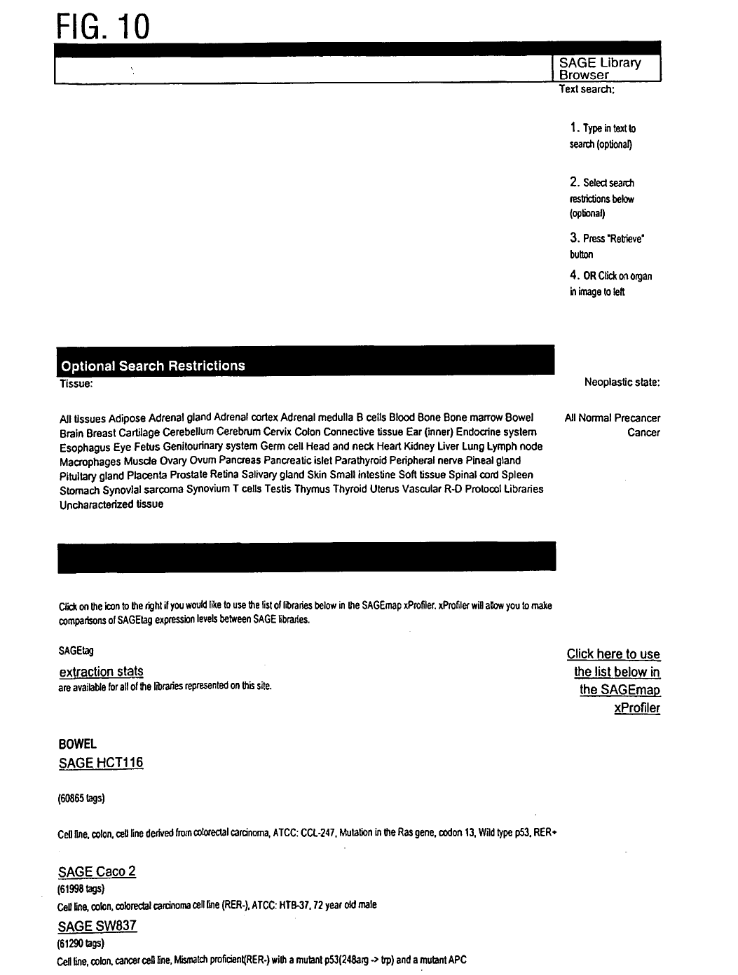

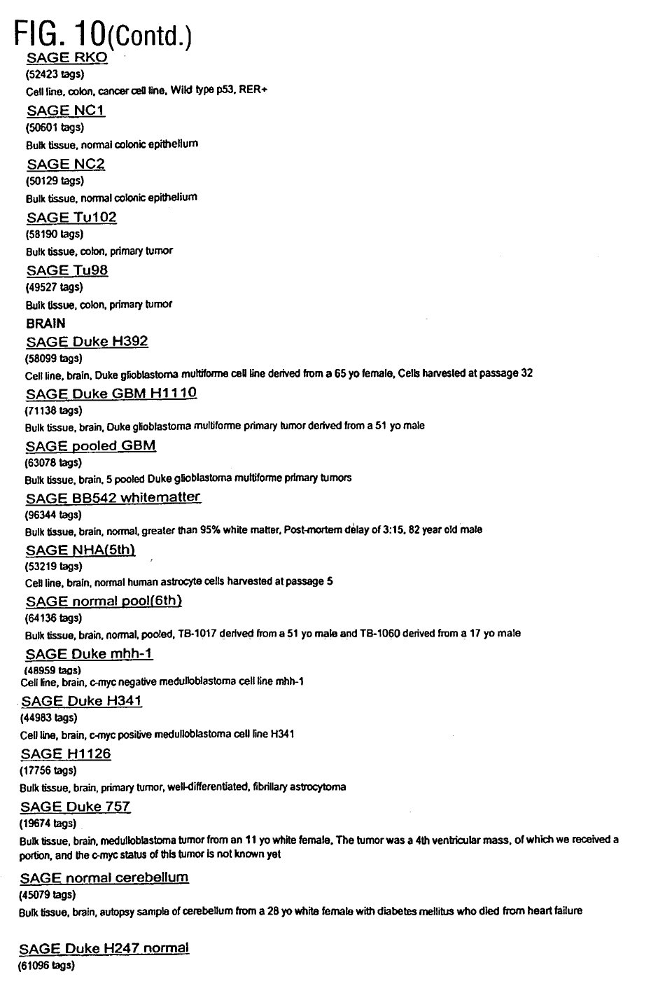

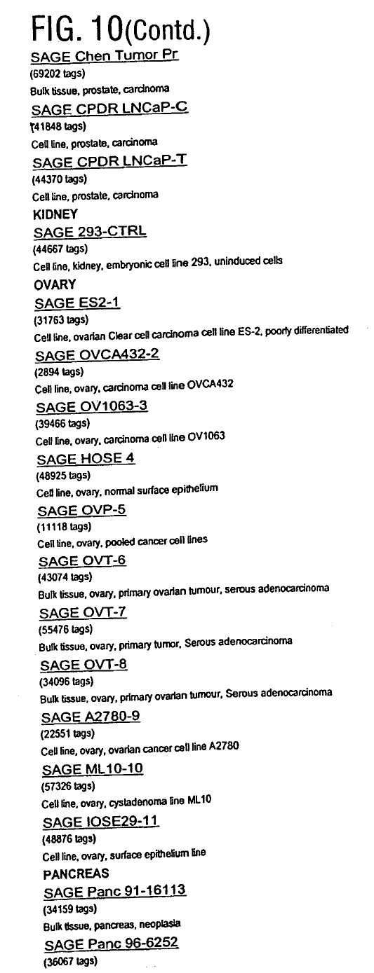

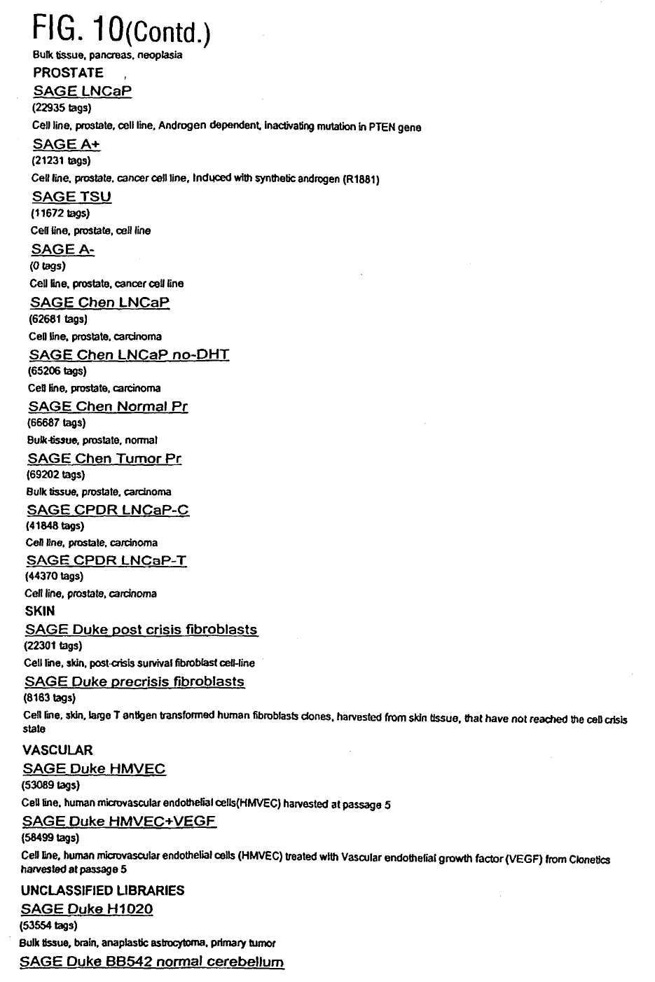

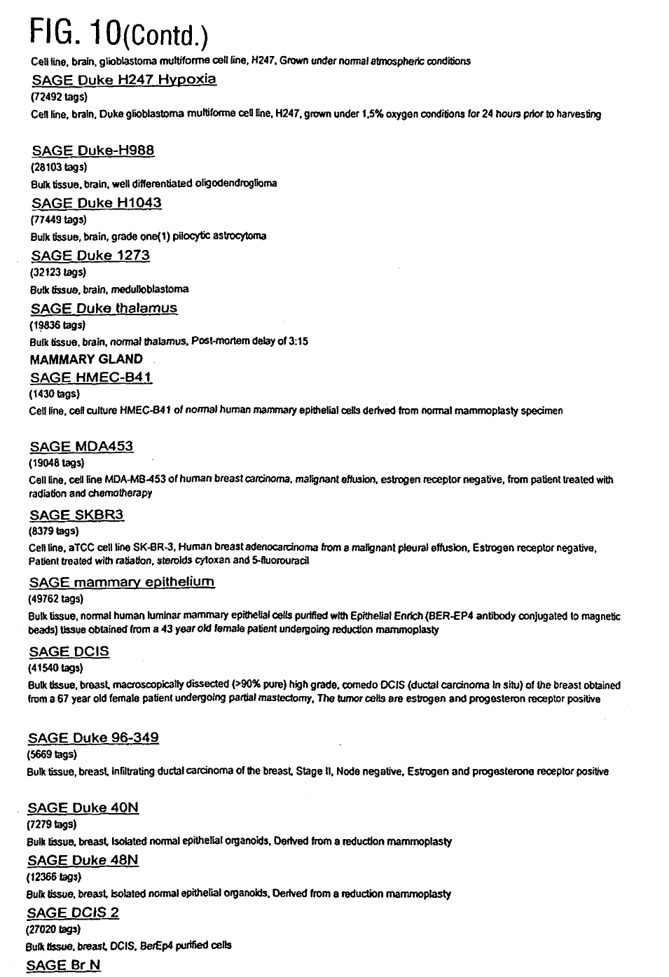

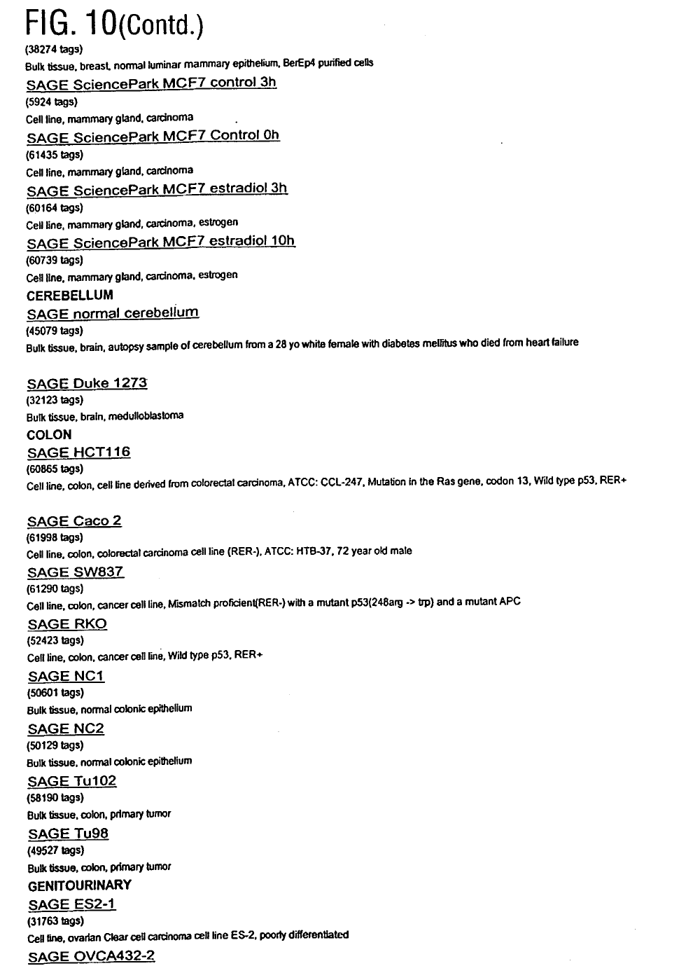

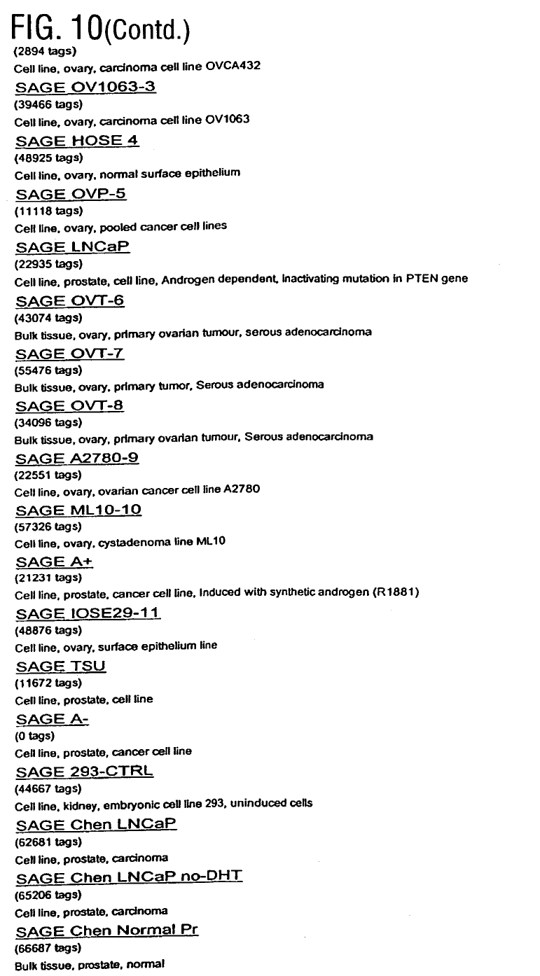

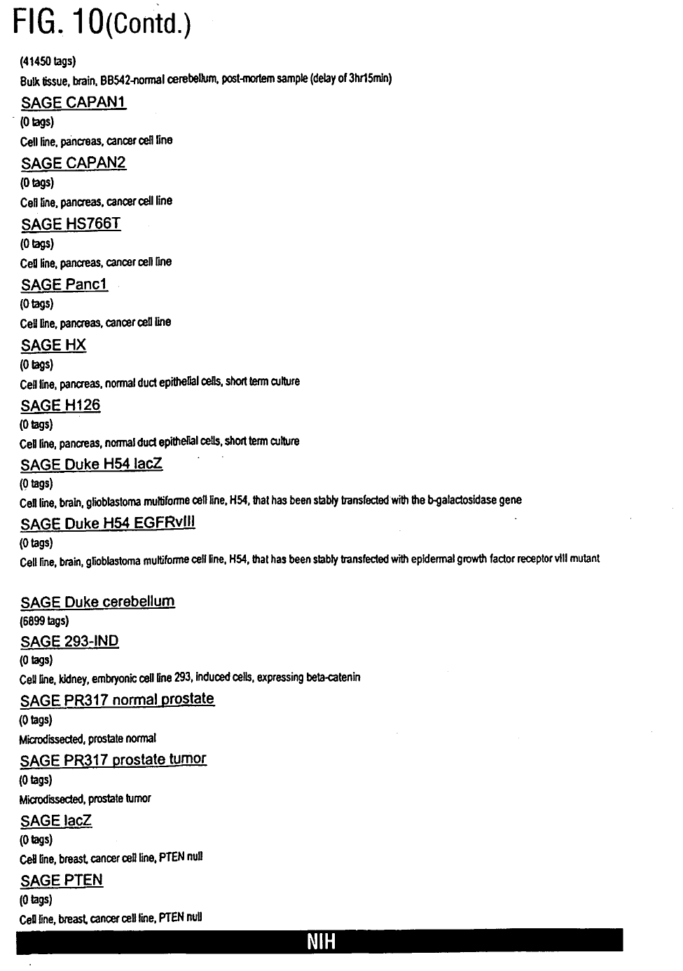

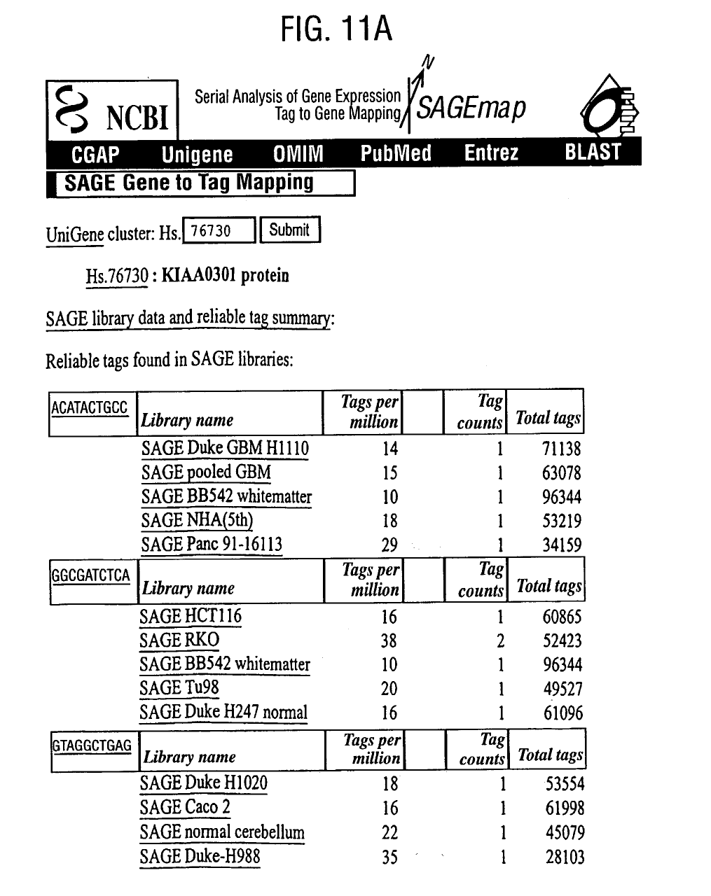

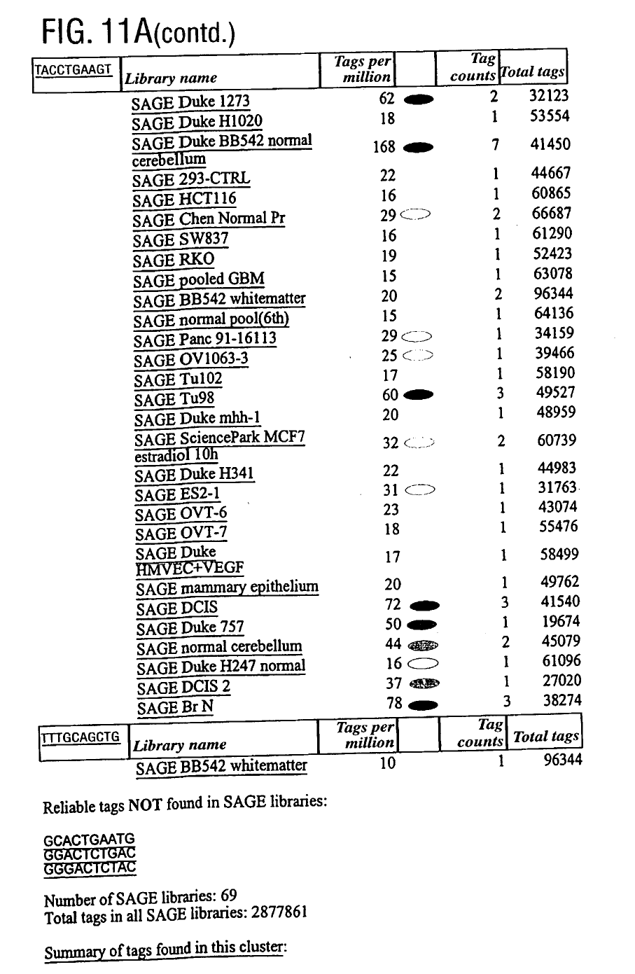

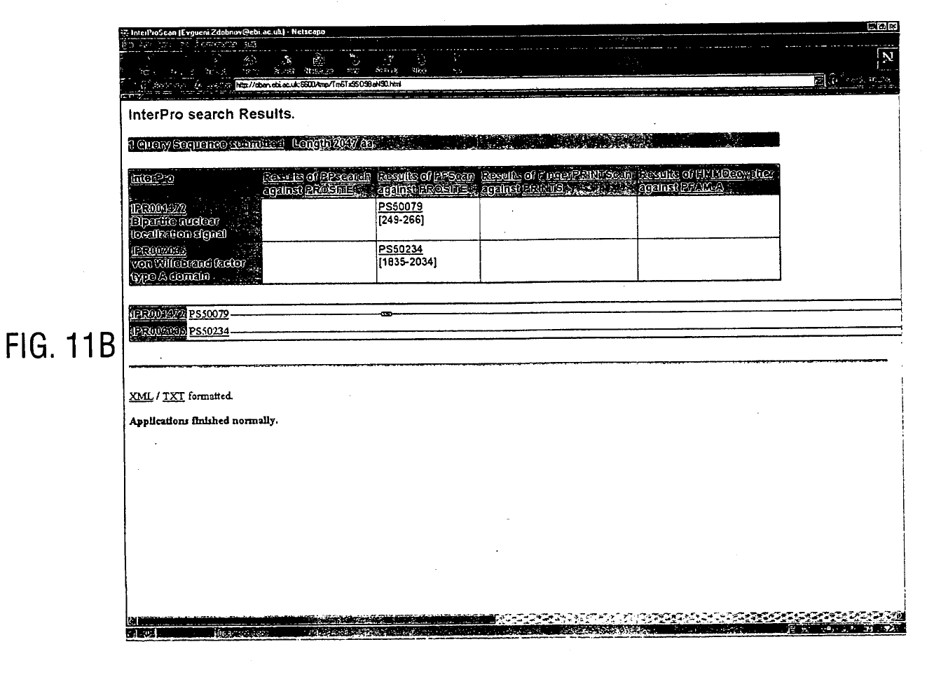

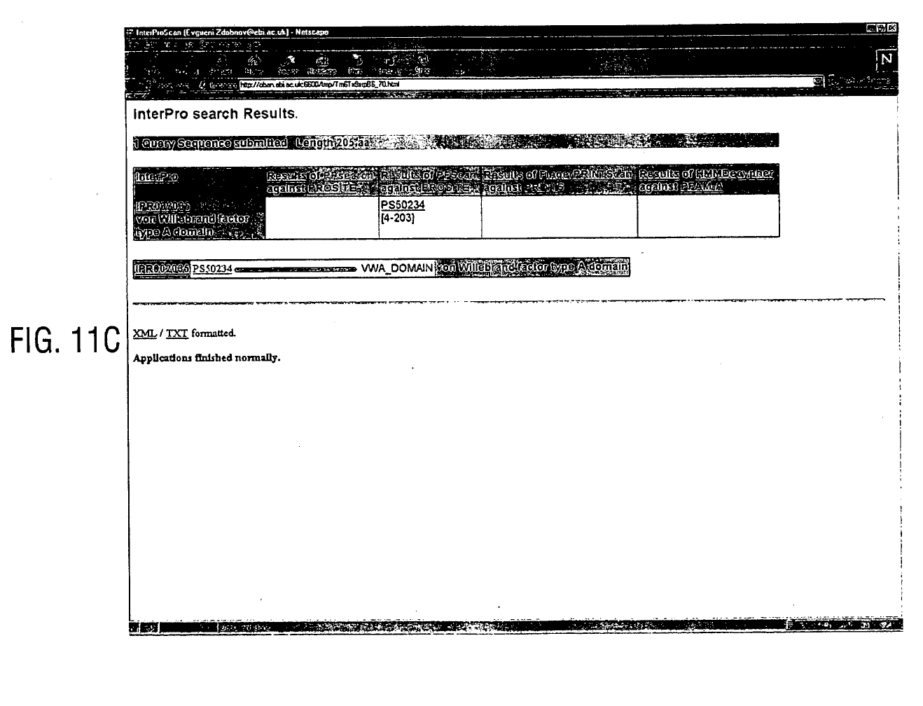

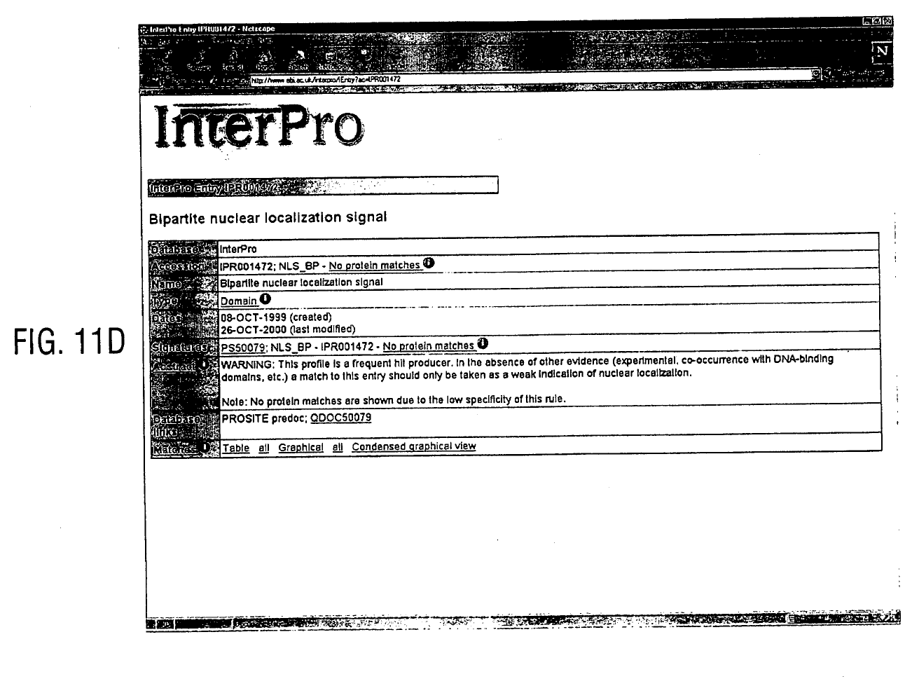

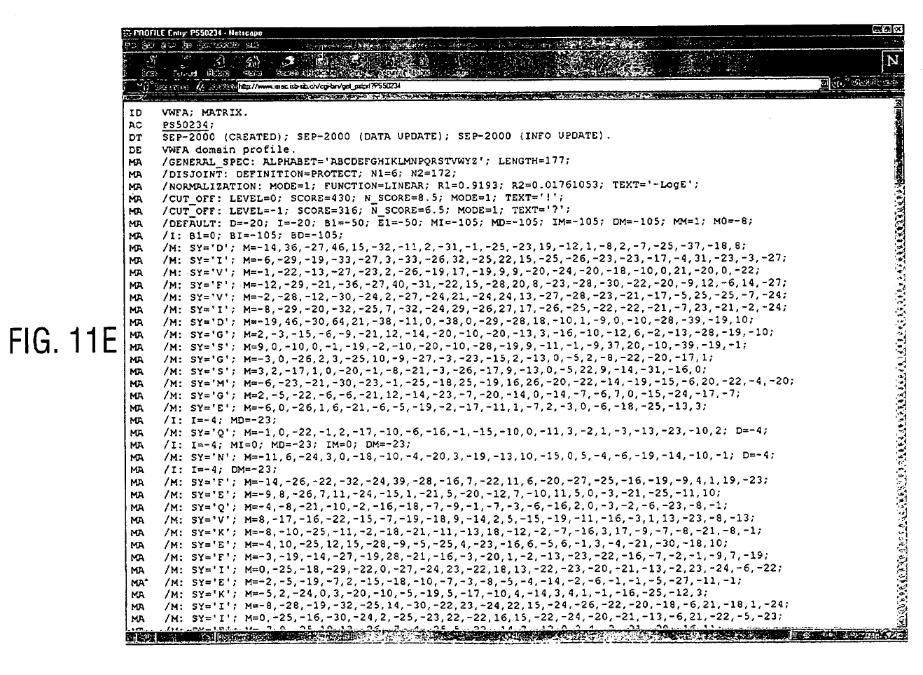

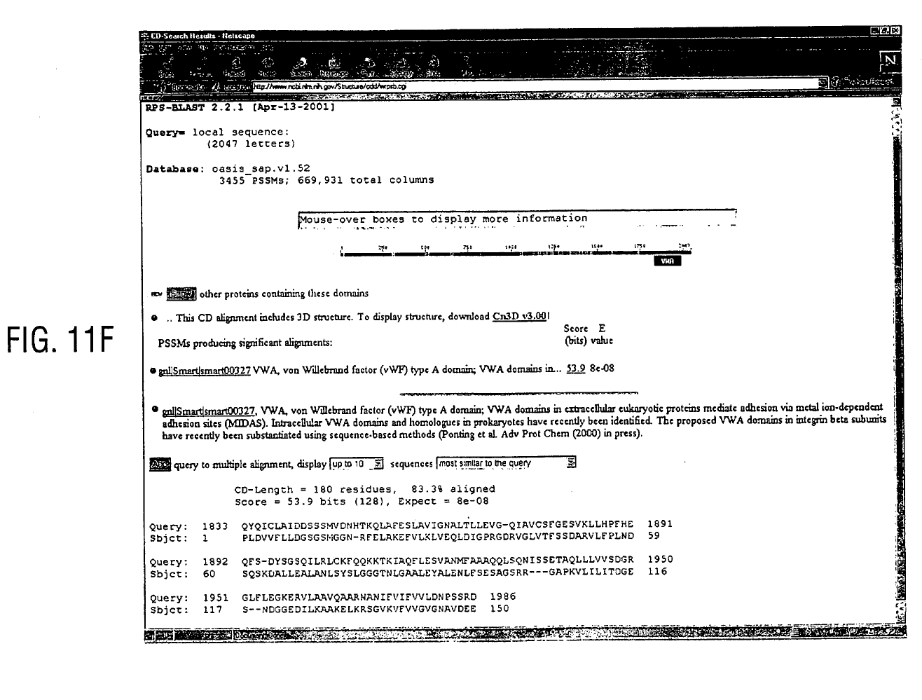

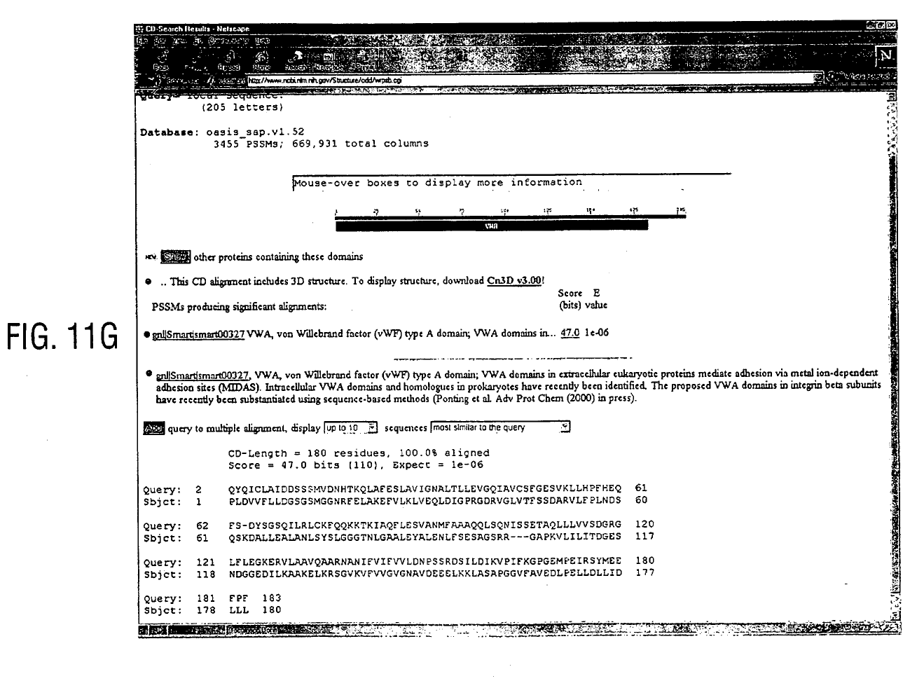

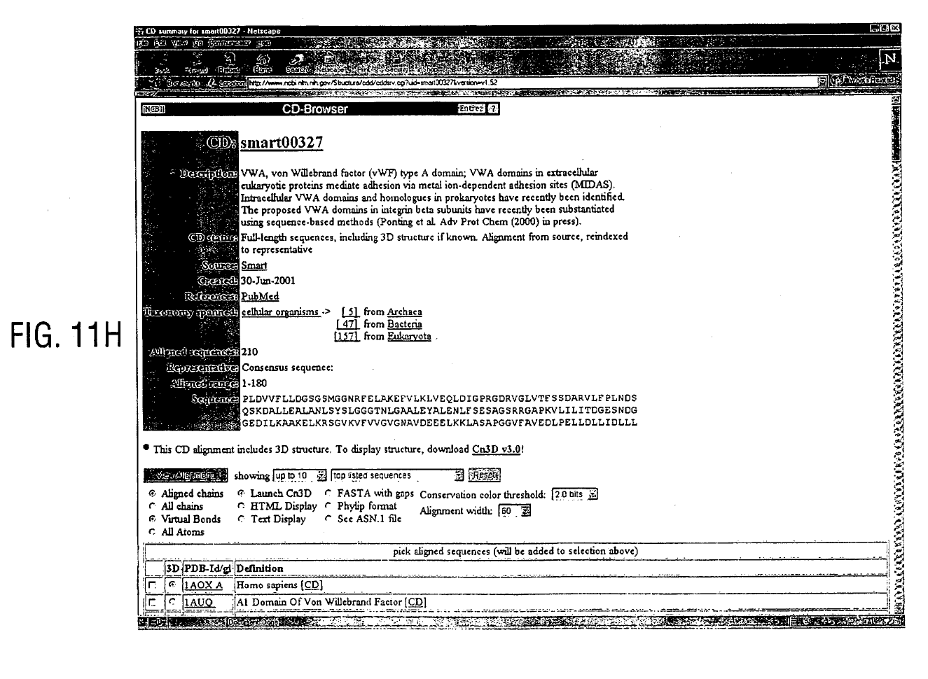

Although the UniGene database gives a rough idea of tissue distribution the Serial Analysis of Gene Expression (SAGE) database gives a direct count of how many times the gene appears in the tissues that have been analysed. FIG. 10 shows a list of all the tissues in the SAGE database. FIG. 11A is a report generated from the SAGE database for BAA20761.1 (AD1), which shows a fairly low level of expression (tags per million) predominately in brain tissue. SAGE tag TACCTGAAGT shows the most striking observation in SAGE DUKE BB542 normal cerebellum library. It is upregulated in this tissue indicating that expression of BAA20761.1 (AD1) may play a role in brain cellular function, both normal and diseased.

FIG. 11B shows the InterPro results for BAA20761.1 (AD1) as of Jul. 24, 2001. InterPro is a public domain annotation tool which combines PROSITE pattern, PROSITE profile, PRINTS and PFAM. InterPro returns two hits. (only the second hit is reported when residues 1832-2036 of BAA20761.1 (AD1), rather than full-length sequence, is inputted into InterPro, see FIG. 11C). The first of the two hits is to the PROSITE profile PS50079, which annotates residues 249-266 of BAA20761.1 (AD1) as being a nuclear localisation signal. A nuclear localisation signal would appear to be inconsistent with a role for BAA20761.1 (AD1) as an adhesion molecule. However, the nuclear localisation signal annotation can be discounted since PS50079 is known to have a high false positive rate, see FIG. 11D. The second InterPro result annotates residues 1835-2034 of BAA20761.1 (AD1) as possessing a von Willebrand factor/I-domain (PROSITE profile PS50234). This public domain annotation of BAA20761.1 (AD1) as possessing an I-domain only became available in September 2000 (see FIG. 11E). Thus this public domain annotation can be considered supporting evidence for the Genome Threader™ annotation of a region including, at the most, residue 1832 to residue 2036 of BAA20761.1 (AD 1), and at the least, residue 1836 to residue 1950 of BAA20761.1 (AD1) as being an I-domain and functioning as an adhesion molecule.

FIG. 11F shows the NCBI Conserved Domain Database (CDD) results for BAA20761.1 (AD1) as of Jul. 24, 2001. CDD returns one hit. (An identical hit is reported when residues 1832-2036 of BAA20761.1 (AD1), rather than the full-length sequence, is inputted into CDD, see FIG. 11G). The match is to the smart00327 profile which annotates residues 1833-1986 of BAA20761.1 (AD1) as possessing a von Willebrand factor/I-domain (smart00327). This public domain annotation of BAA20761.1 (AD1) as possessing an I-domain only became available on Jun. 30, 2001 (see FIG. 11H). Thus this public domain annotation can be considered supporting evidence for the Genome Threader™ annotation of a region including, at the most, residue 1832 to residue 2036 of BAA20761.1 (AD1), and at the least, residue 1836 to residue 1950 of BAA20761.1 (AD1) as being an I-domain and functioning as an adhesion molecule.

### Example 2 G7c (CAB52192.1)

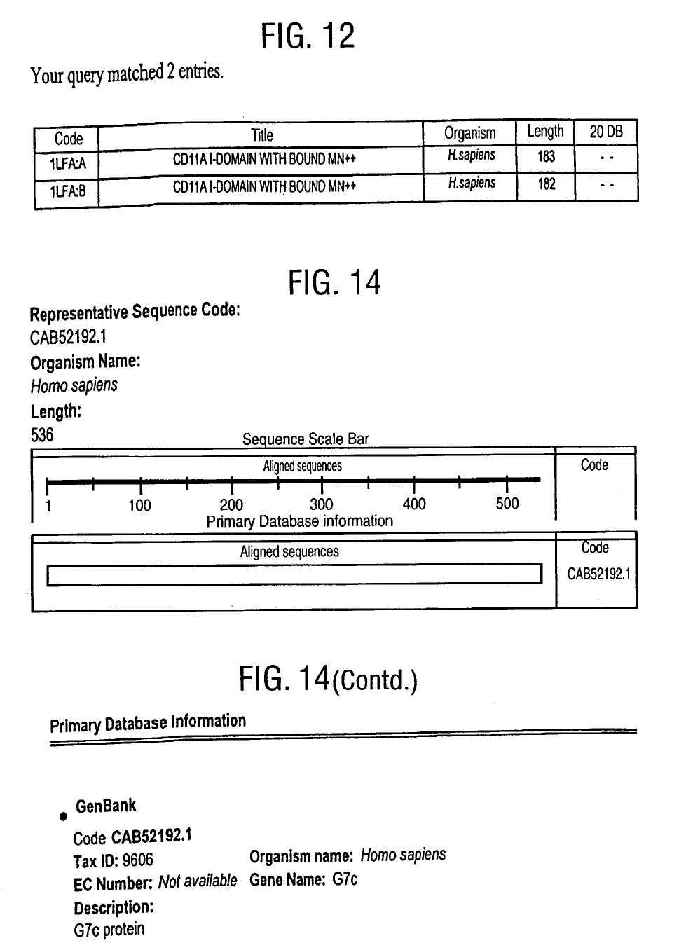

To initiate the search, the I domain (insertion domain) of LFA-1, PDB code 1LFA:A (FIG. 12) is again chosen.

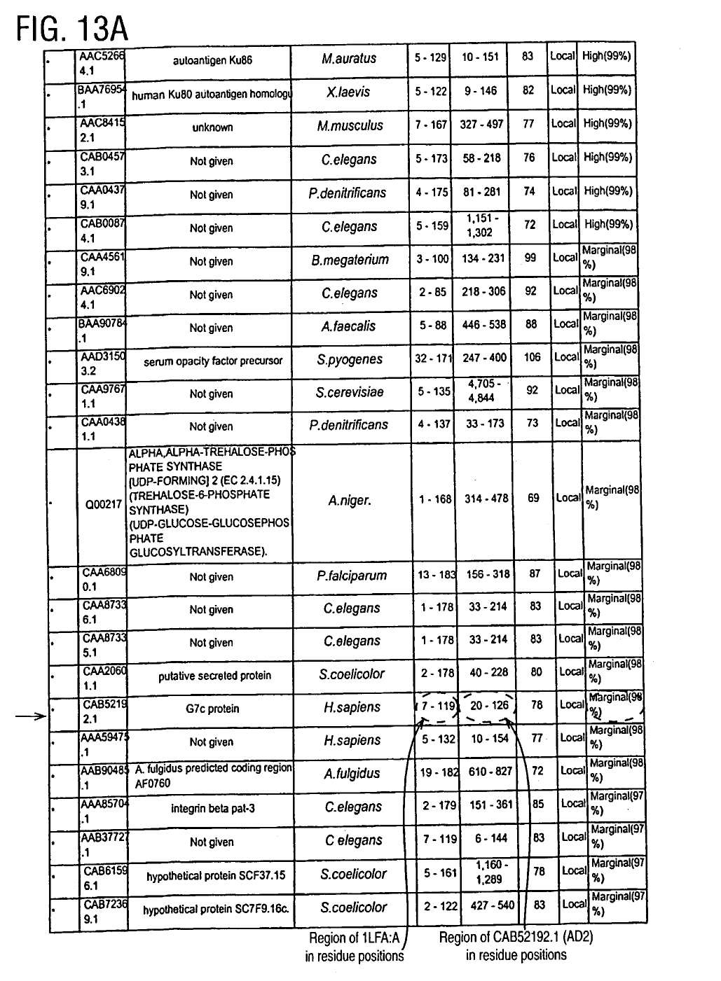

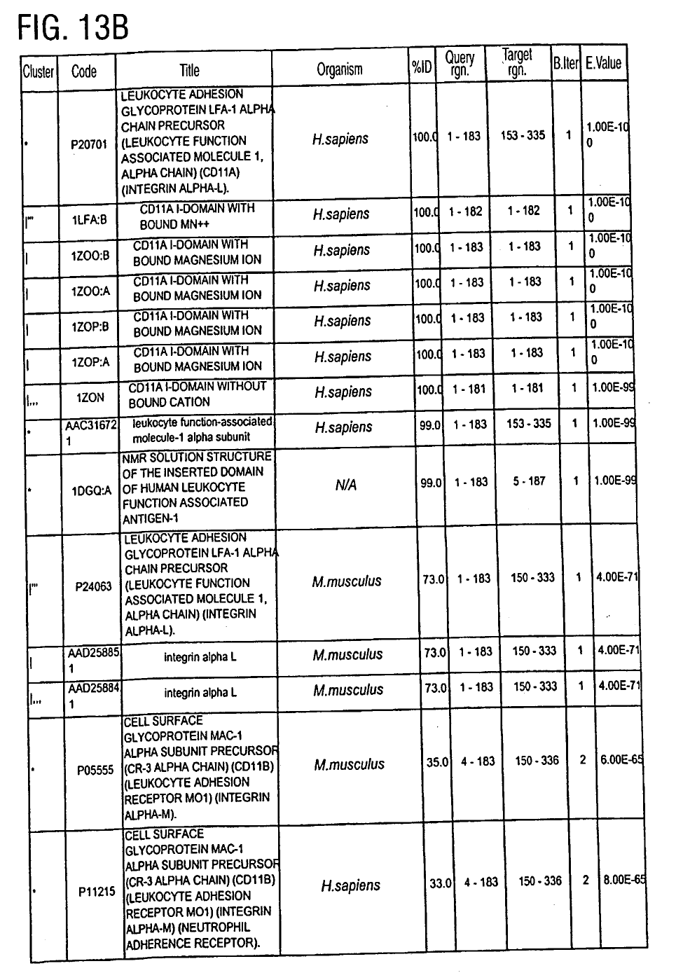

Among the known I domain containing adhesion molecules/proteins appears a protein of apparently unknown function, G7c (CAB52192.1; AD2, FIG. 13A). The Inpharmatica Genome Threader™ has identified residues 20-126 of this sequence as having a structure similar to the I domain of LFA-1 (PDB code 1LFA:A), the known interaction domain between LFA-1 and ICAM. Having a structure similar to this I domain suggests that G7c (AD2) is a protein that functions in cellular adhesion. The Inpharmatica Genome Threader™ identifies this with 98% confidence.

PSI-Blast (FIG. 13B) is unable to identify this relationship; it is only the Inpharmatica Genome Threader™ that is able to identify CAB52192.1 (AD2) as having an I domain. PSI-Blast does identify LFA-1 itself and other related integrins with varying degrees of probability (E value) as would be expected.

In order to view what is known in the public domain databases about CAB52192.1 (AD2), the Redundant Sequence Display Page (FIG. 14) is viewed. G7c (AD2) is a Homo sapiens sequence, its GenBank protein ID is CAB52192.1, and it is 536 amino acids in length. There are no associated PROSITE or PRINTS hits for this sequence. PROSITE and PRINTS are databases that help to describe proteins of similar families. Returning 0 hits from both databases means that BAA20761.1 (AD2) is unidentifiable as an I domain containing adhesion molecule using PROSITE or PRINTS.

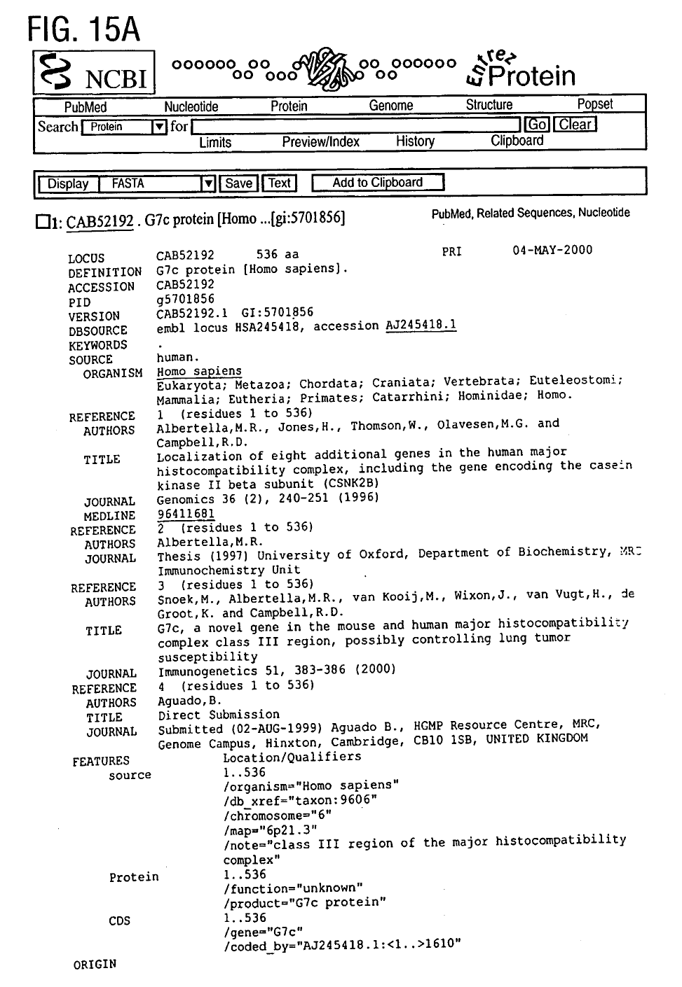

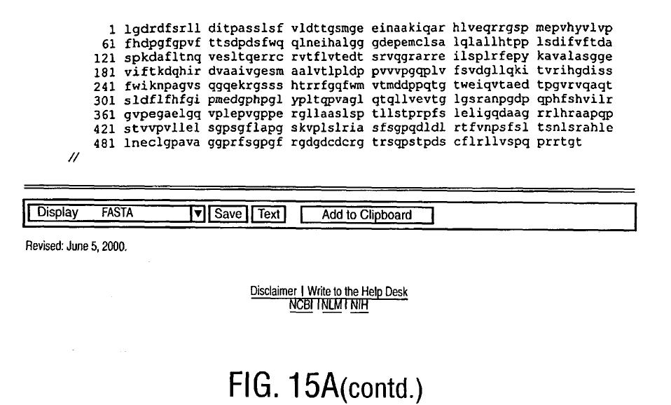

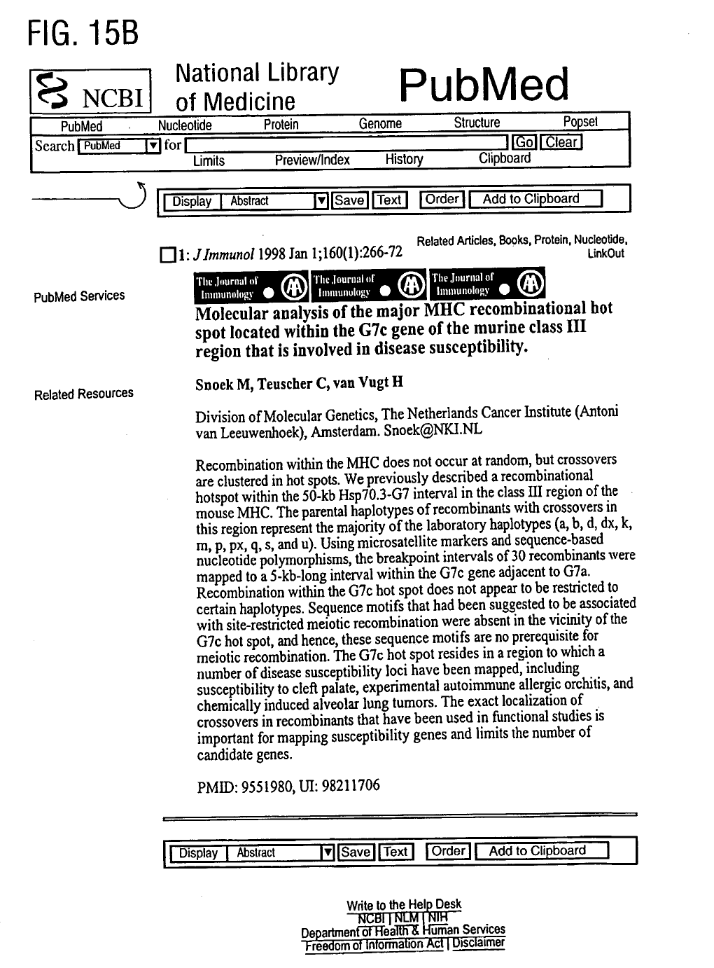

The National Center for Biotechnology Information (NCBI) GenBank protein database is viewed to examine if there is any further information that is known in the public domain relating to CAB52192.1 (AD2). This is the U.S. public domain database for protein and gene sequence deposition (FIG. 15A). Several groups have cloned CAB52192.1 but its function remains unknown. CAB52192.1 (AD2) is a gene sequence located in the human major histocompatibility complex (MHC). The public domain information for this gene does not annotate it as an integrin or an I domain-containing protein. Snoek et al. (J. Immunol. 1998 160(1):266-72; FIG. 15B herein) identify the G7c (AD2) gene as being located in a MHC recombinatorial hot spot that is associated with a number of disease susceptibility loci, including susceptibility to cleft palate, experimental autoimmune allergic orchitis, and chemically induced alveolar lung tumours. Defects in adhesion molecules could explain all of these disorders.

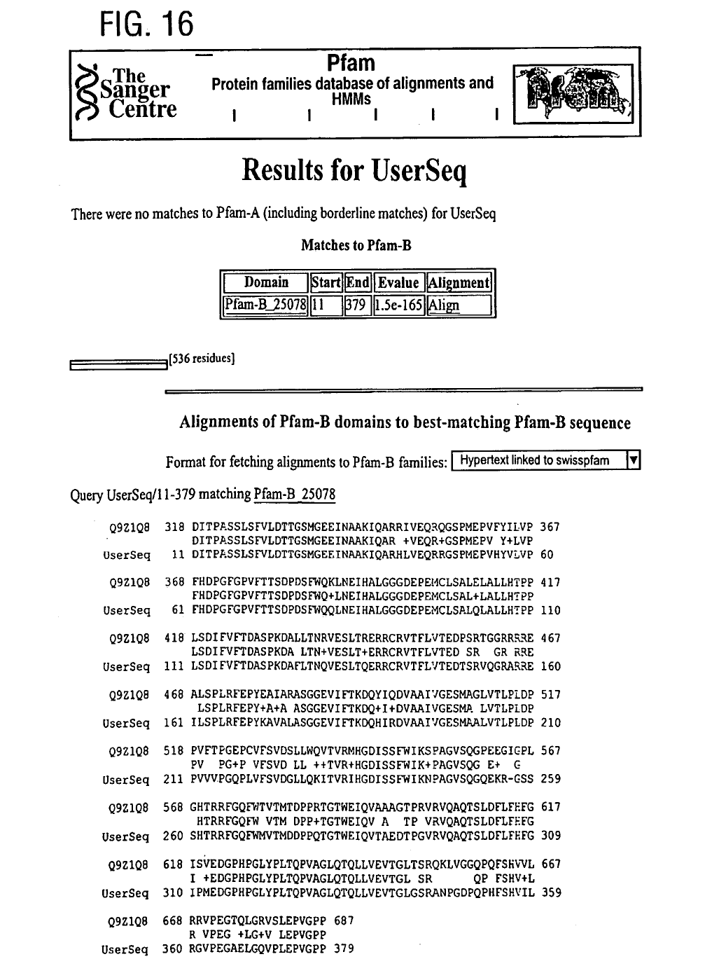

In order to identify whether any other public domain annotation vehicle is able to annotate CAB52192.1 (AD2) as an I domain containing protein, the CAB52192.1 protein sequence is searched against the Protein Family Database of Alignment and HMM's (PFAM) database (FIG. 16). The results identify that CAB52192.1 (AD2) has no identifiable PFAMs of high quality (PFAM-A) hits. It does identify a PFAM-B hit, but these are of low quality and unknown function. PFAM does not identify CAB52192.1 (AD2) as having an I domain. Therefore using all public domain annotation tools CAB52192.1 (AD2) is not annotated as a protein involved in adhesion through the presence of an I domain. Only the Inpharmatica Genome Threader™ is able to annotate it as having an I domain. Interestingly, public domain literature (FIG. 15B) implicates G7c (AD2) in several disease processes in which adhesion molecules are known to be important.

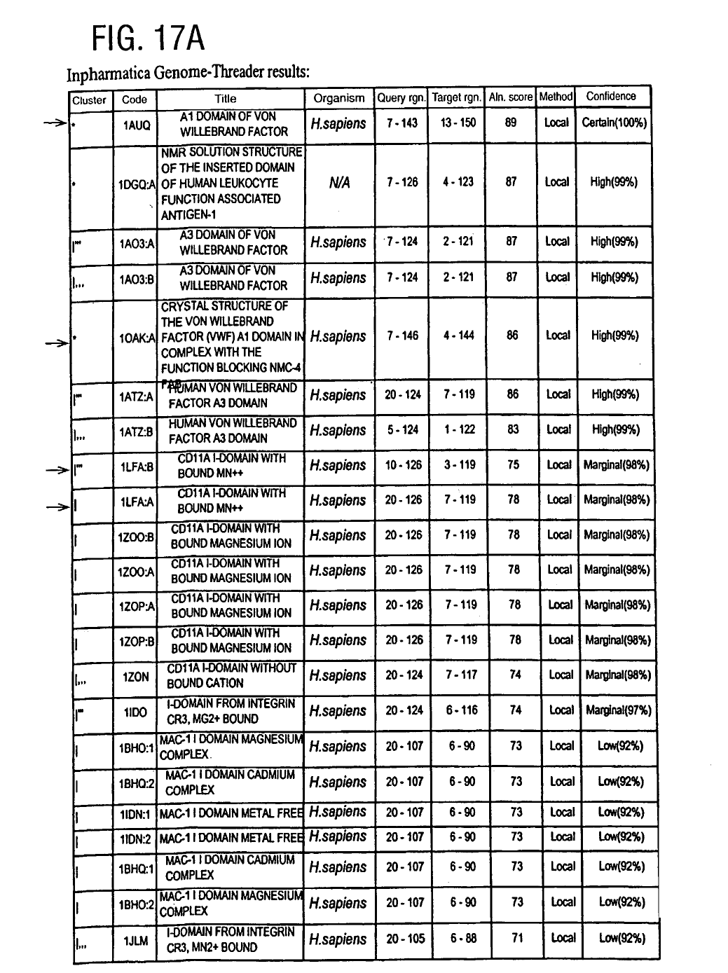

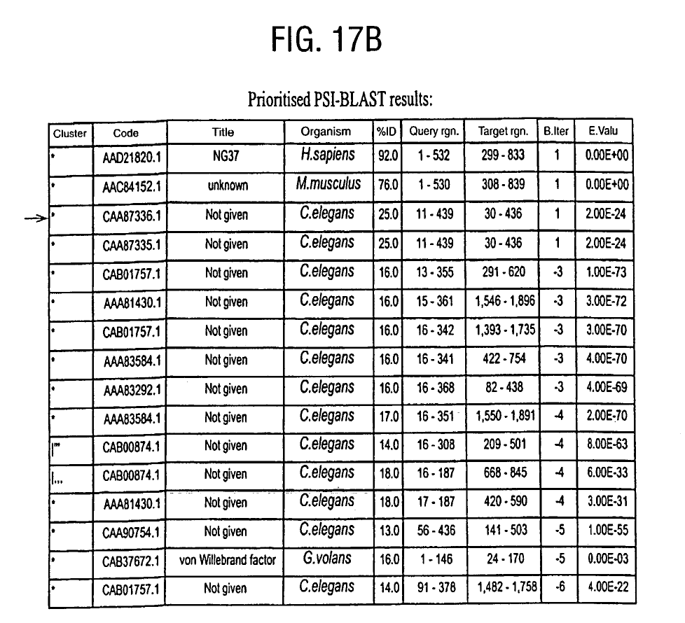

The reverse search is now carried out. CAB52192.1 (G7c; AD2) is now used as the query sequence in the Biopendium™. The Inpharmatica Genome Threader™ identifies 22 hits (FIG. 17A) while PSI-Blast returns 16 hits (FIG. 17B). The Inpharmatica Genome Threader™ (FIG. 17A, arrow \mathring{1}) identifies residues 10-126 of CAB52192.1 (AD2) as having a structure the same as the I-domain of the Integrin LFA-1 (1LFA:B) with a confidence of 98%. The Inpharmatica Genome Threader™ (FIG. 17A, arrow \mathring{2}) also identifies residues 20-105 of CAB52192.1 (AD2) as having a structure the same as the I-domain of the Integrin MAC-1 (1JLM) with a confidence of 92%. Thus a region from residues 10-20 to residues 105-126 of CAB52192.1 (AD2) has been identified as adopting an equivalent fold to a range of I-domains including those of the adhesion molecules LFA-1 and MAC-1.

Forward PSI-Blast does not return this result. PSI-Blast is only able to identify a relationship with von Willebrand factor (CAB37672.1) in the negative iteration, which the Biopendium™ computes through its all by all calculation. Interestingly, reverse maximised Psi-Blast identifies a homologue of AD2 in C. elegans, CAA87336.1. Therefore, it is only the Inpharmatica Genome Threader™ and negative iteration PSI-Blast that is able to identify the I domain of CAB52192.1.

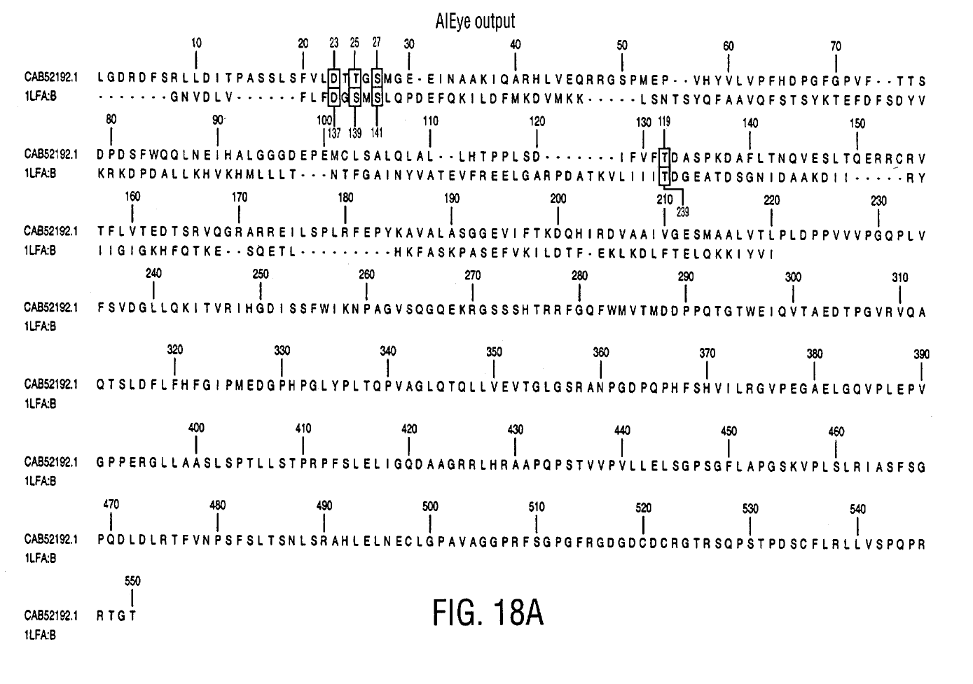

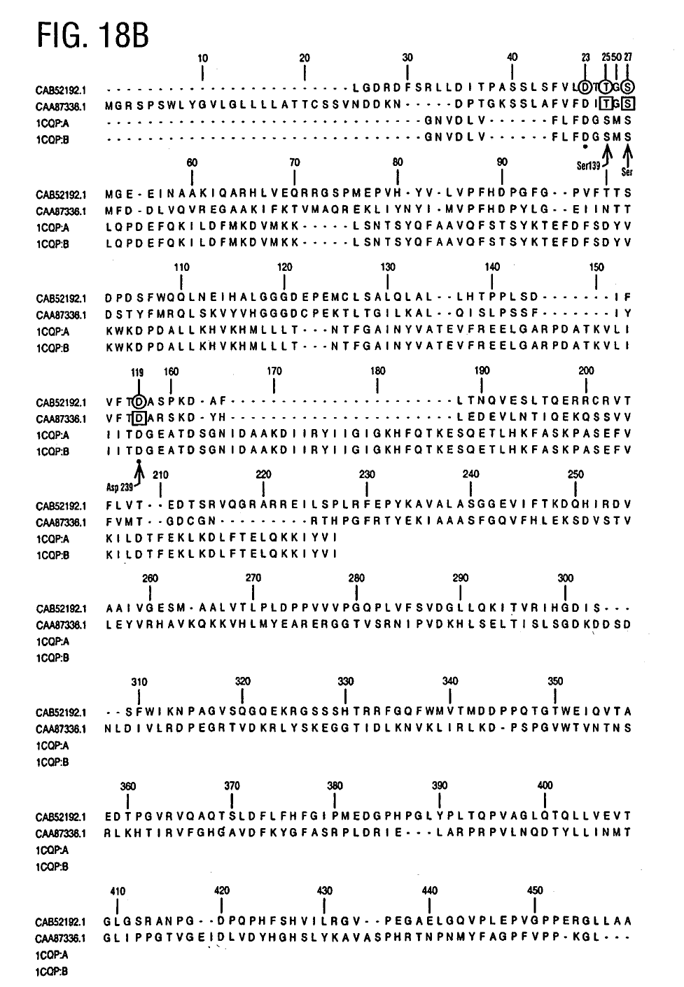

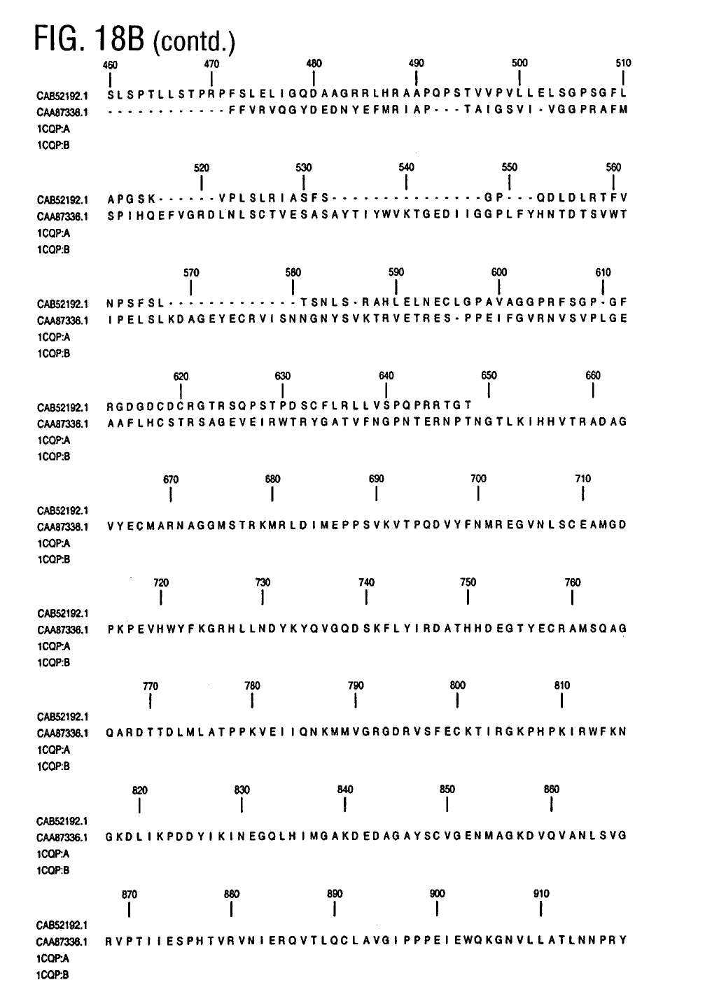

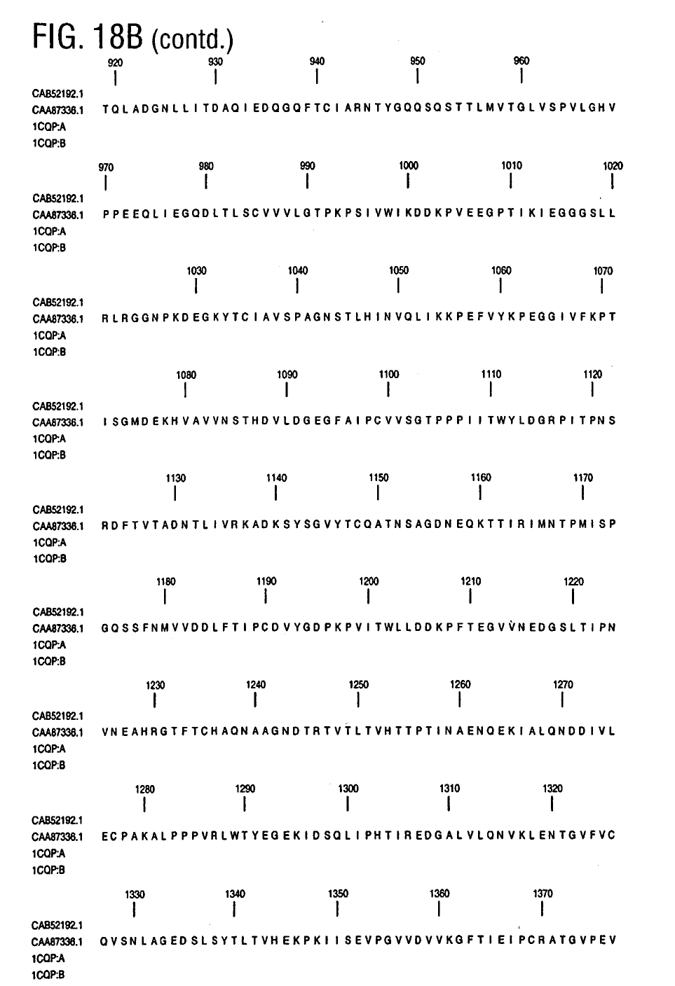

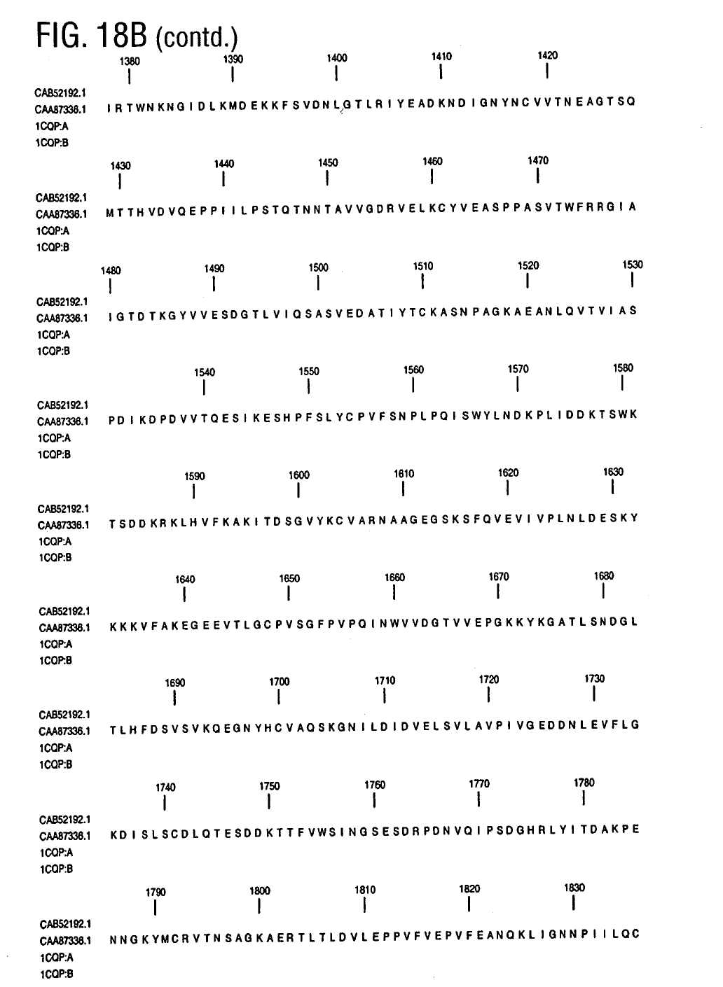

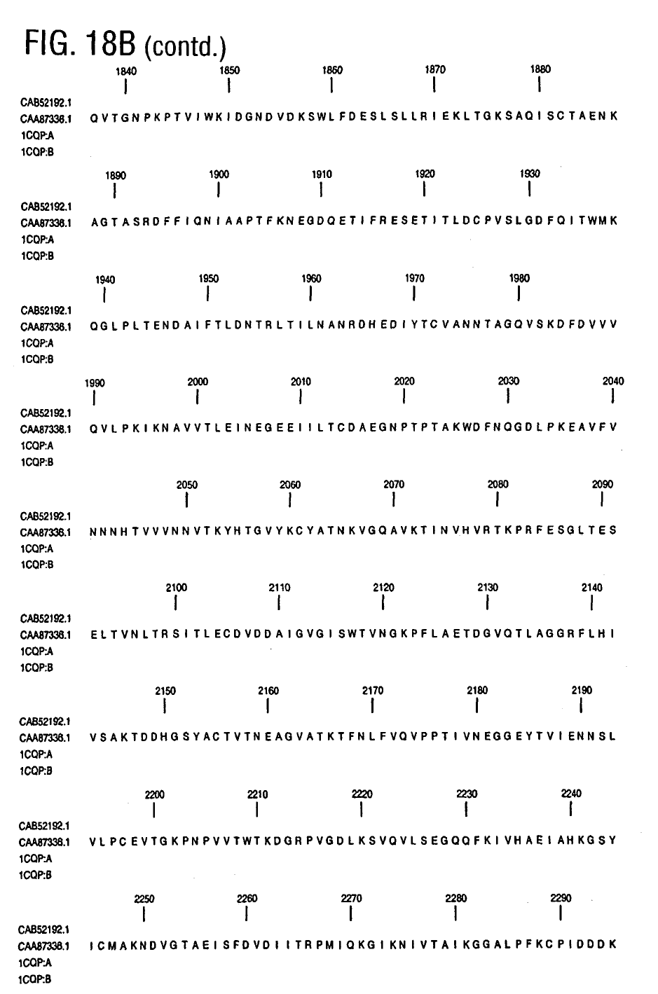

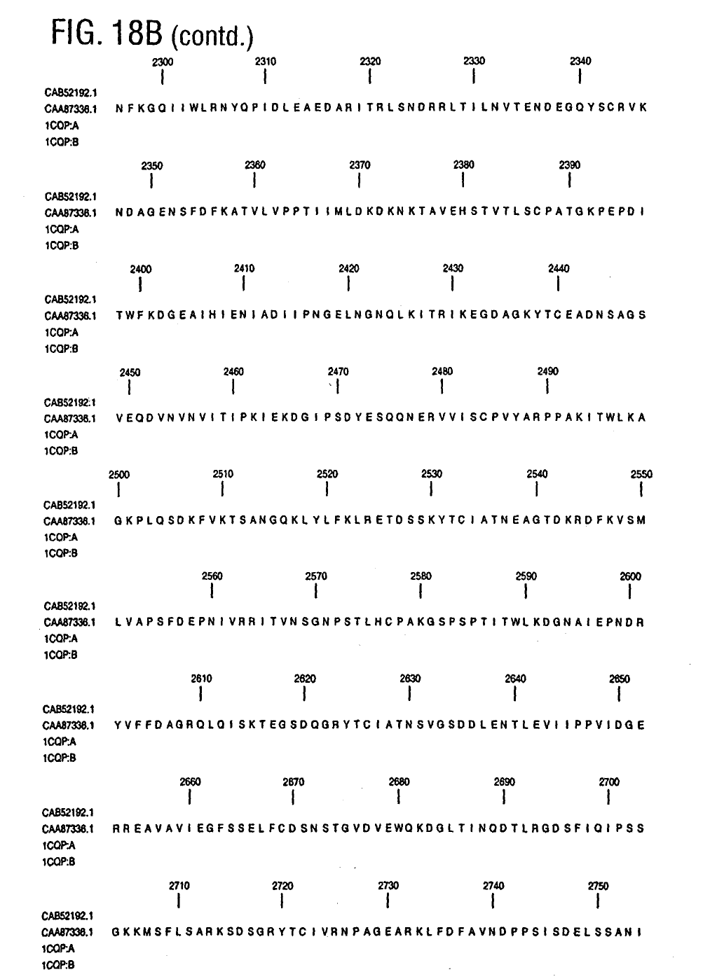

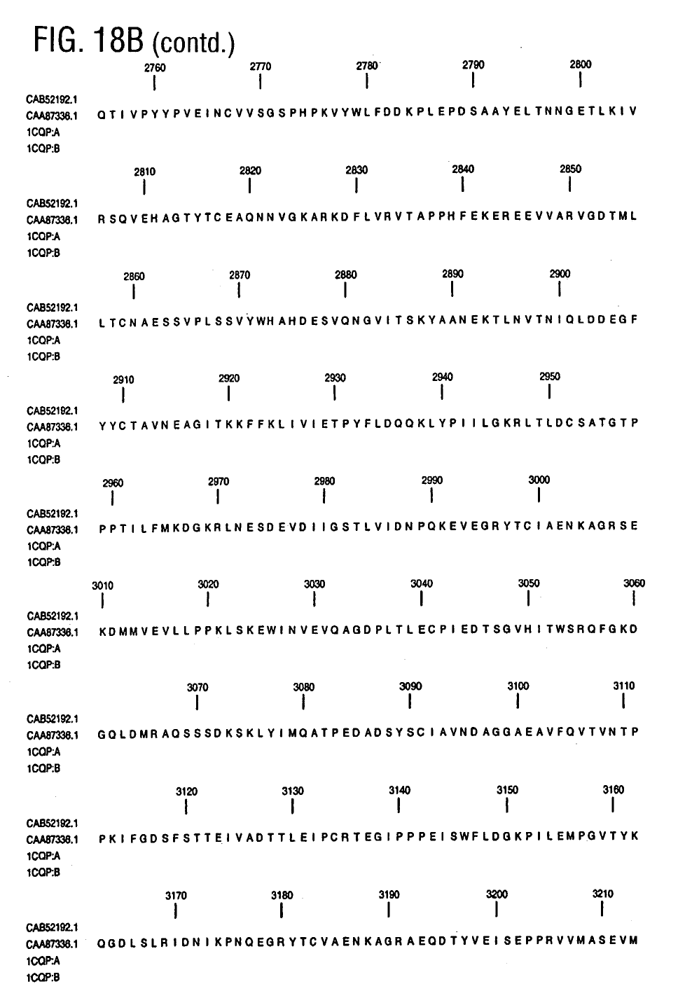

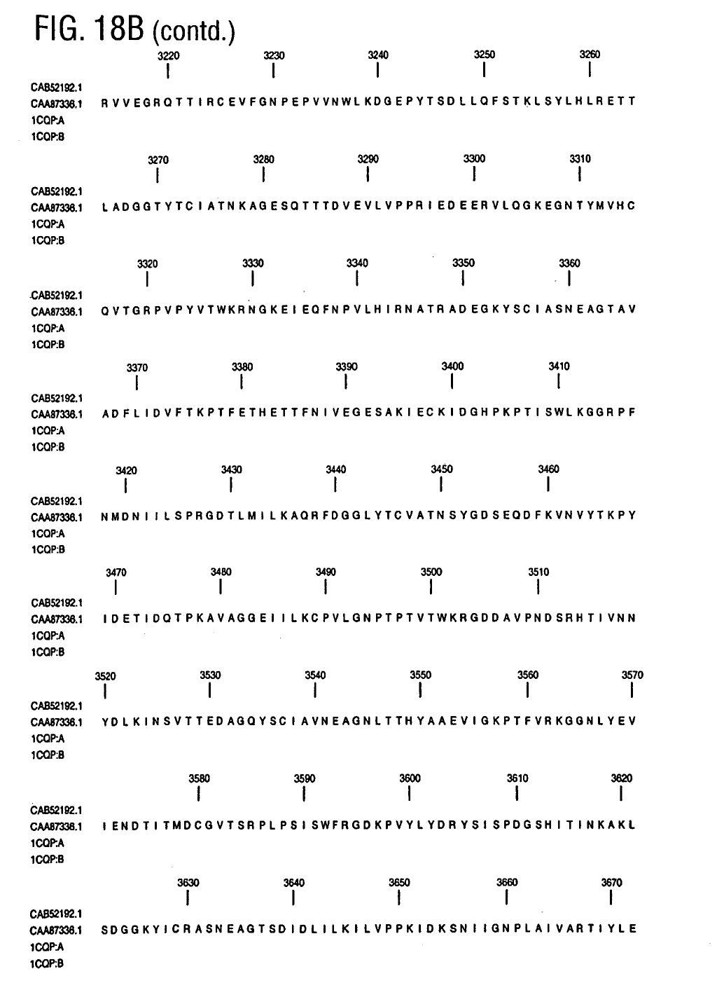

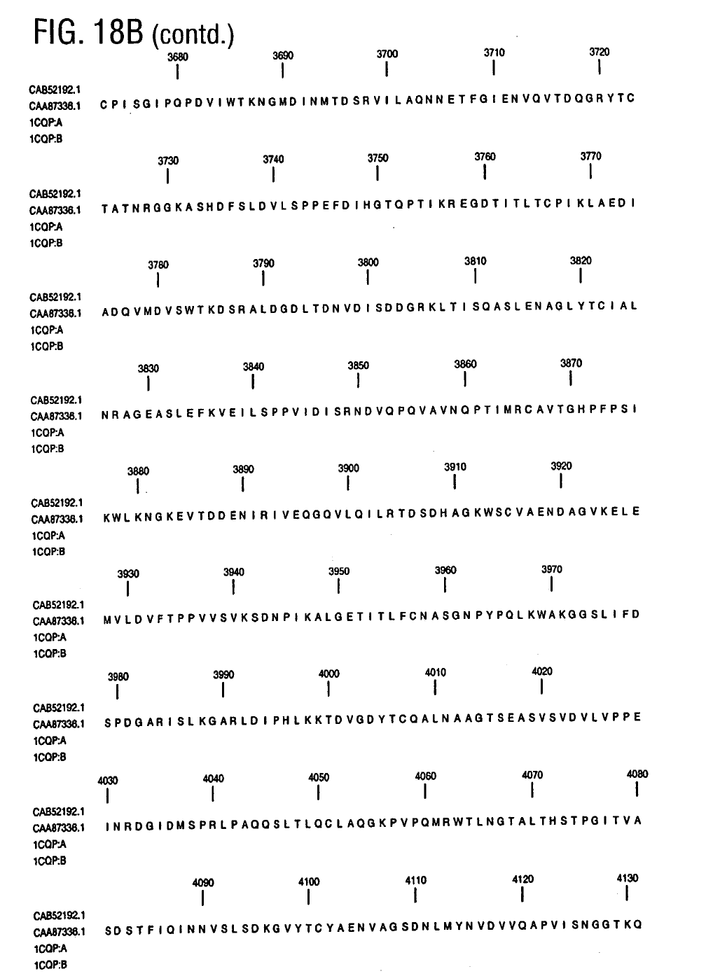

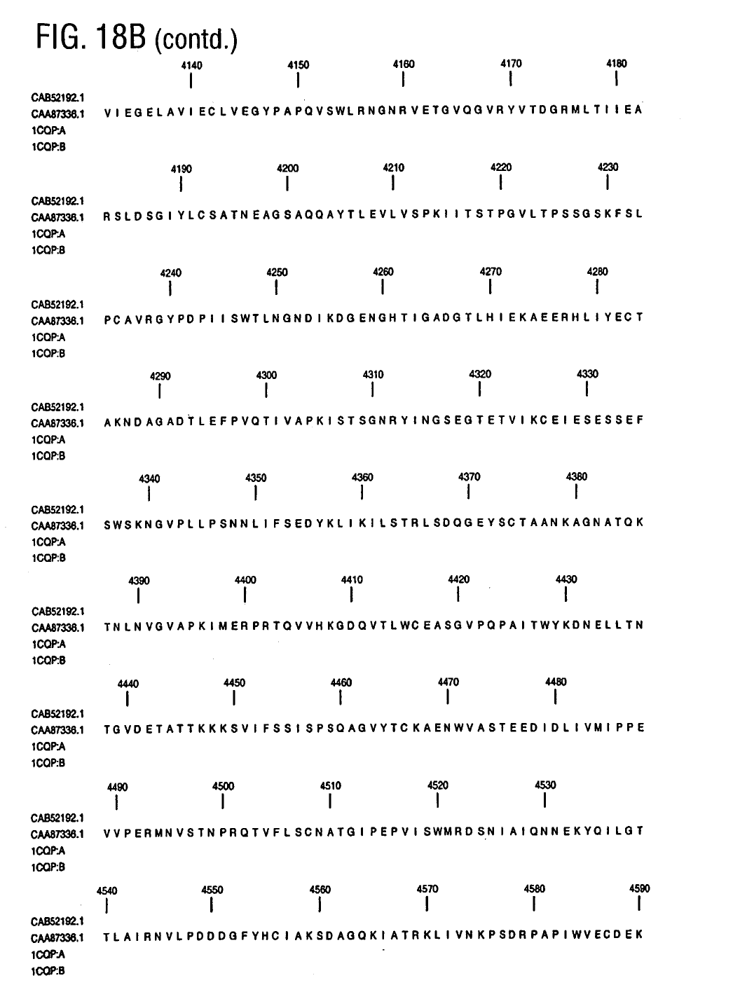

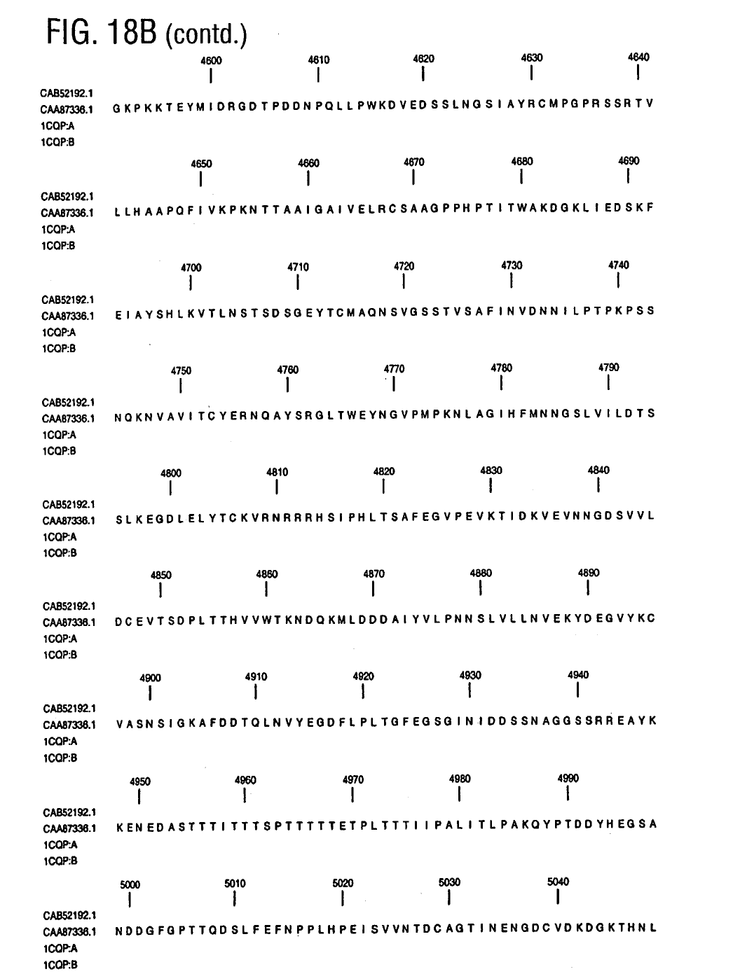

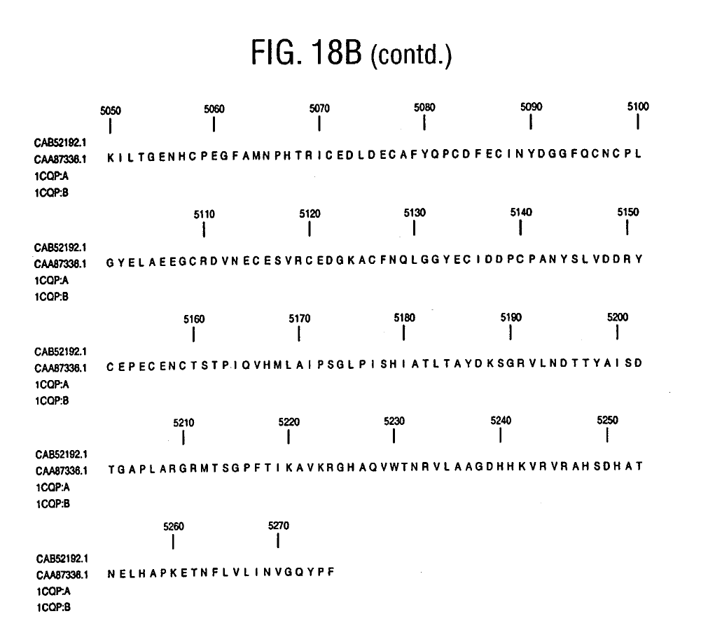

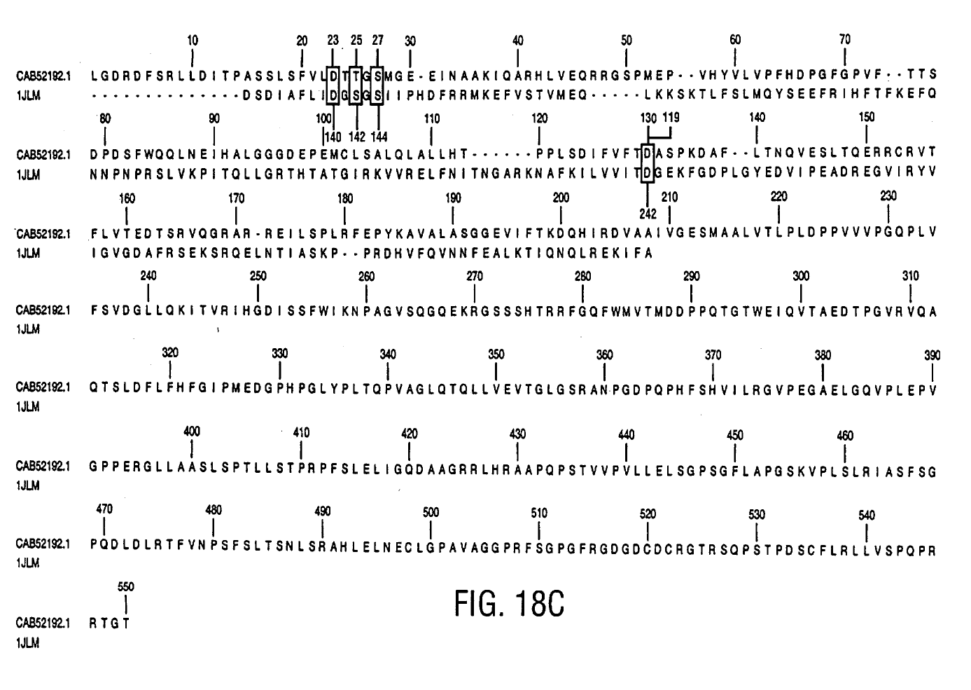

Among the I-domains that the Inpharmatica Genome Threader™ returns is the I-domain of LFA (1LFA:B). 1LFA:B is chosen against which to view the sequence alignment of BAA20761.1 (AD2). Viewing the alignment (FIG. 18A) of the query protein against the proteins identified as being of a similar structure helps to visualize the areas of homology. FIG. 18A illustrates the point that the divalent cation binding residues of 1LFA;B (Ser139, Ser141, and Asp239) are conserved as Thr25 (a conservative substitution), Ser27 and Asp119, respectively, in BAA20761.1 (AD2).

FIG. 18B shows the alignment of BAA20761.1 (AD2) with another set of LFA-1 structures (1CQP:A and 1CQP:B). FIG. 18B reinforces the point that the divalent cation binding residues of LFA-1 (1CQP:A and 1CQP:B) are conserved in CAB52192.1 (AD2). The alignment on FIG. 18B also shows that the C. elegans homologue CAA87336.1 has th functionally important divalent cation binding residues conserved. This indicates that these residues are of functional importance to the protein, indicating that CAB52192.1 does indeed contain an I domain. Residues which are essential for the function of a protein will be conserved in homologues of that protein. Thus the precise conservation of identified functional I domain residues strongly supports the annotation of CAB52192.1 as an I domain-containing protein.

FIG. 18C shows the alignment of BAA20761.1 (AD2) with the Integrin MAC-1 (1JLM). FIG. 18C illustrates the point that the divalent cation binding residues of MAC-1 (Ser142, Ser144, and Asp242) are conserved as Thr25 (a conservative substitution), Ser27 and Asp119, respectively, in BAA20761.1 (AD2).

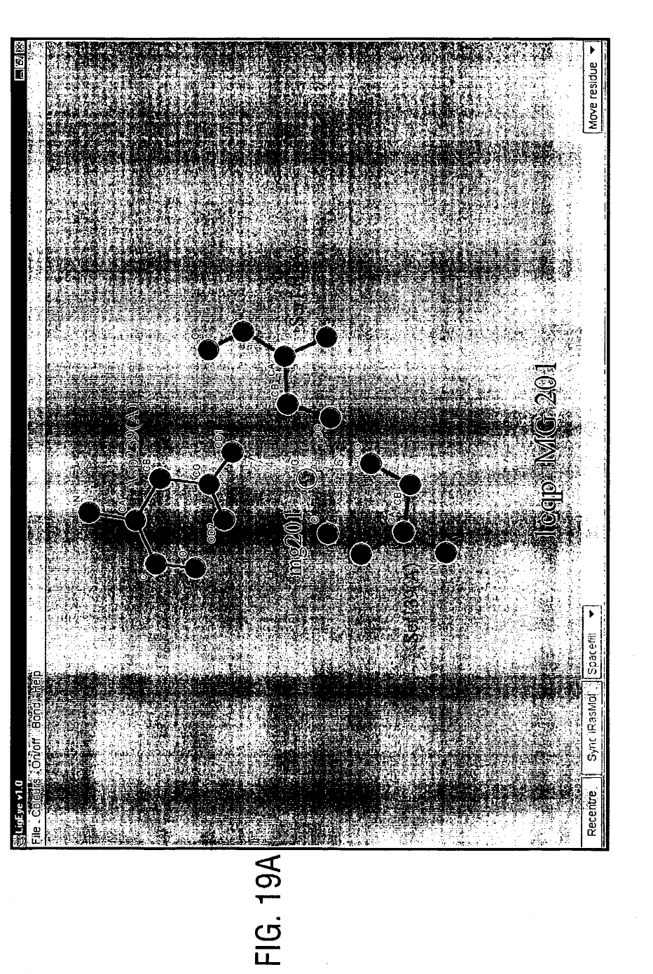

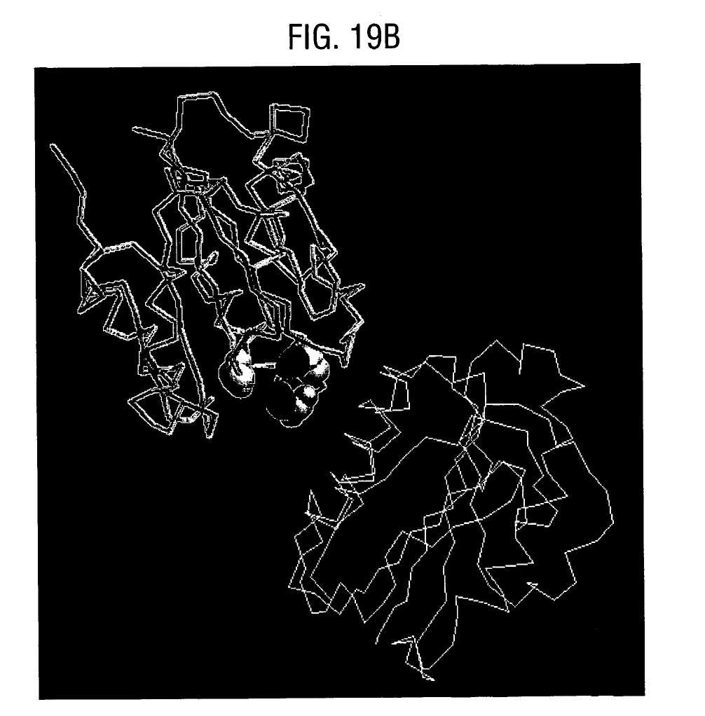

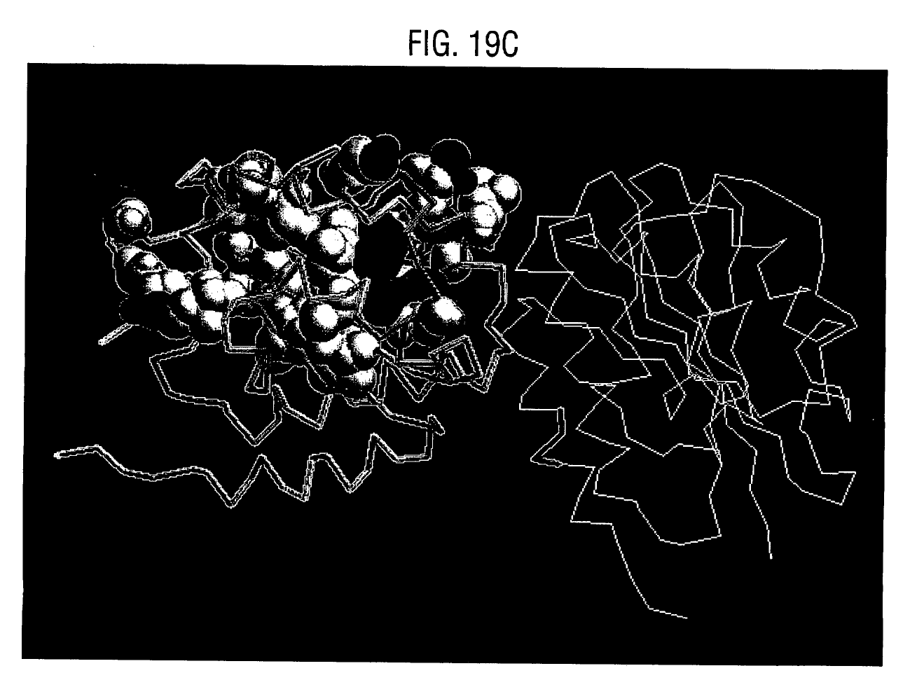

In order to ensure that the protein identified is a homologue of the query sequence LigEye (FIG. 19A) and RasMol (FIG. 19B) are used. These visualization tools identify the active site of known protein structures by indicating the amino acids that known small molecule inhibitors interact with at the active site. These interactions are through either a direct hydrogen bond or hydrophobic interactions. In this manner, one can see if the active site fold/structure is conserved between the identified homologue and the chosen protein of known structure. This is shown with 1CQP, which illustrates the sites of interaction of a magnesium ion with the I domain of lymphocyte function-associated antigen (FIG. 19A). The magnesium ion sees 3 different amino acids in the I domain of LFA-1 (1CQP). The three amino acids of LFA-1 (SER139, SER141, and Asp239) are partially conserved in CAB52192.1 (AD2) (a threonine substitutes for SER139 in CAB52192.1, FIG. 19B, ball structures). FIG. 19C further identifies the amino acids that are conserved in LFA-1 (1CQP) and CAB52192.1. The conservation of amino acids indicates that the fold is very similar. This indicates that indeed as predicted by the Inpharmatica Genome Threader™, CAB52162.1 (AD2) folds in a similar manner to 1LFA and as such is identified as an I domain containing protein.

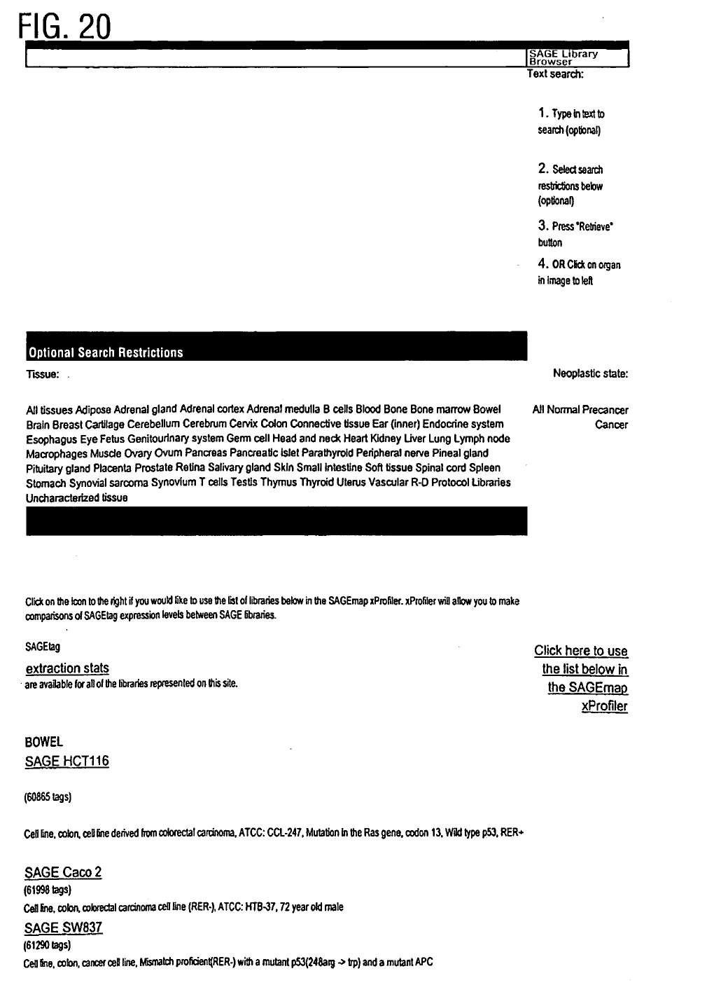

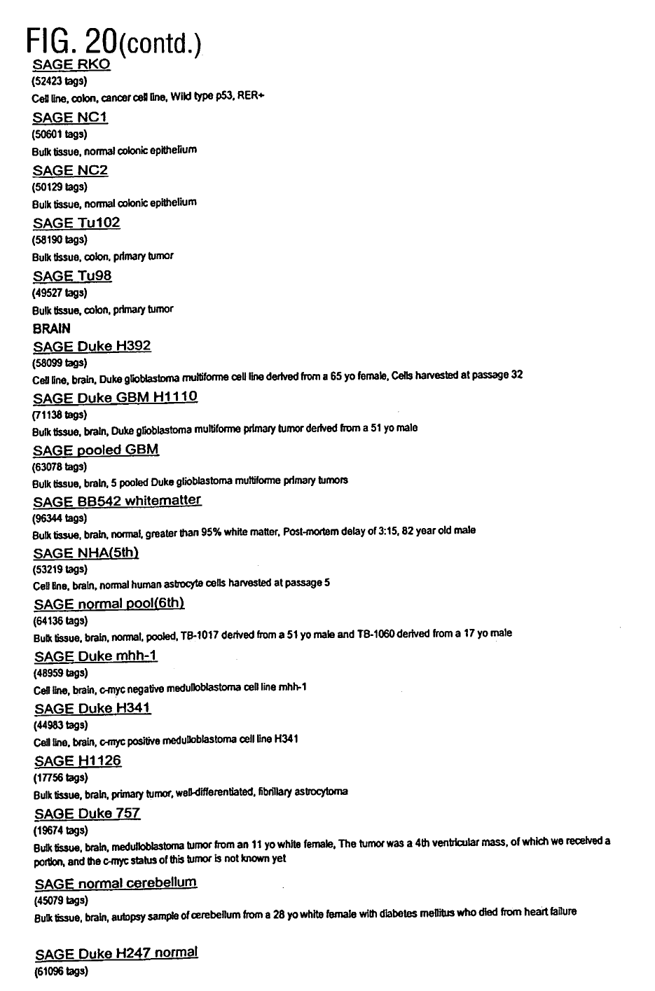

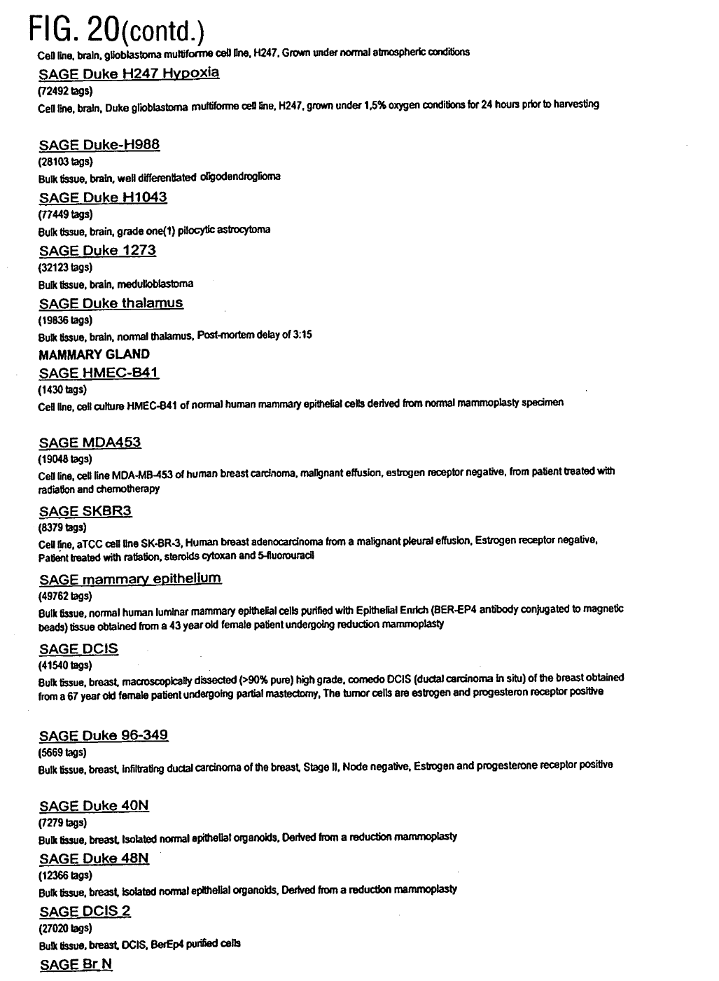

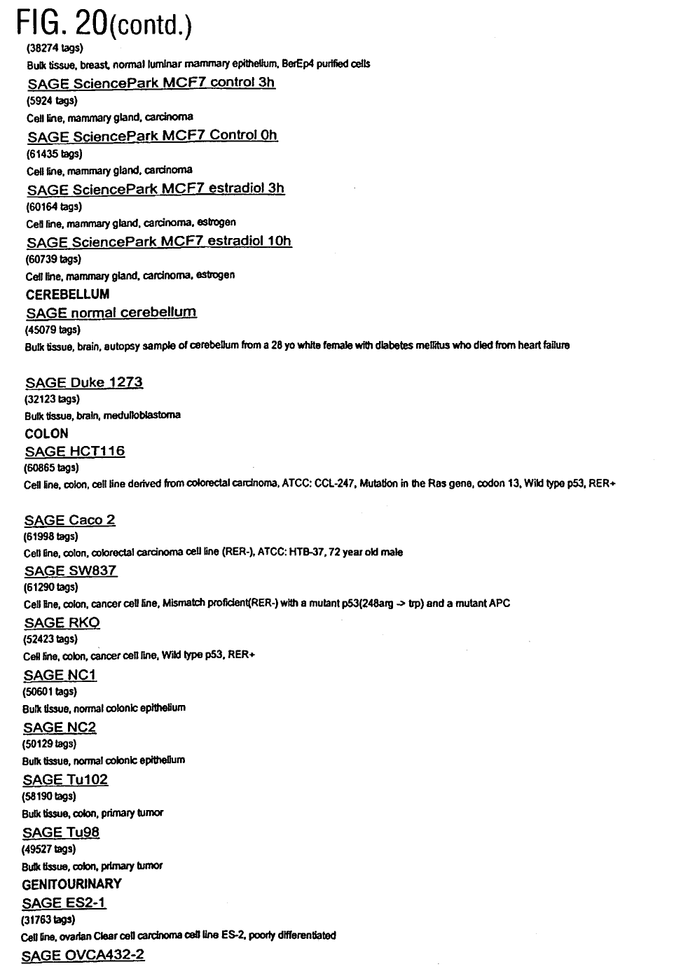

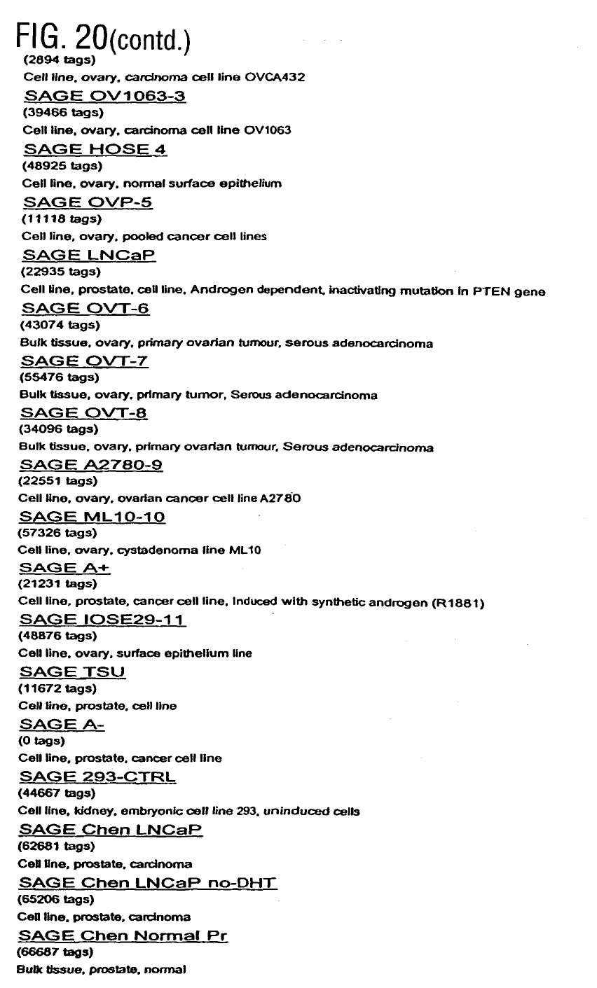

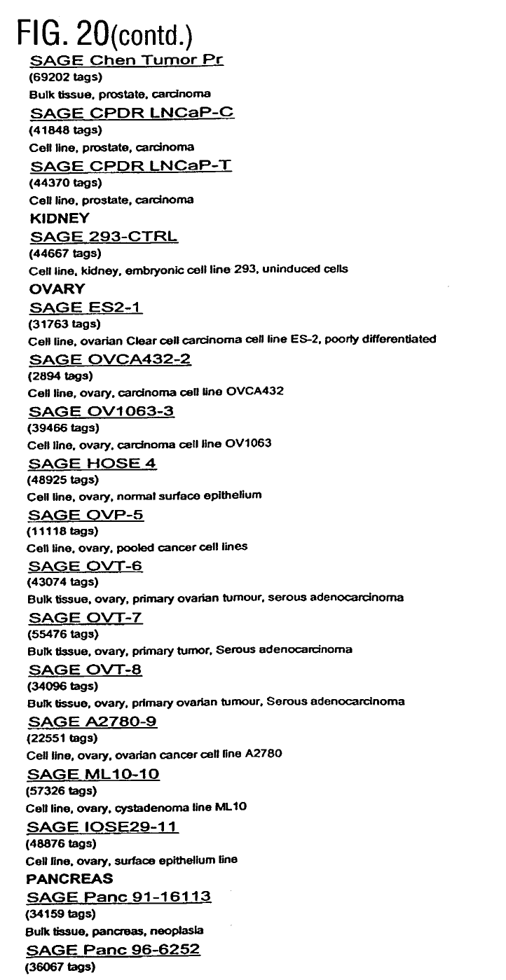

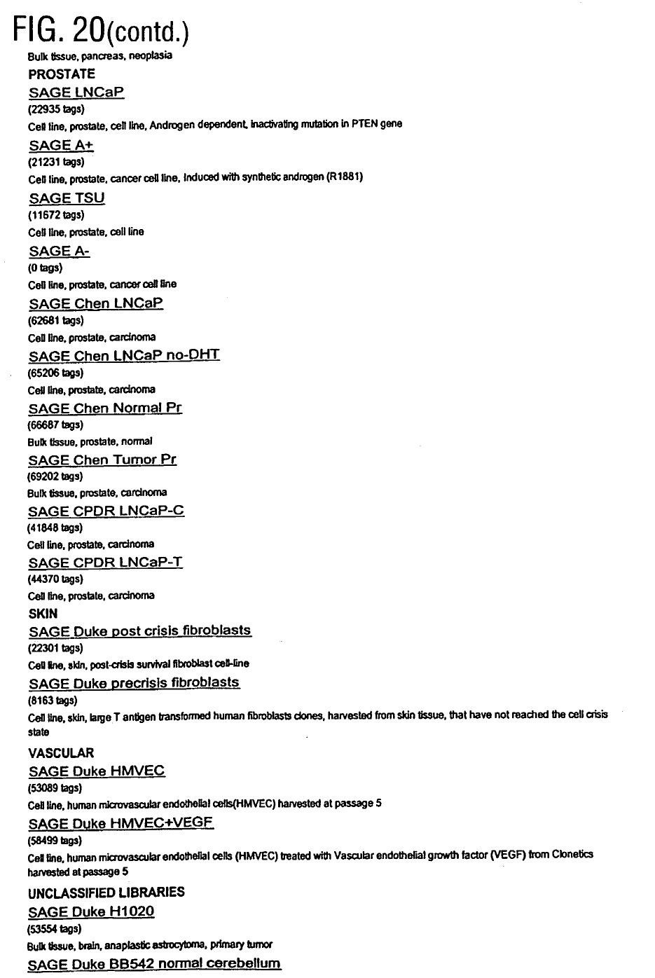

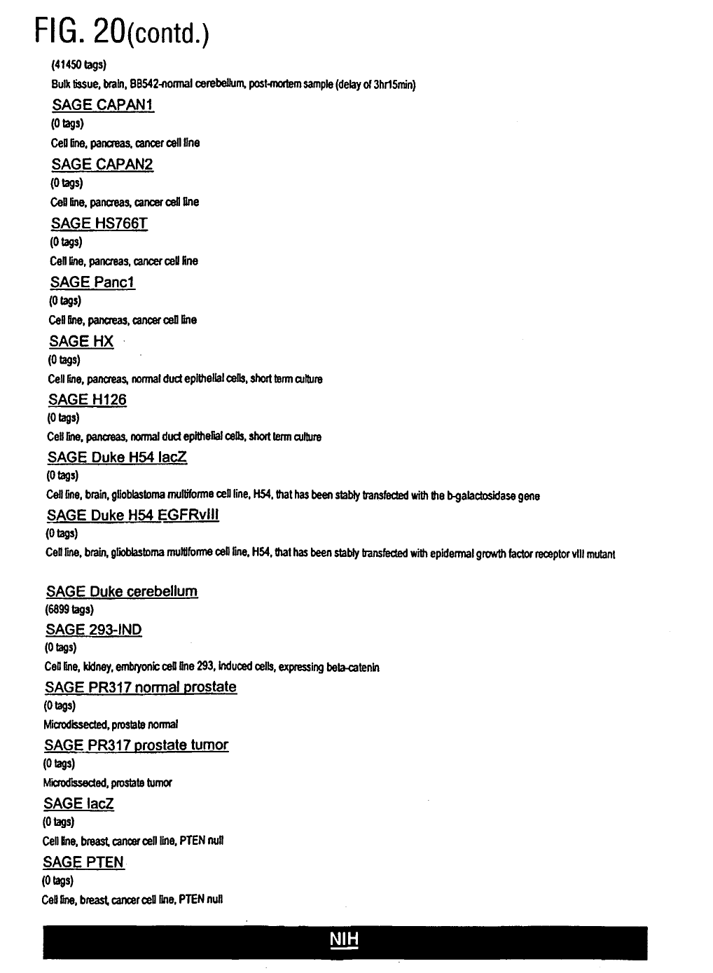

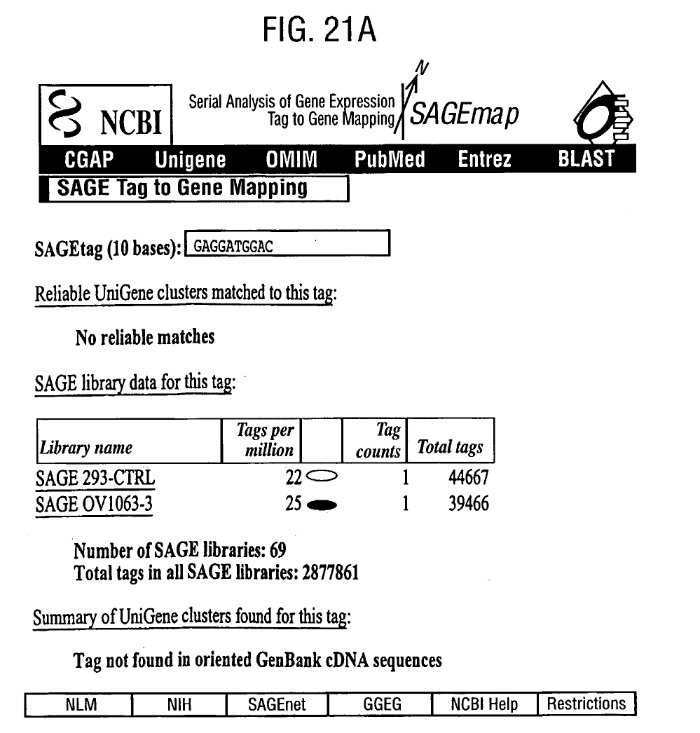

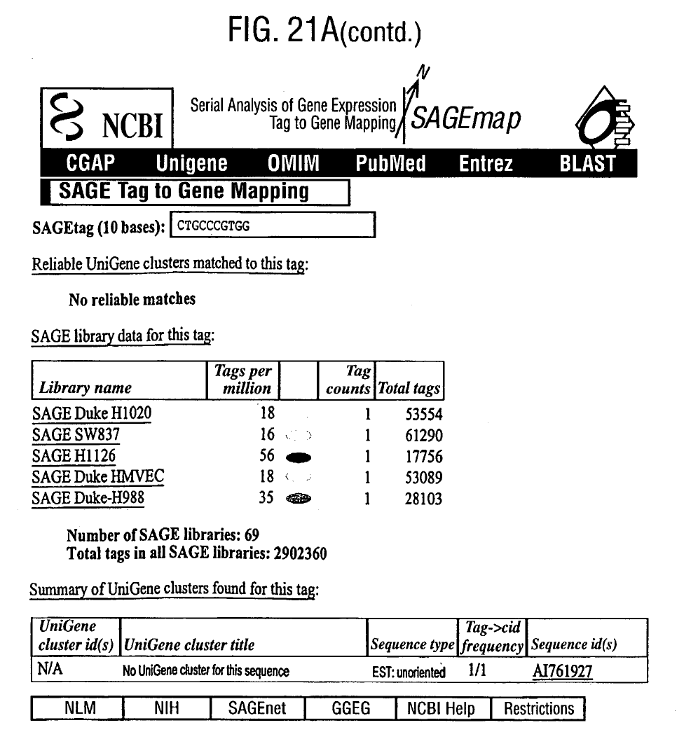

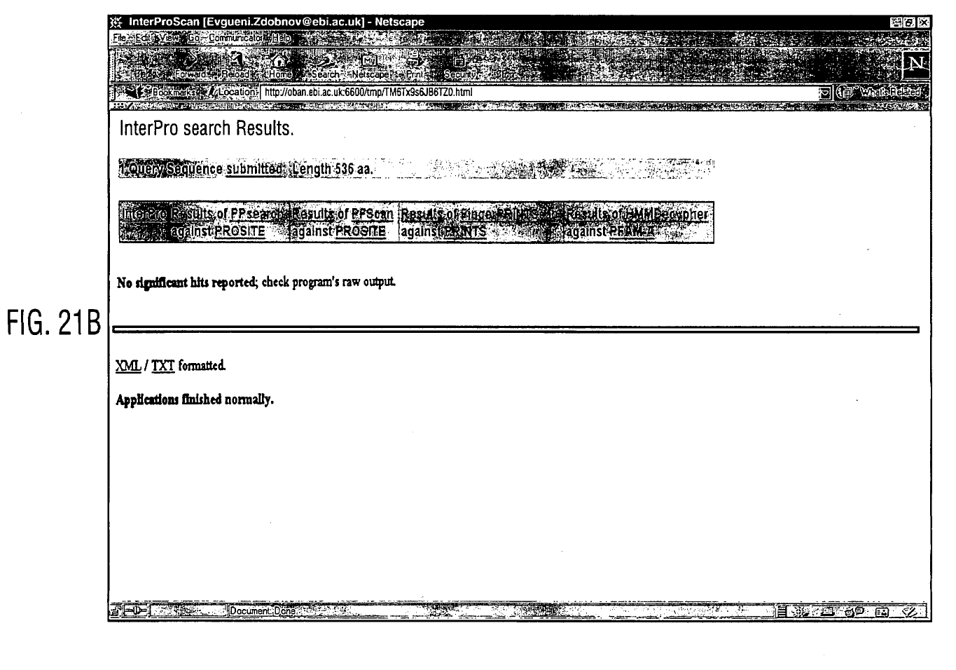

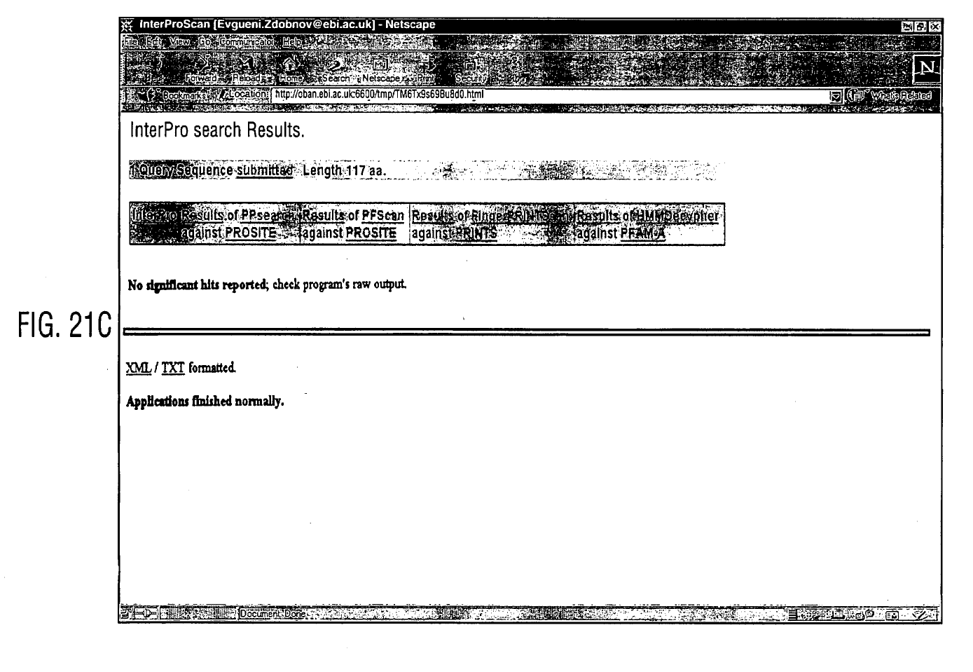

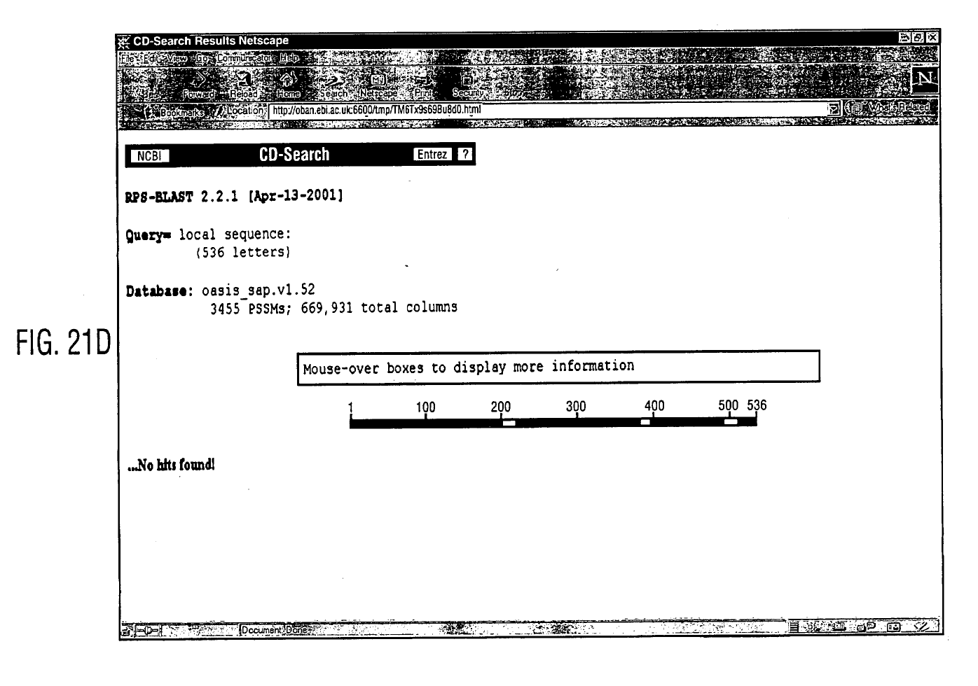

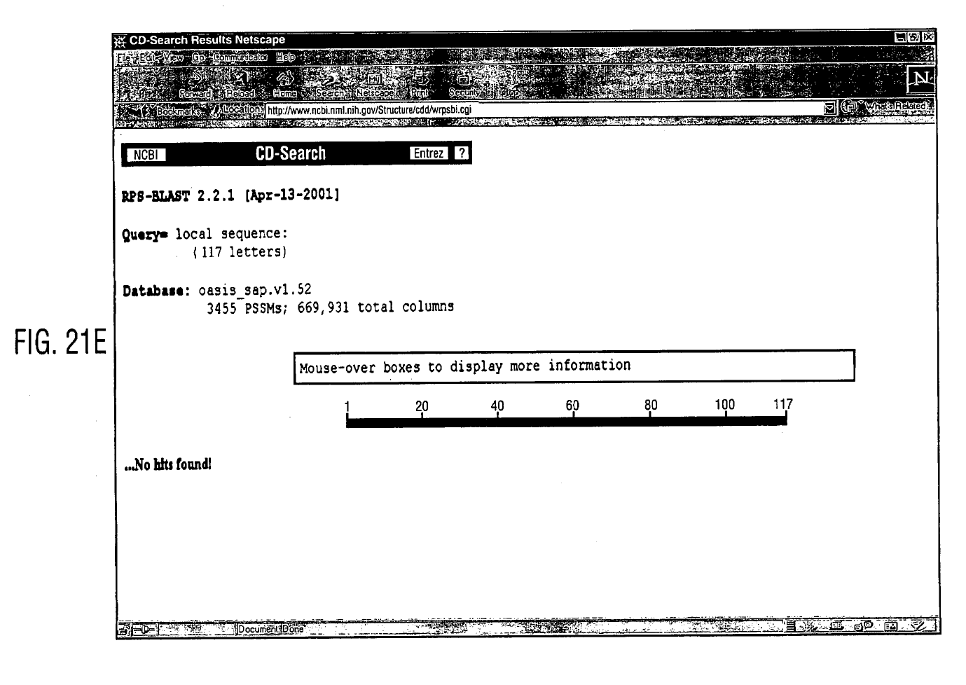

The Serial Analysis of Gene Expression (SAGE) database, curated through the NCBI, gives a direct count of how many times a gene appears in the tissues that they have analysed. FIG. 20 shows a list of all the tissues in the SAGE database. FIG. 21A is a report generated from the SAGE database for G7c (CAB52192.1; AD2), which shows a fairly low level of expression (tags per million) and is present in only a few of the SAGE libraries. This may indicate a very limited tissue distribution or very low levels of expression.

FIG. 21B shows the InterPro results for CAB52162.1 (AD2) as of Jul. 24, 2001. InterPro is a public domain annotation tool which combines PROSITE pattern, PROSITE profile, PRINTS and PFAM. InterPro returns no hits. (no hits are also reported when residues 10-126 of CAB52162.1 (AD2), rather than full-length sequence, is inputted into InterPro, see FIG. 21C). The fact that InterPro returns no results for CAB52162.1 (AD2) on the Jul. 24, 2001 demonstrates that on the date of PCT filing, CAB52162.1 (AD2) could still only be annotated as an adhesion molecule by Inpharmatica Genome Threader™.

FIG. 21D shows the NCBI Conserved Domain Database (CDD) results for CAB52162.1 (AD2) as of Jul. 24, 2001. CDD returns no hits. (no hits are also reported when residues 10-126 of CAB52162.1 (AD2), rather than the full-length sequence, is inputted into CDD, see FIG. 21E). The fact that CDD returns no results for CAB52162.1 (AD2) on the Jul. 24, 2001 demonstrates that on the date of PCT filing, CAB52162.1 (AD2) could still only be annotated as an adhesion molecule by Inpharmatica Genome Threader™.

### Example 3 KIAA0564 (AD3)

In order to initiate a search for novel, distantly related integrins, an archetypal family member, Leukocyte Function Associated Molecule-1 (LFA), alpha subunit is chosen. More specifically, the search is initiated using a structure from the Protein Data Bank (PDB) which is operated by the Research Collaboratory for Structural Bioinformatics.

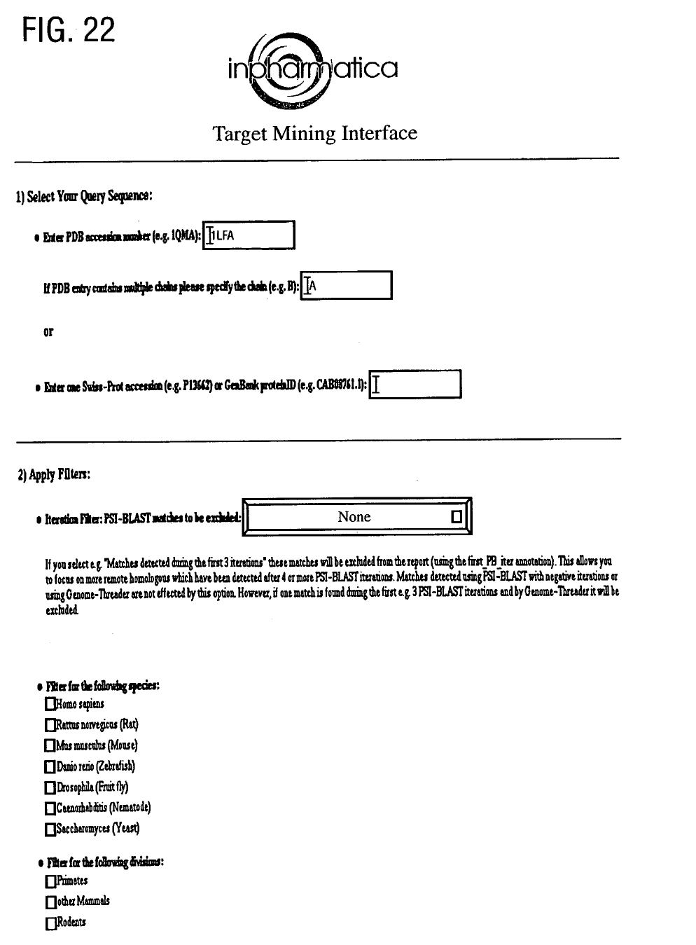

The structure chosen represents the I domain (insertion domain) of LFA-1, PDB code 1LFA:A (FIG. 22). Lymphocyte function-associated antigen 1 (LFA-1) is a leukocyte integrin that supports inflammatory and immune responses by mediating cell adhesion, the trafficking of leukocytes, and the augmentation of signalling through the T cell receptor. This integrin consists of a CD11a and a CD18 chain and binds to the cell surface ligands intercellular adhesion molecule 1 (ICAM-1), ICAM-2, and ICAM-3. Mutational studies indicate that ICAM-I interacts with LFA-1 through a module of approximately 200 residues designated the I domain that is located in CD11. The I domain is the site of interaction between integrins and intercellular adhesion molecules. Integrin I domains are homologous to the A-domains present in von Willebrand factor, several collaggen and complement proteins, and cartilage matrix protein, all proteins with adhesive functions (Huth, J. R. , et al. Proc Natl Acad Sci USA. 2000 97(10):5231-6).

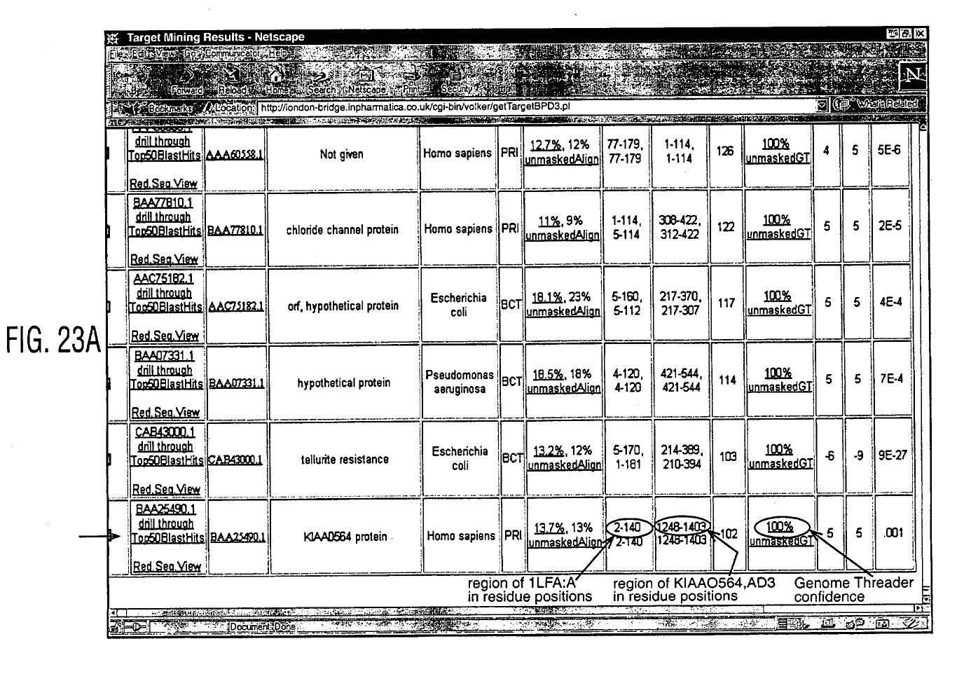

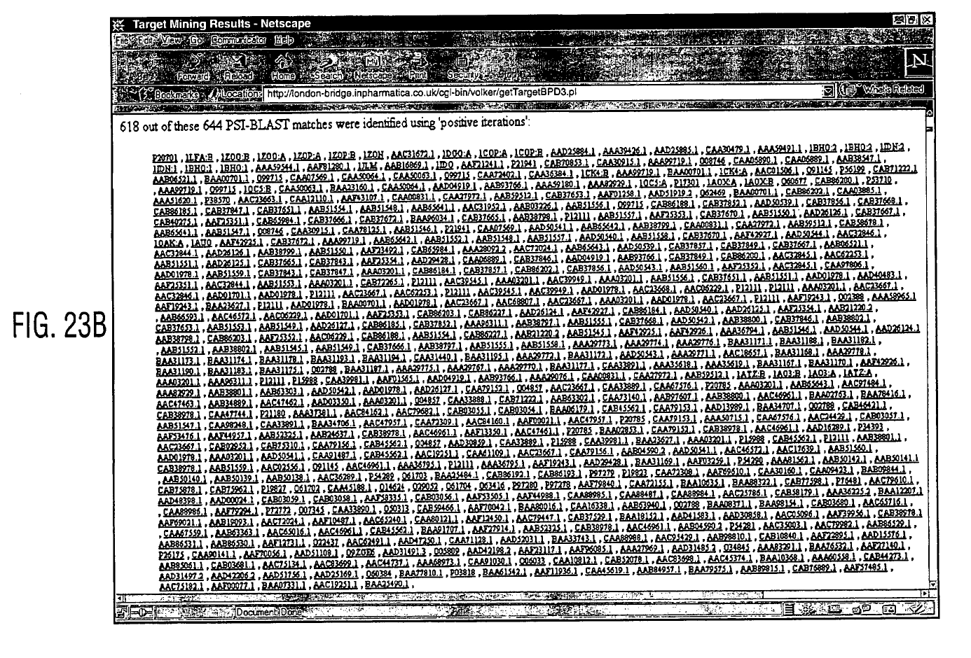

A search of the Biopendium for homologues of 1LFA takes place and returns 557 Inpharmatica Genome Threader™ results (selection given in FIG. 23A) and 420 PSI-Blast results (selection in FIG. 23B). The 557 Genome Threader™ results include examples of other I domain containing integrins, such as H. sapiens MAC-1 and LFA-1 as well as Collagen alpha 1 and Von Willebrand Factor. Among the known I domain containing adhesion molecules/proteins appears a protein of apparently unknown function, KIAA0564 (BAA25490.1; AD3, FIG. 23A).

The Inpharmatica Genome Threader™ has thus identified residues 1248-1403 of a sequence, KIAA0564 (AD3), as having an equivalent structure to residues 2-140 of the I domain of LFA-1 (1LFA:A), the known interaction domain between LFA-1 and ICAM. Having a structure similar to this domain suggests that KIAA0564 (AD3) is a protein that functions in cellular adhesion. The Inpharmatica Genome Threader™ identifies this with 100% confidence.

PSI-Blast (FIG. 23B) is unable to identify this relationship; it is only the Inpharmatica Genome Threader™ that is able to identify residues 1248-1403 of KIAA0564 (AD3) as having an I domain. PSI-Blast does identify LFA-1 itself and other related integrins with varying degrees of probability (E value) as would be expected.

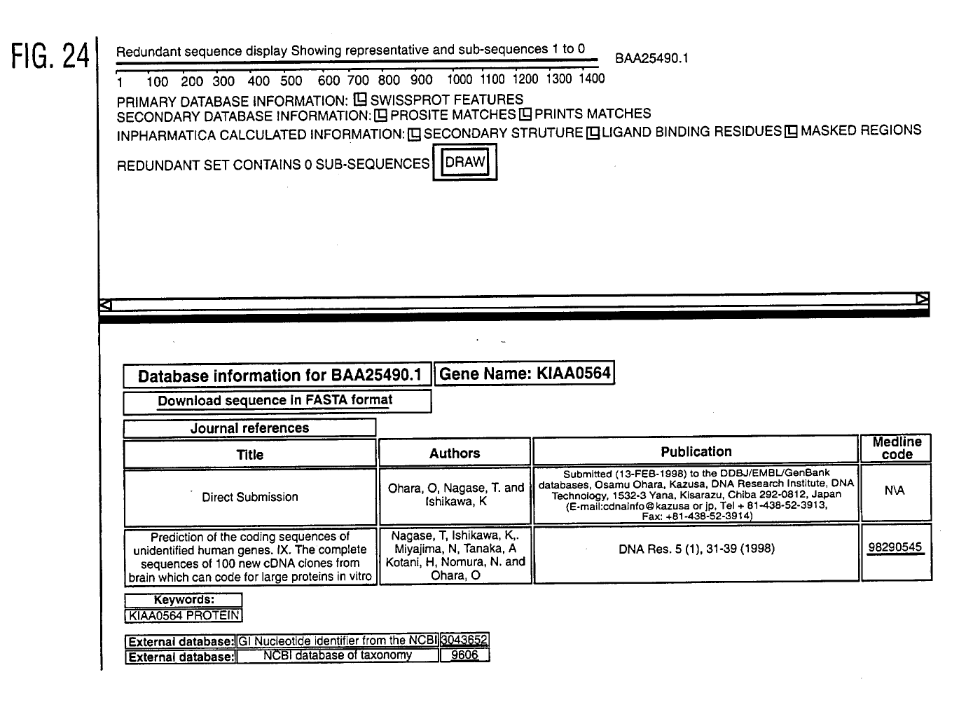

In order to view what is known in the public domain databases about KIAA0564 (AD3), the Redundant Sequence Display Page (FIG. 24) is viewed. KIAA0564 (AD3) is a Homo sapiens sequence, its GenBank protein ID is BAA25490.1 and it is 2047 amino acids in length. There are no associated PROSITE or PRINTS hits for this sequence. PROSITE and PRINTS are databases that help to describe proteins of similar families. Returning zero hits from both databases means that KIAA0564 (AD3) is unidentifiable as an I domain containing adhesion molecule using PROSITE or PRINTS.

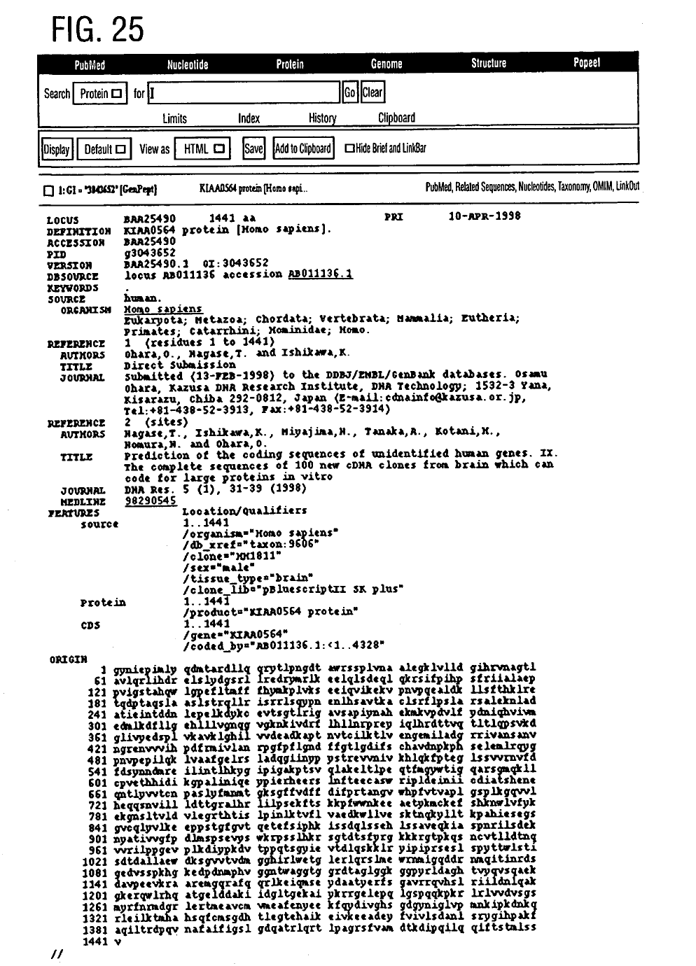

The National Centre for Biotechnology Information (NCBI) GenBank protein database is viewed to examine if there is any further information that is known in the public domain relating to KIAA0564 (AD3). This is the U.S. public domain database for protein and gene sequence deposition (FIG. 25). KIAA0564 was cloned by a group of scientists in Chiba, Japan (Nagase, T. et al, (1998) DNA Res. 5(1), 31-39). There is no further annotation for KIAA0564 except that the KIAA0564 gene was cloned from brain tissue. The public domain information for this gene does not annotate it as an integrin or an I domain-containing protein, or indeed, contain any suggestion whatsoever for the function of this protein.

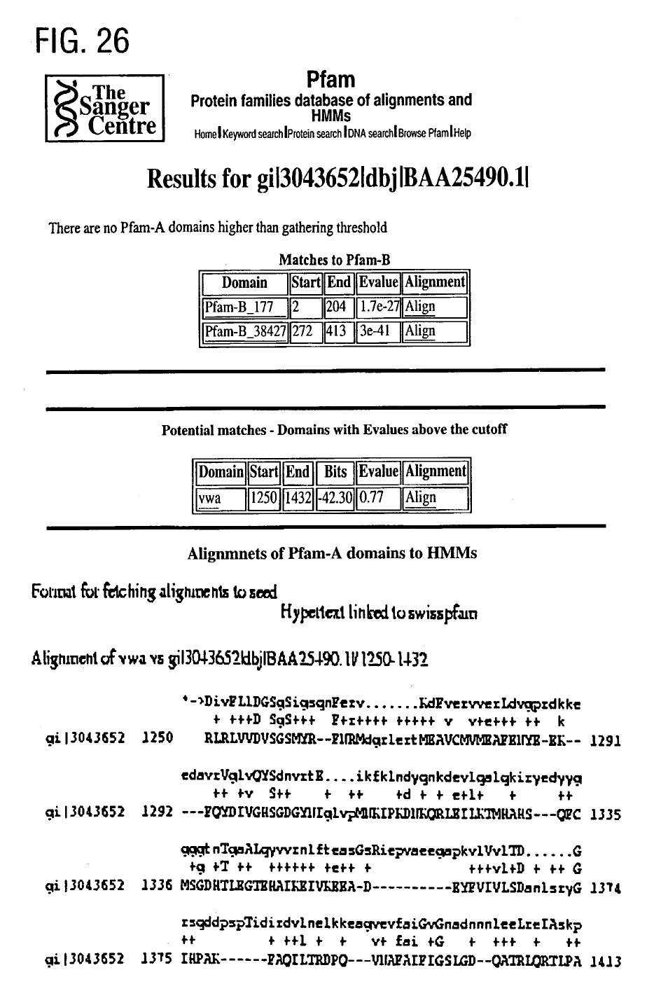

In order to identify whether any other public domain annotation vehicle is able to annotate KIAA0564 as an I domain containing protein, the KIAA0564 protein sequence is searched against the Protein Family Database of Alignment and HMM's (PFAM) database (FIG. 26). The results identify that KIAA0564 has no identifiable PFAMs. It may have a Von Willebrand domain (VWA) but this is below the threshold of credibility: the certainty of this is very low (E=0.77) and as such is not reliable. PFAM does not identify KIAA0564 (AD3) as having an I domain.

Therefore using all public domain annotation tools KIAA0564 (AD3) is not annotated as a protein involved in adhesion through the presence of an I domain. Only the Inpharmatica Genome Threader™ is able to annotate this protein as possessing an I domain.

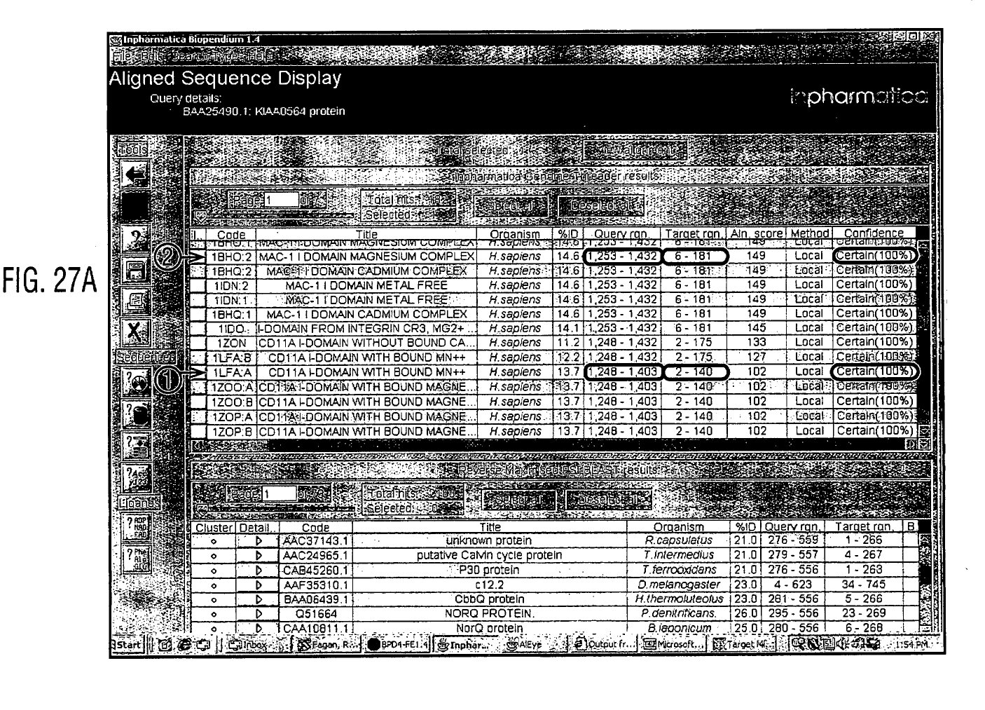

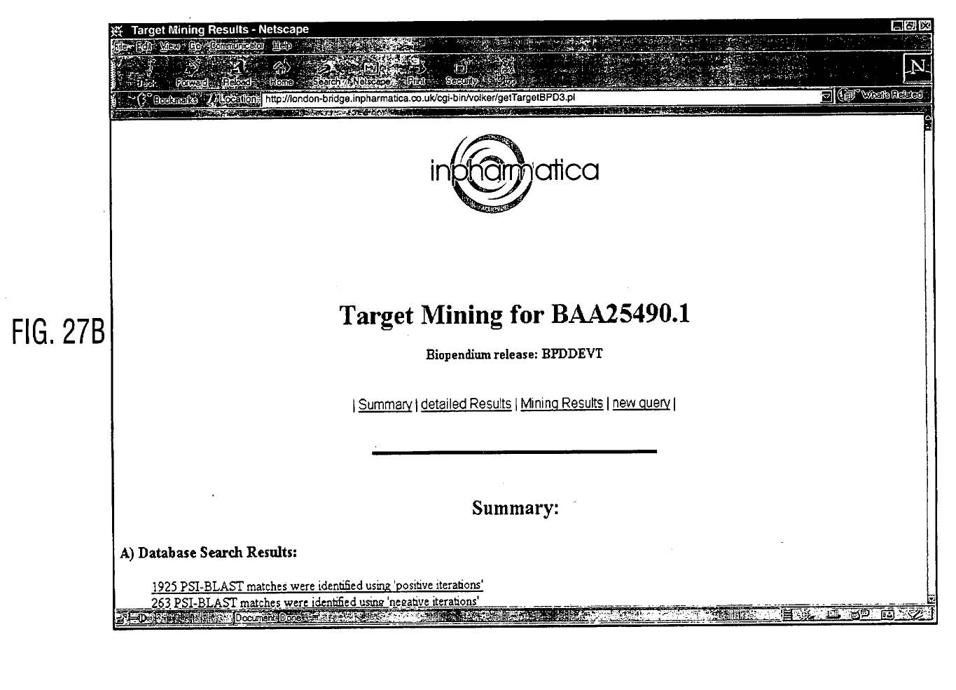

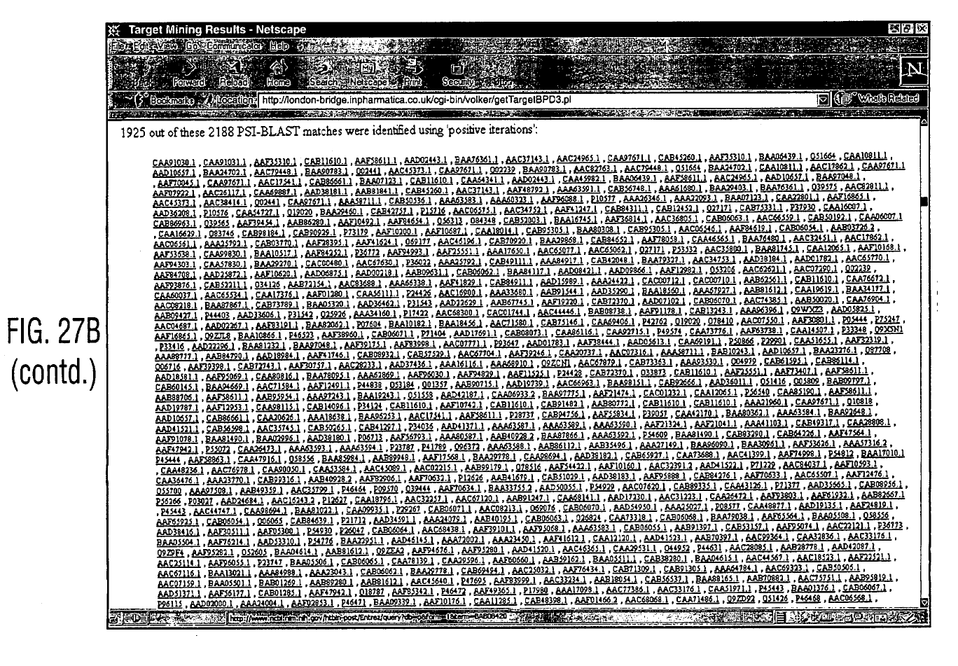

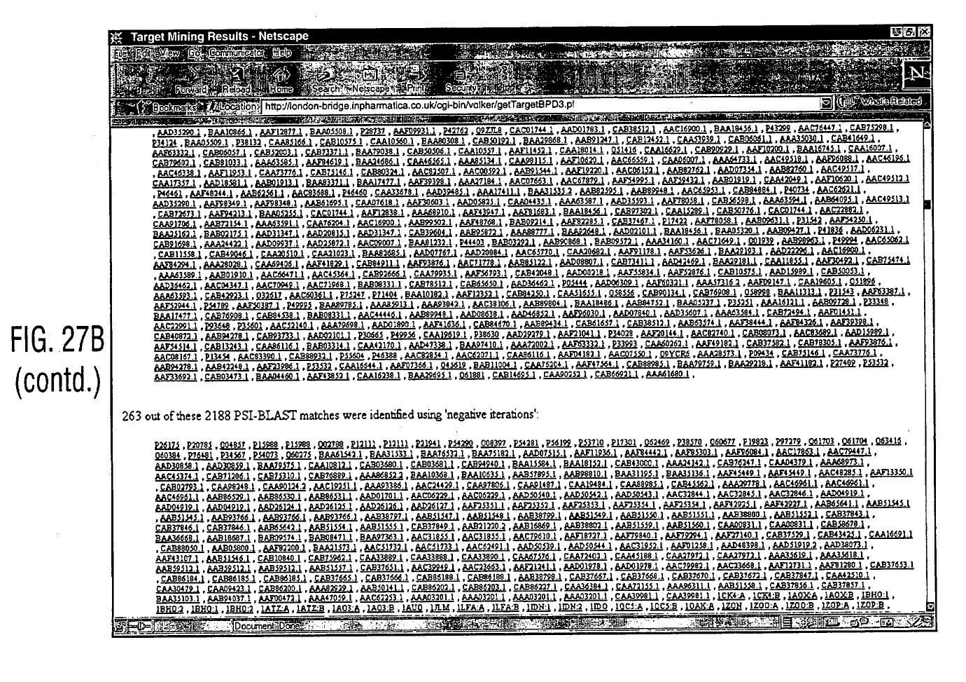

The reverse search is now carried out. KIAA0564 (BAA25490.1; AD3) is now used as the query sequence in the Biopendium™. The Inpharmatica Genome Threader™ identifies 182 hits (FIG. 27A) while PSI-Blast returns 2188 hits (FIG. 27B). The Inpharmatica Genome Threader™ (FIG. 27A, arrow \mathring{1}) identifies residues 1248-1403 of KIAA0564 (AD3) as having a structure the same as the I domain of the integrin LFA-1 (1LFA:A) with a confidence of 100%. The Inpharmatica Genome Threader™ (FIG. 27A, arrow \mathring{2}) also identifies residues 1253-1432 of KIAA0564 (AD3) as having a structure the same as the I domain of the integrin MAC-1 (1BHO:2) with a confidence of 100%. Thus a region from residues 1248-1253 to residues 1403-1432 of KIAA0564 (AD3) has been identified as adopting an equivalent fold to a range of I-domains including those of the adhesion molecules LFA-1 and MAC-1.

PSI-Blast does not return this result. It is only the Inpharmatica Genome Threader™ and that is able to identify this relationship.

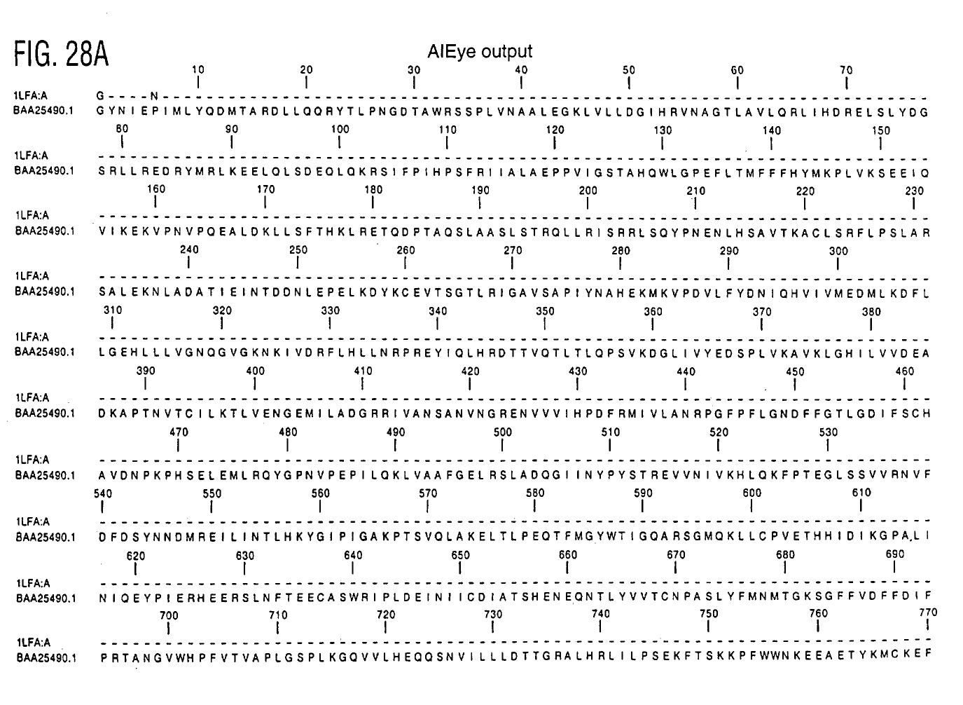

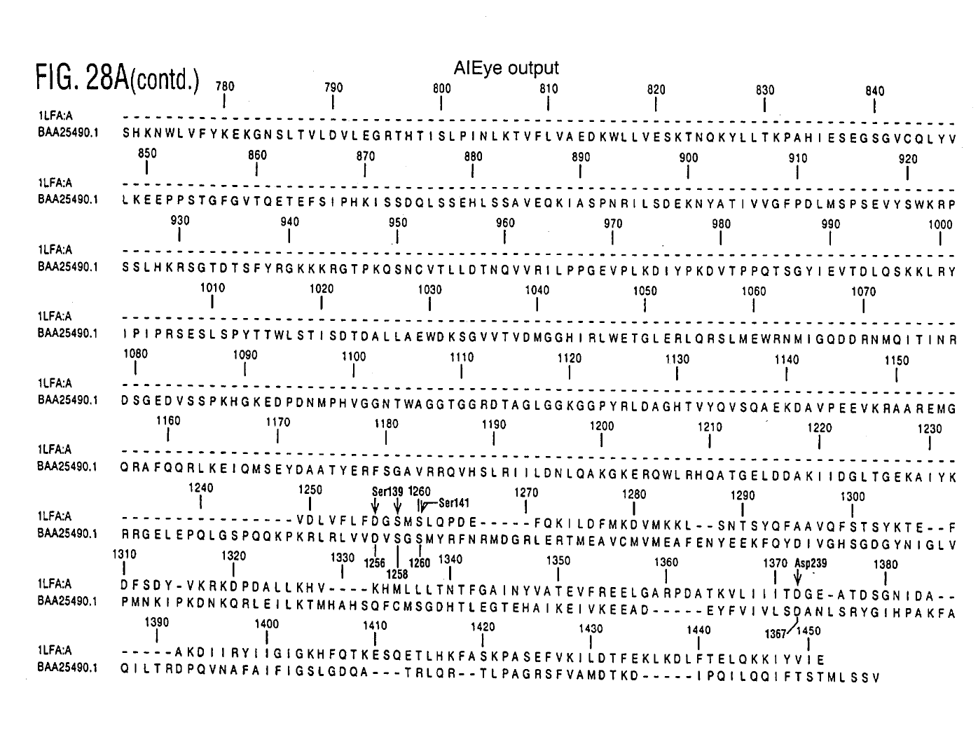

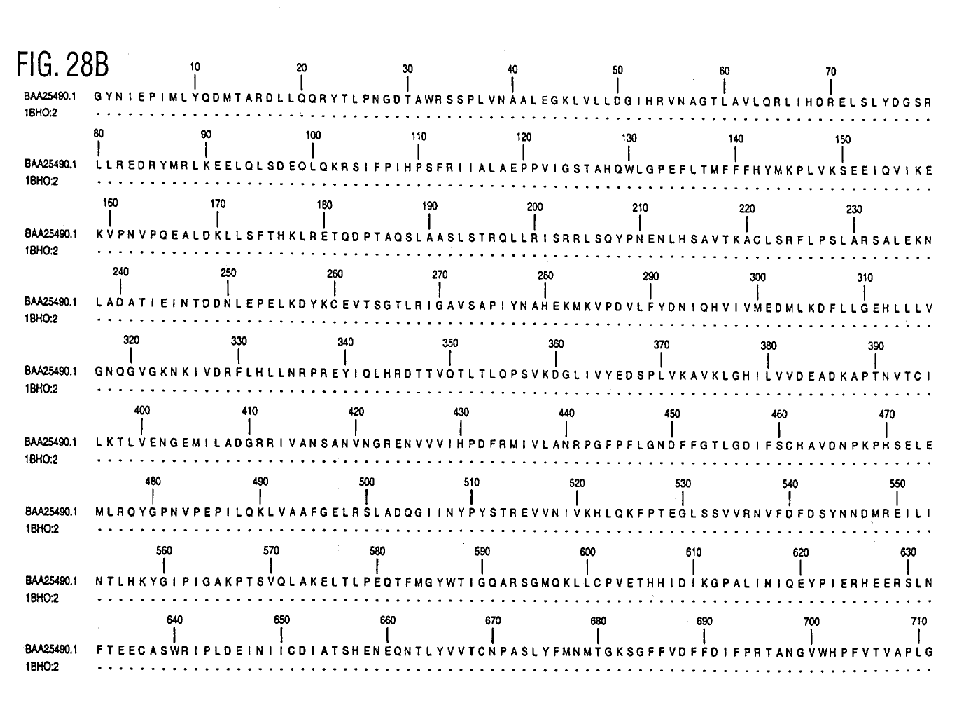

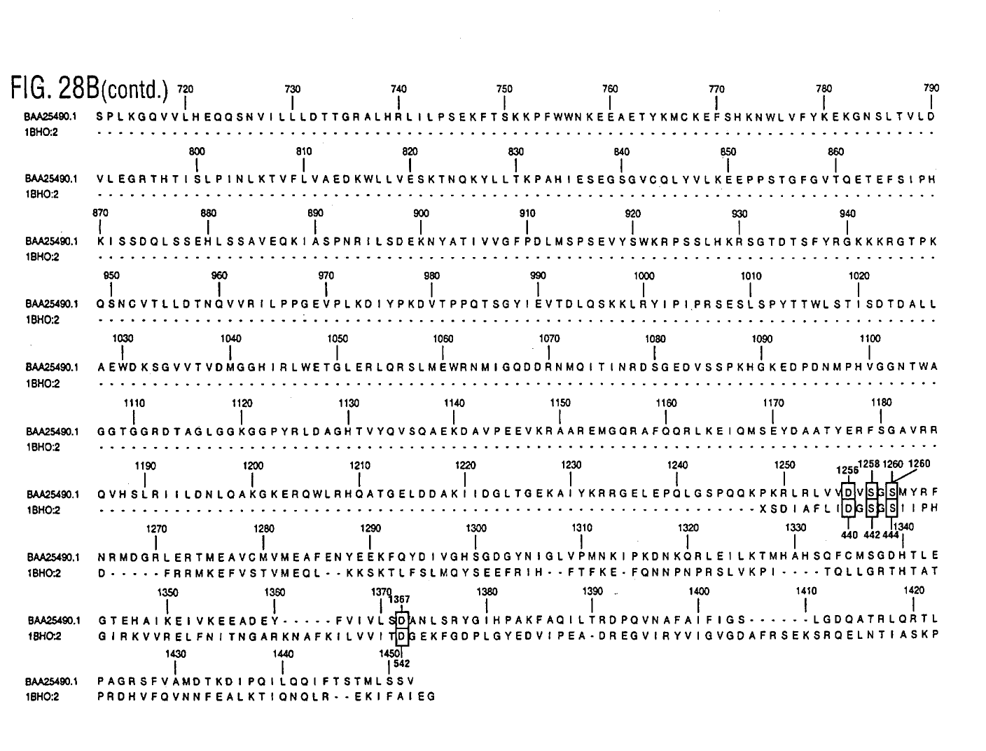

Among the integrin I domains that the Inpharmatica Genome Threader™ returns is the I domain from integrin LFA-1 (1LFA:A). This is chosen (highlighted) against which to view the sequence alignment of KIAA0564 (AD3). Viewing the alignment (FIG. 28A) of the query protein against the proteins identified as being of a similar structure helps to visualize the areas of homology. FIG. 28A illustrates the point that the divalent cation binding residues of 1LFA:A (Ser139, Ser141, and Asp239) is conserved as Ser1258, Ser1260 and Asp1367, respectively, in KIAA0564 (AD3).

FIG. 28B shows the Genome Threader™alignment of KIAA0564 (AD3) with the Integrin MAC-1 I-domain (1BHO:2). FIG. 28B illustrates that the metal binding residues of 1BHO:2 (Ser442, Ser444 and Asp542) are conserved as Ser1258, Ser1260 and Asp1367, respectively, in KIAA0564 (AD3).

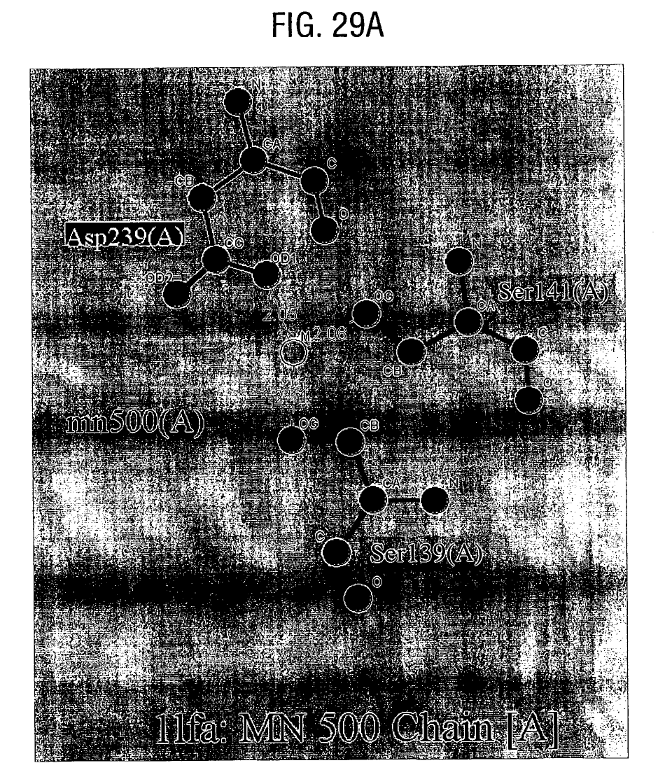

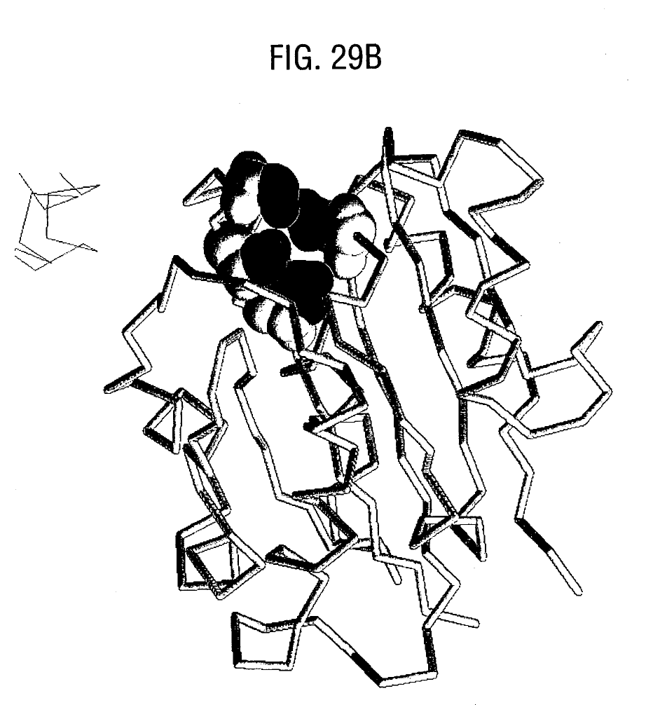

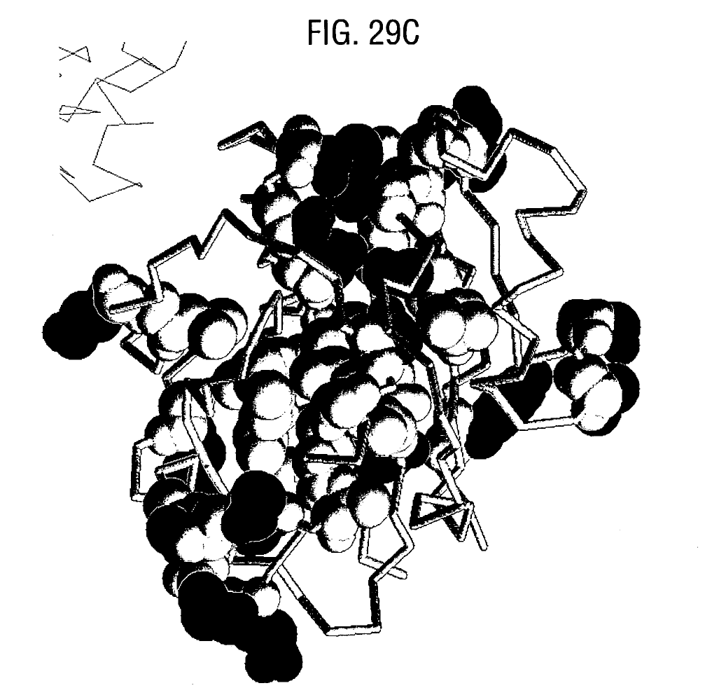

In order to ensure that the protein identified is a homologue of the query sequence, the visualisation program LigEye (FIG. 29A) and RasMol (FIG. 29B) are used. These visualisation tools identify the active site of known protein structures by indicating the amino acids with which known small molecule inhibitors interact at the active site. These interactions are through either a direct hydrogen bond or hydrophobic interactions. In this manner one can see if the active site fold/structure is conserved between the identified homologue and the chosen protein of known structure.

This visualisation is shown with 1LFA, which illustrates the sites of interaction of a mangenese (MN) ion with the I domain of integrin Alpha 2-Beta 1 (FIG. 29A). The MN ion sees 3 different amino acids in the I domain of 1LFA. The three amino acids of 1LFA (SER139, SER141, and Asp239) are conserved in KIAA0564 (FIG. 29B, ball structures). FIG. 29C further identifies the amino acids that are conserved in 1LFA and KIAA0564 conservation occurs throughout the structure though it is especially marked in the metal ion binding region and the central core of the protein. The conservation of amino acids indicates that the fold is identical. This indicates that indeed as predicted by the Inpharmatica Genome Threader™, KIAA0564 (AD3) folds in a similar manner to 1LFA and as such is identified as an I domain containing protein.

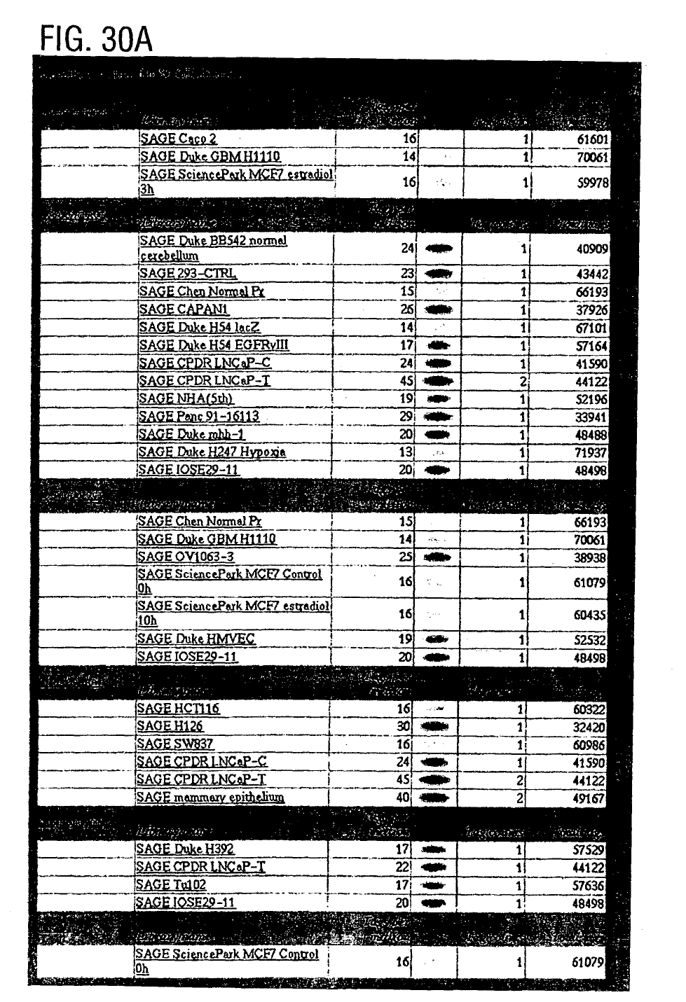

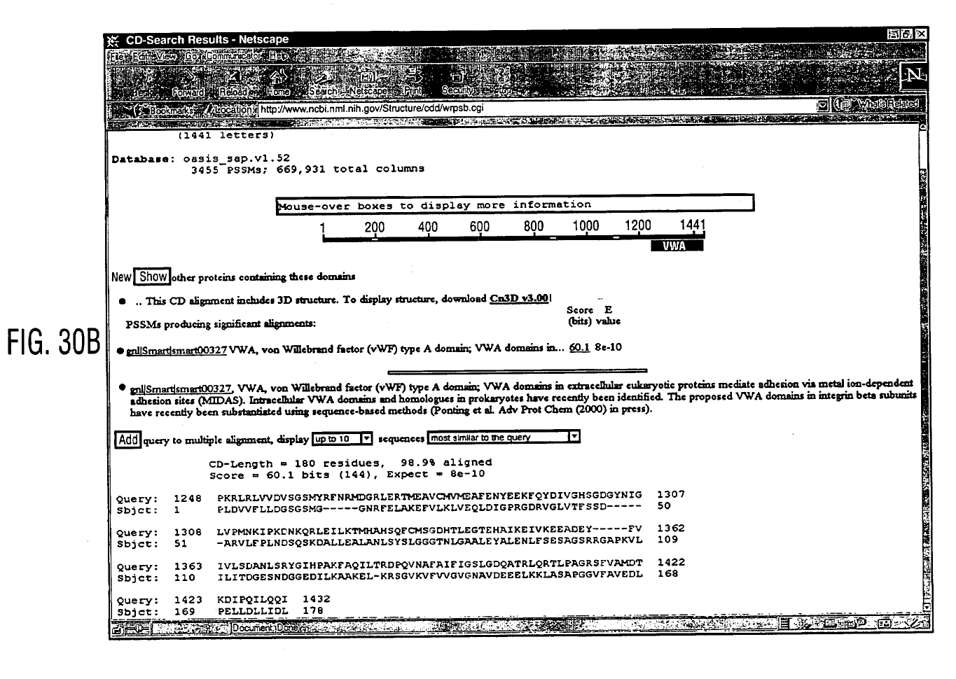

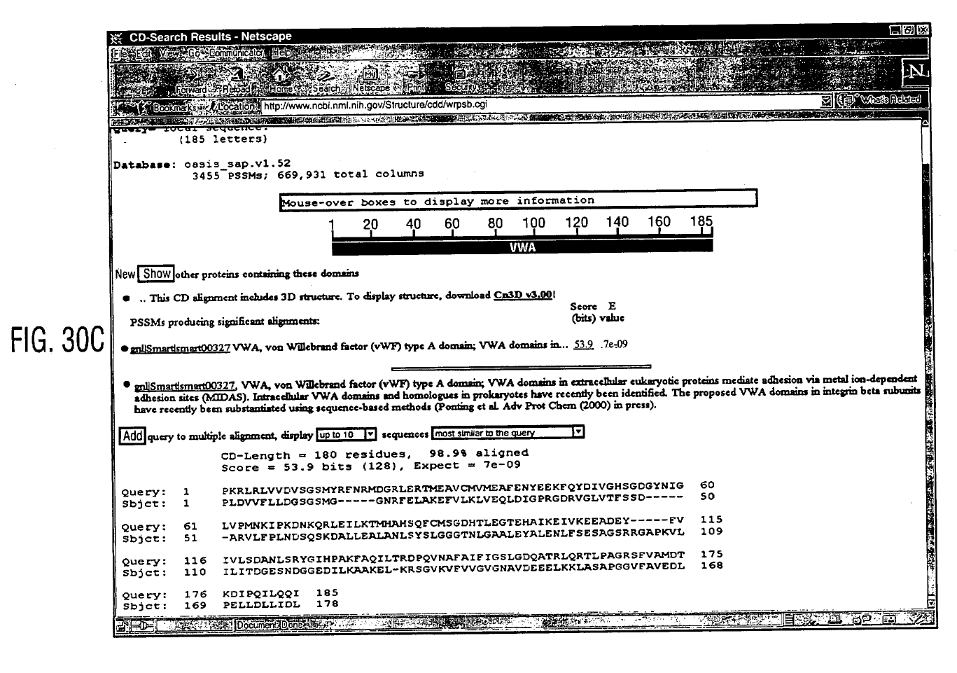

The Serial Analysis of Gene Expression (SAGE) database gives a direct count of how many times the gene appears in the tissues that have been analysed. FIG. 30A is a report generated from the SAGE database for KIAA0564 (AD3), which shows a fairly low level of expression (tags per million) in a variety of tissues.

FIG. 30B shows the NCBI Conserved Domain Database (CDD) results for KIAA0564 (AD3) as of Jul. 24, 2001. CDD returns one hit. (An identical hit is reported when residues 1248-1432 of KIAA0564 (AD3), rather than the full-length sequence, is inputted into CDD, see FIG. 30C). The match is to the smart00327 profile which annotates residues 1248-1432 of KIAA0564 (AD3) as possessing a von Willebrand factor/I-domain (smart00327). This public domain annotation of KIAA0564 (AD3) as possessing an I-domain only became available on Jun. 30, 2001 (see FIG. 11H). Thus this public domain annotation can be considered supporting evidence for the Genome Threader™ annotation of a region including, at the most, residue 1248 to residue 1432 of KIAA0564 (AD3), and at the least, residue 1253 to residue 1403 of KIAA0564 (AD3) as being an I-domain and functioning as an adhesion molecule.

### Example 4 NG37 (AD4)

In order to initiate a search for novel, distantly related integrins, an archetypal family member, Mac-1, is chosen. More specifically, the search is initiated using a structure from the Protein Data Bank (PDB) which is operated by the Research Collaboratory for Structural Bioinformatics.

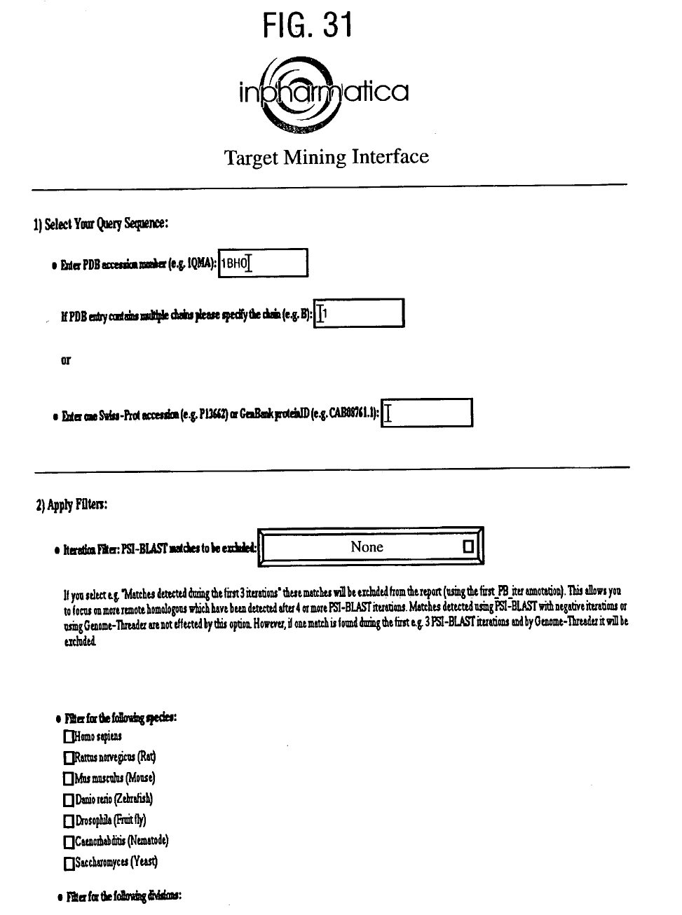

The structure chosen represents the I domain (insertion domain) of Mac-1, PDB code 1BHO:1 (FIG. 31). This integrin consists of a CD11b alpha chain and a beta chain. Mac-1 binds to receptors via a module of approximately 200 residues designated the I domain that is located in CD11b. The I domain is the site of interaction between integrins and intercellular adhesion molecules. Integrin I domains are homologous to the A-domains present in von Willebrand factor, several collaggen and complement proteins, and cartilage matrix protein, all proteins with adhesive functions (Huth, J. R. , et al., Proc Natl Acad Sci USA. 2000 May 9;97(10):5231-6).

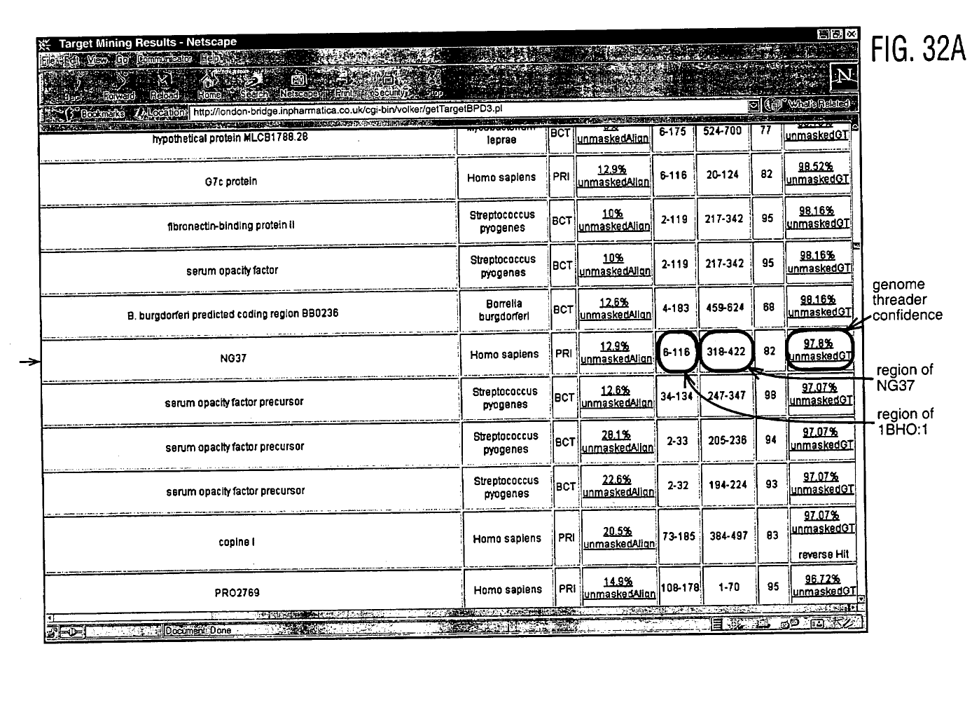

A search of the Biopendium for homologues of 1BHO:1 takes place and returns 621 Inpharmatica Genome Threader™ results (selection given in FIG. 32A) and 431 PSI-Blast results (selection in FIG. 32B). The 621 Genome Threader™ results include examples of other I domain-containing integrins, such as LFA-1 as well as Collagen alpha 1 and Von Willebrand Factor. Among the known I domain containing adhesion molecules/proteins appears a protein of apparently unknown function, NG37 (AAD21820.1; AD4, FIG. 32A).

The Inpharmatica Genome Threader™ has thus identified residues 318-422 of a sequence, NG37 (AD4), as having an equivalent structure to residues 6-116 of the I domain of MAC-1 (1BHO:1), the known interaction domain of MAC-1. Having a structure similar to this domain suggests that NG37 (AD4) is a protein that functions in cellular adhesion. The Inpharmatica Genome Threader™ identifies this with 97.8% confidence.

PSI-Blast (FIG. 32B) is unable to identify this relationship; it is only the Inpharmatica Genome Threader™ that is able to identify residues 318-422 of NG37 (AD4) as having an I domain. PSI-Blast does identify MAC-1 itself and other related integrins with varying degrees of probability (E value) as would be expected.

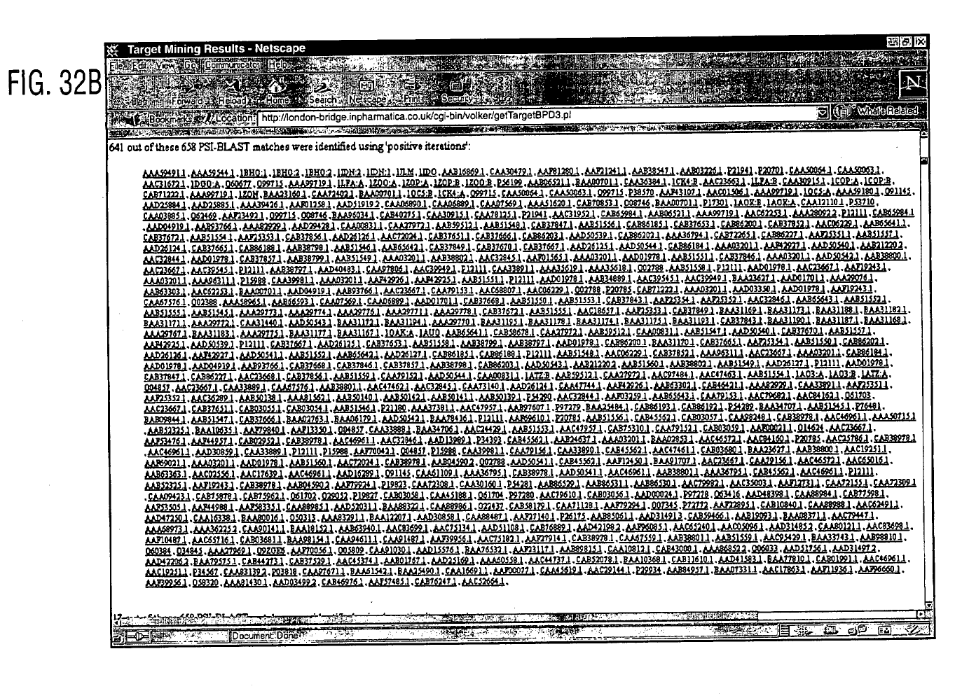

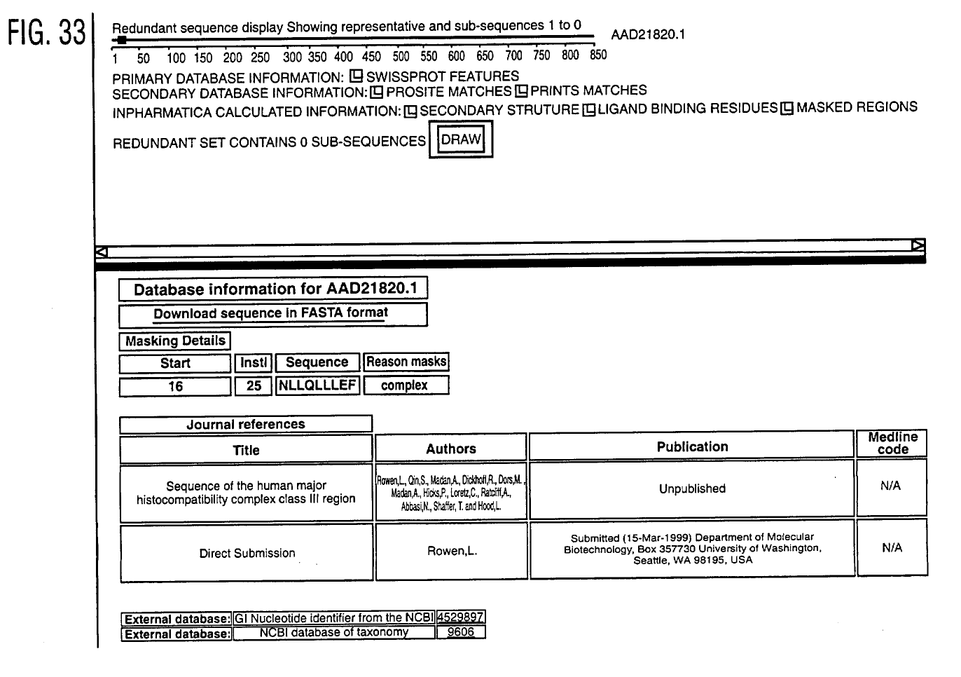

In order to view what is known in the public domain databases about NG37 (AD4), the Redundant Sequence Display Page (FIG. 33) is viewed. NG37 (AD4) is a Homo sapiens sequence, its GenBank protein ID is AAD21820.1 and it is 852 amino acids in length. There are no associated PROSITE or PRINTS hits for this sequence. PROSITE and PRINTS are databases that help to describe proteins of similar families. Returning zero hits from both databases means that NG37 (AD4) is unidentifiable as an I domain containing adhesion molecule using PROSITE or PRINTS.

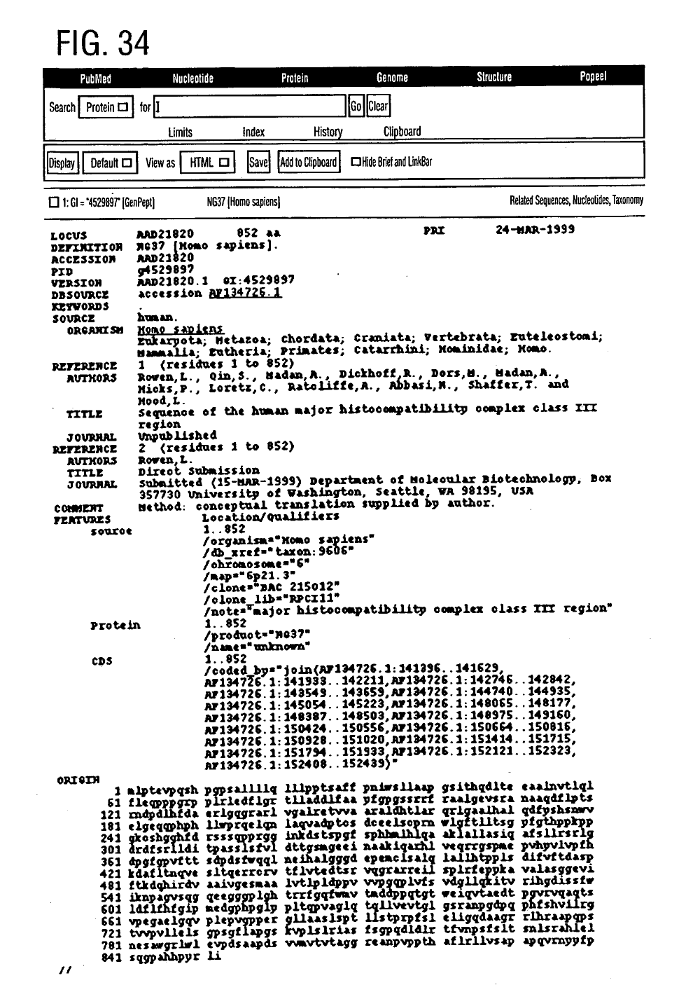

The National Centre for Biotechnology Information (NCBI) GenBank protein database is viewed to examine if there is any further information that is known in the public domain relating to NG37 (AD4). This is the U.S. public domain database for protein and gene sequence deposition (FIG. 34). NG37 was cloned by a group of scientists at the University of Washington (Unpublished). There is no further annotation for NG37 except that the NG37 gene was cloned the human major histocompatibility complex class III region. The public domain information for this gene does not annotate it as an integrin or an I domain-containing protein, or indeed, contain any suggestion whatsoever for the function of this protein other than it originates from the major histocompatibility complex class III region.

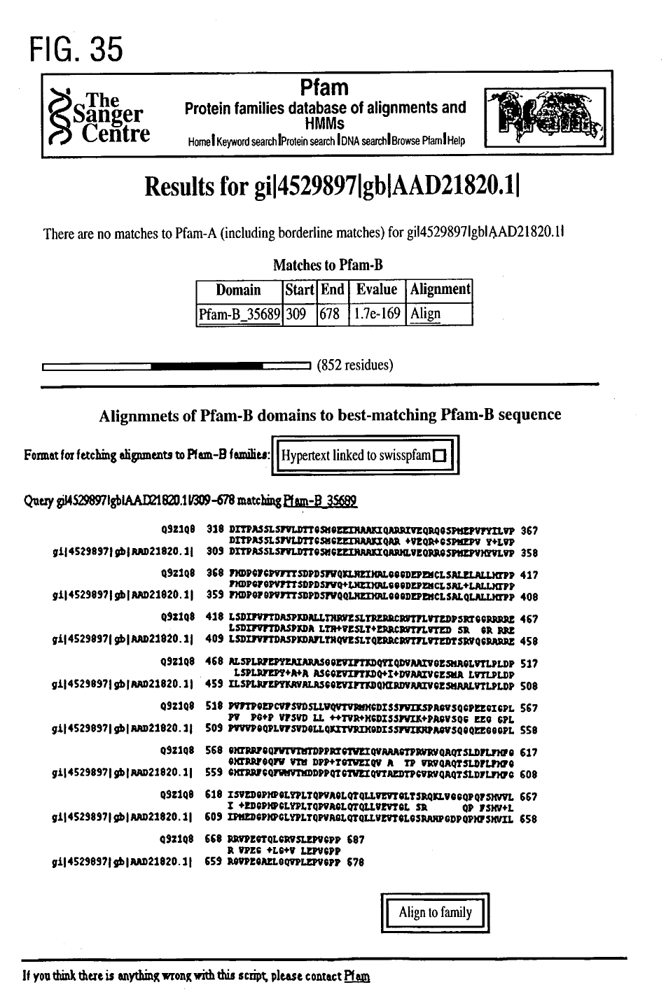

In order to identify whether any other public domain annotation vehicle is able to annotate NG37 as an I domain containing protein, the NG37 protein sequence is searched against the Protein Family Database of Alignment and HMM's (PFAM) database (FIG. 35). PFAM does not identify NG37 (AD4) as having an I domain or any other functional information.

Therefore using all public domain annotation tools NG37 (AD4) is not annotated as a protein involved in adhesion through the presence of an I domain. Only the Inpharmatica Genome Threader™ is able to annotate this protein as possessing an I domain.

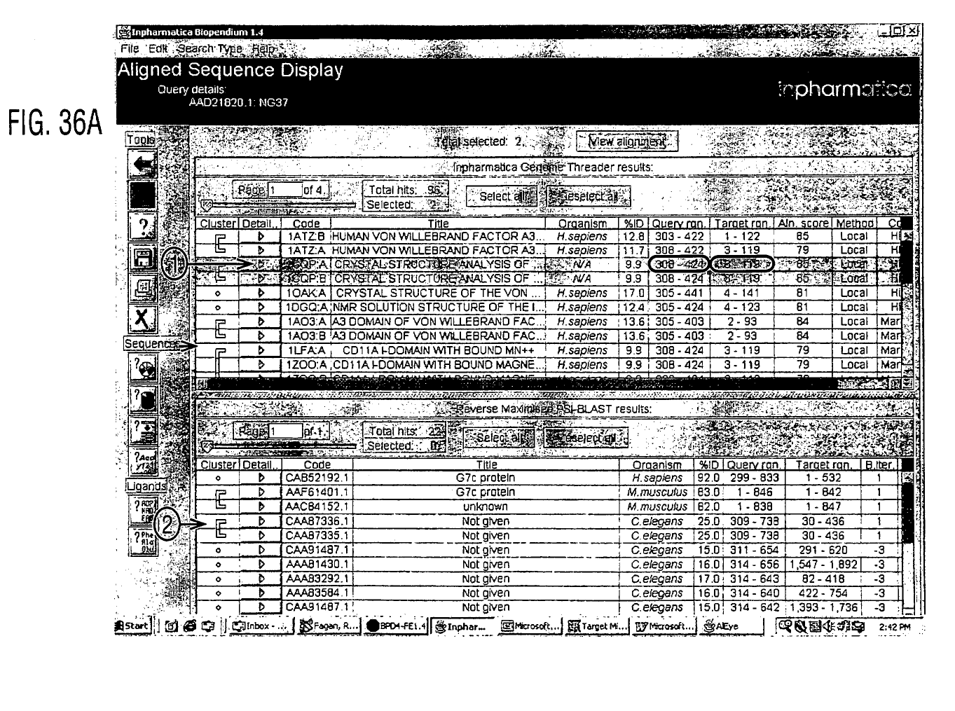

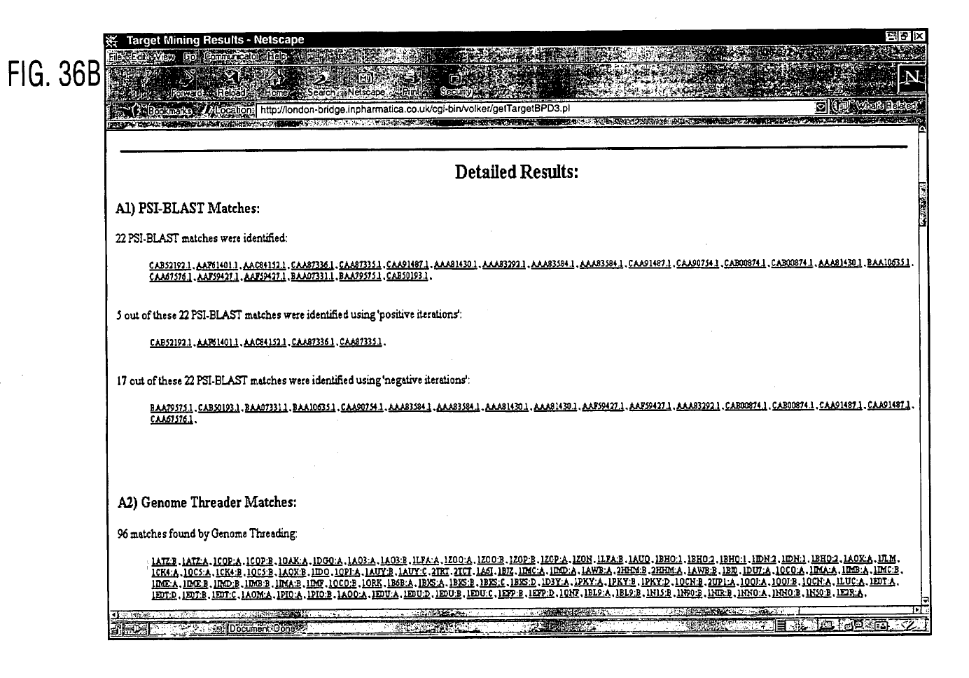

The reverse search is now carried out. NG37 (AAD21820.1; AD4) is now used as the query sequence in the Biopendium™. The Inpharmatica Genome Threader™ identifies 96 hits (FIG. 36A) while PSI-Blast returns 22 hits (FIG. 36B). The Inpharmatica Genome Threader™ (FIG. 36A, arrow \mathring{1}) identifies residues 308-424 of NG37 (AD4) as having a structure the same as the I domain of the Integrin LFA-1 (1CQP:A). PSI-Blast does not return this result. PSI-Blast is only able to identify this relationship in the negative iteration, which the Biopendium™ computes through its all by all calculation.

It is only the Inpharmatica Genome Threader™ and negative iteration PSI-Blast that is able to identify this relationship.

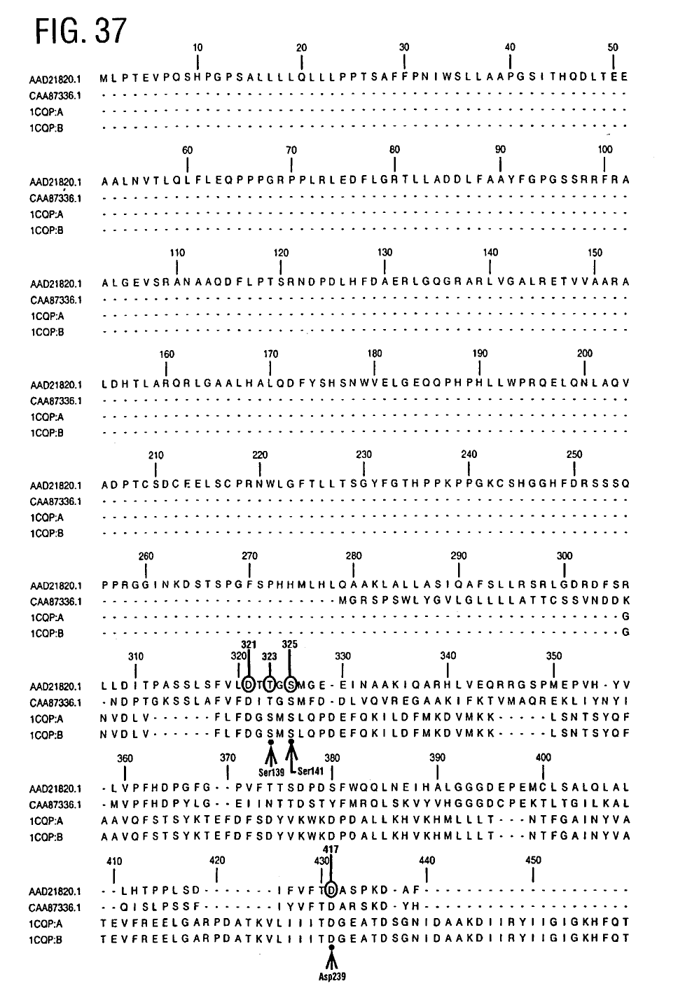

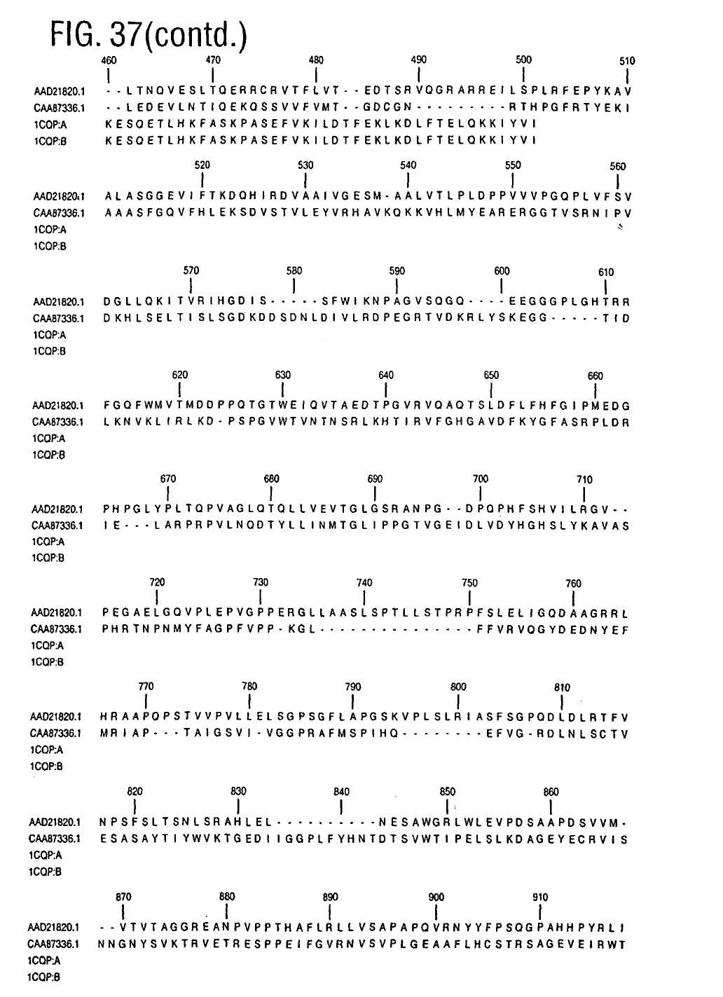

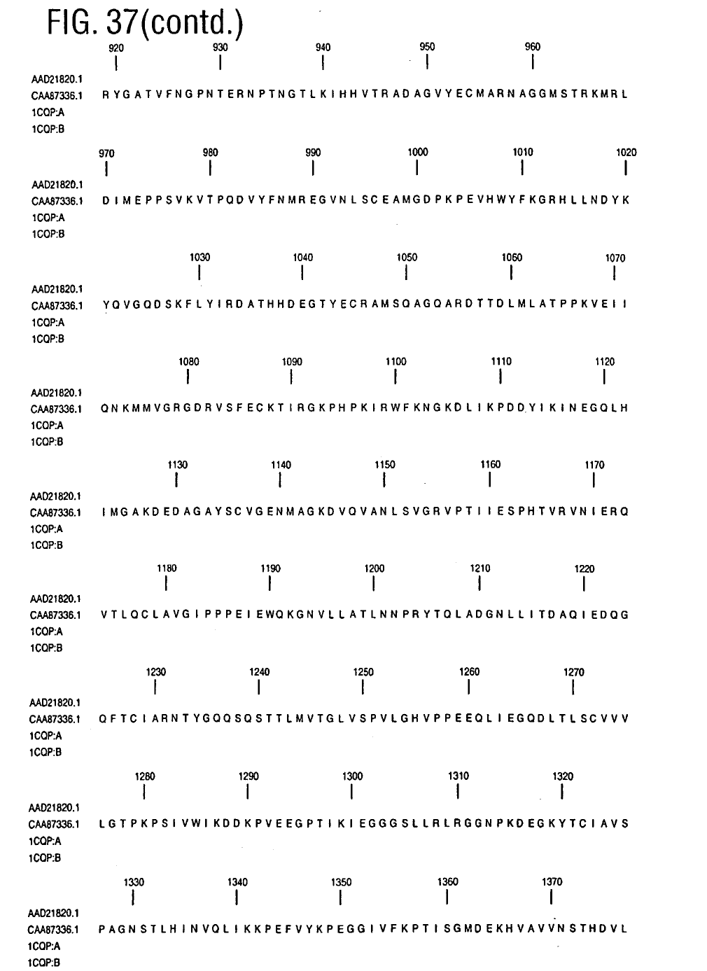

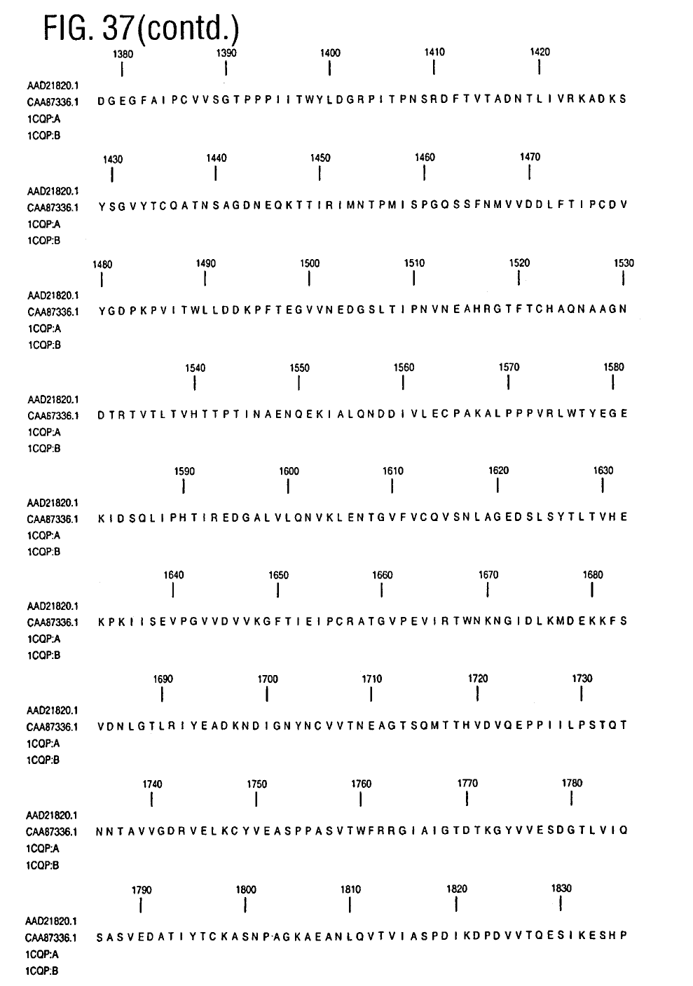

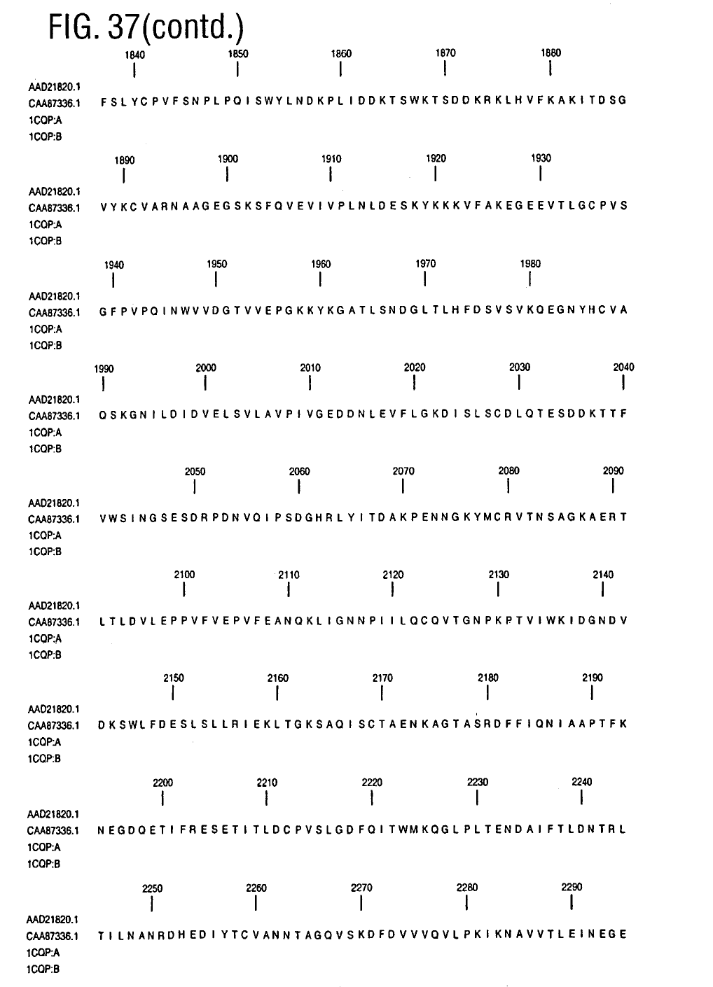

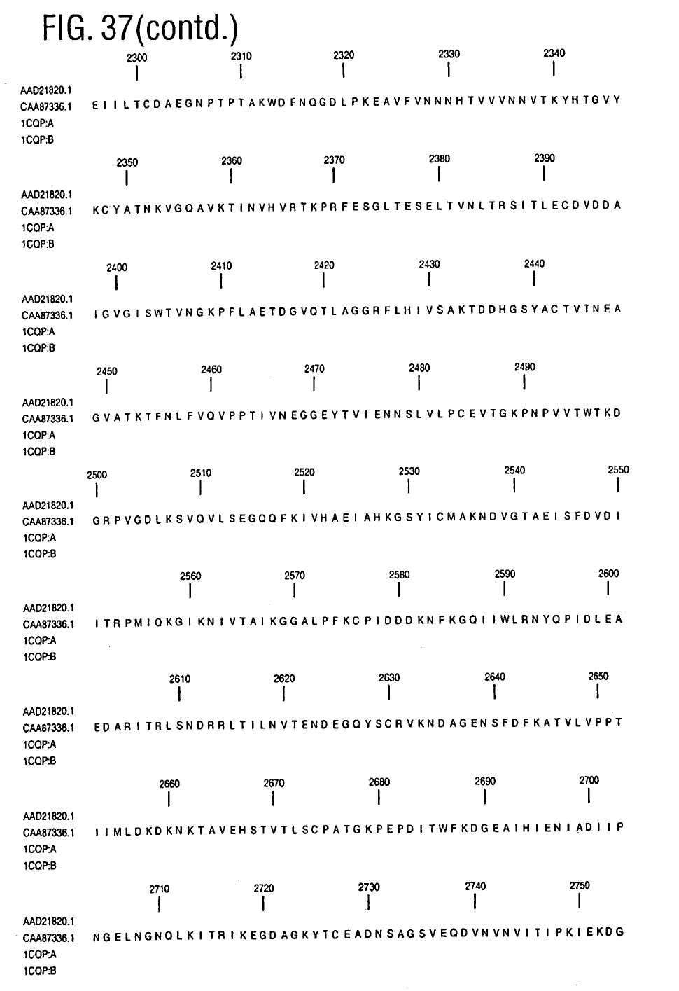

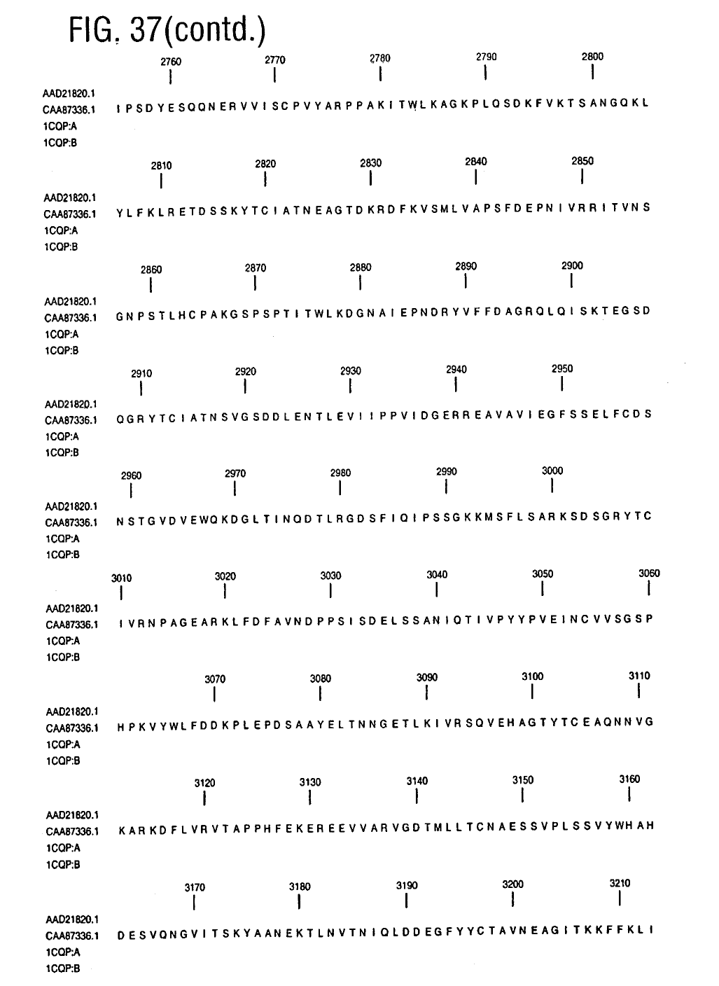

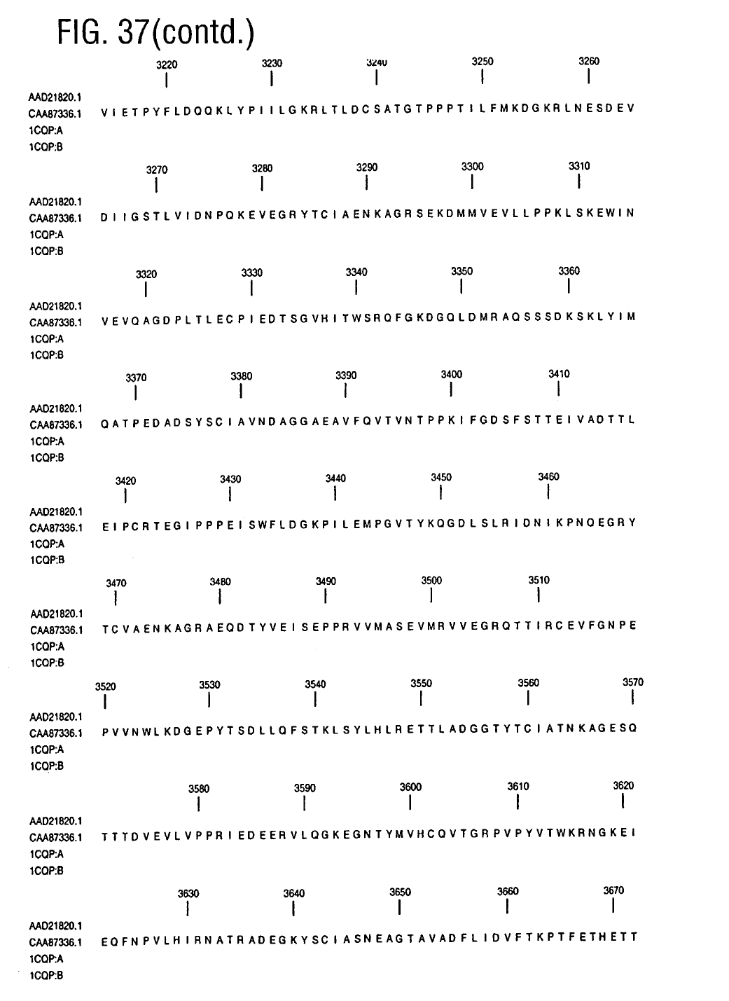

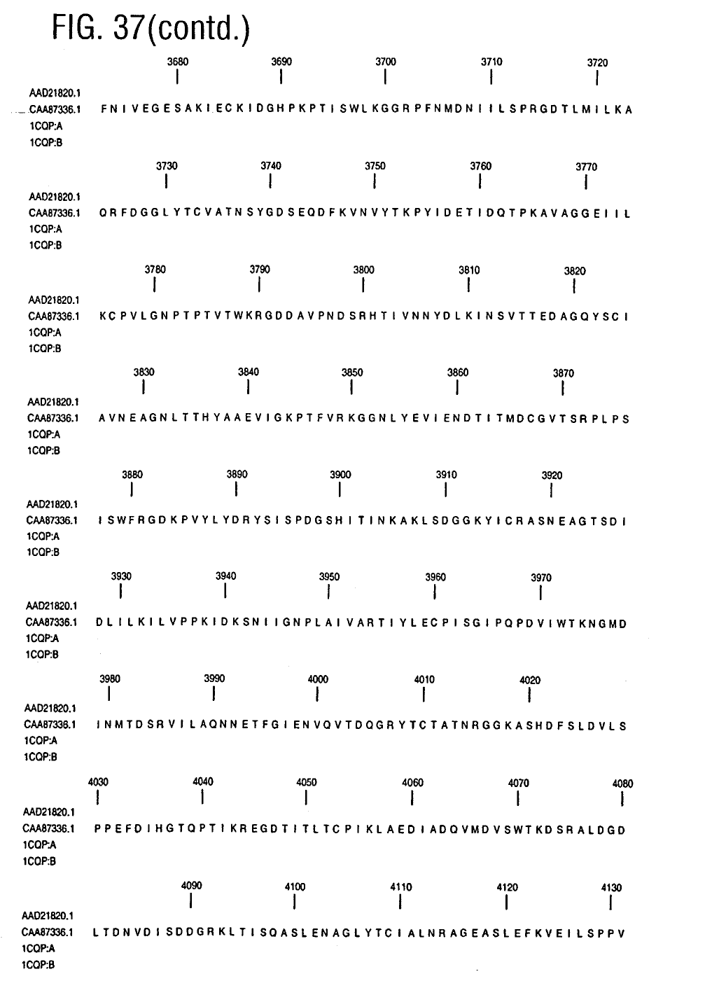

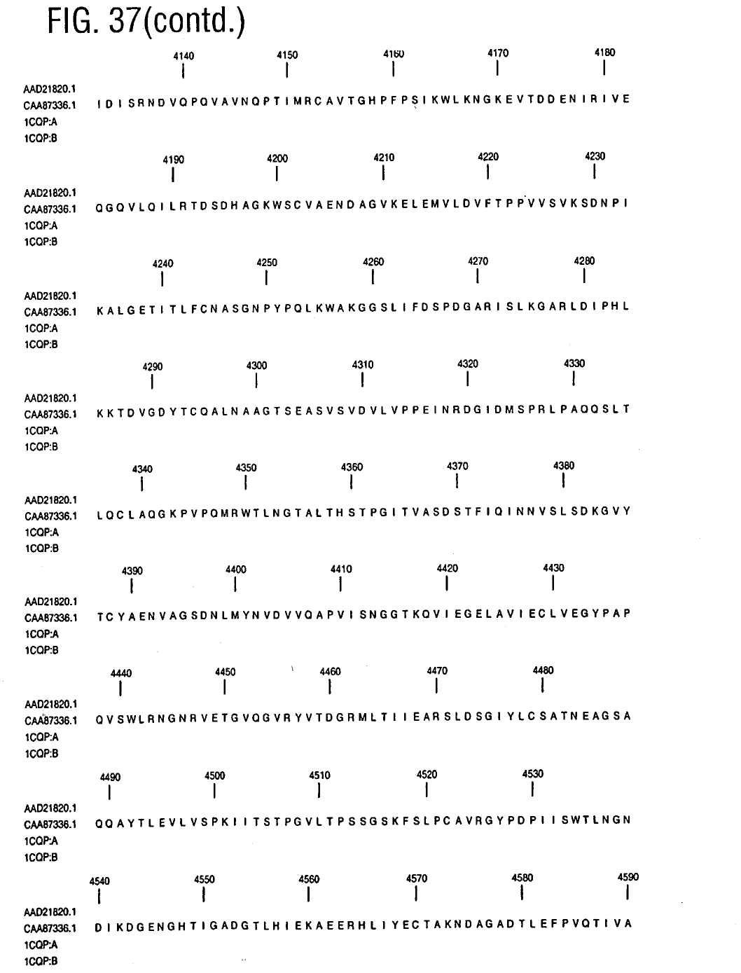

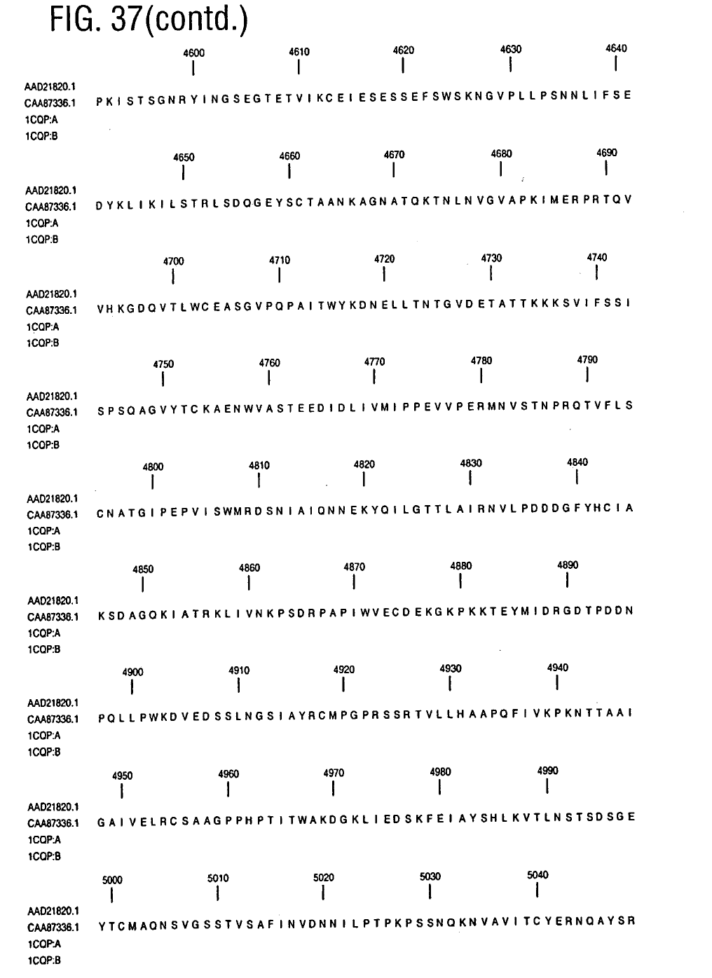

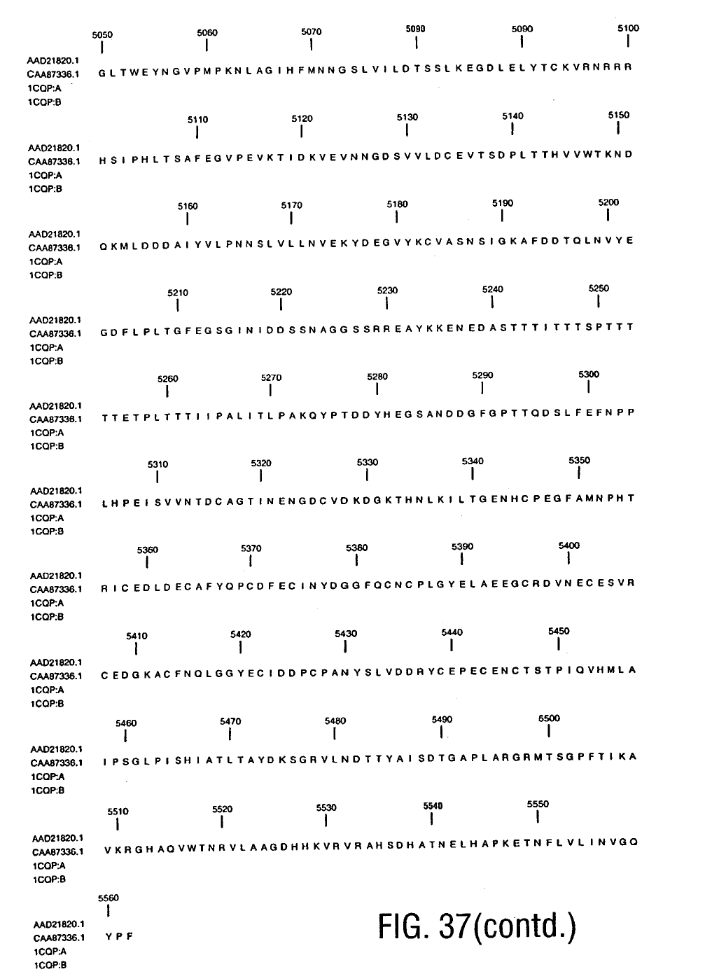

Among the integrin I domains that the Inpharmatica Genome Threader™ (FIG. 36A, arrow \mathring{2}) returns is the I domain from integrin LFA-1 (1CQP:A). Reverse maximised PSI-Blast (FIG. 27A, arrow \mathring{2}) identifies a homologue of AAD21820.1 in C. elegans, CAA87336.1. These are chosen (highlighted) against which to view the sequence alignment of NG37 (AD4). Viewing the alignment (FIG. 37) of the query protein against the proteins identified as being of a similar structure helps to visualize the areas of homology. FIG. 37 illustrates the point that the divalent cation binding residues of 1CQP:A and 1CQP:B (Ser139, Ser141 and Asp239 are conserved as Thr323 (conservative substitution), Ser325 and Asp417 in NG37 (AD4). This divalent ion binding region, termed the MIDAS (metal ion-dependent adhesion site) motif is highly conserved in I domain containing integrins. It plays an important role in integrin activation. The motif consists of a DXSXS consensus sequence and conserved aspartate and threonine residues which bind metal ions to provide an adhesion site.

FIG. 37 shows the conservation of ASP137 and SER141 of the DXSXS consensus sequence. SER139 the other member of the sequence is replaced by THR which is likely to be functionally equivalent, ASP239, the other metal ion ligand, is also conserved. These residues are also precisley conserved in the C. elegans homologue, CAA87336.1. This indicates that these residues are of functional importance to the protein, indicating that AAD21820.1 does indeed contain an I domain. Residues which are essential for the function of a protein will be conserved in homologues of that protein. Thus the precise conservation of identified functional I domain residues strongly supports the annotation of AAD21820.1 as an I domain-containing protein.

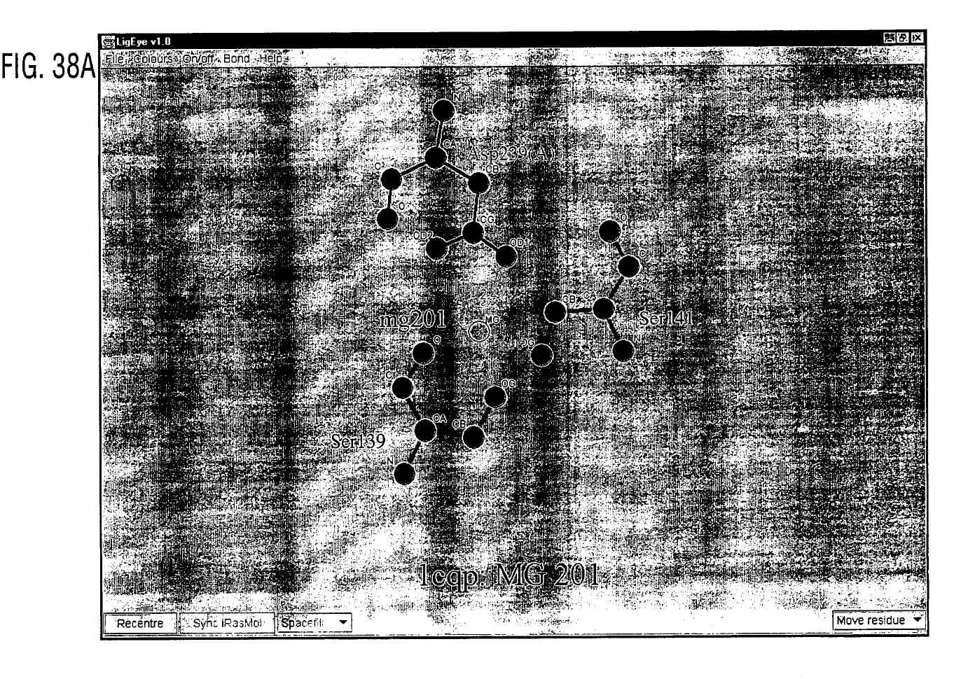

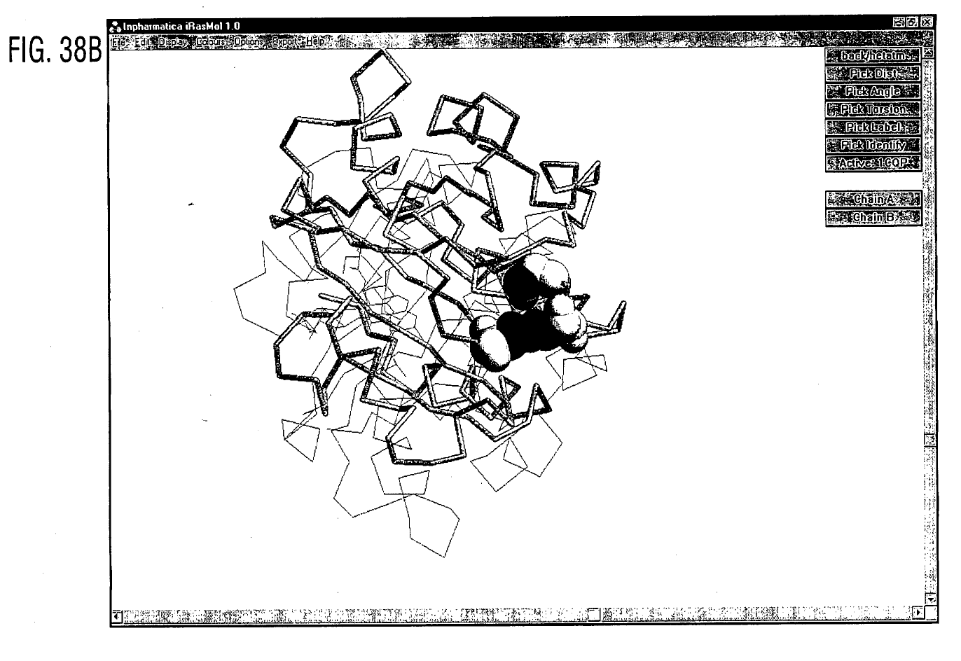

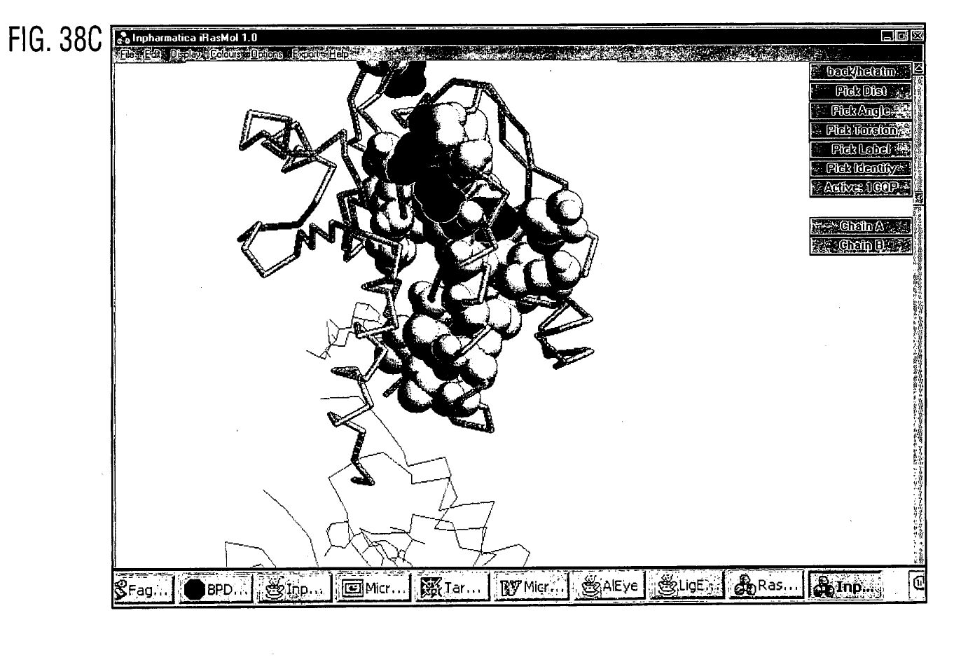

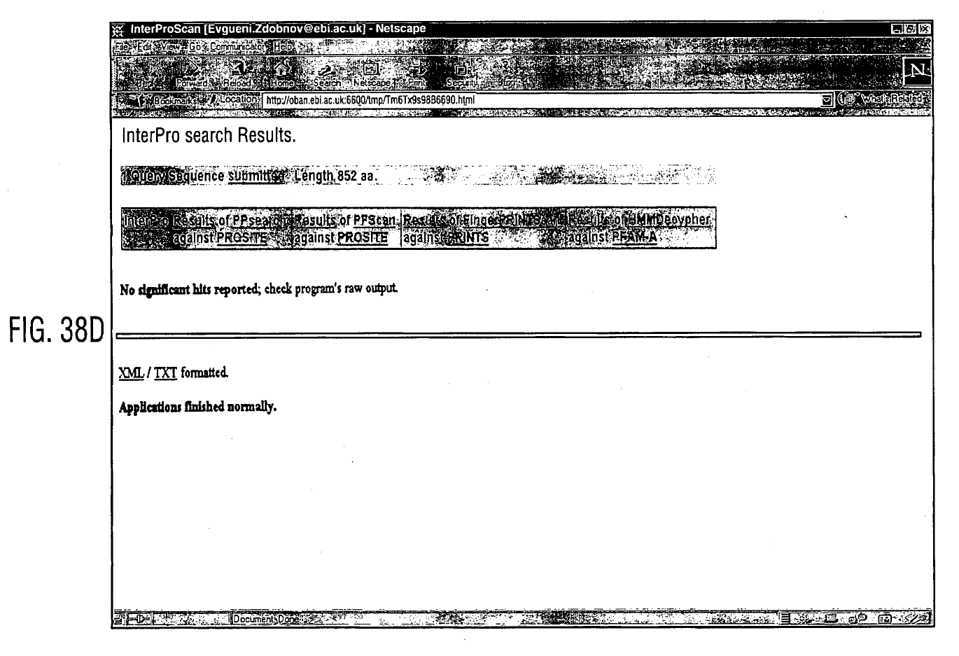

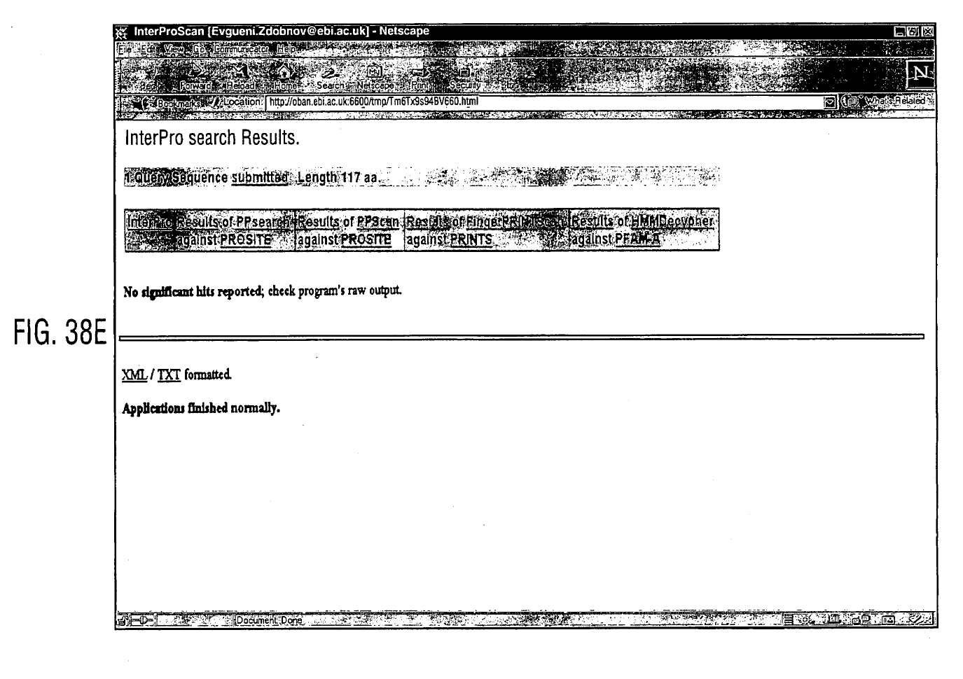

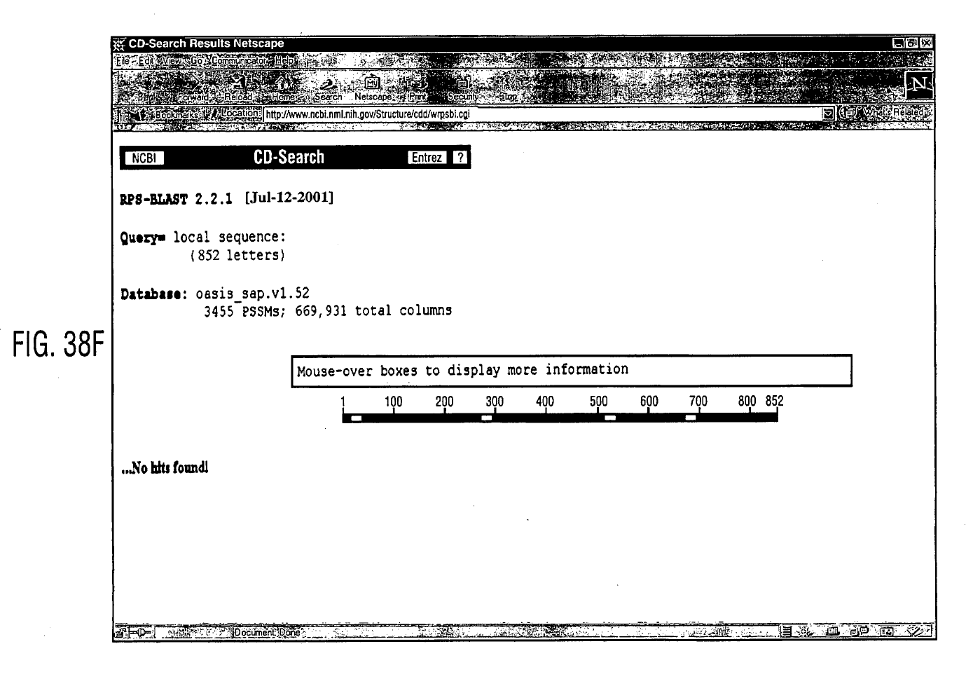

In order to ensure that the protein identified is a homologue of the query sequence, the visualisation program LigEye (FIG. 38A) and RasMol (FIG. 38B) are used. These visualization tools identify the active site of known protein structures by indicating the amino acids with which known small molecule inhibitors interact at the active site. These interactions are through either a direct hydrogen bond or hydrophobic interactions. In this manner one can see if the active site fold/structure is conserved between the identified homologue and the chosen protein of known structure.

This visualisation is shown with 1CQP, which illustrates the sites of interaction of a magnesium ion with the I domain of integrin LFA-1 (FIG. 38A). The magnesium ion sees 3 different amino acids in the I domain of LFA-1. The three amino acids of LFA-1 (SER139, SER141, and ASP239) are conserved in NG37 though SER139 is substituted by THR (FIG. 38B, ball structures). FIG. 38C further identifies the amino acids that are conserved in LFA-1 and NG37. The conservation of amino acids indicates that the fold is identical. This indicates that indeed as predicted by the Inpharmatica Genome Threader™, NG37 (AD4) folds in a similar manner to LFA-1 and as such is identified as an I domain-containing protein.

FIG. 38D shows the InterPro results for NG37 (AD4) as of Jul. 24, 2001. InterPro is a public domain annotation tool which combines PROSITE pattern, PROSITE profile, PRINTS and PFAM. InterPro returns no hits. (no hits are also reported when residues 308-424 of NG37 (AD4), rather than full-length sequence, is inputted into InterPro, see FIG. 38E). The fact that InterPro returns no results for NG37 (AD4) on the Jul. 24, 2001 demonstrates that on the date of PCT filing, NG37 (AD4) could still only be annotated as an adhesion molecule by Inpharmatica Genome Threader™.

FIG. 38F shows the NCBI Conserved Domain Database (CDD) results for NG37 (AD4) as of Jul. 24, 2001. CDD returns no hits. (no hits are also reported when residues 308-424 of NG37 (AD4), rather than the full-length sequence, is inputted into CDD, see FIG. 38G). The fact that CDD returns no results for NG37 (AD4) on the Jul. 24, 2001 demonstrates that on the date of PCT filing, NG37 (AD4) could still only be annotated as an adhesion molecule by Inpharmatica Genome Threader™.

### Example 5: CAB01991.1 (AD5)

In order to initiate a search for novel, distantly related integrins, an archetypal family member, alpha 2 beta 1, is chosen. More specifically, the search is initiated using a structure from the Protein Data Bank (PDB) which is operated by the Research Collaboratory for Structural Bioinformatics.

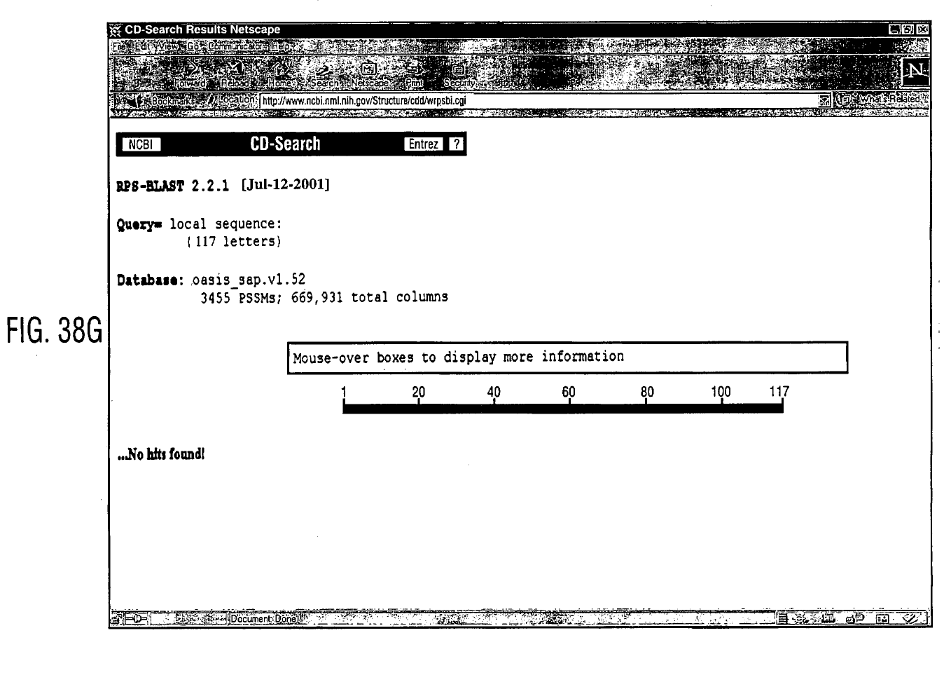

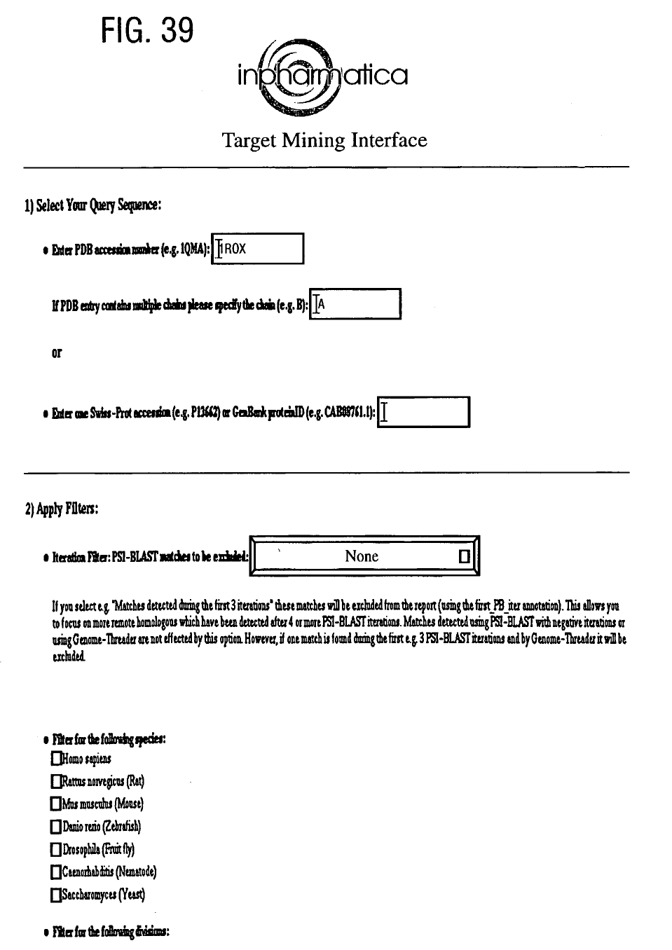

The structure chosen represents the I domain (insertion domain) of alpha 2 beta 1, PDB code 1AOX:A (FIG. 39). Alpha 2 beta 1 is expressed on a variety of cell types and acts as the collagen receptor on endothelial and epithelial cells. This integrin consists of a beta 1 and an alpha 2 chain and binds to collagen fibres in the extracellular matrix. Mutational studies indicate that the I domain that is located in the alpha 2 chain is the site of collagen binding. Integrin I domains are homologous to the A-domains present in von Willebrand factor, several collagen and complement proteins, and cartilage matrix protein, all proteins with adhesive functions (Huth, J. R., et al., Proc Natl Acad Sci USA. 2000 97(10):5231-6).

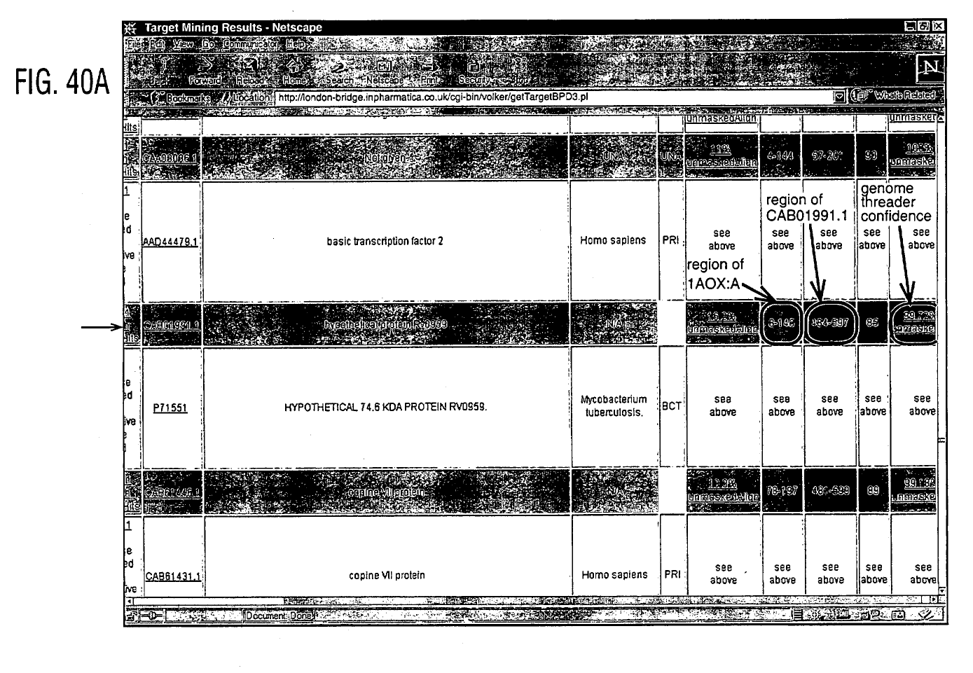

A search of the Biopendium for homologues of 1AOX:A takes place and returns 529 Inpharmatica Genome Threader™ results (selection given in FIG. 40A) and 646 PSI-Blast results (selection in FIG. 40B). The 529 Genome Threader™ results include examples of other I domain containing integrins, such as LFA-1 as well as Collagen alpha 1 and Von Willebrand Factor. Among the known I domain containing adhesion molecules/proteins appears a protein of apparently unknown function, (FIG. 40A) CAB01991.1 (AD5).

The Inpharmatica Genome Threader™ has thus identified a sequence, CAB01991.1 (AD5), as having an equivalent structure to the I domain of integrin alpha 2 beta 1, the known interaction domain between it and collagen. Having a structure similar to this domain suggests that CAB01991.1 (AD5) is a protein that functions in cellular adhesion. The Inpharmatica Genome Threader™ identifies this with 99.72% confidence.

PSI-Blast (FIG. 40B) is unable to identify this relationship; it is only the Inpharmatica Genome Threader™ that is able to identify CAB01991.1 (AD5) as having an I domain. PSI-Blast does identify other related integrins with varying degrees of probability (E value) as would be expected.

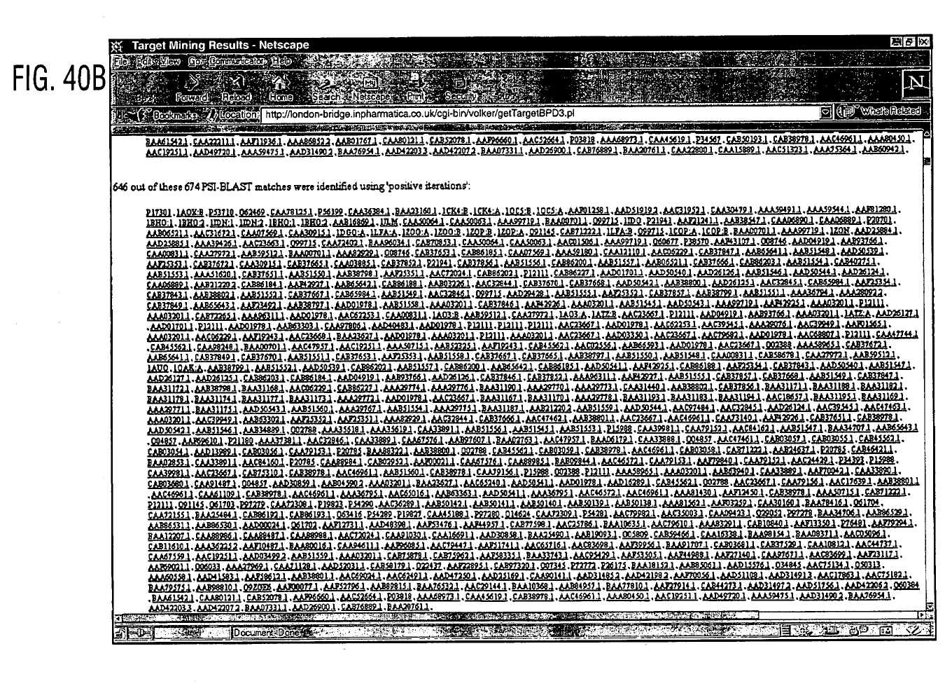

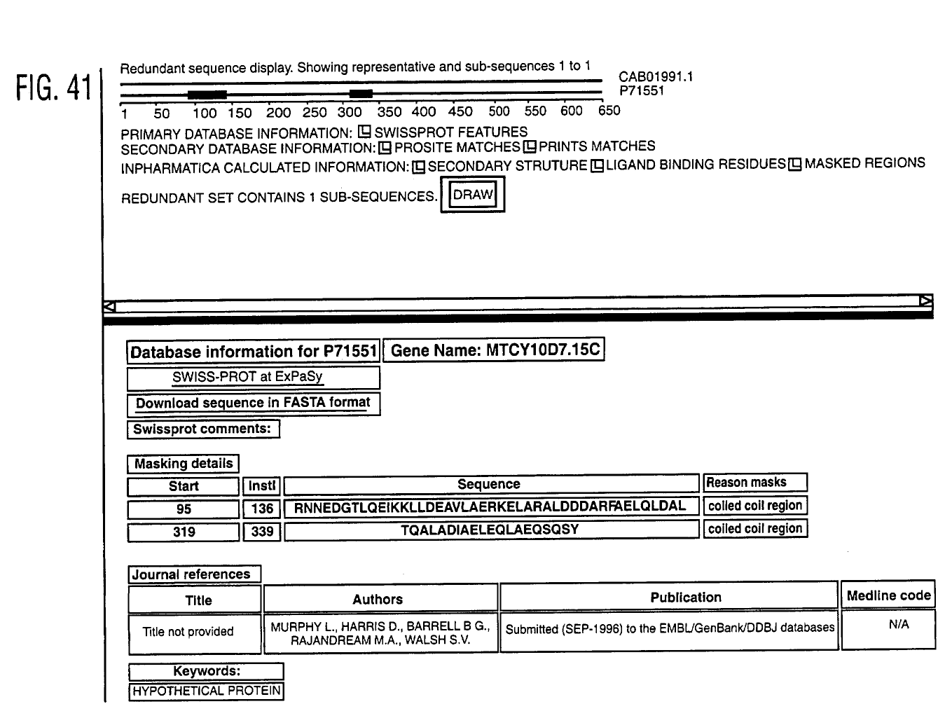

In order to view what is known in the public domain databases about CAB01991.1 (AD5), the Redundant Sequence Display Page is viewed (FIG. 41). CAB01991.1 (AD5) is a Mycobacterium tuberculosis sequence, and is 672 amino acids in length. There are no associated PROSITE or PRINTS hits for the sequences. PROSITE and PRINTS are databases that help to describe proteins of similar families. Returning zero hits from both databases means that CAB01991.1 (AD5) is unidentifiable as an I domain-containing adhesion molecule using PROSITE or PRINTS.

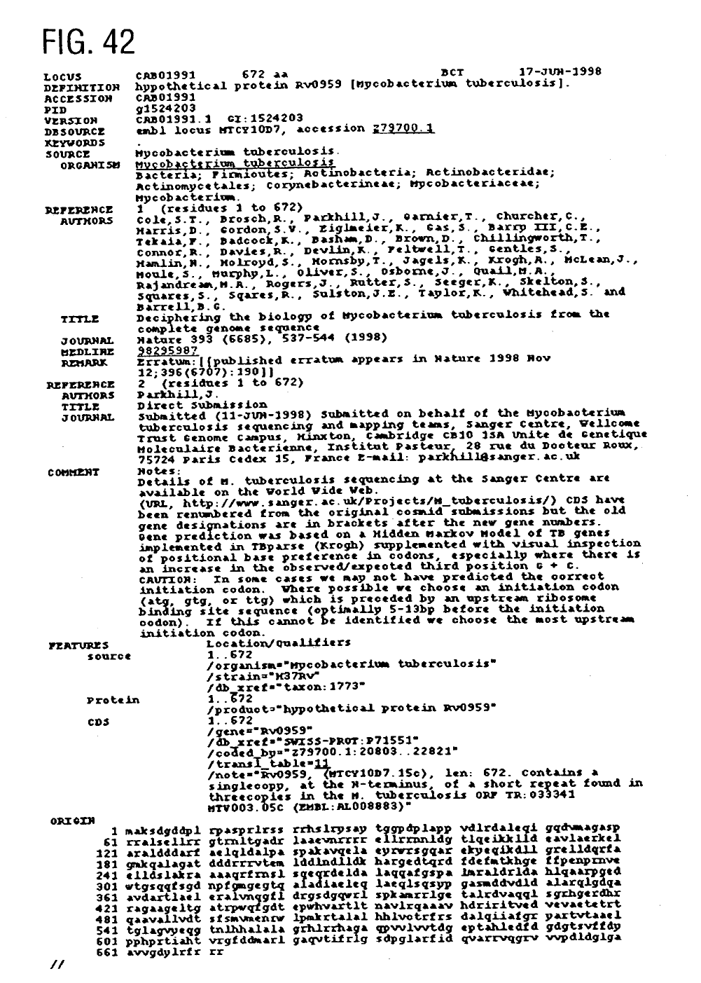

The National Centre for Biotechnology Information (NCBI) GenBank protein database is viewed to examine if there is any further information that is known in the public domain relating to CAB01991.1 (AD5). This is the U.S. public domain database for protein and gene sequence deposition (FIG. 42). CAB01991.1 (AD5) is derived from part of the Mycobacterium tuberculosis genome sequenced by the Sanger Centre at Cambridge and other members of the Mycobacterium tuberculosis genome sequencing team (Cole, S. T. et al Nature 393(6685):537-544 (1998). The public domain information for CAB01991.1 (AD5) does not annotate it as an integrin or I domain-containing protein, or indeed, contain any suggestion whatsoever for the function of this protein.

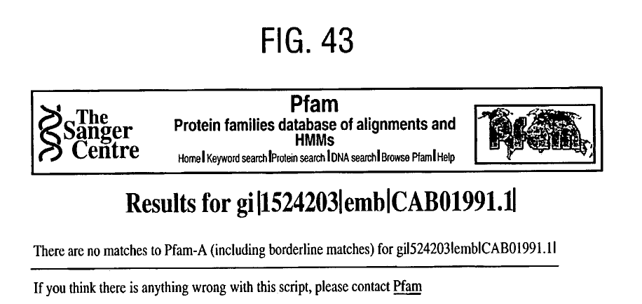

In order to identify whether any other public domain annotation vehicle is able to annotate CAB01991.1 (AD5) as I domain-containing proteins, the CAB01991.1 (AD5) protein sequence is searched against the Protein Family Database of Alignment and HMM's (PFAM) database (FIG. 43). The results identify that CAB01991.1 (AD5) has no identifiable PFAM-A matches.

Therefore using all public domain annotation tools CAB01991.1 (AD5) cannot be annotated as a protein involved in adhesion through the presence of an I domain. Only the Inpharmatica Genome Threader™ is able to annotate this protein as possessing an I domain.

The reverse search is now carried out. CAB01991.1 (AD5) is now used as the query sequence in the Biopendium™. The Inpharmatica Genome Threader™ identifies 439 hits (Selection in FIG. 44A) while PSI-Blast returns 330 hits (Selection FIG. 44B).

The Inpharmatica Genome Threader™ (FIG. 44A, arrow \mathring{1}) identifies residues 484-646 of CAB01991.1 (AD5) as having a structure the same as the I domain of the integrin LFA-1 (1CQP:A) with a confidence of 100%. The Inpharmatica Genome Threader™ (FIG. 44A, arrow \mathring{2}) also identifies residues 482-646 of CAB01991.1 (AD5) as having a structure the same as the I domain of the integrin MAC-1 (1BHO:2) with a confidence of 100%. Thus a region from residues 482-484 to residue 646 of CAB01991.1 (AD5) has been identified as adopting an equivalent fold to a range of I-domains including those of the adhesion molecules LFA-1 and MAC-1. PSI-Blast is only able to return this result above iteration seven and in the negative iteration, which the Biopendium™ computes through its all by all calculation. It is only the Inpharmatica Genome Threader™ and negative iteration PSI-Blast that is able to confidently identify the relationship between CAB01991.1 (AD5) and the I domain. Reverse maximised PSI-Blast (FIG. 44A, arrow \mathring{3}) identifies a homologue of CAB01991.1 in D. radiodurans, AAF11936.1, annotated as conserved hypothetical protein.

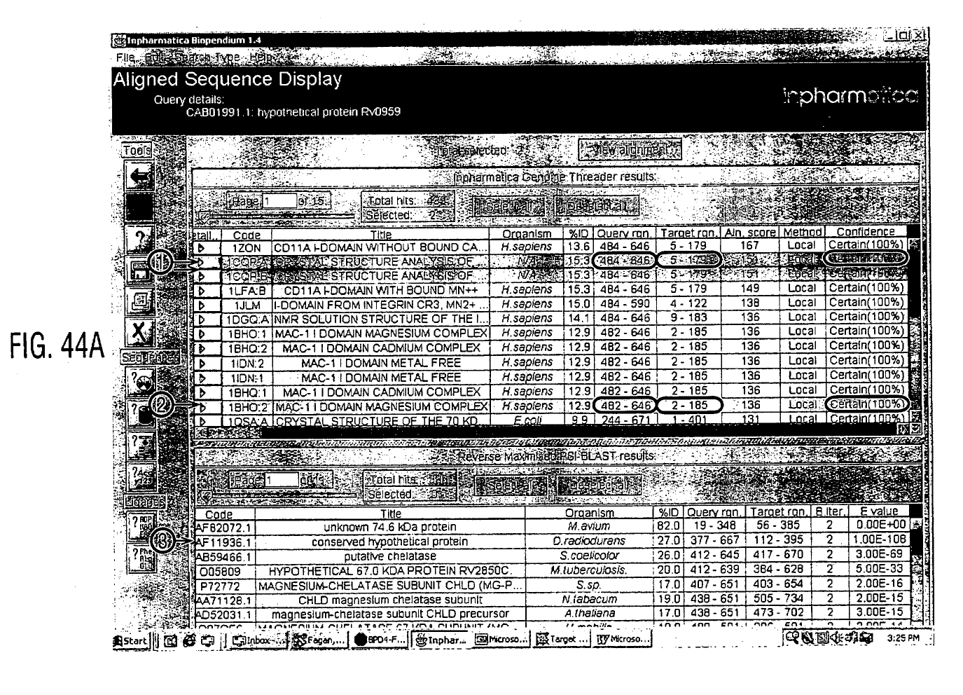

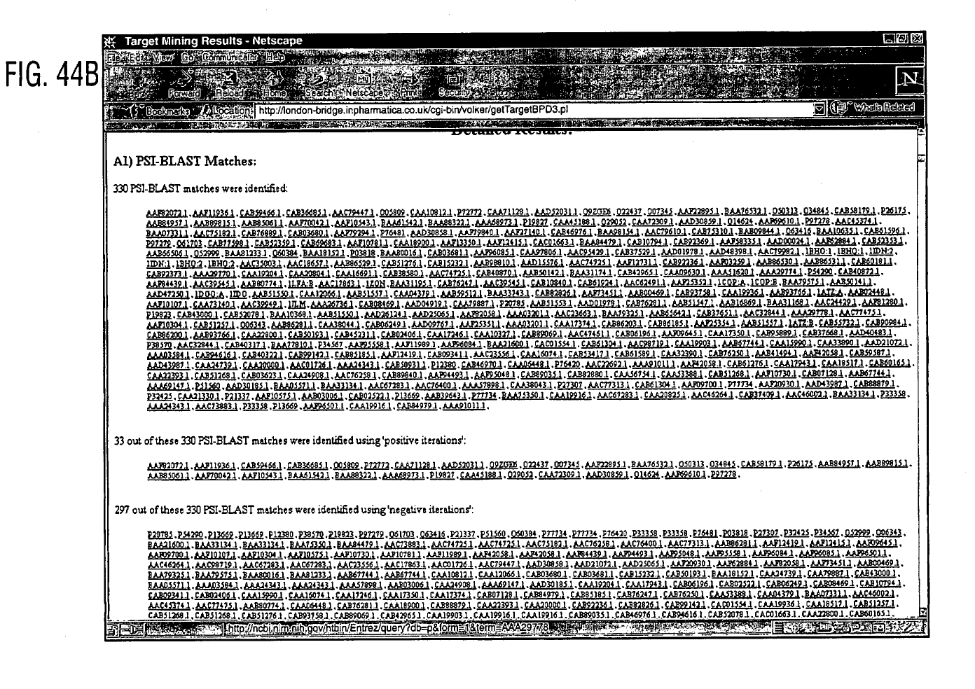

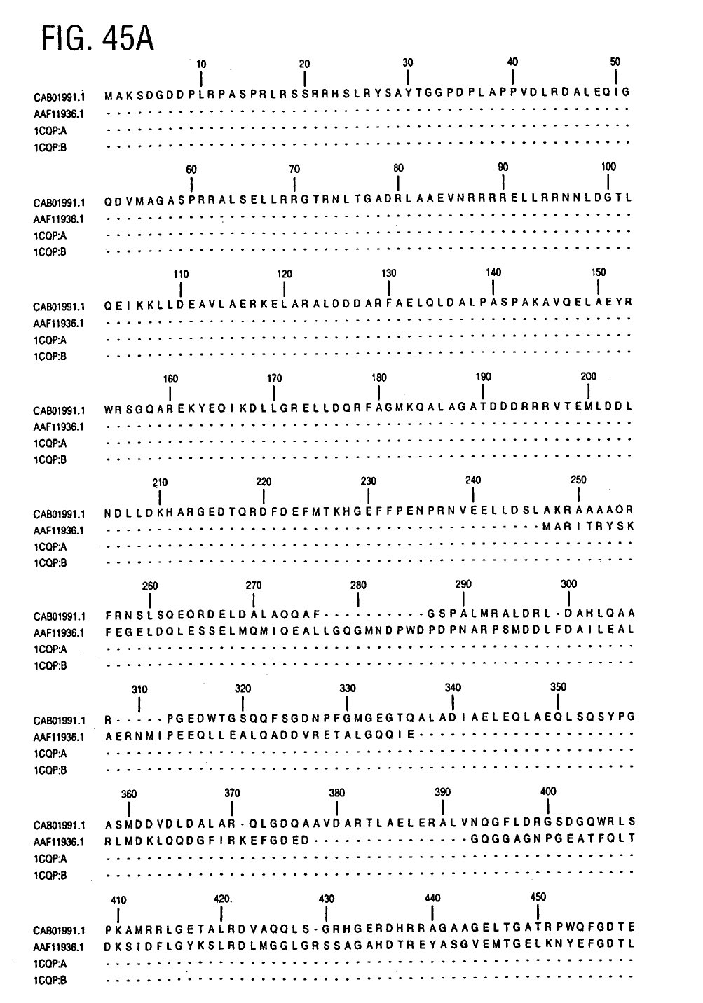

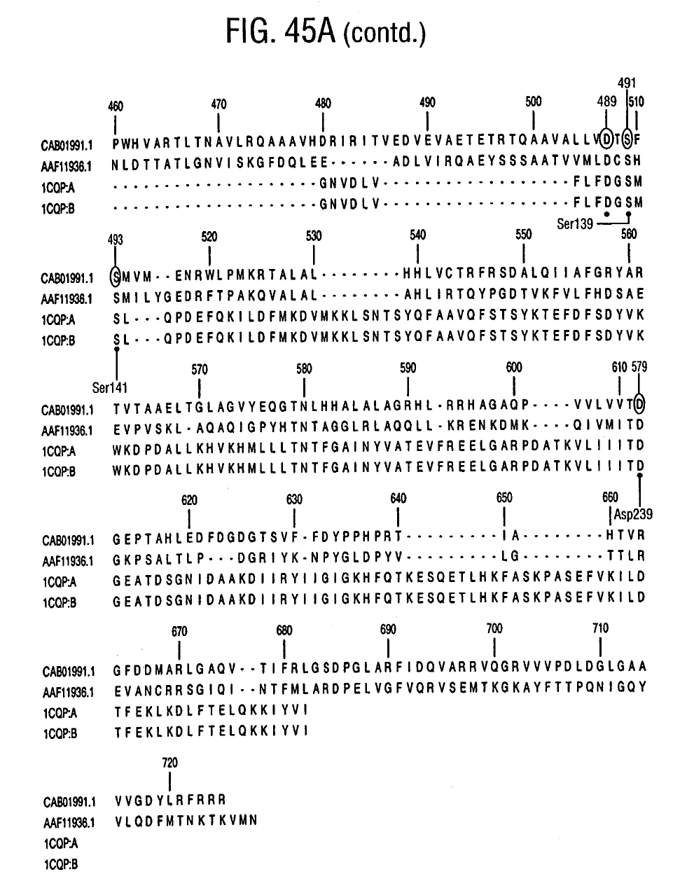

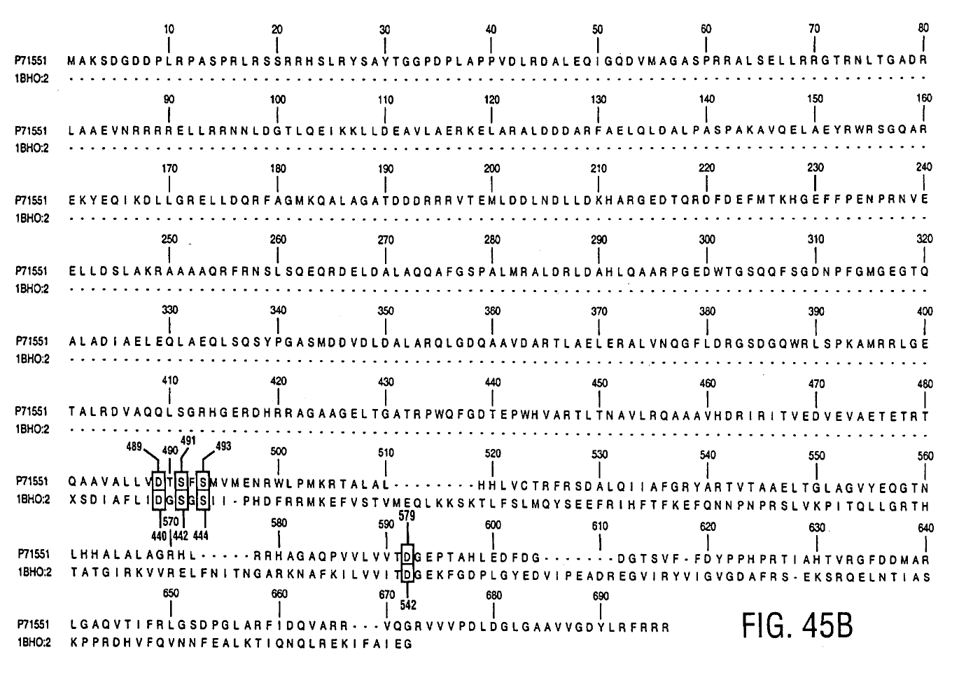

Among the integrin I domains that the Inpharmatica Genome Threader™ returns is the I domain from integrin LFA-1 (1CQP:A and 1CQP:B). These structures and AAF1 1936.1 are chosen against which to view the sequence alignment of CAB01991.1 (AD5). Viewing the alignment (FIG. 45A) of the query protein against the protein identified as being of a similar structure helps to visualize the areas of homology. FIG. 45A illustrates the point that the divalent cation binding residues of 1CQP:A and 1CQP:B (Ser139, Ser141 and Asp239) are conserved as Ser491, Ser493, and Asp579 in CAB01991.1 (AD5).

FIG. 45B shows the Genome Threader™ alignment of CAB01991.1 (which has the alternative identifier P71551; AD5) with the Integrin MAC-1 I-domain (1BHO:2). FIG. 45B illustrates that the metal ion binding residues of 1BHO:2 (Ser442, Ser444 and Asp542) are conserved as Ser 491, Ser493 and Asp579 in CAB01991.1 (AD5).

This divalent ion binding region, termed the MIDAS (metal ion-dependent adhesion site) motif is highly conserved in I domain containing integrins. It plays an important role in integrin activation. The motif consists of a DXSXS consensus sequence and conserved aspartate and threonine residues, which bind metal ions to provide an adhesion site.

Thus the precise conservation of identified functional I domain residues strongly supports the annotation of CAB01991.1 as an I domain-containing protein.

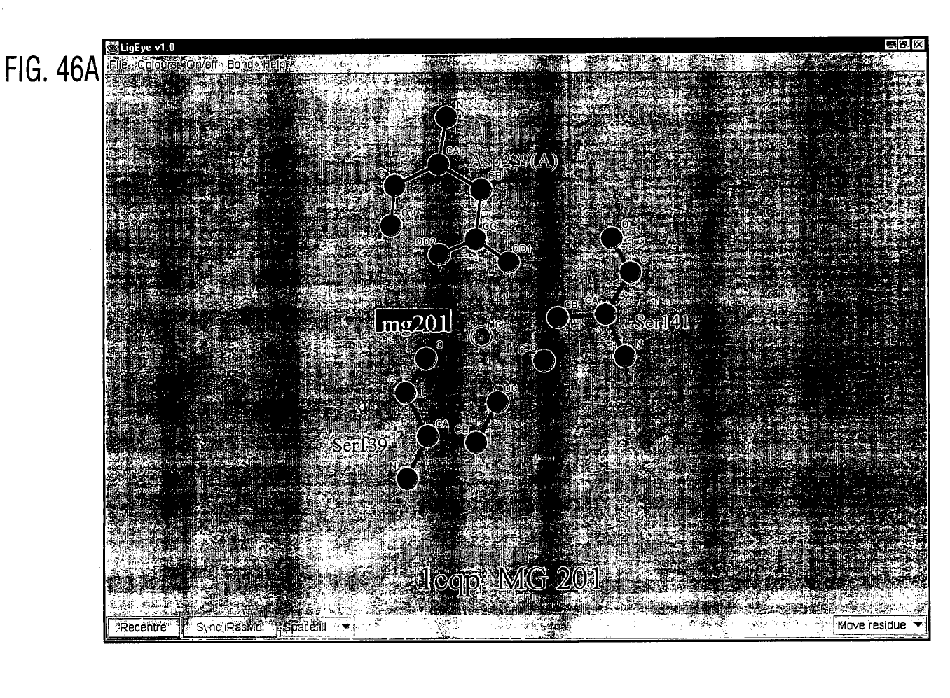

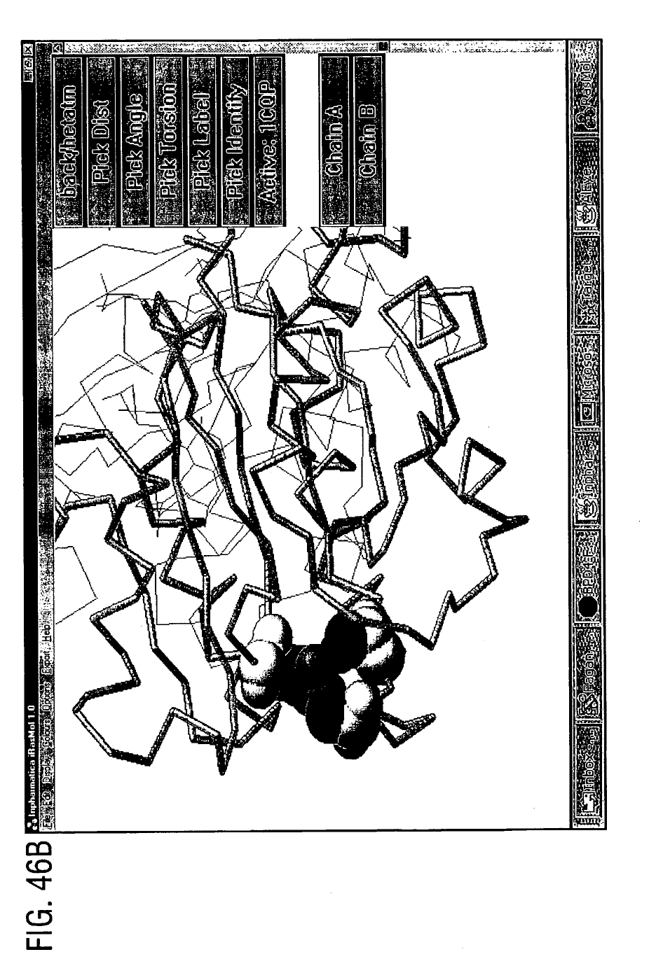

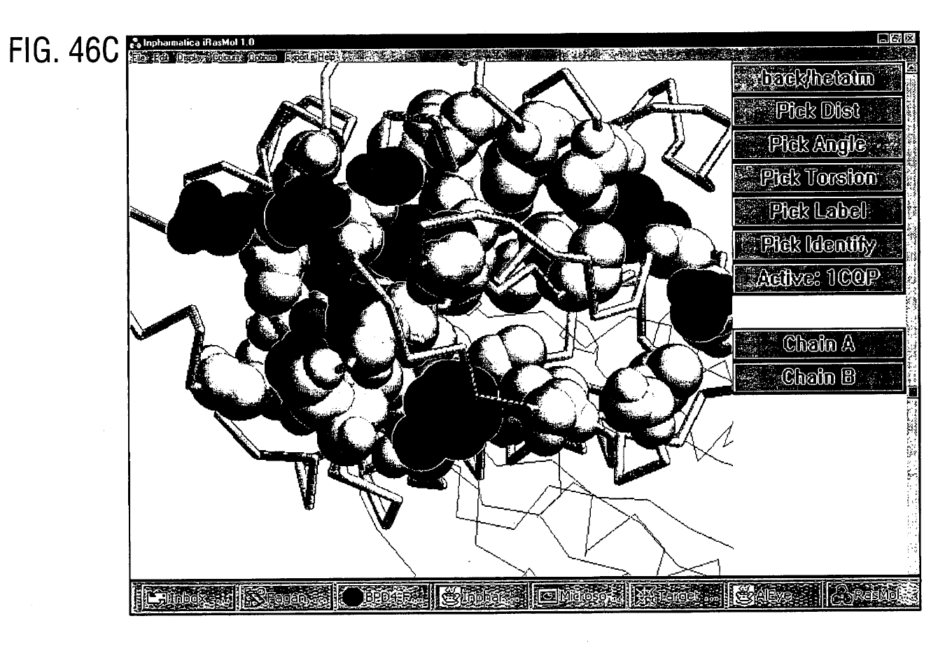

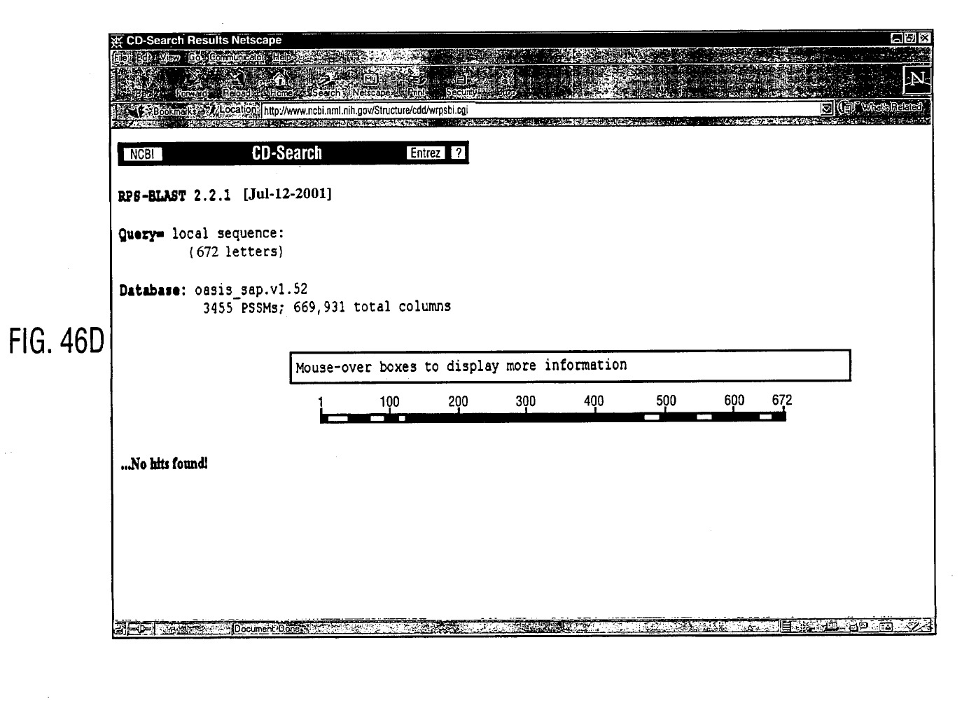

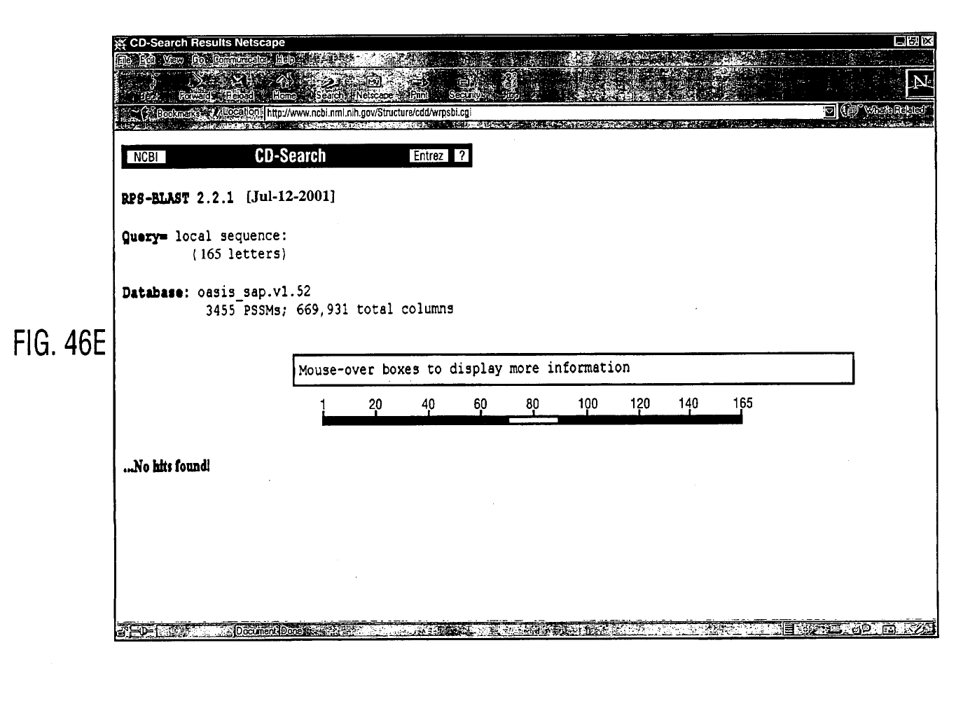

In order to ensure that the protein identified is a homologue of the query sequence, the visualisation program LigEye (FIG. 46A) and RasMol (FIG. 46B) are used. These visualization tools identify the active site of known protein structures by indicating the amino acids with which known small molecule inhibitors interact at the active site. These interactions are through either a direct hydrogen bond or hydrophobic interactions. In this manner one can see if the active site fold/structure is conserved between the identified homologue and the chosen protein of known structure.

This visualisation is shown with 1CQP, which illustrates the sites of interaction of a magnesium ion with the I domain of integrin LFA-1 (FIG. 46A). The magnesium ion sees 3 different amino acids in the I domain of LFA-1. These three amino acids of LFA-1 (SER139, SER141, and ASP239) are conserved in CAB01991.1 (AD5) (FIG. 46B, ball structures). FIG. 46C further identifies the amino acids that are conserved between 1CQP and CAB01991.1 (AD5). The conservation of amino acids indicates that the fold is identical. This indicates that indeed as predicted by the Inpharmatica Genome Threader™, CAB01991.1 (AD5) folds in a similar manner to 1AOX and as such is identified as an I domain-containing protein.

FIG. 46D shows the NCBI Conserved Domain Database (CDD) results for CAB01991.1 (AD5) as of Jul. 24, 2001. CDD returns no hits. (no hits are also reported when residues 482-646 of CAB01991.1 (AD5), rather than the full-length sequence, is inputted into CDD, see FIG. 46E). The fact that CDD returns no results for CAB01991.1 (AD5) on the Jul. 24, 2001 demonstrates that on the date of PCT filing, CAB01991.1 (AD5) could still only be annotated as an adhesion molecule by Inpharmatica Genome Threader™.

### Example 6: Rv0368c (AD6)

In order to initiate a search for novel, distantly related integrins, an archetypal family member, alpha 2 beta 1, is chosen. More specifically, the search is initiated using a structure from the Protein Data Bank (PDB) which is operated by the Research Collaboratory for Structural Bioinformatics.

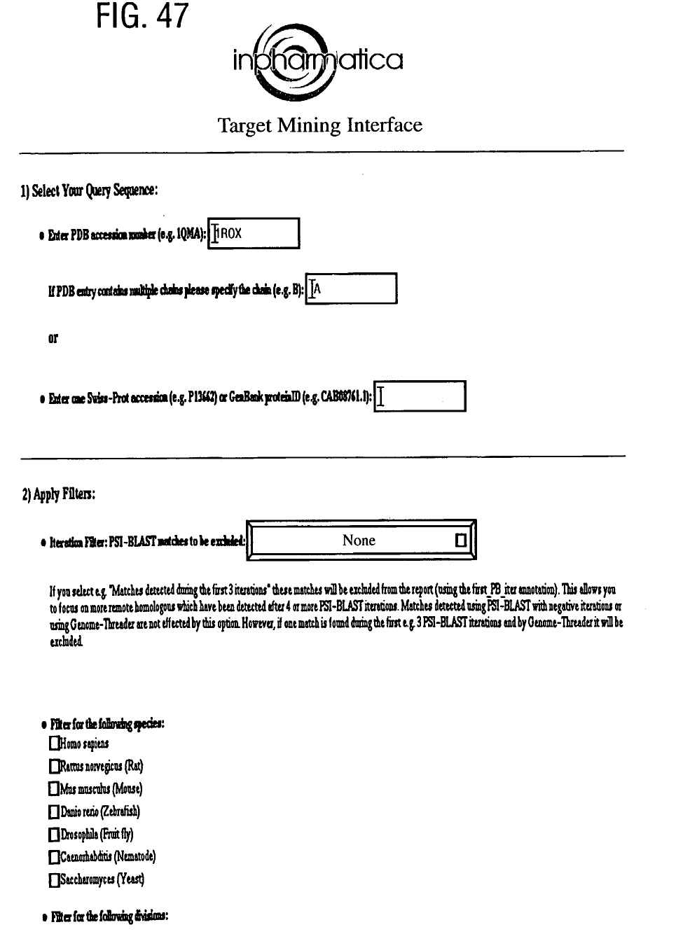

The structure chosen represents the I domain (insertion domain) of alpha 2 beta 1, PDB code 1AOX:A (FIG. 47). Alpha 2 beta 1 is expressed on a variety of cell types and acts as the collagen receptor on endothelial and epithelial cells. This integrin consists of a beta 1 and an alpha 2 chain and binds to collagen fibres in the extracellular matrix. Mutational studies indicate that the I domain that is located in the alpha 2 chain is the site of collagen binding. Integrin I domains are homologous to the A-domains present in von Willebrand factor, several collagen and complement proteins, and cartilage matrix protein, all proteins with adhesive functions (Huth, J. R., et al., Proc Natl Acad Sci USA. 2000 97(10):5231-6).

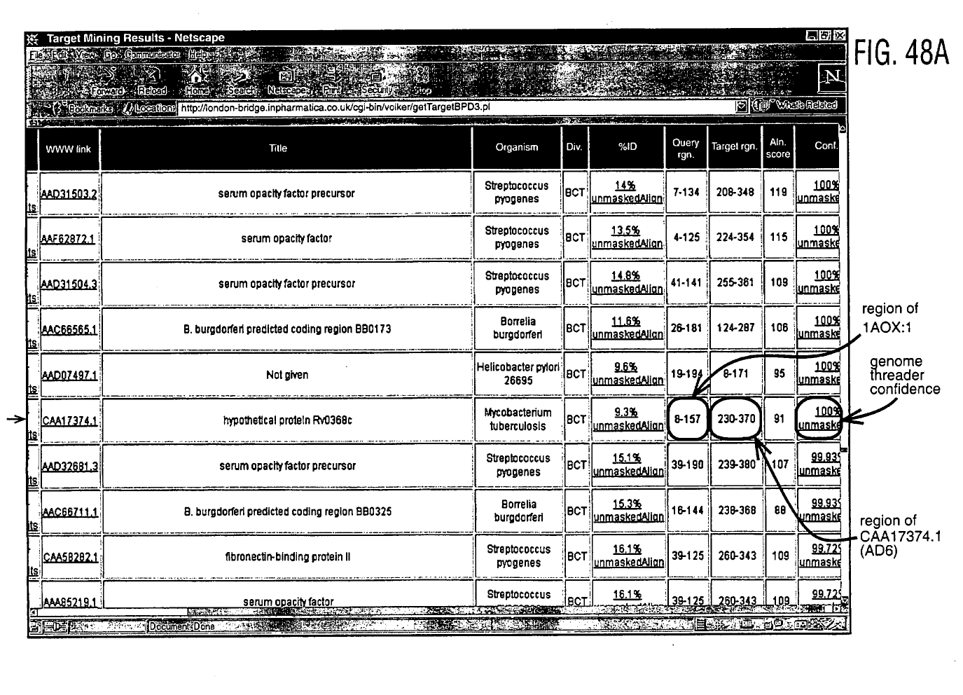

A search of the Biopendium for homologues of 1AOX:A takes place and returns 529 Inpharmatica Genome Threader™ results (selection given in FIG. 48A) and 674 PSI-Blast results (selection in FIG. 48B). The 529 Genome Threader results include examples of other I domain containing integrins, such as LFA-1 as well as Collagen alpha 1 and Von Willebrand Factor. Among the known I domain containing adhesion molecules/proteins appears a protein of apparently unknown function, Rv0368c (CAA17374.1; AD6) FIG. 48A).

The Inpharmatica Genome Threader™ has thus identified residues230-370 of a sequence, Rv0368c (AD6) as having a structure similar to residues 8-157 of the I domain of integrin alpha 2 beta 1 (1AOX:A), the known interaction domain between it and collagen. Having a structure similar to this domain suggests that Rv0368c (AD6) is a protein that functions in cellular adhesion. The Inpharmatica Genome Threader™ identifies this with 100% confidence.

PSI-Blast (FIG. 48B) is unable to identify this relationship; it is only the Inpharmatica Genome Threader™ that is able to identify residues 8-157 of Rv0368c (AD6) as having an I domain. PSI-Blast does identify other related integrins with varying degrees of probability (E value) as would be expected.

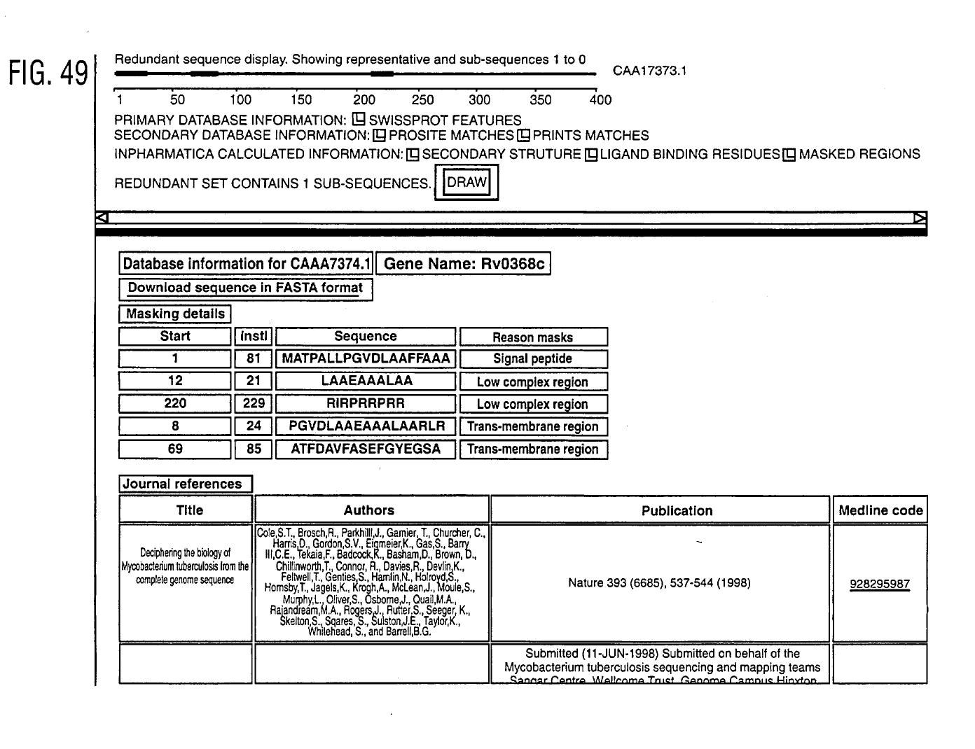

In order to view what is known in the public domain databases about Rv0368c (AD6), the Redundant Sequence Display Page is viewed (FIG. 49). Rv0368c (AD6) is a Mycobacterium tuberculosis sequence, and is 403 amino acids in length. There are no associated PROSITE or PRINTS hits for the sequence. PROSITE and PRINTS are databases that help to describe proteins of similar families. Returning zero hits from both databases means that Rv0368c (AD6) is unidentifiable as an I domain containing adhesion molecule using PROSITE or PRINTS.

The National Centre for Biotechnology Information (NCBI) GenBank protein database is viewed to examine if there is any further information that is known in the public domain relating to Rv0368c (AD6). This is the U.S. public domain database for protein and gene sequence deposition (FIG. 50). Rv0368c (AD6) is derived from part of the Mycobacterium tuberculosis genome sequenced by the Sanger Centre at Cambridge and other members of the Mycobacterium tuberculosis genome sequencing team (Cole, S. T. et al Nature 393 (6685):537-544 (1998). A similarity is noted between Rv0368c and another protein of unknown function, but no functional annotation for this protein is given. The public domain information for this gene does not annotate it as an integrin or I domain-containing protein, or indeed, contain any suggestion whatsoever for the function of this protein.

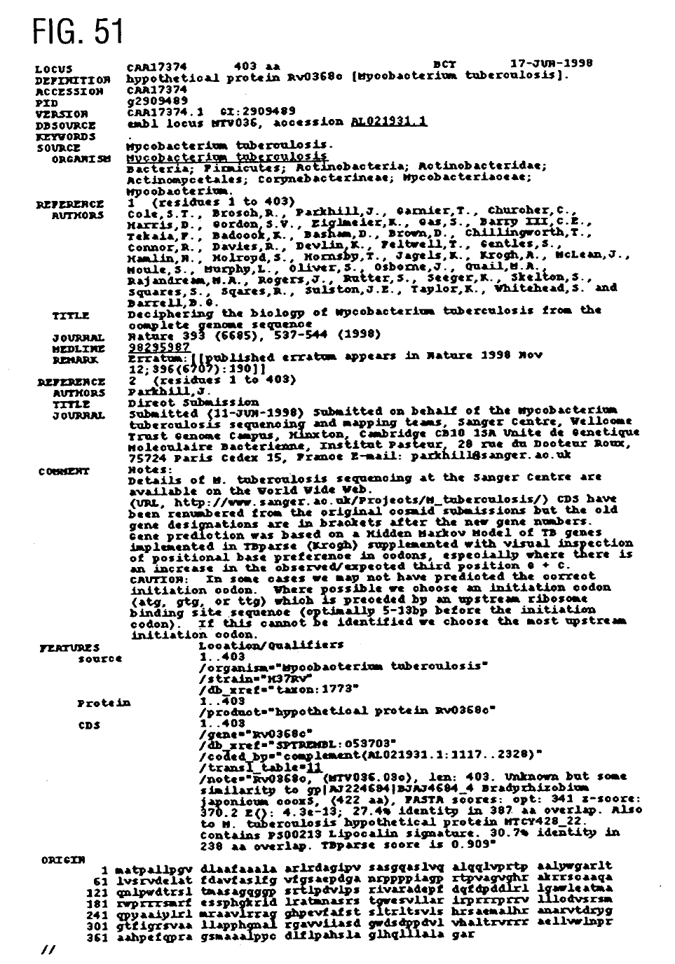

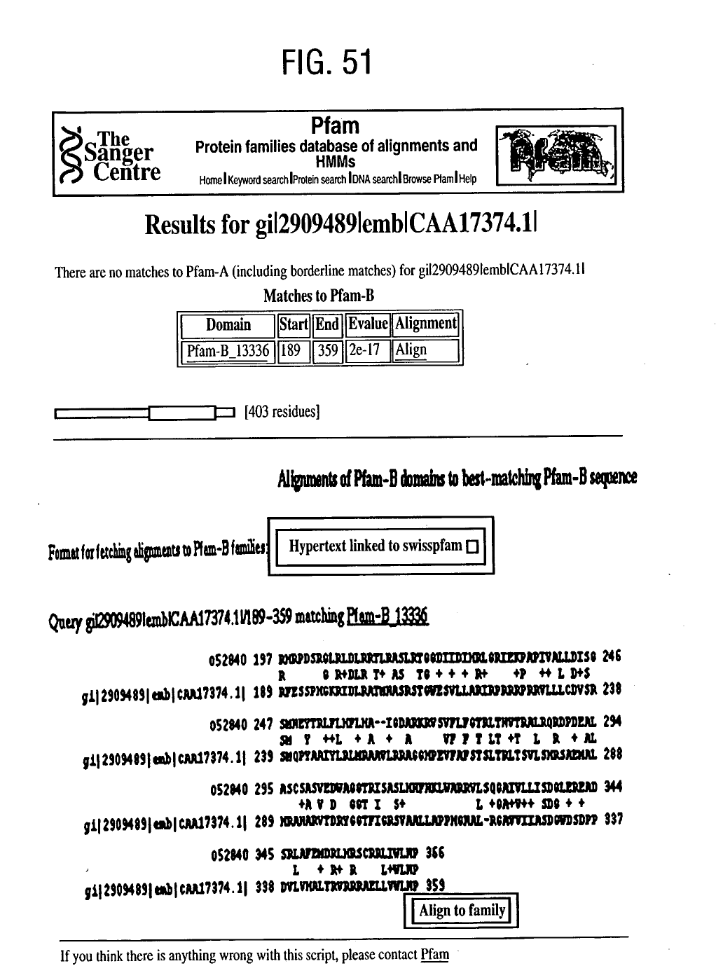

In order to identify whether any other public domain annotation vehicle is able to annotate Rv0368c (AD6) as an I domain containing protein, the Rv0368c (AD6) protein sequence is searched against the Protein Family Database of Alignment and HMM's (PFAM) database (FIG. 51). The results identify that Rv0368c (AD6) has no identifiable PFAM-A matches. The PFAM-B match has no functional information.

Therefore using all public domain annotation tools Rv0368c (AD6) is not annotated as a protein involved in adhesion through the presence of an I domain. Only the Inpharmatica Genome Threader™ is able to annotate this protein as possessing an I domain.

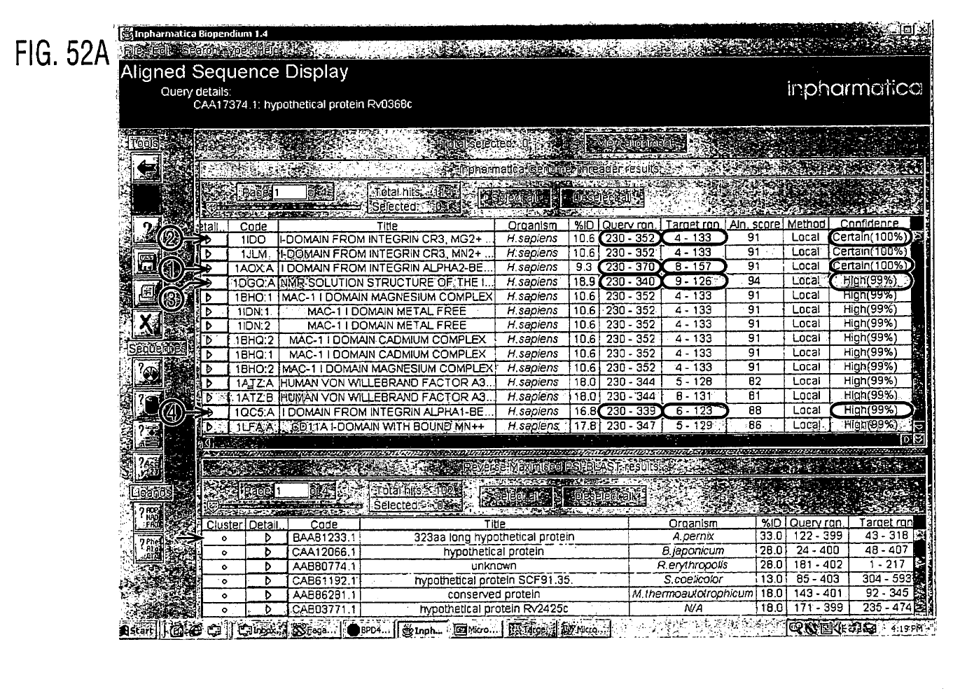

The reverse search is now carried out. Rv0368c (AD6) is now used as the query sequence in the Biopendium™. The Inpharmatica Genome Threader™ identifies 114 hits (FIG. 52A) while PSI-Blast returns 102 hits (FIG. 52B).

The Inpharmatica Genome Threader™ (FIG. 52A, arrow \mathring{1}) identifies residues 230-370 of Rv0368c (AD6) as having a structure the same as the I domain of the Integrin Alpha2 (1AOX:A) with 100% confidence. The Inpharmatica Genome Threader™ (FIG. 52A, arrow \mathring{2}) also identifies residues 230-352 of Rv0368c (AD6) as having a structure the same as the I domain of the Integrin MAC-1 (1IDO) with 100% confidence. The Inpharmatica Genome Threader™ (FIG. 52A, arrow \mathring{3}) also identifies residues 230-340 of Rv0368c (AD6) as having a structure the same as the I domain of the Integrin LFA-1 (1DGQ:A) with 99% confidence. The Inpharmatica Genome Threader™ (FIG. 52A, arrow \mathring{4}) also identifies residues 230-339 of Rv0368c (AD6) as having a structure the same as the I domain of the Integrin Alphal (1QC5:A) with 99% confidence. Thus a region from residue 230 to residues 339-370 of Rv0368c (AD6) has been identified as adopting an equivalent fold to a range of I-domains including those of the adhesion molecules Integrin Alpha2, MAC-1, LFA-1 and Integrin Alpha1.

PSI-Blast does not return this result, and only finds matches to other proteins of unknown function. PSI-blast is able to identify a homologue of Rv0368c (AD6) in A. pernix, BAA81233.1. It is only the Inpharmatica Genome Threader™ and negative iteration PSI-Blast that is able to confidently identify the relationship between Rv0368c (AD6) and the I domain.

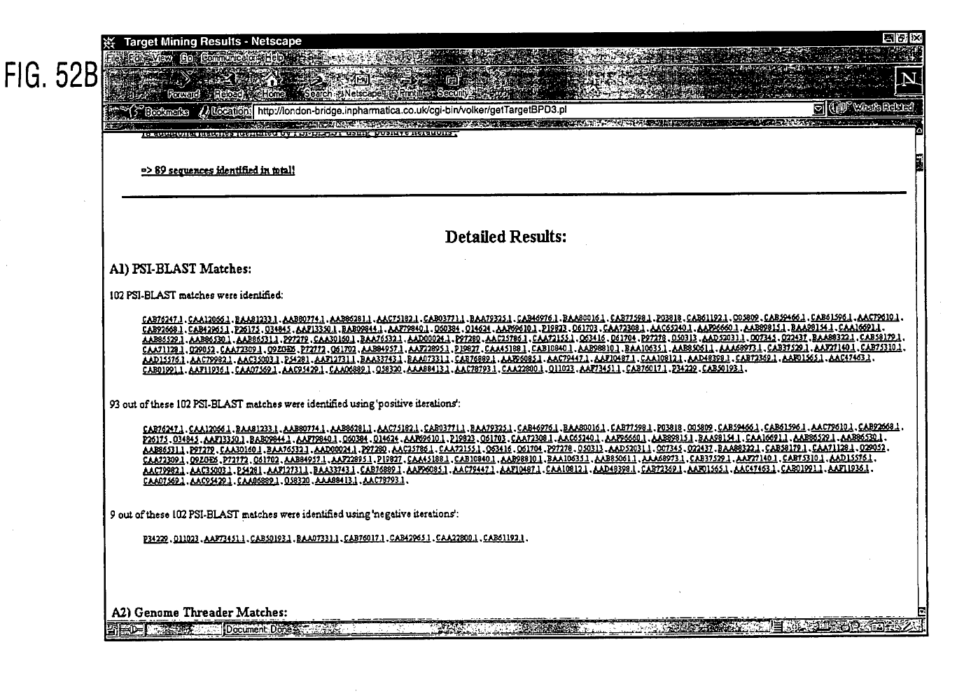

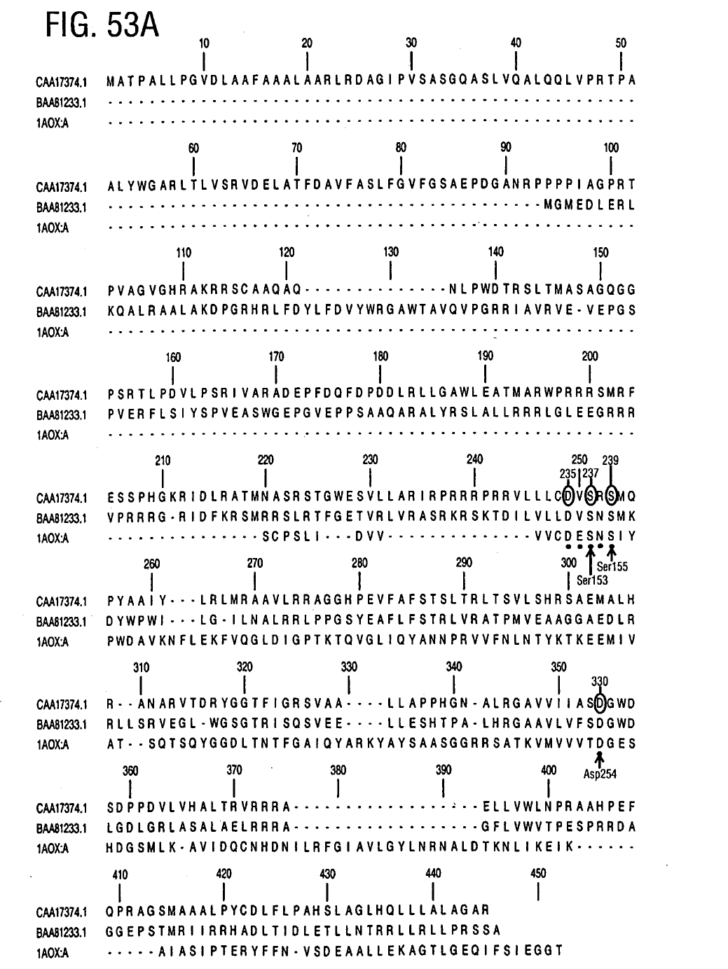

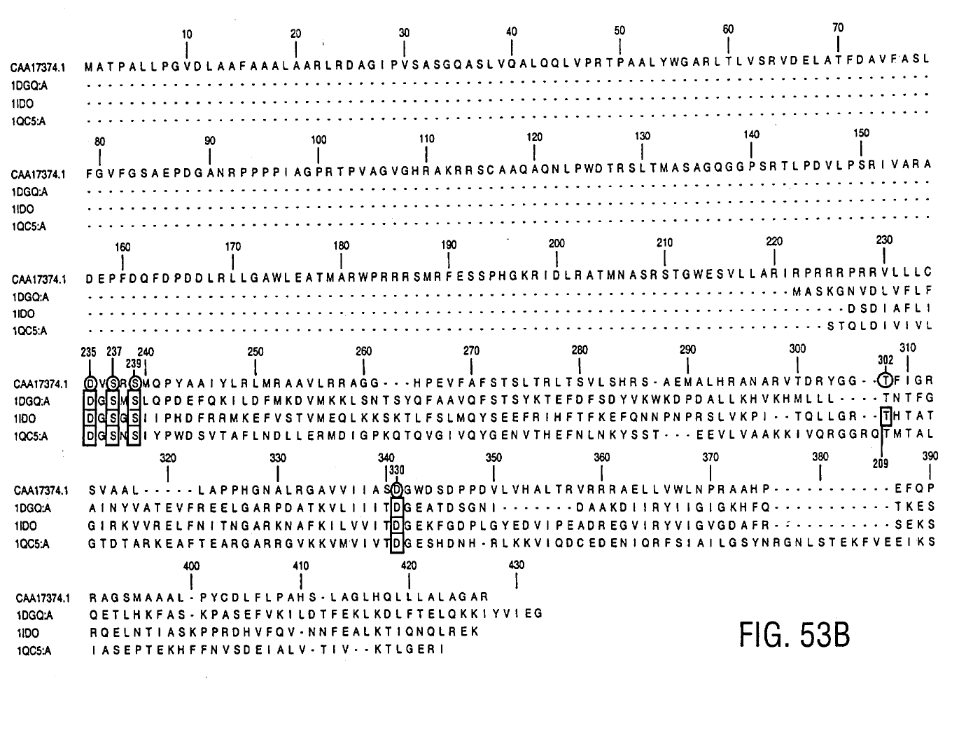

Among the integrin I domains that the Inpharmatica Genome Threader™ returns is the I domain from integrin Alpha 2/Beta 1 (1AOX:A). This structure and BAA81233.1 are chosen against which to view the sequence alignment of Rv0368c (AD6). Viewing the alignment (FIG. 53A) of the query protein against the proteins identified as being of a similar structure helps to visualize the areas of homology. FIG. 53A illustrates the point that the divalent cation binding residues of 1AOX:A (Ser153, Ser155 and Asp254) are conserved as Ser237, Ser239 and Asp330 in Rv0368c (AD6). This divalent ion binding region, termed the MIDAS (metal ion-dependent adhesion site) motif is highly conserved in I domain-containing integrins. It plays an important role in integrin activation. The motif consists of a DXSXS consensus sequence and conserved aspartate and threonine residues, which bind metal ions to provide an adhesion site.

FIG. 53A shows the conservation of the ASP151, SER153 and SER155 consensus sequence, the other metal ion ligand, ASP254, is also conserved and the fifth member of the MIDAS motif, THR221, is also conserved. Precise conservation of those residues is also observed in BAA81233.1. This indicates that these residues are of functional importance to the protein, indicating that Rv0368c (AD6) does indeed contain an I domain. Residues which are essential for the function of a protein will be conserved in homologues of that protein.

FIG. 53B shows the Genome threader™ alignment of Rv0368c (AD6) with the I-domains of LFA-1 (1DGQ:A), MAC-1 (1IDO) and Alpha1 (1QC5:A). FIG. 53B illustrates that the metal ion binding residues of 1DGQ:A (Ser139, Ser141 and Asp239) are conserved as Ser237, Ser239 and Asp330 in Rv0368c (AD6). FIG. 53B also illustrates that the metal ion binding residues of 1QC5:A (Ser42, Ser44 and Asp143) are conserved as Ser237, Ser239 and Asp330 in Rv0368c (AD6). FIG. 53B also illustrates that the metal ion binding residues of 1IDO (Ser142, Ser144 and Thr209) are conserved as Ser237, Ser239 and Thr302 in Rv0368c (AD6). Thus Rv0368c (AD6) has two potential metal ion binding triads (Ser237, Ser239 and Asp330) or (Ser237, Ser239 and Thr302).

Thus the precise conservation of identified functional I domain residues strongly supports the annotation of Rv0368c (AD6) as an I domain-containing protein.

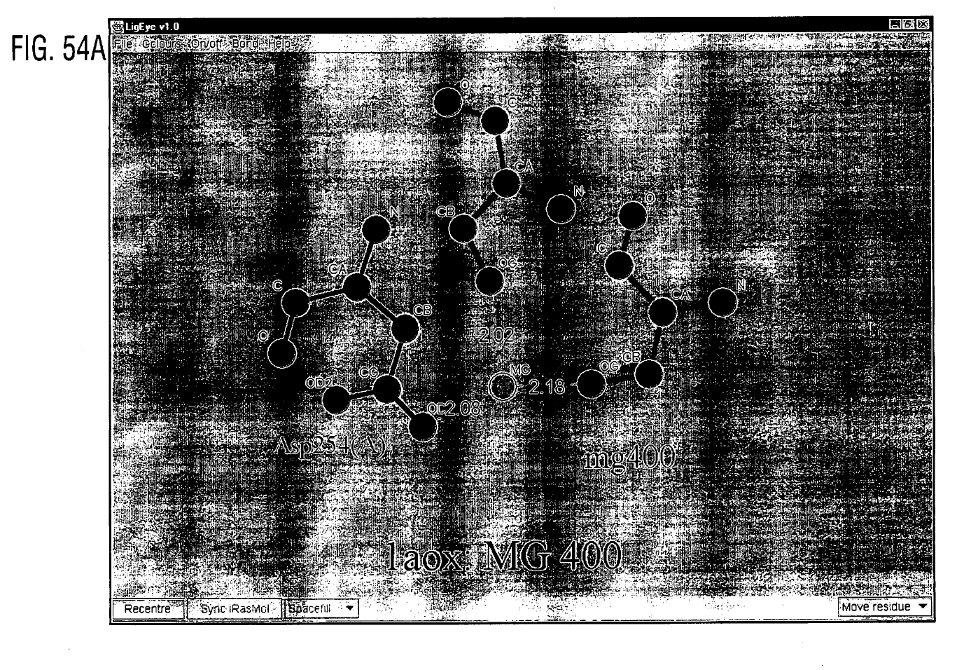

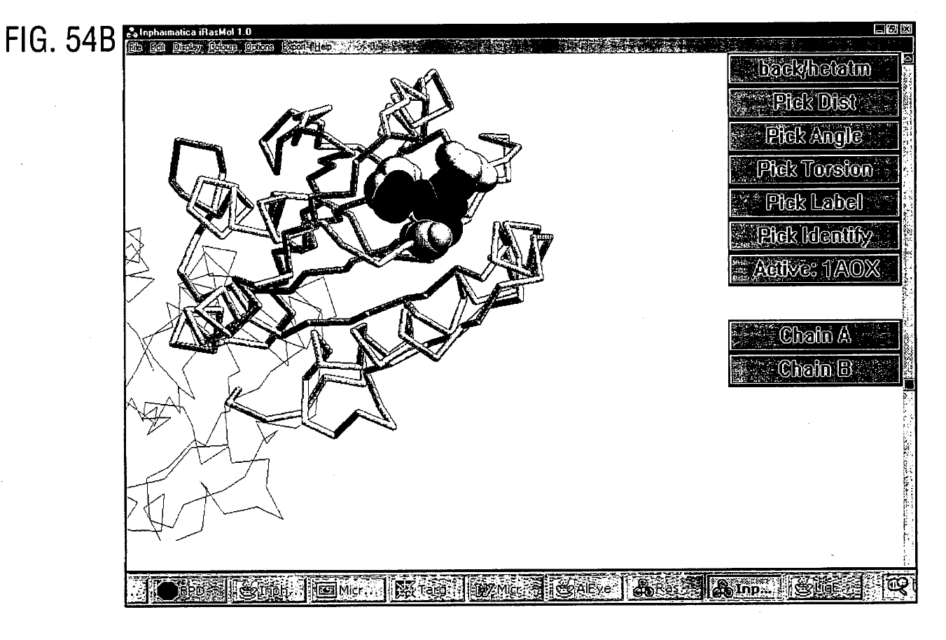

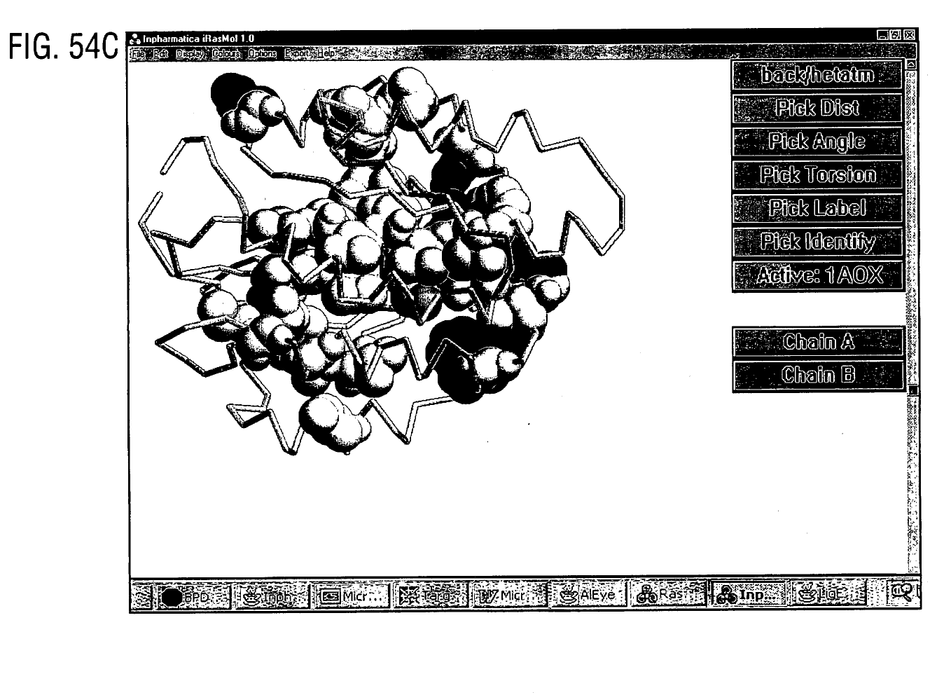

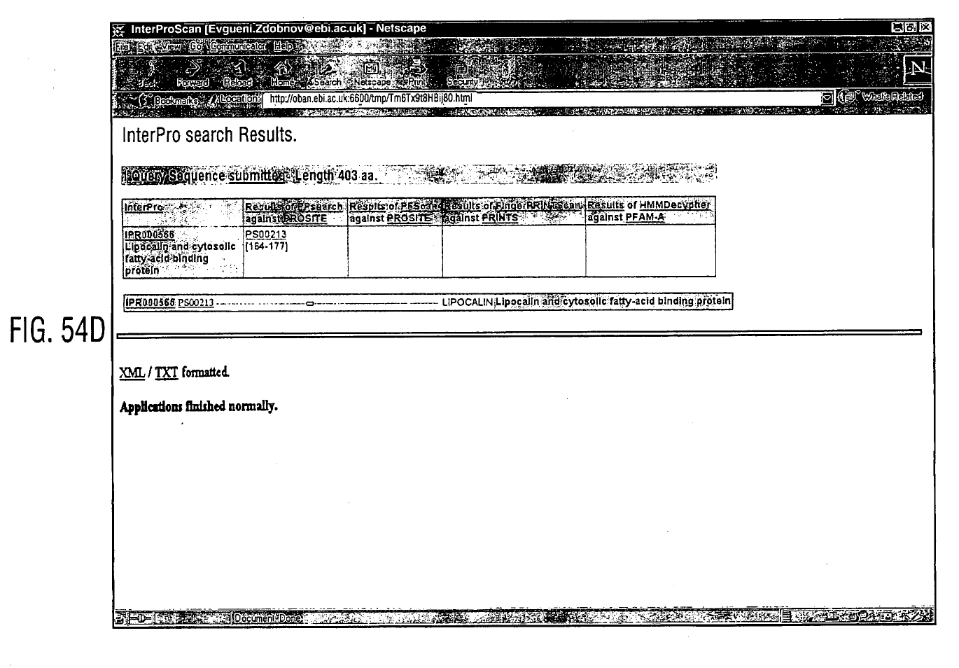

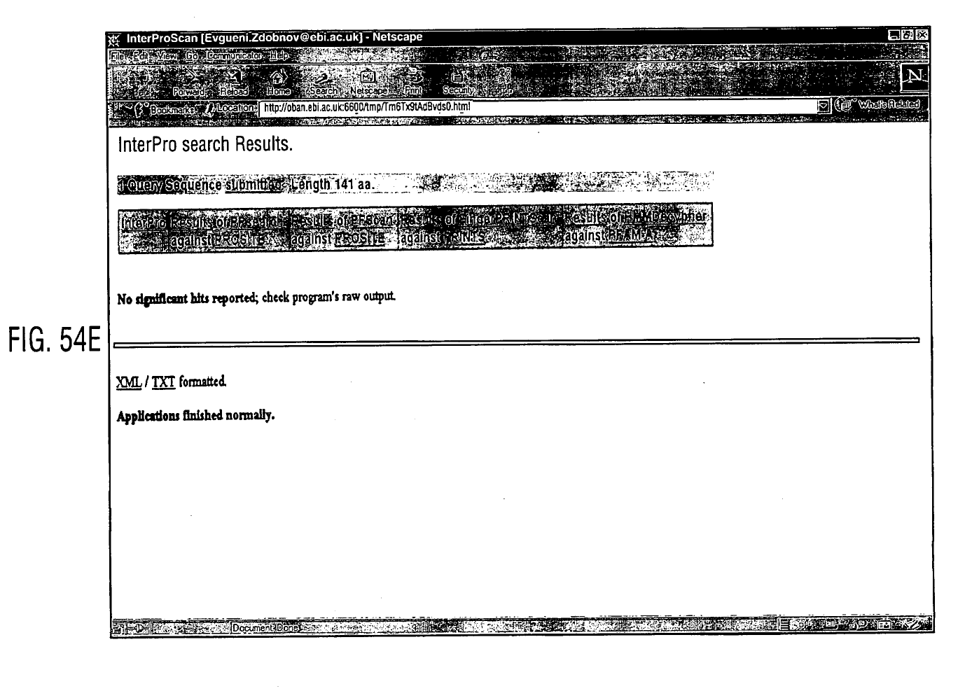

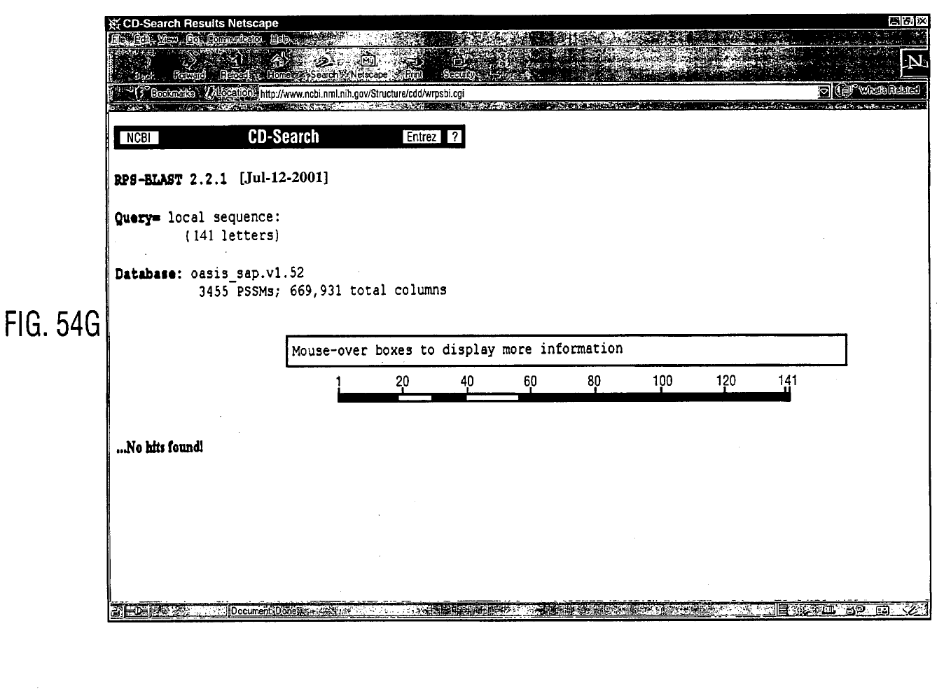

In order to ensure that the protein identified is a homologue of the query sequence, the visualisation program LigEye (FIG. 54A) and RasMol (FIG. 54B) are used. These visualization tools identify the active site of known protein structures by indicating the amino acids with which known small molecule inhibitors interact at the active site. These interactions are through either a direct hydrogen bond or hydrophobic interactions. In this manner one can see if the active site fold/structure is conserved between the identified homologue and the chosen protein of known structure.

This visualisation is shown with 1AOX, which illustrates the sites of interaction of a magnesium ion with the I domain of integrin Alpha 2-Beta 1 (FIG. 54A). The magnesium ion sees 3 different amino acids in the I domain of 1AOX. These three amino acids of 1AOX (SER153, SER155, and ASP254) are conserved in Rv0368c (AD6) (FIG. 54B, ball structures). FIG. 54C farther identifies the amino acids that are conserved between 1AOX and Rv0368c (AD6). The conservation of amino acids indicates that the fold is identical. This indicates that indeed as predicted by the Inpharmatica Genome Threader™, Rv0368c (AD6) folds in a similar manner to 1AOX and as such is identified as an I domain-containing protein.

FIG. 54D shows the InterPro results for Rv0368c (AD6) as of Jul. 24, 2001. InterPro is a public domain annotation tool which combines PROSITE pattern, PROSITE profile, PRINTS and PFAM. InterPro returns a hit to a lipocalin, but in a different region to the region of Rv0368c that is annotated herein as possessing activity as an adhesion molecule (the same hit is not reported when residues 230-370 of Rv0368c (AD6), rather than full-length sequence, is inputted into InterPro, see FIG. 54E). The fact that InterPro returns no adhesion molecule hits for Rv0368c (AD6) on the Jul. 24, 2001 demonstrates that on the date of PCT filing, Rv0368c (AD6) could still only be annotated as an adhesion molecule by Inpharmatica Genome Threader™.

FIG. 54F shows the NCBI Conserved Domain Database (CDD) results for Rv0368c (AD6) as of Jul. 24, 2001. CDD returns no bits. (no hits are also reported when residues 230-370 of Rv0368c (AD6), rather than the full-length sequence, is inputted into CDD, see FIG. 54G). The fact that CDD returns no results for Rv0368c (AD6) on the Jul. 24, 2001 demonstrates that on the date of PCT filing, Rv0368c (AD6) could still only be annotated as an adhesion molecule by Inpharmatica Genome Threader™.

The invention will now be further described by way of the following numbered paragraphs:

1. A polypeptide, which polypeptide: has the amino acid sequence as recited in SEQ ID NO:2, SEQ ID NO:4, SEQ ID NO:6, SEQ ID NO:8, SEQ ID NO: 10 or SEQ ID NO: 12;

is a fragment thereof having adhesion molecule activity or having an antigenic determinant in common with the polypeptide of (i); or

is a functional equivalent of (i) or (ii).

2. A polypeptide which is a fragment according to paragraph 1(ii), which includes the adhesion molecule region of the AD1 polypeptide, said adhesion molecule region being defined as including, at the most, between residues 1832 and 2036 inclusive, or at the least, between residues 1836 and 1950 inclusive, of the amino acid sequence recited in SEQ ID NO:2, wherein said fragment possesses the catalytic residues Ser1843, Ser1845 and Asp1912, or equivalent residues, or the trio Ser1843, Ser1845 and Thr1912, or equivalent residues, and possesses adhesion molecule activity.

3. A polypeptide which is a functional equivalent according to paragraph 1(iii), is homologous to the amino acid sequence as recited in SEQ ID NO:2, possesses the catalytic residues Ser1843, Ser1845 and Asp 1912, or equivalent residues, or the trio Ser1843, Ser1845 and Thr1912, or equivalent residues, and has adhesion molecule activity.

4. A polypeptide according to paragraph 3, wherein said functional equivalent is homologous to the adhesion molecule region of the AD1 polypeptide.

5. A polypeptide which is a fragment according to paragraph 1(ii), which includes the adhesion molecule region of the AD2 polypeptide, said adhesion molecule region being defined as including, at the most, between residue 10 and residue 126, and at the least, between residue 20 and residue 105 of the amino acid sequence recited in SEQ ID NO:4, wherein said fragment possesses the catalytic residues Thr25, Ser27 and Asp119, or equivalent residues, and possesses adhesion molecule activity.

6. A polypeptide which is a functional equivalent according to paragraph 1(iii), is homologous to the amino acid sequence as recited in SEQ ID NO:4, possesses the catalytic residues Thr25, Ser27 and Asp 119, or equivalent residues, and has adhesion molecule activity.

7. A polypeptide according to paragraph 6, wherein said functional equivalent is homologous to the adhesion molecule region of the AD2 polypeptide.

8. A polypeptide which is a fragment according to paragraph 1(ii), which includes the adhesion molecule region of the AD3 polypeptide, said adhesion molecule region being defined as including, at the most, between residue 1248 and residue 1432, and at the least, between residue 1253 and residue 1403 of the amino acid sequence recited in SEQ ID NO:6, wherein said fragment possesses the catalytic residues Ser1258, Ser1260 and Asp1367, or equivalent residues, and possesses adhesion molecule activity.

9. A polypeptide which is a functional equivalent according to paragraph 1(iii), is homologous to the amino acid sequence as recited in SEQ ID NO:6, possesses the catalytic residues Ser1258, Ser1260 and Asp1367, or equivalent residues, and has adhesion molecule activity.

10. A polypeptide according to paragraph 9, wherein said functional equivalent is homologous to the adhesion molecule region of the AD3 polypeptide.

11. A polypeptide which is a fragment according to paragraph 1(ii), which includes the adhesion molecule region of the AD4 polypeptide, said adhesion molecule region being defined as including between residue 308 and residue 424 of the amino acid sequence recited in SEQ ID NO:8, wherein said fragment possesses the catalytic residues Thr323, Ser325 and Asp417, or equivalent residues, and possesses adhesion molecule activity.

12. A polypeptide which is a functional equivalent according to paragraph 1(iii), is homologous to the amino acid sequence as recited in SEQ ID NO:8, possesses the catalytic residues Thr323, Ser325 and Asp417, or equivalent residues, and has adhesion molecule activity.

13. A polypeptide according to paragraph 12, wherein said functional equivalent is homologous to the adhesion molecule region of the AD4 polypeptide.

14. A polypeptide which is a fragment according to paragraph 1(ii), which includes the adhesion molecule region of the AD5 polypeptide, said adhesion molecule region being defined as including, at the most, between residue 482 and residue 646, and at the least; between residue 484 and residue 646 of the amino acid sequence recited in SEQ ID NO:10, wherein said fragment possesses the catalytic residues Ser491, Ser493 and Asp579, or equivalent residues, and possesses adhesion molecule activity.

15. A polypeptide which is a functional equivalent according to paragraph 1(iii), is homologous to the amino acid sequence as recited in SEQ ID NO:10, possesses the catalytic residues Ser491, Ser493 and Asp579, or equivalent residues, and has adhesion molecule activity.

16. A polypeptide according to paragraph 15, wherein said functional equivalent is homologous to the adhesion molecule region of the AD5 polypeptide.

17. A polypeptide which is a fragment according to paragraph 1(ii), which includes the adhesion molecule region of the AD6 polypeptide, said adhesion molecule region being defined as including, at the most, between residue 230 and residue 370, and at the least, between residue 230 and residue 339 of the amino acid sequence recited in SEQ ID NO:12, wherein said fragment possesses the catalytic residues Ser237, Ser239 and Asp330, or equivalent residues; or the trio of Ser237, Ser239 and Thr302, or equivalent residues, and possesses adhesion molecule activity.

18. A polypeptide which is a functional equivalent according to paragraph 1(iii), is homologous to the amino acid sequence as recited in SEQ ID NO:12, possesses the catalytic residues Ser237, Ser239 and Asp330, or equivalent residues; or the trio of Ser237, Ser239 and Thr302, or equivalent residues, and has adhesion molecule activity.

19. A polypeptide according to paragraph 18, wherein said functional equivalent is homologous to the adhesion molecule region of the AD6 polypeptide.

20. A fragment or functional equivalent according to any one of paragraphs 1-19, which has greater than 30% sequence identity with an amino acid sequence as recited in any one of SEQ ID NO:2, SEQ ID NO:4, SEQ ID NO:6, SEQ ID NO:8, SEQ ID NO: 10 and SEQ ID NO:12, or with a fragment thereof that possesses adhesion molecule activity, preferably greater than 40%, 50%, 60%, 70%, 80%, 90%, 95%, 98% or 99% sequence identity, as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=I].

21. A functional equivalent according to any one of paragraphs 1-19, which exhibits significant structural homology with a polypeptide having the amino acid sequence given in any one of SEQ ID NO:2, SEQ ID NO:4, SEQ ID NO:6, SEQ ID NO:8, SEQ ID NO:10 and SEQ ID NO:12, or with a fragment thereof that possesses adhesion molecule activity.

22. A fragment as recited in paragraph 1, 2, 5, 8, 11, 14, 17 or 20, having an antigenic determinant in common with the polypeptide of paragraph 1(i), which consists of 7 or more (for example, 8, 10, 12, 14, 16, 18, 20 or more) amino acid residues from the sequence of SEQ ID NO:2, SEQ ID NO:4, SEQ ID NO:6, SEQ ID NO:8, SEQ ID NO:10 or SEQ ID NO:12.

23. A purified nucleic acid molecule which encodes a polypeptide according to any one of the preceding paragraphs.

24. A purified nucleic acid molecule according to paragraph 23, which has the nucleic acid sequence as recited in SEQ ID NO:1, SEQ ID NO:3, SEQ ID NO:5, SEQ ID NO:7, SEQ ID NO:9 or SEQ ID NO: 11, or is a redundant equivalent or fragment thereof.

25. A fragment of a purified nucleic acid molecule according to paragraph 23 or paragraph 24, which comprises, at the most, between nucleotides 5495 and 6109, and at the least, between nucleotides 5507 and 5851 of SEQ ID NO:1, or is a redundant equivalent thereof.

26. A fragment of a purified nucleic acid molecule according to paragraph 23 or paragraph 24, which comprises, at the most, between nucleotides 30 and 380, and at the least, between nucleotides 60 and 317 of SEQ ID NO:3, or is a redundant equivalent thereof.

27. A fragment of a purified nucleic acid molecule according to paragraph 23 or paragraph 24, which comprises, at the most, between nucleotides 3744 and 4298, and at the least, between nucleotides 3759 and 4211 of SEQ ID NO:5, or is a redundant equivalent thereof.

28. A fragment of a purified nucleic acid molecule according to paragraph 23 or paragraph 24, which comprises between nucleotides 922 and 1272 of SEQ ID NO:7, or is a redundant equivalent thereof.

29. A fragment of a purified nucleic acid molecule according to paragraph 23 or paragraph 24, which comprises, at the most, between nucleotides 1444 and 1938, and at the least, between nucleotides 1450 and 1938 of SEQ ID NO:9, or is a redundant equivalent thereof.

30. A fragment of a purified nucleic acid molecule according to paragraph 23 or paragraph 24, which comprises, at the most, between nucleotides 688 and 1110, and at the least, between nucleotides 688 and 1017 of SEQ ID NO: 11, or is a redundant equivalent thereof.

31. A purified nucleic acid molecule which hydridizes under high stringency conditions with a nucleic acid molecule according to any one of paragraphs 23-30.

32. A vector comprising a nucleic acid molecule as recited in any one of paragraphs 23-31.

33. A host cell transformed with a vector according to paragraph 32.

34. A ligand which binds specifically to, and which preferably inhibits the adhesion molecule activity of, a polypeptide according to any one of paragraphs 1-22.

35. A ligand according to paragraph 34, which is an antibody.

36. A compound that either increases or decreases the level of expression or activity of a polypeptide according to any one of paragraphs 1-22.

37. A compound according to paragraph 36 that binds to a polypeptide according to any one of paragraphs 1-22 without inducing any of the biological effects of the polypeptide.

38. A compound according to paragraph 36 or paragraph 37, which is a natural or modified substrate, ligand, enzyme, receptor or structural or functional mimetic.

39. A polypeptide according to any one of paragraphs 1-22, a nucleic acid molecule according to any one of paragraphs 23-31, a vector according to paragraph 32, a ligand according to paragraph 34 or 35, or a compound according to any one of paragraphs 36-38, for use in therapy or diagnosis of disease.

40. A method of diagnosing a disease in a patient, comprising assessing the level of expression of a natural gene encoding a polypeptide according to any one of paragraphs 1-22, or assessing the activity of a polypeptide according to any one of paragraph 1-22, in tissue from said patient and comparing said level of expression or activity to a control level, wherein a level that is different from said control level is indicative of disease.

41. A method according to paragraph 40 that is carried out in vitro.

42. A method according to paragraph 40 or paragraph 41, which comprises the steps of (a) contacting a ligand according to paragraph 34 or paragraph 35 with a biological sample under conditions suitable for the formation of a ligand-polypeptide complex; and (b) detecting said complex.

43. A method according to paragraph 40 or paragraph 41, comprising the steps of:

a. contacting a sample of tissue from the patient with a nucleic acid probe under stringent conditions that allow the formation of a hybrid complex between a nucleic acid molecule according to any one of paragraphs 23-31 and the probe;

b. contacting a control sample with said probe under the same conditions used in step a); and

c. detecting the presence of hybrid complexes in said samples;

wherein detection of levels of the hybrid complex in the patient sample that differ from levels of the hybrid complex in the control sample is indicative of disease.

44. A method according to paragraph 40 or paragraph 41, comprising:

a. contacting a sample of nucleic acid from tissue of the patient with a nucleic acid primer under stringent conditions that allow the formation of a hybrid complex between a nucleic acid molecule according to any one of paragraphs 23-31 and the primer;

b. contacting a control sample with said primer under the same conditions used in step a); and

c. amplifying the sampled nucleic acid; and

d. detecting the level of amplified nucleic acid from both patient and control samples;

wherein detection of levels of the amplified nucleic acid in the patient sample that differ significantly from levels of the amplified nucleic acid in the control sample is indicative of disease.

45. A method according to paragraph 40 or paragraph 41 comprising:

a. obtaining a tissue sample from a patient being tested for disease;

b. isolating a nucleic acid molecule according to any one of paragraphs 23-31 from said tissue sample; and

c. diagnosing the patient for disease by detecting the presence of a mutation which is associated with disease in the nucleic acid molecule as an indication of the disease.

46. The method of paragraph 45, further comprising amplifying the nucleic acid molecule to form an amplified product and detecting the presence or absence of a mutation in the amplified product.

47. The method of either paragraph 45 or 46, wherein the presence or absence of the mutation in the patient is detected by contacting said nucleic acid molecule with a nucleic acid probe that hybridises to said nucleic acid molecule under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation.

48. A method according to any one of paragraphs 40-47, wherein said disease is a cardiovascular disease, including atherosclerosis, ischaemia, restenosis, reperfusion injury, sepsis; a haematological disease such as leukaemia; a blood clotting disorder, such as thrombosis; cancer, including lung, prostate, breast, colorectal and brain tumours, metastasis; an inflammatory disease such as rhinitis; a gastrointestinal disease, including inflammatory bowel disease, ulcerative colitis, Crohn's disease; a respiratory disease, including asthma; chronic obstructive pulmonary disease (COPD); respiratory distress syndrome; pulmonary fibrosis; immune disorders, including autoimmune diseases, rheumatoid arthritis, transplant rejection; allergy; liver diseases, such as cirrhosis; endocrine diseases, such as diabetes; bone diseases such as osteoporosis; neurological diseases, including stroke, multiple sclerosis, spinal cord injury; burns and wound healing; bacterial infection, particularly Mycobacterium tuberculosis infection, and virus infection.

49. Use of a polypeptide according to any one of paragraphs 1-22 as an adhesion molecule.

50. Use of a nucleic acid molecule according to any one of paragraphs 23-31 to express a protein that possesses adhesion molecule activity.

51. A method for effecting cell-cell adhesion, utilising a polypeptide according to any one of paragraphs 1-22.

52. A pharmaceutical composition comprising a polypeptide according to any one of paragraphs 1-22, a nucleic acid molecule according to any one of paragraphs 23-31, a vector according to paragraph 32, a ligand according to paragraph 34 or 35, or a compound according to any one of paragraphs 36-38.

53. A vaccine composition comprising a polypeptide according to any one of paragraphs 1-22 or a nucleic acid molecule according to any one of paragraphs 23-31.

54. A polypeptide according to any one of paragraphs 1-22, a nucleic acid molecule according to any one of paragraphs 23-31, a vector according to paragraph 32, a ligand according to paragraph 34 or 35, a compound according to any one of paragraphs 36-38, or a pharmaceutical composition according to paragraph 52 for use in the manufacture of a medicament for the treatment of a cardiovascular disease, including atherosclerosis, ischaemia, restenosis, reperfusion injury, sepsis; a haematological disease such as leukaemia; a blood clotting disorder, such as thrombosis; cancer, including lung, prostate, breast, colorectal and brain tumours, metastasis; an inflammatory disease such as rhinitis; a gastrointestinal disease, including inflammatory bowel disease, ulcerative colitis, Crohn's disease; a respiratory disease, including asthma; chronic obstructive pulmonary disease (COPD); respiratory distress syndrome; pulmonary fibrosis; immune disorders, including autoimmune diseases, rheumatoid arthritis, transplant rejection; allergy; liver diseases, such as cirrhosis; endocrine diseases, such as diabetes; bone diseases such as osteoporosis; neurological diseases, including stroke, multiple sclerosis, spinal cord injury; burns and wound healing; bacterial infection, particularly Mycobacterium tuberculosis infection, or a virus infection.

55. A method of treating a disease in a patient, comprising administering to the patient a polypeptide according to any one of paragraphs 1-22, a nucleic acid molecule according to any one of paragraphs 23-31, a vector according to paragraph 32, a ligand according to paragraph 34 or 35, a compound according to any one of paragraphs 36-38, or a pharmaceutical composition according to paragraph 52.

56. A method according to paragraph 55, wherein, for diseases in which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

57. A method according to paragraph 55, wherein, for diseases in which the expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

58. A method of monitoring the therapeutic treatment of disease in a patient, comprising monitoring over a period of time the level of expression or activity of a polypeptide according to any one of paragraphs 1-22, or the level of expression of a nucleic acid molecule according to any one of paragraphs 23-31 in tissue from said patient, wherein altering said level of expression or activity over the period of time towards a control level is indicative of regression of said disease.

59. A method for the identification of a compound that is effective in the treatment and/or diagnosis of disease, comprising contacting a polypeptide according to any one of paragraphs 1-22, a nucleic acid molecule according to any one of paragraphs 23-31, or a host cell according to paragraph 33 with one or more compounds suspected of possessing binding affinity for said polypeptide or nucleic acid molecule, and selecting a compound that binds specifically to said nucleic acid molecule or polypeptide.

60. A kit useful for diagnosing disease comprising a first container containing a nucleic acid probe that hybridises under stringent conditions with a nucleic acid molecule according to any one of paragraphs 23-31; a second container containing primers useful for amplifying said nucleic acid molecule; and instructions for using the probe and primers for facilitating the diagnosis of disease.

61. The kit of paragraph 60, further comprising a third container holding an agent for digesting unhybridised RNA.

62. A kit comprising an array of nucleic acid molecules, at least one of which is a nucleic acid molecule according to any one of paragraphs 23-31.

63. A kit comprising one or more antibodies that bind to a polypeptide as recited in any one of paragraphs 1-22; and a reagent useful of the detection of a binding reaction between said antibody and said polypeptide.

64. A transgenic or knockout non-human animal that has been transformed to express higher, lower or absent levels of a polypeptide according to any one of paragraphs 1-22.

65. A method for screening for a compound effective to treat disease, by contacting a non-human transgenic animal according to paragraph 64 with a candidate compound and determining the effect of the compound on the disease of the animal.

1. A polypeptide, which polypeptide:
(i) has the amino acid sequence as recited in SEQ ID NO:2, SEQ ID NO:4, SEQ ID NO:6, SEQ ID NO:8, SEQ ID NO: 10 or SEQ ID NO: 12;
(ii) is a fragment thereof having adhesion molecule activity or having an antigenic determinant in common with the polypeptide of (i); or
(iii) is a functional equivalent of (i) or (ii).

2. A polypeptide which is a fragment according to claim 1(ii), which includes the adhesion molecule region of the AD1 polypeptide, said adhesion molecule region being defined as including, at the most, between residues 1832 and 2036 inclusive, or at the least, between residues 1836 and 1950 inclusive, of the amino acid sequence recited in SEQ ID NO:2, wherein said fragment possesses the catalytic residues Ser1843, Ser1845 and Asp1912, or equivalent residues, or the trio Ser1843, Ser1845 and Thr1912, or equivalent residues, and possesses adhesion molecule activity.

3. A polypeptide which is a functional equivalent according to claim 1(iii), is homologous to the amino acid sequence as recited in SEQ ID NO:2, possesses the catalytic residues Ser1843, Ser1845 and Asp1912, or equivalent residues, or the trio Ser1843, Ser1845 and Thr1912, or equivalent residues, and has adhesion molecule activity.

4. A polypeptide according to claim 3, wherein said functional equivalent is homologous to the adhesion molecule region of the AD1 polypeptide.

5. A polypeptide which is a fragment according to claim 1(ii), which includes the adhesion molecule region of the AD2 polypeptide, said adhesion molecule region being defined as including, at the most, between residue 10 and residue 126, and at the least, between residue 20 and residue 105 of the amino acid sequence recited in SEQ ID NO:4, wherein said fragment possesses the catalytic residues Thr25, Ser27 and Asp119, or equivalent residues, and possesses adhesion molecule activity.

6. A polypeptide which is a functional equivalent according to claim 1(iii), is homologous to the amino acid sequence as recited in SEQ ID NO:4, possesses the catalytic residues Thr25, Ser27 and Asp119, or equivalent residues, and has adhesion molecule activity.

7. A polypeptide according to claim 6, wherein said functional equivalent is homologous to the adhesion molecule region of the AD2 polypeptide.

8. A polypeptide which is a fragment according to claim 1(ii), which includes the adhesion molecule region of the AD3 polypeptide, said adhesion molecule region being defined as including, at the most, between residue 1248 and residue 1432, and at the least, between residue 1253 and residue 1403 of the amino acid sequence recited in SEQ ID NO:6, wherein said fragment possesses the catalytic residues Ser1258, Ser1260 and Asp1367, or equivalent residues, and possesses adhesion molecule activity.

9. A polypeptide which is a functional equivalent according to claim 1(iii), is homologous to the amino acid sequence as recited in SEQ ID NO:6, possesses the catalytic residues Ser1258, Ser1260 and Asp1367, or equivalent residues, and has adhesion molecule activity.

10. A polypeptide according to claim 9, wherein said functional equivalent is homologous to the adhesion molecule region of the AD3 polypeptide.

11. A polypeptide which is a fragment according to claim 1(ii), which includes the adhesion molecule region of the AD4 polypeptide, said adhesion molecule region being defined as including between residue 308 and residue 424 of the amino acid sequence recited in SEQ ID NO:8, wherein said fragment possesses the catalytic residues Thr323, Ser325 and Asp417, or equivalent residues, and possesses adhesion molecule activity.

12. A polypeptide which is a functional equivalent according to claim 1(iii), is homologous to the amino acid sequence as recited in SEQ ID NO:8, possesses the catalytic residues Thr323, Ser325 and Asp417, or equivalent residues, and has adhesion molecule activity.

13. A polypeptide according to claim 12, wherein said functional equivalent is homologous to the adhesion molecule region of the AD4 polypeptide.

14. A polypeptide which is a fragment according to claim 1(ii), which includes the adhesion molecule region of the AD5 polypeptide, said adhesion molecule region being defined as including, at the most, between residue 482 and residue 646, and at the least; between residue 484 and residue 646 of the amino acid sequence recited in SEQ ID NO:10, wherein said fragment possesses the catalytic residues Ser491, Ser493 and Asp579, or equivalent residues, and possesses adhesion molecule activity.

15. A polypeptide which is a functional equivalent according to claim 1(iii), is homologous to the amino acid sequence as recited in SEQ ID NO:10, possesses the catalytic residues Ser491, Ser493 and Asp579, or equivalent residues, and has adhesion molecule activity.

16. A polypeptide according to claim 15, wherein said functional equivalent is homologous to the adhesion molecule region of the AD5 polypeptide.

17. A polypeptide which is a fragment according to claim 1(ii), which includes the adhesion molecule region of the AD6 polypeptide, said adhesion molecule region being defined as including, at the most, between residue 230 and residue 370, and at the least, between residue 230 and residue 339 of the amino acid sequence recited in SEQ ID NO:12, wherein said fragment possesses the catalytic residues Ser237, Ser239 and Asp330, or equivalent residues; or the trio of Ser237, Ser239 and Thr302, or equivalent residues, and possesses adhesion molecule activity.

18. A polypeptide which is a functional equivalent according to claim 1(iii), is homologous to the amino acid sequence as recited in SEQ ID NO:12, possesses the catalytic residues Ser237, Ser239 and Asp330, or equivalent residues; or the trio of Ser237, Ser239 and Thr302, or equivalent residues, and has adhesion molecule activity.

19. A polypeptide according to claim 18, wherein said functional equivalent is homologous to the adhesion molecule region of the AD6 polypeptide.

20. A fragment or functional equivalent according to any one of claims 1-19, which has greater than 30% sequence identity with an amino acid sequence as recited in any one of SEQ ID NO:2, SEQ ID NO:4, SEQ ID NO:6, SEQ ID NO:8, SEQ ID NO: 10 and SEQ ID NO:12, or with a fragment thereof that possesses adhesion molecule activity, preferably greater than 40%, 50%, 60%, 70%, 80%, 90%, 95%, 98% or 99% sequence identity, as determined using BLAST version 2.1.3 using the default parameters specified by the National Center for Biotechnology Information.

21. A functional equivalent according to claim 1, which exhibits significant structural homology with a polypeptide having the amino acid sequence given in any one of SEQ ID NO:2, SEQ ID NO:4, SEQ ID NO:6, SEQ ID NO:8, SEQ ID NO:10 and SEQ ID NO:12, or with a fragment thereof that possesses adhesion molecule activity.

22. A fragment as recited in claim 1, having an antigenic determinant in common with the polypeptide of claim 1(i), which consists of 7 or more amino acid residues from the sequence of SEQ ID NO:2, SEQ ID NO:4, SEQ ID NO:6, SEQ ID NO:8, SEQ ID NO:10 or SEQ ID NO:12.

23. A purified nucleic acid molecule which encodes a polypeptide according to claim 1.

24. A purified nucleic acid molecule according to claim 23, which has the nucleic acid sequence as recited in SEQ ID NO: 1, SEQ ID NO:3, SEQ ID NO:5, SEQ ID NO:7, SEQ ID NO:9 or SEQ ID NO: 11, or is a redundant equivalent or fragment thereof.

25. A fragment of a purified nucleic acid molecule according to claim 23, which comprises, at the most, between nucleotides 5495 and 6109, and at the least, between nucleotides 5507 and 5851 of SEQ ID NO:1, or is a redundant equivalent thereof.

26. A fragment of a purified nucleic acid molecule according to claim 23, which comprises, at the most, between nucleotides 30 and 380, and at the least, between nucleotides 60 and 317 of SEQ ID NO:3, or is a redundant equivalent thereof.

27. A fragment of a purified nucleic acid molecule according to claim 23, which comprises, at the most, between nucleotides 3744 and 4298, and at the least, between nucleotides 3759 and 4211 of SEQ ID NO:5, or is a redundant equivalent thereof.

28. A fragment of a purified nucleic acid molecule according to claim 23, which comprises between nucleotides 922 and 1272 of SEQ ID NO:7, or is a redundant equivalent thereof.

29. A fragment of a purified nucleic acid molecule according to claim 23, which comprises, at the most, between nucleotides 1444 and 1938, and at the least, between nucleotides 1450 and 1938 of SEQ ID NO:9, or is a redundant equivalent thereof.

30. A fragment of a purified nucleic acid molecule according to claim 23, which comprises, at the most, between nucleotides 688 and 1110, and at the least, between nucleotides 688 and 1017 of SEQ ID NO: 11, or is a redundant equivalent thereof.

31. A purified nucleic acid molecule which hydridizes under high stringency conditions with a nucleic acid molecule according to claim 23.

32. A vector comprising a nucleic acid molecule as recited in claim 23.

33. A host cell transformed with a vector according to claim 32.

34. A ligand which binds specifically to, and which preferably inhibits the adhesion molecule activity of, a polypeptide according to claim 1.

35. A ligand according to claim 34, which is an antibody.

36. A compound that either increases or decreases the level of expression or activity of a polypeptide according to claim 1.

37. A compound that either increases or decreases the level of expression or activity of a polypeptide according to claim 1, wherein the compound binds to the polypeptide without inducing any of the biological effects of the polypeptide.

38. A compound according to claim 36, which is a natural or modified substrate, ligand, enzyme, receptor or structural or functional mimetic.

39. A polypeptide according to claim 1, for use in therapy or diagnosis of disease.

40. A nucleic acid molecule according to claim 23, for use in therapy or diagnosis of disease.

41. A vector according to claim 32, for use in therapy or diagnosis of disease.

42. A ligand according to claim 34, for use in therapy or diagnosis of disease.

43. A compound according claim 36, for use in therapy or diagnosis of disease.

44. A method of diagnosing a disease in a patient, comprising assessing the level of expression of a natural gene encoding a polypeptide according to claim 1, or assessing the activity of a polypeptide according to claim 1, in tissue from said patient and comparing said level of expression or activity to a control level, wherein a level that is different to said control level is indicative of disease.

45. A method according to claim 44 that is carried out in vitro.

46. A method of diagnosing a disease in a patient, comprising assessing the level of expression of a natural gene encoding a polypeptide according to claim 1, or assessing the activity of a polypeptide according to claim 1, in tissue from said patient and comparing said level of expression or activity to a control level, wherein a level that is different to said control level is indicative of disease, wherein the method comprises the steps of
(a) contacting a ligand which binds specifically to, and which preferably inhibits the adhesion molecule activity of, a polypeptide according to claim 1 with a biological sample under conditions suitable for the formation of a ligand-polypeptide complex; and
(b) detecting said complex.

47. A method of diagnosing a disease in a patient, comprising assessing the level of expression of a natural gene encoding a polypeptide according to claim 1, or assessing the activity of a polypeptide according to claim 1, in tissue from said patient and comparing said level of expression or activity to a control level, wherein a level that is different to said control level is indicative of disease, comprising the steps of:
a) contacting a sample of tissue from the patient with a nucleic acid probe under stringent conditions that allow the formation of a hybrid complex between a nucleic acid molecule which encodes a polypeptide according to claim 1 and the probe;
b) contacting a control sample with said probe under the same conditions used in step a); and
c) detecting the presence of hybrid complexes in said samples;
wherein detection of levels of the hybrid complex in the patient sample that differ from levels of the hybrid complex in the control sample is indicative of disease.

48. A method of diagnosing a disease in a patient, comprising assessing the level of expression of a natural gene encoding a polypeptide according to claim 1, or assessing the activity of a polypeptide according to claim 1, in tissue from said patient and comparing said level of expression or activity to a control level, wherein a level that is different to said control level is indicative of disease, comprising:
a) contacting a sample of nucleic acid from tissue of the patient with a nucleic acid primer under stringent conditions that allow the formation of a hybrid complex between a nucleic acid molecule which encodes a polypeptide according to claim 1 and the primer;
b) contacting a control sample with said primer under the same conditions used in step a); and
c) amplifying the sampled nucleic acid; and
d) detecting the level of amplified nucleic acid from both patient and control samples;
wherein detection of levels of the amplified nucleic acid in the patient sample that differ significantly from levels of the amplified nucleic acid in the control sample is indicative of disease.

49. A method of diagnosing a disease in a patient, comprising assessing the level of expression of a natural gene encoding a polypeptide according to claim 1, or assessing the activity of a polypeptide according to claim 1, in tissue from said patient and comparing said level of expression or activity to a control level, wherein a level that is different to said control level is indicative of disease, comprising:
a) obtaining a tissue sample from a patient being tested for disease;
b) isolating a nucleic acid molecule which encodes a polypeptide according to claim 1 from said tissue sample; and
c) diagnosing the patient for disease by detecting the presence of a mutation which is associated with disease in the nucleic acid molecule as an indication of the disease.

50. The method of claim 49, further comprising amplifying the nucleic acid molecule to form an amplified product and detecting the presence or absence of a mutation in the amplified product.

51. The method of either claim 49, wherein the presence or absence of the mutation in the patient is detected by contacting said nucleic acid molecule with a nucleic acid probe that hybridises to said nucleic acid molecule under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation.

52. A method according to claim 44, wherein said disease is a cardiovascular disease, atherosclerosis, ischaemia, restenosis, reperfusion injury, sepsis, a haematological disease, leukaemia, a blood clotting disorder, thrombosis, cancer, lung cancer, prostate cancer, breast cancer, colorectal tumors, brain tumors, metastasis, an inflammatory disease, rhinitis, a gastrointestinal disease, inflammatory bowel disease, ulcerative colitis, Crohn's disease, a respiratory disease, asthma, chronic obstructive pulmonary disease (COPD), respiratory distress syndrome, pulmonary fibrosis, immune disorders, autoimmune diseases, rheumatoid arthritis, transplant rejection, allergy, liver diseases, cirrhosis, endocrine diseases, diabetes, bone diseases, osteoporosis, neurological diseases, stroke, multiple sclerosis, spinal cord injury, burns, wound healing, bacterial infection, or virus infection.

53. The method of claim 52, wherein the bacterial infection is a Mycobacterium tuberculosis infection.

54. A method of using a polypeptide according to claim 1 as an adhesion molecule.

55. A method of using a nucleic acid molecule according to claim 23 to express a protein that possesses adhesion molecule activity.

56. A method for effecting cell-cell adhesion, utilizing a polypeptide according to claim 1.

57. A pharmaceutical composition comprising a polypeptide according to claim 1.

58. A pharmaceutical composition comprising a nucleic acid molecule according to claim 23.

59. A pharmaceutical composition comprising a vector according to claim 32.

60. A pharmaceutical composition comprising a ligand according to claim 34.

61. A pharmaceutical composition comprising a compound according to claim 36.

62. A vaccine composition comprising a polypeptide according to claim 1.

63. A vaccine composition comprising a nucleic acid molecule according to claim 23.

64. A pharmaceutical composition according to claim 57 for use in the manufacture of a medicament for the treatment of a cardiovascular disease, atherosclerosis, ischaemia, restenosis, reperfusion injury, sepsis, a haematological disease, leukaemia, a blood clotting disorder, thrombosis, cancer, lung cancer, prostate cancer, breast cancer, colorectal tumors, brain tumors, metastasis, an inflammatory disease, rhinitis, a gastrointestinal disease, inflammatory bowel disease, ulcerative colitis, Crohn's disease, a respiratory disease, asthma, chronic obstructive pulmonary disease (COPD), respiratory distress syndrome, pulmonary fibrosis, immune disorders, autoimmune diseases, rheumatoid arthritis, transplant rejection, allergy, liver diseases, cirrhosis, endocrine diseases, diabetes, bone diseases, osteoporosis, neurological diseases, stroke, multiple sclerosis, spinal cord injury, burns, wound healing, bacterial infection, or virus infection.

65. A polypeptide according to claim 1 for use in the manufacture of a medicament for the treatment of wherein said disease is a cardiovascular disease, atherosclerosis, ischaemia, restenosis, reperfusion injury, sepsis, a haematological disease, leukaemia, a blood clotting disorder, thrombosis, cancer, lung cancer, prostate cancer, breast cancer, colorectal tumors, brain tumors, metastasis, an inflammatory disease, rhinitis, a gastrointestinal disease, inflammatory bowel disease, ulcerative colitis, Crohn's disease, a respiratory disease, asthma, chronic obstructive pulmonary disease (COPD), respiratory distress syndrome, pulmonary fibrosis, immune disorders, autoimmune diseases, rheumatoid arthritis, transplant rejection, allergy, liver diseases, cirrhosis, endocrine diseases, diabetes, bone diseases, osteoporosis, neurological diseases, stroke, multiple sclerosis, spinal cord injury, burns, wound healing, bacterial infection, or virus infection.

66. A nucleic acid molecule according to claim 23 for use in the manufacture of a medicament for the treatment of wherein said disease is a cardiovascular disease, atherosclerosis, ischaemia, restenosis, reperfusion injury, sepsis, a haematological disease, leukaemia, a blood clotting disorder, thrombosis, cancer, lung cancer, prostate cancer, breast cancer, colorectal tumors, brain tumors, metastasis, an inflammatory disease, rhinitis, a gastrointestinal disease, inflammatory bowel disease, ulcerative colitis, Crohn's disease, a respiratory disease, asthma, chronic obstructive pulmonary disease (COPD), respiratory distress syndrome, pulmonary fibrosis, immune disorders, autoimmune diseases, rheumatoid arthritis, transplant rejection, allergy, liver diseases, cirrhosis, endocrine diseases, diabetes, bone diseases, osteoporosis, neurological diseases, stroke, multiple sclerosis, spinal cord injury, burns, wound healing, bacterial infection, or virus infection.

67. A vector according to claim 32 for use in the manufacture of a medicament for the treatment of a wherein said disease is a cardiovascular disease, atherosclerosis, ischaemia, restenosis, reperfusion injury, sepsis, a haematological disease, leukaemia, a blood clotting disorder, thrombosis, cancer, lung cancer, prostate cancer, breast cancer, colorectal tumors, brain tumors, metastasis, an inflammatory disease, rhinitis, a gastrointestinal disease, inflammatory bowel disease, ulcerative colitis, Crohn's disease, a respiratory disease, asthma, chronic obstructive pulmonary disease (COPD), respiratory distress syndrome, pulmonary fibrosis, immune disorders, autoimmune diseases, rheumatoid arthritis, transplant rejection, allergy, liver diseases, cirrhosis, endocrine diseases, diabetes, bone diseases, osteoporosis, neurological diseases, stroke, multiple sclerosis, spinal cord injury, burns, wound healing, bacterial infection, or virus infection.

68. A ligand according to claim 34 for use in the manufacture of a medicament for the treatment of a wherein said disease is a cardiovascular disease, atherosclerosis, ischaemia, restenosis, reperfusion injury, sepsis, a haematological disease, leukaemia, a blood clotting disorder, thrombosis, cancer, lung cancer, prostate cancer, breast cancer, colorectal tumors, brain tumors, metastasis, an inflammatory disease, rhinitis, a gastrointestinal disease, inflammatory bowel disease, ulcerative colitis, Crohn's disease, a respiratory disease, asthma, chronic obstructive pulmonary disease (COPD), respiratory distress syndrome, pulmonary fibrosis, immune disorders, autoimmune diseases, rheumatoid arthritis, transplant rejection, allergy, liver diseases, cirrhosis, endocrine diseases, diabetes, bone diseases, osteoporosis, neurological diseases, stroke, multiple sclerosis, spinal cord injury, burns, wound healing, bacterial infection, or virus infection.

69. A compound according to claim 36 for use in the manufacture of a medicament for the treatment of a wherein said disease is a cardiovascular disease, atherosclerosis, ischaemia, restenosis, reperfusion injury, sepsis, a haematological disease, leukaemia, a blood clotting disorder, thrombosis, cancer, lung cancer, prostate cancer, breast cancer, colorectal tumors, brain tumors, metastasis, an inflammatory disease, rhinitis, a gastrointestinal disease, inflammatory bowel disease, ulcerative colitis, Crohn's disease, a respiratory disease, asthma, chronic obstructive pulmonary disease (COPD), respiratory distress syndrome, pulmonary fibrosis, immune disorders, autoimmune diseases, rheumatoid arthritis, transplant rejection, allergy, liver diseases, cirrhosis, endocrine diseases, diabetes, bone diseases, osteoporosis, neurological diseases, stroke, multiple sclerosis, spinal cord injury, burns, wound healing, bacterial infection, or virus infection.

70. The method of claim 64, wherein the bacterial infection is a Mycobacterium tuberculosis infection.

71. The method of claim 65, wherein the bacterial infection is a Mycobacterium tuberculosis infection.

72. The method of claim 66, wherein the bacterial infection is a Mycobacterium tuberculosis infection.

73. The method of claim 67, wherein the bacterial infection is a Mycobacterium tuberculosis infection.

74. The method of claim 68, wherein the bacterial infection is a Mycobacterium tuberculosis infection.

75. The method of claim 69, wherein the bacterial infection is a Mycobacterium tuberculosis infection.

76. A method of treating a disease in a patient, comprising administering to the patient a polypeptide according to claim 1.

77. A method of treating a disease in a patient, comprising administering to the patient a nucleic acid molecule according to claim 23.

78. A method of treating a disease in a patient, comprising administering to the patient a vector according to claim 32.

79. A method of treating a disease in a patient, comprising administering to the patient a ligand according to claim 34.

80. A method of treating a disease in a patient, comprising administering to the patient a compound according to claim 36.

81. A method of treating a disease in a patient, comprising administering to the patient a pharmaceutical composition according to claim 57.

82. A method according to claim 76, wherein, for diseases in which the expression of the natural gene or the activity of the polypeptide is lower in a diseased patient when compared to the level of expression or activity in a healthy patient, the polypeptide administered to the patient is an agonist or an antagonist.

83. A method according to claim 77, wherein, for diseases in which the expression of the natural gene or the activity of the polypeptide is lower in a diseased patient when compared to the level of expression or activity in a healthy patient, the nucleic acid molecule administered to the patient is an agonist or an antagonist.

84. A method according to claim 78, wherein, for diseases in which the expression of the natural gene or the activity of the polypeptide is lower in a diseased patient when compared to the level of expression or activity in a healthy patient, the vector administered to the patient is an agonist or an antagonist.

85. A method according to claim 79, wherein, for diseases in which the expression of the natural gene or the activity of the polypeptide is lower in a diseased patient when compared to the level of expression or activity in a healthy patient, the ligand administered to the patient is an agonist or an antagonist.

86. A method according to claim 80, wherein, for diseases in which the expression of the natural gene or the activity of the polypeptide is lower in a diseased patient when compared to the level of expression or activity in a healthy patient, the compound administered to the patient is an agonist or an antagonist.

87. A method according to claim 81, wherein, for diseases in which the expression of the natural gene or the activity of the polypeptide is lower in a diseased patient when compared to the level of expression or activity in a healthy patient, the composition administered to the patient is an agonist or an antagonist.

88. A method of monitoring the therapeutic treatment of disease in a patient, comprising monitoring over a period of time the level of expression or activity of a polypeptide according to claim 1, in tissue from said patient, wherein altering said level of expression or activity over the period of time towards a control level is indicative of regression of said disease.

89. A method of monitoring the therapeutic treatment of disease in a patient, comprising monitoring over a period of time the level of expression of a nucleic acid molecule according to claim 23 in tissue from said patient, wherein altering said level of expression or activity over the period of time towards a control level is indicative of regression of said disease.

90. A method for the identification of a compound that is effective in the treatment and/or diagnosis of disease, comprising contacting a polypeptide according to claim 1 with one or more compounds suspected of possessing binding affinity for said polypeptide, and selecting a compound that binds specifically to said polypeptide.

91. A method for the identification of a compound that is effective in the treatment and/or diagnosis of disease, comprising contacting a nucleic acid molecule according to claim 23 with one or more compounds suspected of possessing binding affinity for nucleic acid molecule, and selecting a compound that binds specifically to said nucleic acid molecule.

92. A method for the identification of a compound that is effective in the treatment and/or diagnosis of disease, comprising contacting a host cell according to claim 33 with one or more compounds suspected of possessing binding affinity for said nucleic acid molecule, and selecting a compound that binds specifically to said nucleic acid molecule.

93. A kit useful for diagnosing disease comprising a first container containing a nucleic acid probe that hybridises under stringent conditions with a nucleic acid molecule according to claim 23; a second container containing primers useful for amplifying said nucleic acid molecule; and instructions for using the probe and primers for facilitating the diagnosis of disease.

94. The kit of claim 93, further comprising a third container holding an agent for digesting unhybridised RNA.

95. A kit comprising an array of nucleic acid molecules, at least one of which is a nucleic acid molecule according to claim 23.

96. A kit comprising one or more antibodies that bind to a polypeptide as recited in claim 1; and a reagent useful for the detection of a binding reaction between said antibody and said polypeptide.

97. A transgenic or knockout non-human animal that has been transformed to express higher, lower or absent levels of a polypeptide according to claim 1.

98. A method for screening for a compound effective to treat disease, by contacting a non-human transgenic animal according to claim 97 with a candidate compound and determining the effect of the compound on the disease of the animal.


SEQUENCE LISTING (57 sequences)

SEQ ID NO: 1 - DNA, 7651 bp, Homo sapiens
aagcggcctgtatagatacaggagcaggaactctaggacagccctgagtgaagaggaggaggaagaacgggagttcagaaaacagttccccctgcatgaaaaggactttgcagatattttggtgcagccaacgttggaggagaacaaaggaacttcagatgggcaagaagaggaagcaggcacaaacccagctctcctctcccagaattcaatgcaggcagtaatgctgatacaccagcaattgtgtctcaactttgctcgatccctctggtatcaacagactctgccgccacatgaagcaaagcattacctcagcctgtttctgtcttgctatcagactggggcatcgcttgtgacacacttctaccccctgatgggagttgaactgaatgaccgactcttgggcagccaacttttggcctgtaccctctcccataacactctttttggggaggcaccctcagacctgatggtgaaacctgatgggccctatgacttctaccagcatcccaatgttccagaagcacggcagtgtcaacctgtgcttcaaggtttctcagaggctgtcagtcacttgctacaggactggccagaacacccagcgcttgaacagctcctggttgtaatggacagaattcgtagtttcccactttccagtcccatctcaaagttcctgaatggcttagagatccttctggcaaaggcacaggattgggaggaaaatgcaagtcgagctttgtctttgcggaaacatcttgatttgatcagtcagatgatcattcggtggcgtaaactggagctgaactgctggtccatgagtttggataatactatgaagcgccacaccgagaaatccaccaagcactggttctccatctatcagatgcttgagaagcacatgcaggaacaaacagaagaacaggaagatgacaaacagatgaccttgatgttgctggtcagcacattacaagcatttattgaaggatcctcgctgggagagttccatgtgcgacttcagatgttactggttttccattgtcatgtcttgctgatgccacaggttgaaggaaaggattcactttgcagtgttctatggaatttgtaccattattacaagcaattctttgaccgggtccaggccaaaattgtggaacttcgttcccccctagaaaaagaacttaaagaatttgttaagatttccaagtggaatgatgtcagcttctggtccattaagcaatctgtagaaaagacacacaggacactctttaaattcatgaagaaatttgaagcagtcctgagtgaaccctgccggtcatccctggtggagagtgacaaggaagaacagcctgactttttgcccaggccaacagatggagctgcaagtgaactgtcttccattcagaatctgaacagggcactgagggagaccctgttagcccaaccagcagctgggcaggccacaattccagagtggtgtcagggcgctgctccttccggcttggaaggggagcttctgcgtcgcttgccaaagctcaggaaacgcatgaggaagatgtgcctgacgttcatgaaggagagccccctgcctcgccttgtggagggccttgatcagttcacaggtgaagtgatttcctctgtgagtgagctgcagagcttaaaggtggaaccctctgcagagaaggagaagcagcggtcagaagccaagcacattctcatgcaaaaacagcgagctttgtcagacctctttaaacaccttgcaaaaattggtttgtcgtatcgcaaaggtcttgcttgggcccgttcaaaaaaccctcaagagatgcttcatcttcacccattagatctccagagcgcattgtccatcgtcagcagcactcaggaggctgattctaggctgcttacagaaatctcgtcttcatgggatggatgccagaagtatttttatcgctctcttgcacggcatgccaggcttaacgcagcactagcaactcctgccaaggaaatgggcatgggcaacgtggagaggtgcagagggttctcagcacatttgatgaagatgctcgtccgacagcggcgctccctgaccacgctcagtgagcagtggatcatcctcaggaacctcctcagctgtgtgcaagagattcacagcaggctgatggggccccaggcctaccccgtggccttcccccctcaggatggcgtgcagcagtggacagagcgcctgcagcacctggccatgcagtgccagatcctgcttgagcagctctcctggctcctccagtgctgccccagtgtagggccagctccaggccatggcaatgtccaggtactggggcagcctcctggcccctgcctggaaggaccagaacttagcaagggacaactttgtggagtagtgctggacctaattccttccaatctgagctacccatctccaatacctggaagtcagctgccctctggttgccggatgcggaaacaggatcacctttggcaacagtcaactacgagattaacagagatgctaaaaaccattaaaacagtgaaagctgacgtcgacaaaattagacagcagtcttgtgagactctctttcattcttggaaagattttgaagtttgctcttctgcgctgagttgcttgtcccaggtgtcagttcatttgcagggcctagagtccttgttcattcttccagggatggaggttgagcaaagagactcacaaatggcactagttgaaagtctggaatatgtaagaggagaaattagtaaagccatggctgactttactacctggaagacccatctgcttacttcagatagccaaggaggaaatcaaatgttggacgaaggatttgtggaagatttttcagagcaaatggaaattgccatccgagccatcctctgtgccatccagaacttagaagaaagaaagaatgaaaaagcagaggagaacactgaccaagcaagcccacaagaagattatgcaggctttgagagactgcaatcaggacatctaacaaaactcttagaggatgacttctgggccgatgtgagcactttgcacgtgcagaaaataatttctgccatctccgagctgttggagaggctgaaatcgtacggtgaggatggcacagcagcaaagcacctgttcttcagccaatcctgttccttgctggtgcgcctggtgccggtcctctccagctactcagacctcgtcctcttcttcctgaccatgtctttagcaactcaccgtagtactgcaaagctgctctctgtgcttgcccaggtctttacagagcttgcccagaagggattttgcttgcccaaagaatttatggaagattcagctggagagggagcaactgagttccatgactatgagggaggtggaattggagaaggcgagggcatgaaggatgtgagtgaccagatcggaaatgaagaacaggtggaagatacatttcagaagggtcaagaaaaagacaaagaggatcctgattcaaaatctgatattaagggcgaggataatgccattgagatgtcggaagattttgatgggaaaatgcatgatggggagcttgaagaacaagaagaggatgatgagaaatcagatagtgagggcggagacctggataaacacatgggcgatctcaatggtgaggaagctgacaaactagatgagaggctttggggtgatgatgatgaggaggaagatgaggaggaagaagacaataaaactgaagaaacaggaccaggaatggatgaggaagattctgaacttgttgctaaagatgacaacttggatagtggcaattcaaacaaagataaaagccagcaagataagaaggaagaaaaggaagaagcagaagctgatgatggtggacaaggtgaagacaaaattaatgaacaaatagatgagagggactatgatgaaaatgaggtggacccttaccatggcaatcaggaaaaggtgccagaacccgaggctttggaccttccagatgacttgaaccttgacagtgaagacaagaatggtggtgaggacaccgacaatgaagaaggagaagaagagaatcctttggagataaaagaaaaaccagaagaagcaggtcatgaagctgaggaaagaggagagaccgagaccgaccagaacgaaagtcagagtccacaggagcctgaggaaggccccagtgaagatgacaaggcagaaggggaagaggaaatggacacaggagctgatgaccaagatggagatgctgctcagcatcctgaagaacactctgaggagcagcagcagtctgtggaggaaaaagacaaggaagccgatgaagaaggtggagagaatggccctgctgaccaaggtttccagccccaggaggaagaagaacgggaggactctgatacagaggagcaggtgccagaggctttggagaggaaggagcatgcctcctgtgggcagactggtgtggagaacatgcagaacacacaggccatggagctggctggggccgcacctgagaaggagcaggggaaagaggaacacggaagtggagctgcagatgcaaaccaggcagaaggccatgaatcgaatttcattgcccagttggcctcccagaagcacaccaggaaaaacacacagagttttaagaggaaacctgggcaggctgacaatgaacgttccatgggtgatcacaatgagcgtgtgcacaagaggctgaggactgtggatacggacagccatgccgagcaggggccagctcagcagccccaggcccaggtggaggatgcagatgcattcgagcacattaaacaaggcagtgacgcatacgatgcacagacctatgatgtggccagcaaagaacagcaacagtctgcaaaagactctggcaaagatcaggaagaggaggagatagaggacacccttatggacacagaggagcaggaggagttcaaagcagcagacgtggagcagctgaagccagaggaaatcaagtcgggcaccacagcacccttgggctttgatgagatggaagtggagatccaaactgttaaaacagaggaagaccaagaccccagaacagacaaagcccataaggagacagaaaatgagaaaccagaaagaagccgagagtctaccattcatacagctcatcaattcctcatggacacgatcttccagccctttttaaaagatgtcaatgagctaagacaggagctggagagacagctggaaatgtggcagccacgtgaatctggaaacccagaggaggagaaggttgcagctgagatgtggcagagttacctgatcttaacagcgcctctttcacaacggttatgtgaagagcttcgtctcatattagagcctacccaggcagccaagctgaaaggagactatcgaactgggaaacgactaaacatacggaaagtcattccatacattgctagtcaatttcggaaagacaagatttggcttcgaaggaccaagcccagtaaacgccagtatcagatttgtttggctatcgatgactcttctagtatggtagacaatcataccaagcagcttgcatttgaatctttggctgtgattggaaatgctctaaccctcctggaagtgggtcagattgcagtgtgtagttttggagaatctgtaaagctgttacacccatttcatgagcagttcagtgattactctgggtcccagattctacgtctctgcaaattccaacaaaagaaaaccaagattgctcagtttctagagtctgttgccaacatgtttgcagctgctcagcagctctcgcagaacatcagttcagaaactgcacaactcctcctggtagtctctgatgggcgaggccttttccttgagggcaaagaaagagtcctggcagcagttcaggctgcccggaatgcaaatatctttgtcatctttgttgtattggacaatcccagttcacgggattctatcttggacattaaagtaccgatatttaaaggacctggagagatgcctgaaatccgatcctacatggaagagttcccattcccatactatatcattcttcgagatgtaaacgcacttcctgagacactcagcgatgccctcagacagtggtttgagttggtgacagcctctgaccacccatagaacagaaagaagagtccaaagtgagacttaactgtggtcagaaggtcacattgcttacccaggtgctcccttttggacaactacaaaaaattttattgtaatatttttattttacaacgtgatcttacagcctacagaatgctctctggctcccggctttgcctgggctgaggtttttataccaaacctggaagcagcagcaggtgcctgaactcgtaactagagaagagttatccttcttccctgccttggaagccctggcctgggaggaggtcataccccaccgttggagcccagctgcctgttttcttttgcaggggatctgggcacctgtgccttgaggagatgctgccaggagcatgggactctgacagtcctttgtataaaggactaaagggagctgcccttttgaccctgttctaagctctgccttgccaagcccatagtgtgtgcccaaaagctgtcaagtggccaagacagctcgtttctggagagtatgagggtgtgttttcttattgtgaaaggaactaccttctcttagagggtaggaagaatgtggtgtgtgtgtgttctcataaagcaactggacattataggtgcccaggtcatctataaaaacgatccttgggctgtgtaaaaatgaagtggcttttcagtatcctctttcacacttgctgcttcgggagactatgcaatgatgggaaggtgattgcccctttatttcattcagtgccatggtccctgttgttgtagtaatttatttgtttagttcatttttttttttcttaacagtcaaggggaagagtgattcctcacactgctttcaagctggactgagccagtctcattctgggaaagaaacgctgtgtccagaactcagcagctccatctattttttccagtcgaaagaaactgatctttaggcagtttttacttggccagaaagcagtgctgaatacttgaaactgtgtgctctgttctacttaatgttctgtcagaatgttcttttgtaggcagtatgtcatgatgtaatcatctatctccttgtctgtttccaagttacactgtgaagtctgcgacccttttgaggtggtcatcaaagacacagattccttgtttaaccaagtgtcccaaagcatgtacctgaagttatatcattttttattctaaaaagctatgcagcttatattctgaaaactattaaaacatataccactgttgttgatgtaatttgtgactcttcttaatggaagatgacaggattgtaaaaggtatgctaggggactgatcttctctgctggatcagtcagtcagctgttactagttgatgctgtgctaacatgatcccctcctacttccatgttgctcttactacaaaggttatcatttgcatttatgtccatggtaggctgagctataatatgctggctttgcagcagaatgaaaaggatgagttggtgtagccttataaggaggcttataaaaataaattatcctccataaatg

SEQ ID NO: 2 - protein, 2047 aa, Homo sapiens
SGLYRYRSRNSRTALSEEEEEEREFRKQFPLHEKDFADILVQPTLEENKGTSDGQEEEAGTNPALLSQNSMQAVMLIHQQLCLNFARSLWYQQTLPPHEAKHYLSLFLSCYQTGASLVTHFYPLMGVELNDRLLGSQLLACTLSHNTLFGEAPSDLMVKPDGPYDFYQHPNVPEARQCQPVLQGFSEAVSHLLQDWPEHPALEQLLVVMDRIRSFPLSSPISKFLNGLEILLAKAQDWEENASRALSLRKHLDLISQMIIRWRKLELNCWSMSLDNTMKRHTEKSTKHWFSIYQMLEKHMQEQTEEQEDDKQMTLMLLVSTLQAFIEGSSLGEFHVRLQMLLVFHCHVLLMPQVEGKDSLCSVLWNLYHYYKQFFDRVQAKIVELRSPLEKELKEFVKISKWNDVSFWSIKQSVEKTHRTLFKFMKKFEAVLSEPCRSSLVESDKEEQPDFLPRPTDGAASELSSIQNLNRALRETLLAQPAAGQATIPEWCQGAAPSGLEGELLRRLPKLRKRMRKMCLTFMKESPLPRLVEGLDQFTGEVISSVSELQSLKVEPSAEKEKQRSEAKHILMQKQRALSDLFKHLAKIGLSYRKGLAWARSKNPQEMLHLHPLDLQSALSIVSSTQEADSRLLTEISSSWDGCQKYFYRSLARHARLNAALATPAKEMGMGNVERCRGFSAHLMKMLVRQRRSLTTLSEQWIILRNLLSCVQEIHSRLMGPQAYPVAFPPQDGVQQWTERLQHLAMQCQILLEQLSWLLQCCPSVGPAPGHGNVQVLGQPPGPCLEGPELSKGQLCGVVLDLIPSNLSYPSPIPGSQLPSGCRMRKQDHLWQQSTTRLTEMLKTIKTVKADVDKIRQQSCETLFHSWKDFEVCSSALSCLSQVSVHLQGLESLFILPGMEVEQRDSQMALVESLEYVRGEISKAMADFTTWKTHLLTSDSQGGNQMLDEGFVEDFSEQMEIAIRAILCAIQNLEERKNEKAEENTDQASPQEDYAGFERLQSGHLTKLLEDDFWADVSTLHVQKIISAISELLERLKSYGEDGTAAKHLFFSQSCSLLVRLVPVLSSYSDLVLFFLTMSLATHRSTAKLLSVLAQVFTELAQKGFCLPKEFMEDSAGEGATEFHDYEGGGIGEGEGMKDVSDQIGNEEQVEDTFQKGQEKDKEDPDSKSDIKGEDNAIEMSEDFDGKMHDGELEEQEEDDEKSDSEGGDLDKHMGDLNGEEADKLDERLWGDDDEEEDEEEEDNKTEETGPGMDEEDSELVAKDDNLDSGNSNKDKSQQDKKEEKEEAEADDGGQGEDKINEQIDERDYDENEVDPYHGNQEKVPEPEALDLPDDLNLDSEDKNGGEDTDNEEGEEENPLEIKEKPEEAGHEAEERGETETDQNESQSPQEPEEGPSEDDKAEGEEEMDTGADDQDGDAAQHPEEHSEEQQQSVEEKDKEADEEGGENGPADQGFQPQEEEEREDSDTEEQVPEALERKEHASCGQTGVENMQNTQAMELAGAAPEKEQGKEEHGSGAADANQAEGHESNFIAQLASQKHTRKNTQSFKRKPGQADNERSMGDHNERVHKRLRTVDTDSHAEQGPAQQPQAQVEDADAFEHIKQGSDAYDAQTYDVASKEQQQSAKDSGKDQEEEEIEDTLMDTEEQEEFKAADVEQLKPEEIKSGTTAPLGFDEMEVEIQTVKTEEDQDPRTDKAHKETENEKPERSRESTIHTAHQFLMDTIFQPFLKDVNELRQELERQLEMWQPRESGNPEEEKVAAEMWQSYLILTAPLSQRLCEELRLILEPTQAAKLKGDYRTGKRLNIRKVIPYIASQFRKDKIWLRRTKPSKRQYQICLAIDDSSSMVDNHTKQLAFESLAVIGNALTLLEVGQIAVCSFGESVKLLHPFHEQFSDYSGSQILRLCKFQQKKTKIAQFLESVANMFAAAQQLSQNISSETAQLLLVVSDGRGLFLEGKERVLAAVQAARNANIFVIFVVLDNPSSRDSILDIKVPIFKGPGEMPEIRSYMEEFPFPYYIILRDVNALPETLSDALRQWFELVTASDHP

SEQ ID NO: 3 - DNA, 1610 bp, Homo sapiens
tcctgggagacagggatttctccaggctgctggacatcaccccagcctccagcctgagctttgtcctggacaccacgggcagcatgggtgaggagatcaacgctgccaaaatccaggctcgccaccttgtggagcagcggagaggcagccccatggagcctgtccactatgtcctggtgccttttcatgacccagggttcggccctgtctttacaaccagtgaccctgacagcttctggcagcagcttaatgagatccatgccttggggggtggagacgagcctgagatgtgcctgtcagccctgcagctggccctgctgcacacacctccactctcagatatctttgtcttcacggatgcctcccccaaggatgcctttctcaccaaccaggtggaatccctgactcaggagcggcgctgccgggtaacattcctggtgactgaagatacatcaagggttcagggtcgagctcggcgtgagatcttgtcccctctgcgttttgagccatacaaagcagtggccctggcctcaggaggagaggtgatcttcaccaaagaccagcacattcgagacgtggcagccattgttggggagagcatggctgccctggtgactcttcccctggaccctcctgttgtggtgcctgggcagccacttgtgttcagcgtggatgggctgctccagaagatcacagtccggatccacggagacatcagcagcttctggatcaagaaccctgcaggggtctcccagggccaggagaagcgggggtcctctagtcacactcgccgctttgggcagttctggatggtgaccatggatgaccctccacagacaggaacctgggagatccaggtcacagctgaggacacccctggggtgagagtgcaagcccagacctccctggacttcctcttccactttgggatccccatggaggatggaccccaccctggcctctaccccctgactcagccagttgcaggtcttcagacccagctgctggtagaagtgacagggttgggttccagagccaatcctggggatcctcagccgcatttctcccacgtcatccttcgaggggtcccagagggtgccgaactaggccaggtgcccttggagcccgtgggacctccggagcgaggtctcctcgcagcctcgctgtcgcccacgctgctgtccacccctagacccttctccctggagctgattggccaggacgcagcggggcggcgcctgcacagggctgcccctcagcctagcactgtagtccctgtccttctggagcttagtggcccctcgggtttcttggccccgggcagcaaagtcccgctcagtctccgcatcgccagcttctcgggccctcaggatcttgaccttaggactttcgtcaaccccagcttctccctcacctccaacctctccagggctcacctggaactgaatgagtgcctggggcccgctgtggctggaggtcccagattcagcggccccggattccgtggtgatggtgactgtgactgcagggggacgagaagccaacccagtacccccgactcgtgctttctccggctcctggtatcgccccagccccgcaggaccggcaca

SEQ ID NO: 4 - protein, 536 aa, Homo sapiens
LGDRDFSRLLDITPASSLSFVLDTTGSMGEEINAAKIQARHLVEQRRGSPMEPVHYVLVPFHDPGFGPVFTTSDPDSFWQQLNEIHALGGGDEPEMCLSALQLALLHTPPLSDIFVFTDASPKDAFLTNQVESLTQERRCRVTFLVTEDTSRVQGRARREILSPLRFEPYKAVALASGGEVIFTKDQHIRDVAAIVGESMAALVTLPLDPPVVVPGQPLVFSVDGLLQKITVRIHGDISSFWIKNPAGVSQGQEKRGSSSHTRRFGQFWMVTMDDPPQTGTWEIQVTAEDTPGVRVQAQTSLDFLFHFGIPMEDGPHPGLYPLTQPVAGLQTQLLVEVTGLGSRANPGDPQPHFSHVILRGVPEGAELGQVPLEPVGPPERGLLAASLSPTLLSTPRPFSLELIGQDAAGRRLHRAAPQPSTVVPVLLELSGPSGFLAPGSKVPLSLRIASFSGPQDLDLRTFVNPSFSLTSNLSRAHLELNECLGPAVAGGPRFSGPGFRGDGDCDCRGTRSQPSTPDSCFLRLLVSPQPRRTGT

SEQ ID NO: 5 - DNA, 5688 bp, Homo sapiens
taggatacaacatagaacctattatgctctatcaggatatgacagcgcgtgatctgctacagcagagatacacccttccaaatggagacactgcctggcggtcctcaccccttgtgaatgctgctctggaaggcaagctggtcctgctggatggcattcaccgggtgaatgcgggcacgcttgctgtattgcaaaggttaatccatgatcgagagctaagcctctatgatggttctaggctgctgagagaagacaggtatatgcgtttaaaggaggagctgcaactgtctgatgaacagctacagaagagatccatttttcctatccatccctccttcagaatcattgccttggcagaaccccctgttattggaagcacagcacaccagtggctgggaccagaattcttaaccatgttctttttccattacatgaaaccacttgtgaaaagtgaagaaatccaagtgattaaggaaaaggtcccaaatgtacctcaggaagctctggataagttattatcatttacacacaaactcagagaaacacaggatccaacggcacaatcattagcggcatcactttctaccagacaactgttgcgaatttctcgtcggctgtcacagtatcctaatgaaaatcttcacagtgctgttactaaagcctgcctttccaggtttttacccagtcttgctaggtcagcattagaaaaaaatctggcagatgctacaatagaaataaatactgatgacaatttggagccagaactgaaggattacaaatgtgaagtaacatctggaactctgaggattggtgctgttagtgcaccgatctataatgcacatgagaaaatgaaagtgcctgatgttcttttctatgacaacattcagcatgtgatagtgatggaagatatgctgaaagactttctccttggagaacacttattattggttggcaaccagggtgtaggaaaaaacaagattgttgacagattccttcacctgctcaacagaccccgagaatatattcagctacacagggataccacagtacaaactcttacgcttcagccttcggttaaagacggacttattgtatatgaagactcacctttggttaaagcagtaaagttgggtcatattctggtagtagatgaggctgacaaagctccaacaaatgtcacgtgtattttaaaaactctagtagaaaatggagaaatgattctagcagatggaagacgcattgttgcaaattctgctaatgtgaatggaagagaaaatgttgtagtgattcatcctgattttaggatgattgttctggcaaatagacctggatttcctttcctaggcaatgatttcttcggtaccttaggtgatatttttagctgccatgcagttgataaccccaaaccccactcggagctcgagatgctcagacagtatggaccaaatgtgcctgagcccatccttcagaagcttgtggctgcctttggagagctgaggagtttggctgaccaagggattattaactatccttattctaccagagaagttgtcaacatagtcaaacatttacagaaatttccgactgaaggtctctccagtgtagttcgaaatgtgtttgactttgattcctacaacaatgacatgagggagatattgattaacactttacacaaatacgggatacctatcggagcaaagcctaccagtgtgcagctggcaaaggagttgactctgccagaacaaacgttcatgggctactggacaattggtcaggcaagaagtgggatgcaaaaactcttgtgtccagtggaaactcatcatatagacataaagggtccagcacttataaatatacaggagtatccaatagaaagacatgaagaaagatccctaaactttactgaagaatgtgcctcatggagaataccattggatgaaattaatataatctgtgacattgctacatcacatgaaaatgagcaaaatactctctatgtagttacatgcaatcccgcttccctgtactttatgaatatgactgggaaaagtggcttctttgtggacttttttgatatcttcccaagaacagccaatggcgtttggcacccttttgtgacagtggcaccgctgggaagtcctctcaaaggtcaagtggttctccatgagcagcagagtaatgttatcctgttgttagatactactggccgggcccttcatcgtctcatcctcccttccgagaagtttacatctaagaaacctttctggtggaacaaagaagaagctgaaacttataaaatgtgtaaagaattttcacacaaaaactggctggtgttctacaaagaaaaagggaacagcctgactgtgctggatgttctagaagggcgaactcacaccatctcacttcccatcaacctcaagacagttttccttgtagcagaggacaaatggcttctggtggagagcaaaacaaatcagaaatatcttttaactaagcctgcacacatcgaatctgagggtagtggggtttgccagttgtatgtgctgaaagaggagccgcccagcacagggtttggagttacacaagaaacagagttcagcatacctcataaaatttccagtgatcaactatcatctgaacatctaagttcagctgtggaacaaaagattgcctctcccaacagaattctctcagatgagaaaaattatgctacaatagttgttggttttccagatctcatgtcacccagtgaagtttattcttggaagagaccatcatctttgcataaacgaagtggcactgatacatcattctatagaggaaagaagaaaagggggactccaaaacaaagcaattgtgtgactcttttagatacgaatcaggtagtgaggattttacccccaggagaagtccctctaaaagatatctacccaaaagatgttactcctccacaaacatctggttatatagaagtcactgatcttcaatcaaagaaactccgatatatccctattcccagatcagaatctctctcgccgtataccacatggctgtcgaccatttcagacacagatgcactgctggctgagtgggacaaaagcggtgttgttactgttgatatgggaggtcacatcaggctttgggaaactggacttgaacgtctgcagcgatcactcatggaatggagaaacatgattggacaagatgacagaaatatgcagataacaatcaacagagacagtggtgaagatgtaagctcccccaaacacgggaaggaggacccagacaacatgcctcacgtgggcggcaacacttgggctggcggaacagggggaagagacacggcaggcctgggtggcaaaggaggcccttaccggctggatgcaggccatacggtgtaccaggtctctcaggctgagaaagatgcagttcccgaagaggtcaagagagctgctagagagatgggccagagagcattccagcagaggctaaaggagatccaaatgagtgaatacgatgctgcaacctatgaaaggttttcaggtgctgttcggcgacaggtgcactccctccgaatcatcctggataatttacaggctaaaggtaaagaaagacaatggctaagacatcaagctactggagaattagatgatgccaagatcattgatgggctgactggagaaaaagccatctacaaacgtcggggtgagctggagccacaacttggcagcccacaacagaaacccaagcgtctgcgcctggtggtagatgtgtctggtagcatgtaccgtttcaacaggatggatggccggcttgagcgcacaatggaggctgtgtgtatggtcatggaagccttcgagaactatgaggagaagttccagtatgacatcgttggacactctggagatggctacaacattggtctggttccaatgaacaaaatccccaaggacaataagcaaagactagaaattctgaagacaatgcatgcccactctcagttctgcatgagtggggaccacacgttagaagggacagaacatgccatcaaggaaattgtcaaagaagaagctgatgagtactttgtcatagtcttgagtgatgcaaatctgtcacgatatggaatacatcctgctaagtttgctcaaatcctcacaagagaccctcaagtaaatgcttttgccatttttattggctctttaggtgatcaagcaaccaggcttcagagaactttaccagctggtcggtctttcgttgccatggataccaaggatatccctcagattttacaacagatcttcacctccaccatgttgtcgagtgtctaagaagtgcccttcatcaccctgatgacaccaggatttgaaataagacaggaataaagagtattctgaaaaaaagaagatatggatgaagtgaacccatgcagtgactggatgattccggcattcctgggtcttcctacacttgctccgtaatgagaattcagagaagcagccagaaggagacttaaacatggaaagatactccactgatgagtttagaagtgattagggcaagctagttgacctgcactttatcaaaggttggggttaaaggaaggtggttttgagaactatgtgtttggtctatttccaaaaacctgagggggagaaaatactttgcttttgccttaacacatcatctggtcacgttagaaaagtgaccccatcaaactgagcctttgatgtcacattctgacacaagatgcaagtctgtgcaaaacccaccaaaatgctcctatccaacaattatttttattttctccctgctctgtattgaccagaaaacaatgattttataatatttaacccaccaaaaagtccctccaacagcaatatcttatgaaaggctggctgttctcaataatttcattttcttggccaaagccaaaaggaatcaaaaaatccttggagtgcttgcctttccacgtgacttttaaaaggctcttaaagagcaaatgtcctcttctccttgctgctagagtgggaggagaaagatgtcttgccttaaaagtttactgtttcttctgttcttctagcatctctcaaaaaattcacagtactccattttggggtccaaactgtaatgctcaaaataataaatgcttacacgaaaattatttattgagaatattcatataaaaattacctaaagcaaagtaaaaaaagtaaaatcaaggtggtatatttgaagtgaatggtgattggaaatttttagctgtaacaaaaagaaagaaaacaactttttttaaagcctcattctcttttctttcaaaatgtaccttattcccacacactcttgggctgacctttattttatcaataagctcaatattactttgtttaaaataagatgcttcagcaaaagtcattctctctttaaccatataatttaaaaactcctcttcacgattgatagcaaaatcagaaacgttagggcaccagtgagttgaaaaaactggtcttaagttggaaaaactattattaataatattatcctatccatccatatctattgaaattgtacaggtccataatttcattttaattaattataggaaagaagaaaagataatacccatttgttctatcacccctctccctatcattaactatcaaataaataaataaaagcaatctgatttcc

SEQ ID NO: 6 - protein, 1441 aa, Homo sapiens
GYNIEPIMLYQDMTARDLLQQRYTLPNGDTAWRSSPLVNAALEGKLVLLDGIHRVNAGTLAVLQRLIHDRELSLYDGSRLLREDRYMRLKEELQLSDEQLQKRSIFPIHPSFRIIALAEPPVIGSTAHQWLGPEFLTMFFFHYMKPLVKSEEIQVIKEKVPNVPQEALDKLLSFTHKLRETQDPTAQSLAASLSTRQLLRISRRLSQYPNENLHSAVTKACLSRFLPSLARSALEKNLADATIEINTDDNLEPELKDYKCEVTSGTLRIGAVSAPIYNAHEKMKVPDVLFYDNIQHVIVMEDMLKDFLLGEHLLLVGNQGVGKNKIVDRFLHLLNRPREYIQLHRDTTVQTLTLQPSVKDGLIVYEDSPLVKAVKLGHILVVDEADKAPTNVTCILKTLVENGEMILADGRRIVANSANVNGRENVVVIHPDFRMIVLANRPGFPFLGNDFFGTLGDIFSCHAVDNPKPHSELEMLRQYGPNVPEPILQKLVAAFGELRSLADQGIINYPYSTREVVNIVKHLQKFPTEGLSSVVRNVFDFDSYNNDMREILINTLHKYGIPIGAKPTSVQLAKELTLPEQTFMGYWTIGQARSGMQKLLCPVETHHIDIKGPALINIQEYPIERHEERSLNFTEECASWRIPLDEINIICDIATSHENEQNTLYVVTCNPASLYFMNMTGKSGFFVDFFDIFPRTANGVWHPFVTVAPLGSPLKGQVVLHEQQSNVILLLDTTGRALHRLILPSEKFTSKKPFWWNKEEAETYKMCKEFSHKNWLVFYKEKGNSLTVLDVLEGRTHTISLPINLKTVFLVAEDKWLLVESKTNQKYLLTKPAHIESEGSGVCQLYVLKEEPPSTGFGVTQETEFSIPHKISSDQLSSEHLSSAVEQKIASPNRILSDEKNYATIVVGFPDLMSPSEVYSWKRPSSLHKRSGTDTSFYRGKKKRGTPKQSNCVTLLDTNQVVRILPPGEVPLKDIYPKDVTPPQTSGYIEVTDLQSKKLRYIPIPRSESLSPYTTWLSTISDTDALLAEWDKSGVVTVDMGGHIRLWETGLERLQRSLMEWRNMIGQDDRNMQITINRDSGEDVSSPKHGKEDPDNMPHVGGNTWAGGTGGRDTAGLGGKGGPYRLDAGHTVYQVSQAEKDAVPEEVKRAAREMGQRAFQQRLKEIQMSEYDAATYERFSGAVRRQVHSLRIILDNLQAKGKERQWLRHQATGELDDAKIIDGLTGEKAIYKRRGELEPQLGSPQQKPKRLRLVVDVSGSMYRFNRMDGRLERTMEAVCMVMEAFENYEEKFQYDIVGHSGDGYNIGLVPMNKIPKDNKQRLEILKTMHAHSQFCMSGDHTLEGTEHAIKEIVKEEADEYFVIVLSDANLSRYGIHPAKFAQILTRDPQVNAFAIFIGSLGDQATRLQRTLPAGRSFVAMDTKDIPQILQQIFTSTMLSSV

SEQ ID NO: 7 - DNA, 2559 bp, Homo sapiens
atgctccccacggaggtcccccaatcccacccgggcccctcagcgttgcttctgctgcagctgttgctgccccccacatctgccttcttccccaacatctggagcctgctggctgcccctggctccatcacccaccaagacctaactgaggaggcagcgctcaacgtcaccctgcagctcttcctggagcagccacccccaggccgcccccctcttcgtcttgaggacttcctgggtcgaacactccttgctgatgacctctttgccgcctactttggacctggttcttctcggcggttccgagcagccttaggtgaggtgtctcgtgccaatgcagcccaggacttcctgccaacttccaggaatgaccccgacctgcactttgatgctgagcgactgggtcagggacgcgcgcgcctggtaggggctctgcgggagaccgtggtggcagccagggcccttgaccacaccctggctcgccagcgcctcggggctgcacttcatgccctgcaggatttctacagtcatagcaactgggtggagctgggcgagcagcagccacaccctcacctcctctggccaaggcaggagctccagaacctggcacaagtggccgatcctacctgctccgattgcgaggagttgagctgccccaggaattggctgggcttcacactcctcacctctggctactttggaactcatcccccgaaacctccagggaaatgtagccacgggggccattttgaccggagcagctcccagccaccgaggggaggcatcaacaaggacagcacatccccaggcttctcccctcaccacatgctgcacctccaggctgcaaaactggcccttctagcctccatccaggccttcagccttctgcgaagccgcctgggagacagggatttctccaggctgctggacatcaccccagcctccagcctgagctttgtcctggacaccacgggcagcatgggtgaggagatcaacgctgccaaaatccaggctcgccaccttgtggagcagcggagaggcagccccatggagcctgtccactatgtcctggtgccttttcatgacccagggttcggccctgtctttacaaccagtgaccctgacagcttctggcaacagcttaatgagatccatgccttggggggtggagacgagcctgagatgtgcctgtcagccctgcagctggccctgctgcacacacctccactctcagatatctttgtcttcacggatgcctcccccaaggatgcctttctcaccaaccaggtggaatccctgactcaggagcggcgctgccgggtaacattcctggtgactgaagatacatcaagggttcagggtcgagctcggcgtgagatcttgtcccctctgcgttttgagccatacaaagcagtggccctggcctcaggaggagaggtgatcttcaccaaagaccagcacattcgagacgtggcagccattgttggggagagcatggctgccctggtgactcttcccctggaccctcctgttgtggtgcctgggcagccacttgtgttcagcgtggatgggctgctccagaagatcacagtccggatccacggagacatcagcagcttctggatcaagaaccctgcaggggtctcccagggccaggaggaaggcgggggtcctctaggtcacactcgccgctttgggcagttctggatggtgaccatggatgaccctccacagacaggaacctgggagatccaggtcacagctgaggacacccctggggtgagagtgcaagcccagacctccctggacttcctcttccactttgggatccccatggaggatggaccccaccctggcctctaccccctgactcagccagttgcaggtcttcagacccagctgctggtagaagtgacagggttgggttccagagccaatcctggggatcctcagccgcatttctcccacgtcatccttcgaggggtcccagagggtgccgaactaggccaggtgcccttggagcccgtgggacctccggagcgaggtctcctcgcagcctcgctgtcgcccacgctgctgtccacccctagacccttctccctggagctgattggccaggacgcagcggggcggcgcctgcacagggctgcccctcagcctagcactgtagtccctgtccttctggagcttagtggcccctcgggtttcttggccccgggcagcaaagtcccgctcagtctccgcatcgccagcttctcgggccctcaggatcttgaccttaggactttcgtcaaccccagcttctccctcacctccaacctctccagggctcacctggaactgaatgagtcggcctggggccgcctgtggctggaggtcccagattcagcggccccggattccgtggtgatggtgactgtgactgcagggggacgagaagccaacccagtacccccgactcatgctttcctccggctcctggtatcggccccagccccgcaggtgaggaactactactttccatcacagggaccggcacaccacccctaccggctcatctga

SEQ ID NO: 8 - protein, 852 aa, Homo sapiens
MLPTEVPQSHPGPSALLLLQLLLPPTSAFFPNIWSLLAAPGSITHQDLTEEAALNVTLQLFLEQPPPGRPPLRLEDFLGRTLLADDLFAAYFGPGSSRRFRAALGEVSRANAAQDFLPTSRNDPDLHFDAERLGQGRARLVGALRETVVAARALDHTLARQRLGAALHALQDFYSHSNWVELGEQQPHPHLLWPRQELQNLAQVADPTCSDCEELSCPRNWLGFTLLTSGYFGTHPPKPPGKCSHGGHFDRSSSQPPRGGINKDSTSPGFSPHHMLHLQAAKLALLASIQAFSLLRSRLGDRDFSRLLDITPASSLSFVLDTTGSMGEEINAAKIQARHLVEQRRGSPMEPVHYVLVPFHDPGFGPVFTTSDPDSFWQQLNEIHALGGGDEPEMCLSALQLALLHTPPLSDIFVFTDASPKDAFLTNQVESLTQERRCRVTFLVTEDTSRVQGRARREILSPLRFEPYKAVALASGGEVIFTKDQHIRDVAAIVGESMAALVTLPLDPPVVVPGQPLVFSVDGLLQKITVRIHGDISSFWIKNPAGVSQGQEEGGGPLGHTRRFGQFWMVTMDDPPQTGTWEIQVTAEDTPGVRVQAQTSLDFLFHFGIPMEDGPHPGLYPLTQPVAGLQTQLLVEVTGLGSRANPGDPQPHFSHVILRGVPEGAELGQVPLEPVGPPERGLLAASLSPTLLSTPRPFSLELIGQDAAGRRLHRAAPQPSTVVPVLLELSGPSGFLAPGSKVPLSLRIASFSGPQDLDLRTFVNPSFSLTSNLSRAHLELNESAWGRLWLEVPDSAAPDSVVMVTVTAGGREANPVPPTHAFLRLLVSAPAPQVRNYYFPSQGPAHHPYRLI

SEQ ID NO: 9 - DNA, 2019 bp, Mycobacterium tuberculosis
atggctaagtctgatggtgacgacccgctgcgcccggcttcgccgcgcttgcgatcgtcacgacggcactcgctacgctactcggcgtacaccggcgggcccgacccgctggccccgccggtggatctgcgggatgcgctggaacagattggccaagacgtcatggcgggcgcctcgccgcgccgggcgctgtccgagctgctgcggcggggcaccaggaacctgaccggcgccgaccggctggcggccgaggtgaaccgccgccgacgggagttgttgcgccgcaacaacttagatggcaccttgcaggagatcaagaagctgctcgacgaggccgtgctggccgaacgcaaggagctggcccgcgcgctagacgacgacgcccgcttcgccgagctgcagctggacgcgcttccggcctcgccggccaaggcagtacaggagctggccgaataccgctggcgcagcgggcaggcccgcgaaaagtatgagcagatcaaggatttgctcggccgtgagctgctcgaccaacgctttgccggcatgaagcaggcgcttgccggtgccaccgacgacgatcgccggcgggtcaccgagatgctcgacgacctcaacgacctgttggataagcacgcccgcggtgaagatacgcagcgggacttcgacgagttcatgaccaagcacggcgagttcttcccggagaacccgcgcaacgtcgaggagctgctggactcgctggccaagcgagccgccgccgcgcagcggttccgcaacagcctgagccaggaacagcgggacgagctggacgcgttggcgcagcaggcatttggctctccggcgttgatgcgggcgctggaccgtttggatgcgcatctgcaggccgcccgtcccggcgaagactggaccggctcgcagcagttctccggtgataatccgttcggcatgggggaaggcacccaggcgctggccgacattgccgagctggagcagctggccgagcagctgtcgcagagctatccgggcgccagcatggacgatgtcgacctggacgcgctggcccgtcagctcggcgaccaggccgccgtcgacgcccggacgctggctgaattggaacgcgcgctggtcaatcagggcttcctggaccgcggttccgacggccagtggcggctctcgccgaaggccatgcgccgcctcggcgaaacggcgttacgcgatgtggcgcaacaactttccgggcgccacggcgagcgtgatcaccggcgtgccggcgccgcgggcgagctgaccggtgcgacgcggccctggcagttcggcgacaccgagccgtggcacgtcgcccgcacgctgaccaatgccgtgctgcgccaagccgcggccgtgcatgaccgcatccggatcaccgtcgaggatgtcgaggtcgccgagaccgaaacgcgcacccaggccgctgttgcgttgttggtggacacctcgttttcgatggtgatggagaatcgctggttgccgatgaagcgcacggcgctggcgctgcaccacctggtgtgcacccggttccgctcggatgccttgcagatcatcgcgtttgggcgctacgcccgcacggtgacggcggccgagctgacggggttggcgggtgtctacgagcagggcaccaacctgcaccatgcgctcgcgctggccggccggcacctgcgccggcacgcaggcgcccagcccgtggtgctggtggtgaccgacggcgagccgaccgcccacctggaggacttcgacggcgacggtacgtcggtgttctttgattacccgccccatccgcgcaccatcgcccacaccgtgcgcgggtttgacgacatggcgcggctgggtgcgcaggtgacgatcttccggttgggcagtgaccccggtctggctcggttcattgaccaggttgcgcgacgggtgcagggccgcgtggtggtgcccgatctcgacgggctgggcgcggcggtggtgggcgactacctgcgcttccggcggcgctag

SEQ ID NO: 10 - protein, 672 aa, Mycobacterium tuberculosis
MAKSDGDDPLRPASPRLRSSRRHSLRYSAYTGGPDPLAPPVDLRDALEQIGQDVMAGASPRRALSELLRRGTRNLTGADRLAAEVNRRRRELLRRNNLDGTLQEIKKLLDEAVLAERKELARALDDDARFAELQLDALPASPAKAVQELAEYRWRSGQAREKYEQIKDLLGRELLDQRFAGMKQALAGATDDDRRRVTEMLDDLNDLLDKHARGEDTQRDFDEFMTKHGEFFPENPRNVEELLDSLAKRAAAAQRFRNSLSQEQRDELDALAQQAFGSPALMRALDRLDAHLQAARPGEDWTGSQQFSGDNPFGMGEGTQALADIAELEQLAEQLSQSYPGASMDDVDLDALARQLGDQAAVDARTLAELERALVNQGFLDRGSDGQWRLSPKAMRRLGETALRDVAQQLSGRHGERDHRRAGAAGELTGATRPWQFGDTEPWHVARTLTNAVLRQAAAVHDRIRITVEDVEVAETETRTQAAVALLVDTSFSMVMENRWLPMKRTALALHHLVCTRFRSDALQIIAFGRYARTVTAAELTGLAGVYEQGTNLHHALALAGRHLRRHAGAQPVVLVVTDGEPTAHLEDFDGDGTSVFFDYPPHPRTIAHTVRGFDDMARLGAQVTIFRLGSDPGLARFIDQVARRVQGRVVVPDLDGLGAAVVGDYLRFRRR

SEQ ID NO: 11 - DNA, 1212 bp, Mycobacterium tuberculosis
atggccacccctgcactgttgccgggcgtcgacctcgcggcgttcgcggcagcgctggcagcgcgccttcgcgacgccgggataccggtgtccgccagcggtcaagcgagtttggtgcaggcgttgcagcagttggtgccgcgtacgccggcggcgctgtattggggcgcgcggttgaccctggtcagccgtgtagacgaactggccacgttcgatgcggtattcgcttcgctgttcggggtatttggcagcgccgaacccgacggtgccaaccgcccaccaccgcccattgcaggcccgcgcacaccggtggccggcgtcgggcaccgcgccaagcggcgatcttgtgccgcccaagcccagaatctgccctgggatactcgctcgctgacgatggccagcgccggtcagggcggacccagccgcacactgcccgatgtcctgcccagccgcattgtcgcccgggccgacgagccattcgaccagttcgatcccgacgatctgcgtctgctcggcgcctggctggaggccacgatggcgcgctggccgcggcggcgcagcatgcgattcgagtccagcccgcacggcaagcgcatcgacctgcgggcgacgatgaacgcgtcgcggtcgactggctgggagtcggtgctgttggcacggatccggccccgccgacgccccaggcgggtgctcctgctctgcgatgtgagccgctcgatgcagccctacgccgccatctatctgcgtctgatgcgggcggcggtgctgcgccgggcagggggccacccggaggttttcgcgttttcgacgtcgctgactcgacttacctcggtgctgtctcatcgctcggccgagatggcgctacatcgggccaacgctagggtgaccgaccgctacggcggtacgttcatcggccgtagtgtcgccgccctgctggccccgccgcatggcaacgcgttacgcggcgcggtggtgatcatcgcctccgacggctgggacagcgatccgcccgacgtgttggtgcacgcactgaccagggtgcgtcgccgcgccgagttgctggtctggctgaaccctcgcgcggcacatccggagttccagccgcgtgccggttcgatggcggcggcgctgccctattgcgacctgttcctgccggcgcactcgctggccggcctgcaccagttgctgctggcgctggccggtgcccgctag

SEQ ID NO: 12 - protein, 403 aa, Mycobacterium tuberculosis
MATPALLPGVDLAAFAAALAARLRDAGIPVSASGQASLVQALQQLVPRTPAALYWGARLTLVSRVDELATFDAVFASLFGVFGSAEPDGANRPPPPIAGPRTPVAGVGHRAKRRSCAAQAQNLPWDTRSLTMASAGQGGPSRTLPDVLPSRIVARADEPFDQFDPDDLRLLGAWLEATMARWPRRRSMRFESSPHGKRIDLRATMNASRSTGWESVLLARIRPRRRPRRVLLLCDVSRSMQPYAAIYLRLMRAAVLRRAGGHPEVFAFSTSLTRLTSVLSHRSAEMALHRANARVTDRYGGTFIGRSVAALLAPPHGNALRGAVVIIASDGWDSDPPDVLVHALTRVRRRAELLVWLNPRAAHPEFQPRAGSMAAALPYCDLFLPAHSLAGLHQLLLALAGAR

SEQ ID NO: 13 - protein, 200 aa, Unknown Organism
  Description of Unknown Organism VWA domain peptide
DIVFLLDGSGSIGSQNFERVKDFVERVVERLDVGPRDKKEEDAVRVGLVQYSDNVRTEIKFKLNDYQNKDEVLQALQKIRYEDYYGGGGTNTGAALQYVVRNLFTEASGSRIEPVAEEGAPKVLVVLTDGRSQDDPSPTIDIRDVLNELKKEAGVEVFAIGVGNADNNNLEELREIASKPDDHVFKVSDFEALDTLQELL

SEQ ID NO: 14 - protein, 15 aa, Unknown Organism
  Description of Unknown Organism Q12019 Pfam-B 16328 peptide
DDYEADFRKLFPDYE

SEQ ID NO: 15 - protein, 13 aa, Unknown Organism
  Description of Unknown Organism Q9YTL7 Pfam-B 1 peptide
EENKKKGDGEEDE

SEQ ID NO: 16 - protein, 240 aa, Unknown Organism
  Description of Unknown Organism Q12019 Pfam-B 16328 peptide
FSSELKSGAIITTILSEDLKNTRIEELKSGSLSAVINTLDAETQSFKNTEVFGNIDFYHDFSIPEFQKAGDIIETVLKSVLKLLKQWPEHATLKELYRVSQEFLNYPIKTPLARQLQKIEQIYTYLAEWEKYASSEVSLNNTVKLITDLIVSWRKLELRTWKGLFNSEDAKTRKSIGKWWFYLYESIVISNFVSEKKETAPNATLLVSSLNLFFSKSTLGEFNARLDLVKAFYKHIQLIG

SEQ ID NO: 17 - protein, 184 aa, Homo sapiens
DSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREK

SEQ ID NO: 18 - protein, 201 aa, Homo sapiens
SCPSLIDVVVVCDESNSIYPWDAVKNFLEKFVQGLDIGPTKTQVGLIQYANNPRVVFNLNTYKTKEEMIVATSQTSQYGGDLTNTFGAIQYARKYAYSAASGGRRSATKVMVVVTDGESHDGSMLKAVIDQCNHDNILRFGIAVLGYLNRNALDTKNLIKEIKAIASIPTERYFFNVSDEAALLEKAGTLGEQIFSIEGGT

SEQ ID NO: 19 - protein, 201 aa, Homo sapiens
RSSCPSLIDVVVVCDESNSIYPWDAVKNFLEKFVQGLDIGPTKTQVGLIQYANNPRVVFNLNTYKTKEEMIVATSQTSQYGGDLTNTFGAIQYARKYAYSAASGGRRSATKVMVVVTDGESHDGSMLKAVIDQCNHDNILRFGIAVLGYLNRNALDTKNLIKEIKAIASIPTERYFFNVSDEAALLEKAGTLGEQIFSIEG

SEQ ID NO: 20 - protein, 183 aa, Homo sapiens
GNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIE

SEQ ID NO: 21 - protein, 4910 aa, Saccharomyces cerevisiae
MSQDRILLDLDVVNQRLILFNSAFPSDAIEAPFHFSNKESTSENLDNLAGTILHSRSITGHVFLYKHIFLEIVARWIKDSKKKDYVLVIEKLASIITIFPVAMPLIEDYLDKENDHFITILQNPSTQKDSDMFKILLAYYRLLYHNKEVFARFIQPDILYQLVDLLTKEQENQVVIFLALKVLSLYLDMGEKTLNDMLDTYIKSRDSLLGHFEGDSGIDYSFLELNEAKRCANFSKLPSVPECFTIEKKSSYFIIEPQDLSTKVASICGVIVPKVHTIHDKVFYPLTFVPTHKTVSSLRQLGRKIQNSTPIMLIGKAGSGKTFLINELSKYMGCHDSIVKIHLGEQTDAKLLIGTYTSGDKPGTFEWRAGVLATAVKEGRWVLIEDIDKAPTDVLSILLSLLEKRELTIPSRGETVKAANGFQLISTVRINEDHQKDSSNKIYNLNMIGMRIWNVIELEEPSEEDLTHILAQKFPILTNLIPKLIDSYKNVKSIYMNTKFISLNKGAHTRVVSVRDLIKLCERLDILFKNNGINKPDQLIQSSVYDSIFSEAADCFAGAIGEFKALEPIIQAIGESLDIASSRISLFLTQHVPTLENLDDSIKIGRAVLLKEKLNIQKKSMNSTLFAFTNHSLRLMEQISVCIQMTEPVLLVGETGTGKTTVVQQLAKMLAKKLTVINVSQQTETGDLLGGYKPVNSKTVAVPIQENFETLFNATFSLKKNEKFHKMLHRCFNKNQWKNVVKLWNEAYKMAQSILKITNTENENENAKKKKRRLNTHEKKLLLDKWADFNDSVKKFEAQSSSIENSFVFNFVEGSLVKTIRAGEWLLLDEVNLATADTLESISDLLTEPDSRSILLSEKGDAEPIKAHPDFRIFACMNPATDVGKRDLPMGIRSRFTEIYVHSPERDITDLLSIIDKYIGKYSVSDEWVGNDIAELYLEAKKLSDNNTIVDGSNQKPHFSIRTLTRTLLYVTDIIHIYGLRRSLYDGFCMSFLTLLDQKSEAILKPVIEKFTLGRLKNVKSIMSQTPPSPGPDYVQFKHYWMKKGPNTIQEQAHYIITPFVEKNMMNLVRATSGKRFPVLIQGPTSSGKTSMIKYLADITGHKFVRINNHEHTDLQEYLGTYVTDDTGKLSFKEGVLVEALRKGYWIVLDELNLAPTDVLEALNRLLDDNRELFIPETQEVVHPHPDFLLFATQNPPGIYGGRKILSRAFRNRFLELHFDDIPQDELEIILRERCQIAPSYAKKIVEVYRQLSIERSASRLFEQKNSFATLRDLFRWALRDAVGYEQLAASGYMLLAERCRTPQEKVTVKKTLEKVMKVKLDMDQYYASLEDKSLEAIGSVTWTKGMRRLSVLVSSCLKNKEPVLLVGETGCGKTTICQLLAQFMGRELITLNAHQNTETGDILGAQRPVRNRSEIQYKLIKSLKTALNIANDQDVDLKELLQLYSKSDNKNIAEDVQLEIQKLRDSLNVLFEWSDGPLIQAMRTGNFFLLDEISLADDSVLERLNSVLEPERSLLLAEQGSSDSLVTASENFQFFATMNPGGDYGKKELSPALRNRFTEIWVPSMEDFNDVNMIVSSRLLEDLKDLANPIVKFSEWFGKKLGGGNATSGVISLRDILAWVEFINKVFPKIQNKSTALIQGASMVFIDALGTNNTAYLAENENDLKSLRTECIIQLLKLCGDDLELQQIETNEIIVTQDELQVGMFKIPRFPDAQSSSFNLTAPTTASNLVRVVRAMQVHKPILLEGSPGVGKTSLITALANITGNKLTRINLSEQTDLVDLFGADAPGERSGEFLWHDAPFLRAMKKGEWVLLDEMNLASQSVLEGLNACLDHRGEAYIPELDISFSCHPNFLVFAAQNPQYQGGGRKGLPKSFVNRFSVVFIDMLTSDDLLLIAKHLYPSIEPDIIAKMIKLMSTLEDQVCKRKLWGNSGSPWEFNLRDTLRWLKLLNQYSICEDVDVFDFVDIIVKQRFRTISDKNKAQLLIEDIFGKFSTKENFFKLTEDYVQINNEVALRNPHYRYPITQNLFPLECNVAVYESVLKAINNNWPLVLVGPSNSGKTETIRFLASILGPRVDVFSMNSDIDSMDILGGYEQVDLTRQISYITEELTNIVREIISMNMKLSPNATAIMEGLNLLKYLLNNIVTPEKFQDFRNRFNRFFSHLEGHPLLKTMSMNIEKMTEIITKEASVKFEWFDGMLVKAVEKGHWLILDNANLCSPSVLDRLNSLLEIDGSLLINECSQEDGQPRVLKPHPNFRLFLTMDPKYGELSRAMRNRGVEIYIDELHSRSTAFDRLTLGFELGENIDFVSIDDGIKKIKLNEPDMSIPLKHYVPSYLSRPCIFAQVHDILLLSDEEPIEESLAAVIPISHLGEVGKWANNVLNCTEYSEKKIAERLYVFITFLTDMGVLEKINNLYKPANLKFQKALGLHDKQLTEETVSLTLNEYVLPTVSKYSDKIKSPESLYLLSSLRLLLNSLNALKLINEKSTHGKIDELTYIELSAAAFNGRHLKNIPRIPIFCILYNILTVMSENLKTESLFCGSNQYQYYWDLLVIVIAALETAVTKDEARLRVYKELIDSWIASVKSKSDIEITPFLNINLEFTDVLQLSRGHSITLLWDIFRKNYPTTSNSWLAFEKLINLSEKFDKVRLLQFSESYNSIKDLMDVFRLLNDDVLNNKLSEFNLLLSKLEDGINELELISNKFLNKRKHYFADEFDNLIRYTFSVDTAELIKELAPASSLATQKLTKLITNKYNYPPIFDVLWTEKNAKLTSFTSTIFSSQFLEDVVRKSNNLKSFSGNQIKQSISDAELLLSSTIKCSPNLLKSQMEYYKNMLLSWLRKVIDIHVGGDCLKLTLKELCSLIEEKTASETRVTFAEYIFPALDLAESSKSLEELGEAWITFGTGLLLLFVPDSPYDPAIHDYVLYDLFLKTKTFSQNLMKSWRNVRKVISGDEEIFTEKLINTISDDDAPQSPRVYRTGMSIDSLFDEWMAFLSSTMSSRQIKELVSSYKCNSDQSDRRLEMLQQNSAHFLNRLESGYSKFADLNDILAGYIYSINFGFDLLKLQKSKDRASFQISPLWSMDPINISCAENVLSAYHELSRFFKKGDMEDTSIEKVLMYFLTLFKFHKRDTNLLEIFEAALYTLYSRWSVRRFRQEQEENEKSNMFKFNDNSDDYEADFRKLFPDYEDTALVTNEKDISSPENLDDIYFKLADTYISVFDKDHDANFSSELKSGAIITTILSEDLKNTRIEELKSGSLSAVINTLDAETQSFKNTEVFGNIDFYHDFSIPEFQKAGDIIETVLKSVLKLLKQWPEHATLKELYRVSQEFLNYPIKTPLARQLQKIEQIYTYLAEWEKYASSEVSLNNTVKLITDLIVSWRKLELRTWKGLFNSEDAKTRKSIGKWWFYLYESIVISNFVSEKKETAPNATLLVSSLNLFFSKSTLGEFNARLDLVKAFYKHIQLIGLRSSKIAGLLHNTIKFYYQFKPLIDERITNGKKSLEKEIDDIILLASWKDVNVDALKQSSRKSHNNLYKIVRKYRDLLNGDAKTIIEAGLLYSNENKLKLPTLKQHFYEDPNLEASKNLVKEISTWSMRAAPLRNIDTVASNMDSYLEKISSQEFPNFADLASDFYAEAERLRKETPNVYTKENKKRLAYLKTQKSKLLGDALKELRRIGLKVNFREDIQKVQSSTTTILANIAPFNNEYLNSSDAFFFKILDLLPKLRSAASNPSDDIPVAAIERGMALAQSLMFSLITVRHPLSEFTNDYCKINGMMLDLEHFTCLKGDIVHSSLKANVDNVRLFEKWLPSLLDYAAQTLSVISKYSATSEQQKILLDAKSTLSSFFVHFNSSRIFDSSFIESYSRFELFINELLKKLENAKETGNAFVFDIIIEWIKANKGGPIKKEQKRGPSVEDVEQAFRRTFTSIILSFQKVIGDGIESISETDDNWLSASFKKVMVNVKLLRSSVVSKNIETALSLLKDFDFTTTESIYVKSVISFTLPVITRYYNAMTVVLERSRIYYTNTSRGMYILSTILHSLAKNGFCSPQPPSEEVDDKNLQEGTGLGDGEGAQNNNKDVEQDEDLTEDAQNENKEQQDKDERDDENEDDAVEMEGDMAGELEDLSNGEENDDEDTDSEEEELDEEIDDLNEDDPNAIDDKMWDDKASDNSKEKDTDQNLDGKNQEEDVQAAENDEQQRDNKEGGDEDPNAPEDGDEEIENDENAEEENDVGEQEDEVKDEEGEDLEANVPEIETLDLPEDMNLDSEHEESDEDVDMSDGMPDDLNKEEVGNEDEEVKQESGIESDNENDEPGPEEDAGETETALDEEEGAEEDVDMTNDEGKEDEENGPEEQAMSDEEELKQDAAMEENKEKGGEQNTEGLDGVEEKADTEDIDQEAAVQQDSGSKGAGADATDTQEQDDVGGSGTTQNTYEEDQEDVTKNNEESREEATAALKQLGDSMKEYHRRRQDIKEAQTNGEEDENLEKNNERPDEFEHVEGANTETDTQALGSATQDQLQTIDEDMAIDDDREEQEVDQKELVEDADDEKMDIDEEEMLSDIDAHDANNDVDSKKSGFIGKRKSEEDFENELSNEHFSADQEDDSEIQSLIENIEDNPPDASASLTPERSLEESRELWHKSEISTADLVSRLGEQLRLILEPTLATKLKGDYKTGKRLNMKRIIPYIASQFRKDKIWLRRTKPSKRQYQIMIALDDSKSMSESKCVKLAFDSLCLVSKTLTQLEAGGLSIVKFGENIKEVHSFDQQFSNESGARAFQWFGFQETKTDVKKLVAESTKIFERARAMVHNDQWQLEIVISDGICEDHETIQKLVRRARENKIMLVFVIIDGITSNESILDMSQVNYIPDQYGNPQLKITKYLDTFPFEFYVVVHDISELPEMLSLILRQYFTDLASS

SEQ ID NO: 22 - protein, 446 aa, Drosophila melanogaster
MDQTEAEEYQHVKEPKNSDKTTLDNATEEQSKKIQHQEDEPPNEEEIEAENVDELMEAEEPAVDPEDDAELEQLGAEKTEQKSDKPSKTEKSKEQLETPEGMEIEGEVVLTMTVPRSSETTAHSNSEILLDKSSHAEDLSSAEQIELRQQYQKQLTTFRVAQPEHEDYETWQGISNRMTQNARELCEQLRLILEPTKCTRLKGDYRTGRRINMKKIIPYIASQFRKDKIWLRRTKPAQRDYKITIAIDDSKSMHHNNSKTLTLEAISLVSQALTLLESGRLSIVSFGEAPQIILNHTEQFDGPRLVNALNFAQDKTKIAGLLNFIRTANAEESGTGGDNGLFENLLLILSDGRNIFSEGAQNVKNAIKLARLQRIFLVYIIIDNPDNKNSILDIQHVAVNADGSVNINSYLDSFPFPYYVIVRDLNQLPLVLSEAMRQWFELVNSE

SEQ ID NO: 23 - DNA, 10 bp, Artificial Sequence
  Description of Artificial Sequence SAGE library tag
acatactgcc

SEQ ID NO: 24 - DNA, 10 bp, Artificial Sequence
  Description of Artificial Sequence SAGE library tag
ggcgatctca

SEQ ID NO: 25 - DNA, 10 bp, Artificial Sequence
  Description of Artificial Sequence SAGE library tag
gtaggctgag

SEQ ID NO: 26 - DNA, 10 bp, Artificial Sequence
  Description of Artificial Sequence SAGE library tag
tacctgaagt

SEQ ID NO: 27 - DNA, 10 bp, Artificial Sequence
  Description of Artificial Sequence Oligonucleotide tag not found in some SAGE libraries
gcactgaatg

SEQ ID NO: 28 - DNA, 10 bp, Artificial Sequence
  Description of Artificial Sequence Oligonucleotide tag not found in some SAGE libraries
ggactctgac

SEQ ID NO: 29 - DNA, 10 bp, Artificial Sequence
  Description of Artificial Sequence Oligonucleotide tag not found in some SAGE libraries
gggactctac

SEQ ID NO: 30 - protein, 150 aa, Unknown Organism
  Description of Unknown Organism VWA domain peptide
PLDVVFLLDGSGSMGGNRFELAKEFVLKLVEQLDIGPRGDRVGLVTFSSDARVLFPLNDSQSKDALLEALANLSYSLGGGTNLGAALEYALENLFSESAGSRRGAPKVLILITDGESNDGGEDILKAAKELKRSGVKVFVVGVGNAVDEE

SEQ ID NO: 31 - protein, 180 aa, Unknown Organism
  Description of Unknown Organism VWA domain peptide
PLDVVFLLDGSGSMGGNRFELAKEFVLKLVEQLDIGPRGDRVGLVTFSSDARVLFPLNDSQSKDALLEALANLSYSLGGGTNLGAALEYALENLFSESAGSRRGAPKVLILITDGESNDGGEDILKAAKELKRSGVKVFVVGVGNAVDEEELKKLASAPGGVFAVEDLPELLDLLIDLLL

SEQ ID NO: 32 - protein, 370 aa, Mus musculus
DITPASSLSFVLDTTGSMGEEINAAKIQARRIVEQRQGSPMEPVFYILVPFHDPGFGPVFTTSDPDSFWQKLNEIHALGGGDEPEMCLSALELALLHTPPLSDIFVFTDASPKDALLTNRVESLTRERRCRVTFLVTEDPSRTGGRRRREALSPLRFEPYEAIARASGGEVIFTKDQYIQDVAAIVGESMAGLVTLPLDPPVFTPGEPCVFSVDSLLWQVTVRMHGDISSFWIKSPAGVSQGPEEGIGPLGHTRRFGQFWTVTMTDPPRTGTWEIQVAAAGTPRVRVQAQTSLDFLFHFGISVEDGPHPGLYPLTQPVAGLQTQLLVEVTGLTSRQKLVGGQPQFSHVVLRRVPEGTQLGRVSLEPVGPP

SEQ ID NO: 33 - protein, 182 aa, Homo sapiens
GNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVI

SEQ ID NO: 34 - protein, 5198 aa, Caenorhabditis elegans
MGRSPSWLYGVLGLLLLATTCSSVNDDKNDPTGKSSLAFVFDITGSMFDDLVQVREGAAKIFKTVMAQREKLIYNYIMVPFHDPYLGEIINTTDSTYFMRQLSKVYVHGGGDCPEKTLTGILKALQISLPSSFIYVFTDARSKDYHLEDEVLNTIQEKQSSVVFVMTGDCGNRTHPGFRTYEKIAAASFGQVFHLEKSDVSTVLEYVRHAVKQKKVHLMYEARERGGTVSRNIPVDKHLSELTISLSGDKDDSDNLDIVLRDPEGRTVDKRLYSKEGGTIDLKNVKLIRLKDPSPGVWTVNTNSRLKHTIRVFGHGAVDFKYGFASRPLDRIELARPRPVLNQDTYLLINMTGLIPPGTVGEIDLVDYHGHSLYKAVASPHRTNPNMYFAGPFVPPKGLFFVRVQGYDEDNYEFMRIAPTAIGSVIVGGPRAFMSPIHQEFVGRDLNLSCTVESASAYTIYWVKTGEDIIGGPLFYHNTDTSVWTIPELSLKDAGEYECRVISNNGNYSVKTRVETRESPPEIFGVRNVSVPLGEAAFLHCSTRSAGEVEIRWTRYGATVFNGPNTERNPTNGTLKIHHVTRADAGVYECMARNAGGMSTRKMRLDIMEPPSVKVTPQDVYFNMREGVNLSCEAMGDPKPEVHWYFKGRHLLNDYKYQVGQDSKFLYIRDATHHDEGTYECRAMSQAGQARDTTDLMLATPPKVEIIQNKMMVGRGDRVSFECKTIRGKPHPKIRWFKNGKDLIKPDDYIKINEGQLHIMGAKDEDAGAYSCVGENMAGKDVQVANLSVGRVPTIIESPHTVRVNIERQVTLQCLAVGIPPPEIEWQKGNVLLATLNNPRYTQLADGNLLITDAQIEDQGQFTCIARNTYGQQSQSTTLMVTGLVSPVLGHVPPEEQLIEGQDLTLSCVVVLGTPKPSIVWIKDDKPVEEGPTIKIEGGGSLLRLRGGNPKDEGKYTCIAVSPAGNSTLHINVQLIKKPEFVYKPEGGIVFKPTISGMDEKHVAVVNSTHDVLDGEGFAIPCVVSGTPPPIITWYLDGRPITPNSRDFTVTADNTLIVRKADKSYSGVYTCQATNSAGDNEQKTTIRIMNTPMISPGQSSFNMVVDDLFTIPCDVYGDPKPVITWLLDDKPFTEGVVNEDGSLTIPNVNEAHRGTFTCHAQNAAGNDTRTVTLTVHTTPTINAENQEKIALQNDDIVLECPAKALPPPVRLWTYEGEKIDSQLIPHTIREDGALVLQNVKLENTGVFVCQVSNLAGEDSLSYTLTVHEKPKIISEVPGVVDVVKGFTIEIPCRATGVPEVIRTWNKNGIDLKMDEKKFSVDNLGTLRIYEADKNDIGNYNCVVTNEAGTSQMTTHVDVQEPPIILPSTQTNNTAVVGDRVELKCYVEASPPASVTWFRRGIAIGTDTKGYVVESDGTLVIQSASVEDATIYTCKASNPAGKAEANLQVTVIASPDIKDPDVVTQESIKESHPFSLYCPVFSNPLPQISWYLNDKPLIDDKTSWKTSDDKRKLHVFKAKITDSGVYKCVARNAAGEGSKSFQVEVIVPLNLDESKYKKKVFAKEGEEVTLGCPVSGFPVPQINWVVDGTVVEPGKKYKGATLSNDGLTLHFDSVSVKQEGNYHCVAQSKGNILDIDVELSVLAVPIVGEDDNLEVFLGKDISLSCDLQTESDDKTTFVWSINGSESDRPDNVQIPSDGHRLYITDAKPENNGKYMCRVTNSAGKAERTLTLDVLEPPVFVEPVFEANQKLIGNNPIILQCQVTGNPKPTVIWKIDGNDVDKSWLFDESLSLLRIEKLTGKSAQISCTAENKAGTASRDFFIQNIAAPTFKNEGDQETIFRESETITLDCPVSLGDFQITWMKQGLPLTENDAIFTLDNTRLTILNANRDHEDIYTCVANNTAGQVSKDFDVVVQVLPKIKNAVVTLEINEGEEIILTCDAEGNPTPTAKWDFNQGDLPKEAVFVNNNHTVVVNNVTKYHTGVYKCYATNKVGQAVKTINVHVRTKPRFESGLTESELTVNLTRSITLECDVDDAIGVGISWTVNGKPFLAETDGVQTLAGGRFLHIVSAKTDDHGSYACTVTNEAGVATKTFNLFVQVPPTIVNEGGEYTVIENNSLVLPCEVTGKPNPVVTWTKDGRPVGDLKSVQVLSEGQQFKIVHAEIAHKGSYICMAKNDVGTAEISFDVDIITRPMIQKGIKNIVTAIKGGALPFKCPIDDDKNFKGQIIWLRNYQPIDLEAEDARITRLSNDRRLTILNVTENDEGQYSCRVKNDAGENSFDFKATVLVPPTIIMLDKDKNKTAVEHSTVTLSCPATGKPEPDITWFKDGEAIHIENIADIIPNGELNGNQLKITRIKEGDAGKYTCEADNSAGSVEQDVNVNVITIPKIEKDGIPSDYESQQNERVVISCPVYARPPAKITWLKAGKPLQSDKFVKTSANGQKLYLFKLRETDSSKYTCIATNEAGTDKRDFKVSMLVAPSFDEPNIVRRITVNSGNPSTLHCPAKGSPSPTITWLKDGNAIEPNDRYVFFDAGRQLQISKTEGSDQGRYTCIATNSVGSDDLENTLEVIIPPVIDGERREAVAVIEGFSSELFCDSNSTGVDVEWQKDGLTINQDTLRGDSFIQIPSSGKKMSFLSARKSDSGRYTCIVRNPAGEARKLFDFAVNDPPSISDELSSANIQTIVPYYPVEINCVVSGSPHPKVYWLFDDKPLEPDSAAYELTNNGETLKIVRSQVEHAGTYTCEAQNNVGKARKDFLVRVTAPPHFEKEREEVVARVGDTMLLTCNAESSVPLSSVYWHAHDESVQNGVITSKYAANEKTLNVTNIQLDDEGFYYCTAVNEAGITKKFFKLIVIETPYFLDQQKLYPIILGKRLTLDCSATGTPPPTILFMKDGKRLNESDEVDIIGSTLVIDNPQKEVEGRYTCIAENKAGRSEKDMMVEVLLPPKLSKEWINVEVQAGDPLTLECPIEDTSGVHITWSRQFGKDGQLDMRAQSSSDKSKLYIMQATPEDADSYSCIAVNDAGGAEAVFQVTVNTPPKIFGDSFSTTEIVADTTLEIPCRTEGIPPPEISWFLDGKPILEMPGVTYKQGDLSLRIDNIKPNQEGRYTCVAENKAGRAEQDTYVEISEPPRVVMASEVMRVVEGRQTTIRCEVFGNPEPVVNWLKDGEPYTSDLLQFSTKLSYLHLRETTLADGGTYTCIATNKAGESQTTTDVEVLVPPRIEDEERVLQGKEGNTYMVHCQVTGRPVPYVTWKRNGKEIEQFNPVLHIRNATRADEGKYSCIASNEAGTAVADFLIDVFTKPTFETHETTFNIVEGESAKIECKIDGHPKPTISWLKGGRPFNMDNIILSPRGDTLMILKAQRFDGGLYTCVATNSYGDSEQDFKVNVYTKPYIDETIDQTPKAVAGGEIILKCPVLGNPTPTVTWKRGDDAVPNDSRHTIVNNYDLKINSVTTEDAGQYSCIAVNEAGNLTTHYAAEVIGKPTFVRKGGNLYEVIENDTITMDCGVTSRPLPSISWFRGDKPVYLYDRYSISPDGSHITINKAKLSDGGKYICRASNEAGTSDIDLILKILVPPKIDKSNIIGNPLAIVARTIYLECPISGIPQPDVIWTKNGMDINMTDSRVILAQNNETFGIENVQVTDQGRYTCTATNRGGKASHDFSLDVLSPPEFDIHGTQPTIKREGDTITLTCPIKLAEDIADQVMDVSWTKDSRALDGDLTDNVDISDDGRKLTISQASLENAGLYTCIALNRAGEASLEFKVEILSPPVIDISRNDVQPQVAVNQPTIMRCAVTGHPFPSIKWLKNGKEVTDDENIRIVEQGQVLQILRTDSDHAGKWSCVAENDAGVKELEMVLDVFTPPVVSVKSDNPIKALGETITLFCNASGNPYPQLKWAKGGSLIFDSPDGARISLKGARLDIPHLKKTDVGDYTCQALNAAGTSEASVSVDVLVPPEINRDGIDMSPRLPAQQSLTLQCLAQGKPVPQMRWTLNGTALTHSTPGITVASDSTFIQINNVSLSDKGVYTCYAENVAGSDNLMYNVDVVQAPVISNGGTKQVIEGELAVIECLVEGYPAPQVSWLRNGNRVETGVQGVRYVTDGRMLTIIEARSLDSGIYLCSATNEAGSAQQAYTLEVLVSPKIITSTPGVLTPSSGSKFSLPCAVRGYPDPIISWTLNGNDIKDGENGHTIGADGTLHIEKAEERHLIYECTAKNDAGADTLEFPVQTIVAPKISTSGNRYINGSEGTETVIKCEIESESSEFSWSKNGVPLLPSNNLIFSEDYKLIKILSTRLSDQGEYSCTAANKAGNATQKTNLNVGVAPKIMERPRTQVVHKGDQVTLWCEASGVPQPAITWYKDNELLTNTGVDETATTKKKSVIFSSISPSQAGVYTCKAENWVASTEEDIDLIVMIPPEVVPERMNVSTNPRQTVFLSCNATGIPEPVISWMRDSNIAIQNNEKYQILGTTLAIRNVLPDDDGFYHCIAKSDAGQKIATRKLIVNKPSDRPAPIWVECDEKGKPKKTEYMIDRGDTPDDNPQLLPWKDVEDSSLNGSIAYRCMPGPRSSRTVLLHAAPQFIVKPKNTTAAIGAIVELRCSAAGPPHPTITWAKDGKLIEDSKFEIAYSHLKVTLNSTSDSGEYTCMAQNSVGSSTVSAFINVDNNILPTPKPSSNQKNVAVITCYERNQAYSRGLTWEYNGVPMPKNLAGIHFMNNGSLVILDTSSLKEGDLELYTCKVRNRRRHSIPHLTSAFEGVPEVKTIDKVEVNNGDSVVLDCEVTSDPLTTHVVWTKNDQKMLDDDAIYVLPNNSLVLLNVEKYDEGVYKCVASNSIGKAFDDTQLNVYEGDFLPLTGFEGSGINIDDSSNAGGSSRREAYKKENEDASTTTITTTSPTTTTTETPLTTTIIPALITLPAKQYPTDDYHEGSANDDGFGPTTQDSLFEFNPPLHPEISVVNTDCAGTINENGDCVDKDGKTHNLKILTGENHCPEGFAMNPHTRICEDLDECAFYQPCDFECINYDGGFQCNCPLGYELAEEGCRDVNECESVRCEDGKACFNQLGGYECIDDPCPANYSLVDDRYCEPECENCTSTPIQVHMLAIPSGLPISHIATLTAYDKSGRVLNDTTYAISDTGAPLARGRMTSGPFTIKAVKRGHAQVWTNRVLAAGDHHKVRVRAHSDHATNELHAPKETNFLVLINVGQYPF

SEQ ID NO: 35 - protein, 182 aa, Homo sapiens
GNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVI

SEQ ID NO: 36 - protein, 182 aa, Homo sapiens; identical to SEQ ID NO: 35

SEQ ID NO: 37 - protein, 187 aa, Homo sapiens
DSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFA

SEQ ID NO: 38 - DNA, 10 bp, Artificial Sequence
  Description of Artificial Sequence SAGE library tag
gaggatggac

SEQ ID NO: 39 - DNA, 10 bp, Artificial Sequence
  Description of Artificial Sequence SAGE library tag
ctgcccgtgg

SEQ ID NO: 40 - protein, 180 aa, Unknown Organism
  Description of Unknown Organism VWA domain peptide
DIVFLLDGSGSIGSQNFERVKDFVERVVERLDVGPRDKKEEDAVRVGLVQYSDNVRTEIKFKLNDYQNKDEVLQALQKIRYEDYYGGGGTNTGAALQYVVRNLFTEASGSRIEPVAEEGAPKVLVVLTDGRSQDDPSPTIDIRDVLNELKKEAGVEVFAIGVGNADNNNLEELREIASKP

SEQ ID NO: 41 - protein, 183 aa, Homo sapiens; identical to SEQ ID NO: 20

SEQ ID NO: 42 - protein, 190 aa, Homo sapiens
  MOD_RES (1): Variable amino acid
XSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEG

SEQ ID NO: 43 - protein, 178 aa, Unknown Organism
  Description of Unknown Organism VWA domain peptide
PLDVVFLLDGSGSMGGNRFELAKEFVLKLVEQLDIGPRGDRVGLVTFSSDARVLFPLNDSQSKDALLEALANLSYSLGGGTNLGAALEYALENLFSESAGSRRGAPKVLILITDGESNDGGEDILKAAKELKRSGVKVFVVGVGNAVDEEELKKLASAPGGVFAVEDLPELLDLLIDL

SEQ ID NO: 44 - protein, 9 aa, Homo sapiens
NLLQLLLEF

SEQ ID NO: 45 - protein, 44 aa, Mycobacterium tuberculosis
RNNEDGTLQEIKKLLDEAVLAERKELARALDDDARFAELQLDAL

SEQ ID NO: 46 - protein, 20 aa, Mycobacterium tuberculosis
TQALADIAELEQLAEQSQSY

SEQ ID NO: 47 - protein, 403 aa, Deinococcus radiodurans
MARITRYSKFEGELDQLESSELMQMIQEALLGQGMNDPWDPDPNARPSMDDLFDAILEALAERNMIPEEQLLEALQADDVRETALGQQIERLMDKLQQDGFIRKEFGDEDGQGGAGNPGEATFQLTDKSIDFLGYKSLRDLMGGLGRSSAGAHDTREYASGVEMTGELKNYEFGDTLNLDTTATLGNVISKGFDQLEEADLVIRQAEYSSSAATVVMLDCSHSMILYGEDRFTPAKQVALALAHLIRTQYPGDTVKFVLFHDSAEEVPVSKLAQAQIGPYHTNTAGGLRLAQQLLKRENKDMKQIVMITDGKPSALTLPDGRIYKNPYGLDPYVLGTTLREVANCRRSGIQINTFMLARDPELVGFVQRVSEMTKGKAYFTTPQNIGQYVLQDFMTNKTKVMN

SEQ ID NO: 48 - protein, 190 aa, Homo sapiens; identical to SEQ ID NO: 42
  MOD_RES (1): Variable amino acid

SEQ ID NO: 49 - protein, 19 aa, Mycobacterium tuberculosis
MATPALLPGVDLAAFFAAA

SEQ ID NO: 50 - protein, 10 aa, Mycobacterium tuberculosis
LAAEAAALAA

SEQ ID NO: 51 - protein, 9 aa, Mycobacterium tuberculosis
RIRPRRPRR

SEQ ID NO: 52 - protein, 17 aa, Mycobacterium tuberculosis
PGVDLAAEAAALAARLR

SEQ ID NO: 53 - protein, 17 aa, Mycobacterium tuberculosis
ATFDAVFASEFGYEGSA

SEQ ID NO: 54 - protein, 170 aa, Bradyrhizobium japonicum
RHRPDSRGLRLDLRRTLRASLRTGGDIIDIHRLGRIEKPAPIVALLDISGSMNEYTRLFLHFLHAIGDARKRVSVFLFGTRLTNVTRALRQRDPDEALASCSASVEDWAGGTRISASLHNFNKLWARRVLSQGAIVLLISDGLEREADSRLAFEMDRLHRSCRRLIWLNP

SEQ ID NO: 55 - protein, 323 aa, Aeropyrum pernix
MGMEDLERLKQALRAALAKDPGRHRLFDYLFDVYWRGAWTAVQVPGRRIAVRVEVEPGSPVERFLSIYSPVEASWGEPGVEPPSAAQARALYRSLALLRRRLGLEEGRRRVPRRRGRIDFKRSMRRSLRTFGETVRLVRASRKRSKTDILVLLDVSNSMKDYWPWILGILNALRRLPPGSYEAFLFSTRLVRATPMVEAAGGAEDLRRLLSRVEGLWGSGTRISQSVEELLESHTPALHRGAAVLVFSDGWDLGDLGRLASALAELRRRAGFLVWVTPESPRRDAGGEPSTMRIIRRHADLTIDLETLLNTRRLLRLLPRSSA

SEQ ID NO: 56 - protein, 188 aa, Homo sapiens
MASKGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEG

SEQ ID NO: 57 - protein, 192 aa, Homo sapiens
STQLDIVIVLDGSNSIYPWDSVTAFLNDLLERMDIGPKQTQVGIVQYGENVTHEFNLNKYSSTEEVLVAAKKIVQRGGRQTMTALGTDTARKEAFTEARGARRGVKKVMVIVTDGESHDNHRLKKVIQDCEDENIQRFSIAILGSYNRGNLSTEKFVEEIKSIASEPTEKHFFNVSDEIALVTIVKTLGERI